(19) United States
(12) Patent Application Publication
(10) Pub. No.: US 2026/0257986 A1
(43) Pub. Date: Sep. 3, 2026

(54) AMINO ACID-BASED CATIONIC LIPID

(71) Applicant: XIAMEN SINOPEG BIOTECH CO., LTD., Xiamen, Fujian (CN)

(72) Inventors: Sheng LIN, Xiamen (CN); Wengui WENG, Xiamen (CN); Chao LIU, Xiamen (CN); Ailan WANG, Xiamen (CN); Linlin WANG, Xiamen (CN); Minggui LIN, Xiamen (CN); Qi ZHU, Xiamen (CN); Guohua WEI, Xiamen (CN)

(73) Assignee: XIAMEN SINOPEG BIOTECH CO., LTD., Xiamen, Fujian (CN)

(21) Appl. No.: 18/871,043

(22) PCT Filed: Jun. 2, 2023
(86) PCT No.: PCT/CN2023/098155

§ 371 Date: Dec. 2, 2024

(30) Foreign Application Priority Data
Jun. 2, 2022 (CN) 202210628341.8

(51) Int. Cl. C07C 229/24; A61K 31/7088; A61K 47/18; A61K 47/22; A61K 47/54; C07D 209/20; C07D 211/26; C07D 233/64; C07D 295/088

(52) U.S. Cl. CPC C07C 229/24; A61K 31/7088; A61K 47/183; A61K 47/22; A61K 47/543; A61K 47/545; C07D 209/20; C07D 211/26; C07D 233/64; C07D 295/088

(57) ABSTRACT

The present invention introduces a novel amino acid-based cationic lipid, represented by the general formula (1), with symbols as defined herein. This lipid is pharmaceutically acceptable, biodegradable, or highly biocompatible, and offers low toxicity, low immunogenicity, and high compatibility. The amino acid or its derivatives used as starting materials are easily accessible, either naturally or through simple synthesis, making the process straightforward, safe, and cost-effective. The lipid structure may include degradable groups between the amino acid residue and lipophilic tail chains. The presence of degradable groups allows lipid nanoparticles (LNPs) to degrade within endosomes, addressing issues of LNP accumulation and endosomal acidification caused by non-degradable lipids in prior art. This facilitates the effective endosomal escape of drug molecules, such as nucleic acids, ensuring their proper cellular function after delivery.

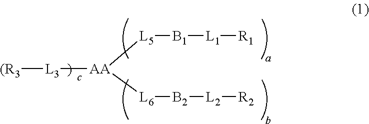

### TECHNICAL FIELD

The present application belongs to the field of drug delivery and specifically relates to a cationic lipid as pharmaceutical carrier, in particular to an amino acid-based cationic lipid, as well as a lipid composition containing the amino acid-based cationic lipid, a lipid pharmaceutical composition, and formulations and applications thereof.

### BACKGROUND

Liposome is the closed vesicle with a bilayer structure characterized by low immunogenicity and high biocompatibility. Liposome can deliver drugs or active molecules into cells and is widely used for delivering nucleic acid drugs, gene vaccines, anti-tumor drugs, small molecule drugs, peptide drugs, or protein drugs. The use of liposomes to deliver drugs or small molecules that are poorly stable and rapidly degraded can achieve the effects of improving efficacy, reducing toxicity, enhancing stability, and improving targeting.

Lipid nanoparticle (LNP) contains lipids and drug molecules or active molecules, and is widely used for small molecules and nucleic acid drugs delivery. LNP has recently gained attention for their great success as a platform for COVID-19 mRNA vaccine delivery. LNP is usually composed of cationic lipids, neutral lipids, steroid lipids and PEGylated lipids.

Cationic lipids and drug molecules (e.g., negatively charged nucleic acids) are electrostatically bound together, while phospholipid lipids play a role in preventing lipid oxidation or connecting ligands to the surface of LNP. Steroid lipids have strong membrane fusion properties, facilitating intracellular uptake and cytoplasmic entry of drug molecules.

PEGylated lipids are located on the surface of LNP, improving their hydrophilicity, avoiding rapid clearance by the immune system, preventing particle aggregation, and increasing stability.

For example, nucleic acid molecules are very fragile and susceptible to be degraded and ineffective during storage and transportation due to factors such as nucleases and temperature.

Ionizable cationic lipids exhibit neutrality at physiological pH, while in acidic environments, they are positively charged and combined with negatively charged nucleic acids to form LNP that enters the endosome together with other lipids. After that, under acidic conditions in the endosome, cationic lipids can ionize and carry partial positive charges to promote the escape of nucleic acid endosomes and release them into the cytoplasm to exert therapeutic effects, improving the transfection rate of nucleic acid drugs.

Although cationic lipids have made the latest progress in drug delivery, there is still a need for alternative cationic lipids that are suitable for regular therapeutic uses in this field.

Reference WO2021026358A1 also reported that nitrogen-containing lipids can be protonated under physiological pH conditions to carry positive or partially positive charges. In existing technologies, the cationic lipid DOTAP can be used for mRNA transfection in various cell types, and although it is effective in vitro, the permanent cationic quaternary ammonium group quickly removes the liposome from the systemic circulation and target organs, and exhibits toxicity. CN104168887 Å discloses amino acid ether lipids, which contain ether bonds that are relatively stable in vivo. Cationic lipids containing only ether bonds inhibit the degradation of LNP, and the accumulation of LNP prevents drug molecules (e.g., nucleic acid molecules) from being released from the endosomal lumen into the cytoplasm. Therefore, in the present application, we use the chemical diversity of amino acids to design and synthesize a series of stable or degradable amino acid-based cationic lipids with amino acid residues as the core structure and containing one or more lipophilic tails.

### SUMMARY

The present application provides novel amino acid-based cationic lipids and preparation methods thereof, comprising a lipid composition containing the amino acid-based cationic lipid, a lipid pharmaceutical composition containing the lipid composition and the preparation thereof, a liposome or LNP containing the lipid composition. Especially the LNP-nucleic acid pharmaceutical composition containing the lipid composition and its preparations have the advantages of high delivery efficiency, safety, low toxicity, and high biocompatibility, which can improve the therapeutic and/or preventive effects of the drugs.

The above-described purposes of this application can be realized via embodiments below.

In one embodiment, provided herein is an amino acid-based cationic lipid:

An amino acid-based cationic lipid; wherein, the structure is represented by the general formula (1).

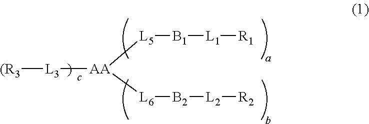

wherein, AA is a residue of an amino acid or an amino acid derivative;
B₁ and B₂ are each independently a linking bond or a C₁₋₃₀ alkylene group at each occurrence;
L₁ and L₂ are each independently a linking bond or a divalent linking group at each occurrence;
L₅ and L₆ are each independently a linking bond or a divalent linking group at each occurrence;
L₃ is independently a linking bond or a divalent linking group at each occurrence;
R₁ and R₂ are each independently —(CH₂)ₜNRₑR_{f}, a linear C₁₋₃₀ alkylene group, a branched C₁₋₃₀ alkylene group, or

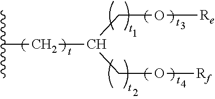

at each occurrence; wherein, t is an integer from 0 to 12; t₁ and t₂ are each independently an integer from 0 to 5; t₃ and t₄ are each independently 0 or 1; t₁, t₂, t₃ and t₄ are not 0 simultaneously; Rₑ and R_{f} are each independently a C₁₋₁₅ alkyl group, a C₂₋₁₅ alkenyl group, or a C₂₋₁₅ alkynyl group;
R₃ is independently a hydrogen atom, —R_{d}, —OR_{d}, a C₃₋₆ carbocyclic group, a nitrogen-containing heterocyclic group, —NR_{d}R_{d}, —SR_{d}, —C(═O)R_{d}, —C(═O)OR_{d}, —OC(═O)R_{d}, —OC(═O)OR_{d} or a functional group R01 that can interact with bio-related substances at each occurrence; wherein, R_{d} is independently a C₁₋₁₂ alkyl group at each occurrence;
a, b, and c are each independently 1 or 2; when fragments -L₅-ϵ₁-L₁-R₁ and/or -L₆-B₂-L₂-R₂ and/or -L₃-R₃ are protruded from the amino terminus of the amino acid, a, b, and c are each independently 1 or 2; when fragments -L₅-B₁-L₁-R₁ and/or -L₆-B₂-L₂-R₂ and/or -L₃-R₃ are protruded from the carboxyl, hydroxyl, or thiol terminus of an amino acid, a, b, and c are each independently 1; when a is 2, two -L₅-B₁-L₁-R₁ fragments are the same or different; when b is 2, two -L₆-B₂-L₂-R₂ fragments are the same or different; when c is 2, two -L₃-R₃ fragments are the same or different; Or a salt, tautomer, stereoisomer, or solvate thereof.

The present application also provides a lipid composition, embodied as follows:

A lipid composition containing an amino acid-based cationic lipid with the structure represented by general formula (1).

The present application also provides a lipid pharmaceutical composition, embodied as follows:

Provided herein is a lipid pharmaceutical composition containing lipid compositions and drugs; wherein, the lipid composition contains amino acid-based cationic lipids with the structure represented by formula (1), and the drug is selected from the group consisting of a nucleic acid drug, a gene vaccine, an antitumor drug, a small molecule drug, a polypeptide drug, or a protein drug.

The present application also provides a formulation of lipid pharmaceutical composition, embodied as follows:

Provided herein is a formulation of lipid pharmaceutical composition containing the aforementioned lipid pharmaceutical composition and pharmaceutically acceptable diluents or excipients.

The present application also provides a liposome or LNP, embodied as follows:

Provided herein is a liposome or LNP containing a lipid composition, the lipid composition contains an amino acid-based cationic lipid with the structure represented by formula (1).

Compared with the prior art, the present application brings the following beneficial effects:

The novel amino acid-based cationic lipid of the present application is a molecule having a hydrophilic moiety and a lipophilic moiety. The hydrophilic moiety can be provided by amino acid residues, and the lipophilic moiety can contain one or more lipophilic tails. Amino acids or amino acid derivatives used in the preparation process are simple and easy to obtain, and can be obtained naturally or by simple synthesis, which has the advantages of simplicity, safety, and cost-saving production.

The tertiary amine moiety of the novel amino acid-based cationic lipid of the present application is derived from amino acids or amino acid residues, and compared with the quaternary amine that will cause toxicity, the tertiary amine can ionize a portion of the positive charge and bind to the drug (e.g., negatively charged nucleic acid molecules), thereby increasing the intracellular transport rate of the drug.

The novel amino acid-based cationic lipid of the present application is a pharmaceutically acceptable, biodegradable or biocompatible lipid with the advantages of low toxicity, low immunogenicity and high biocompatibility.

The novel amino acid-based cationic lipid of the present application can contain degradable groups between the amino acid residue and the lipophilic tail chain, and the presence of the degradable group enable the lipid composition prepared therefrom to be degraded in vivo in due course, which solves the problem that lipid compositions prepared from the non-degradable lipids in the prior art will store and acidify the endosomal environment in the endosome and hinders the endosome escape of drug molecules (such as nucleic acids), and solves the problem that drugs delivered into the cell cannot produce an effect.

The novel amino acid-based cationic lipid of the present application can contain a targeting group, and the lipid compositions prepared from the cationic lipids can also have targeting function, further enhance the therapeutic and/or diagnostic effect of the drug.

The terminus of the novel amino acid-based cationic lipids in the present application can be chemically diverse by substitutions at the N-terminus, C-terminus, hydroxyl-terminus or thiol-terminus of the amino acid or amino acid derivative, the amino acid can also be a polypeptide formed from 2 to 20 same or different amino acid residues.

The amino acid-based cationic lipids provided by the present application have stimulus responsiveness and can mediate biorecognition events. Lipid compositions and lipid pharmaceutical compositions prepared from amino acid-based cationic lipids have a certain morphology in the normal organism environment. After entering the body to reach the lesion site, due to the change of the biological microenvironment of the lesion site, the assembled morphology and bond mode of the lipid composition or lipid pharmaceutical composition will change, and the loaded drug molecules can be released to exert therapeutic or preventive effects.

The amino acid-based cationic lipid of the present application may also be coupled with targeting groups, and lipid compositions and lipid pharmaceutical compositions prepared thereof can better achieve targeted delivery and controlled release of the drug.

### DETAILED DESCRIPTION OF THE APPLICATION

### Description of Terms

In the present application, unless otherwise described, all technical and scientific terms used herein have the same meaning as those generally understood by those of ordinary skill in the art. The disclosures of all patents and other publications referenced herein are incorporated in their entirety by reference. In the event of a conflict between any description of the terms herein and any document incorporated by reference, the description and interpretation of the terms described below shall prevail. Unless otherwise indicated, the terms have the following meanings.

In the present application and unless otherwise specified, a structure with isomers may refer to any form of the isomers. For example, when cis- and trans-isomers are present, it can refer to either a cis-structure or a trans-structure; when E/Z isomers are present, it can refer to either an (E)-structure or a (Z)-structure; and when optical activity are present, it can refer to either a laevoisomer or a dextroisomer.

In the present application, the numerical interval includes both the numerical interval marked by the dash line (e.g., 0-12), and the numerical interval marked by the wavy line such as (0-12), and the “to” marked numerical interval (e.g., 0 to 12, 1 to 12). In the present application and unless otherwise specified, an integer interval represents the group of all integers within the range of the interval, and the range includes two endpoints as well. For example, the integer interval 0-12 represents the group composed of 0, 1, 2, 3, 4, 5, 6, 7, 8, 9, 10, 11 and 12. The numerical interval in the present application includes but is not limited to the numerical intervals represented by integers, non-integers, percentages and fractions, and all of the foregoing numerical intervals include two endpoints unless otherwise specified.

In the present application, “about” or “approximately” followed by a numerical value generally suggests a ±10% numerical range; in some cases, the numerical range can be enlarged to ±15%, but no more than ±20%. For example, when the molar percentage of steroid lipids among the total lipids in a solution containing the solvent is about 40%, it can be considered that, generally, the molar percentage of steroid lipids is 30%-50%.

In the present application, unless otherwise specified, “any” includes any one, any two, and any two or more.

The terms “include”, “contain”, and similar expressions in the present application shall be interpreted, unless otherwise specified, as “including but not limited to”, “include but are not limited to”, or “includes but is not limited to”, etc., in the description and claims with openness and inclusiveness.

In the present application, when two or more objects are “each independently preferably” selected from multiple levels of preferable options, the objects are not necessarily selected from preferable options of the same level. It is allowed that one is selected from a wider range of options while another one is selected from a narrower range of options. It is also allowed that one is selected from the maximum range of options while another is selected from any allowable options. It is also allowed that the objects are selected from preferable options of the same level.

In the present application, “each independently at each occurrence” not only means that different groups can be each independently selected from the definitions, but also means that the same group at different positions can be independently selected from the definitions; the object including but not limited to the groups mentioned above. For example, “are each independently selected from the group consisting of a linking bond, —OC(═O)—, —C(═O)O—, —OC(═O)O—, —C(═O)—, —O—, —NH—, —O(CR_{c}R_{c})ₛO—, —S—, —C(═O)S—, —SC(═O)—, —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, and —NR_{c}C(═O)S—; wherein R_{c} is independently, at each occurrence, a hydrogen atom or a C₁₋₁₂ alkyl group”, two R_{c} in “—NR_{c}C(═O)NR_{c}-” can be the same or different, which are each independently a hydrogen atom or a C₁₋₁₂ alkyl group.

In the present application and unless otherwise specified, divalent linking group e.g., a hydrocarbylene group, an alkylene group, an arylene group, an amide bond, and the like, either one of the two linking ends could be chosen to be linked to another group. For example, when an amide bond serves as a divalent linking group between GroupA and GroupB, both GroupA-C(═O)NH-GroupB and GroupB—NHC(═O)-GroupA are allowable.

In the present application, when distinguishing the terminus from the substituents of a linking group becomes questionable, “ [symbol] ” is used to indicate the connection location between the linking group and the other group. For example, in structural formulas

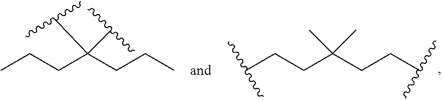

[symbol]

are used to indicate the connection locations between the divalent linking groups and the other groups; the two structural formulas mentioned above represent —CH(CH₂CH₂CH₃)₂- and —CH₂CH₂CH(CH₃)₂-CH₂CH₂—, respectively.

In the present application, a number or a numerical interval written in the subscript of “C” can be used to indicate the number of carbon atoms of a group. For example, a C₁₋₁₂ group is a group having 1 to 12 carbon atoms; C₁₋₃₀ indicates “having 1 to 30 carbon atoms”. “substituted C₁₋₁₂ alkyl group” refers to a C₁₋₁₂ alkyl group with one or more hydrogen atoms being substituted. “C₁₋₁₂ substituted alkyl group” means an alkyl group with one or more hydrogen atoms being substituted has 1 to 12 carbon atoms remaining. As another example, when a group can be selected from C₁₋₁₂ alkylene groups, it can be any alkylene group with the number of carbon atoms in the range indicated by the subscript, that is, the group can be selected from the group consisting of C₁, C₂, C₃, C₄, C₅, C₆, C₇, C₈, C₉, C₁₀, C₁₁, and C₁₂ alkylene groups. In the present application and unless otherwise specified, a subscript being a numerical interval indicates that the subscript can be any integer within the interval which includes two endpoints.

In the present application, heteroatoms are not particularly limited, including but not limited to O, S, N, P, Si, F, Cl, Br, I, B, etc.

In the present application, the heteroatom used for substitution is referred to as “substituent atom”, and the group used for substitution is referred to as “substituent group”.

In the present application, a “substituted” group indicates that at least one hydrogen atom of the aforementioned group (e.g., aliphatic hydrocarbon groups, hydrocarbon groups, alkyl groups, or alkylene groups) is replaced by a bond connected to a non-hydrogen atom, the non-hydrogen atom including but not limited to a halogen atom (F, Cl, Br, or I), an oxo group (═O), a hydroxyl group (—OH), a hydrocarbyloxy group (—OR_{d}, wherein R_{d} is a C₁₋₁₂ alkyl group), a carboxyl group (—COOH), an amine group (—NR_{c}R_{c}, wherein both R_{c} are each independently a hydrogen atom or a C₁₋₁₂ alkyl group), a C₁₋₁₂ alkyl group, and a cycloalkyl group. In some embodiments, the substituent group is a C₁₋₁₂ alkyl group. In another embodiment, the substituent is a cycloalkyl group. In another embodiment, the substituent is a halide group, e.g., fluoride. In another embodiment, the substituent is an oxo group. In another embodiment, the substituent is a hydroxyl group. In another embodiment, the substituent is an alkoxy group. In another embodiment, the substituent is a carboxyl group. In another embodiment, the substituent is an amine group.

In the present application, “optional” or “optionally” (e.g., optionally substituted) means that the event of the situation described thereafter may or may not occur, and the description includes instances in which the event or situation occurs as well as instances in which the event or situation does not occur. For example, “optionally substituted hydrocarbyl” means that the hydrocarbyl group may or may not be substituted, and the description includes both substituted and unsubstituted hydrocarbyl groups.

In the present application, “carbon chain linker” or “carbon chain linking group” refers to the linking group whose main-chain atoms are all carbon atoms, allowing heteroatoms or groups containing heteroatoms in the side chains which substitute for hydrogen atoms connected to main-chain carbon atoms. When a “main-chain atom” is a heteroatom, it can also be called “main-chain heteroatom”. For example, A-S—CH₂—B, AO—CH₂—B, and

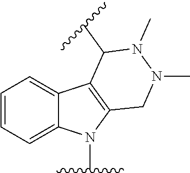 [SMILES: *C1c2c(n(*)c3ccccc23)CN(C)N1C]

(wherein the atomic spacing is 4) are considered to contain main-chain heteroatoms. Carbon chain linking groups can be divided into hydrocarbylene groups and those whose pendant groups contain heteroatoms; the carbon chain linking groups whose pendant groups contain heteroatoms include but are not limited to those substituted by an oxo group (═O), those substituted by a thioxo group (=S), those substituted by an imino group (connected to the main chain carbon through a carbon-nitrogen double bond), an oxa-hydrocarbon group with an ether bond, a thia-hydrocarbon group with a thioether bond, and an aza-hydrocarbon group with a tertiary amine group, etc. The main chain of the “carbon chain linking group” is entirely composed of carbon atoms, and the pendant groups of the carbon chain are allowed to contain heteroatoms, that is, the main chain is constructed by connecting methylene groups or substituted methylene groups. The substituted methylene groups can be substituted by one monovalent substituent, two monovalent substituents, or one divalent substituent (e.g., a divalent oxygen atom, or one that forms a three-membered ring

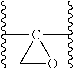 [SMILES: *C1(*)CO1]

with a divalent methylene group). The substituted methylene group can have one hydrogen atom being substituted (e.g., —CH(CH₃)—), two hydrogen atoms being respectively substituted (e.g., —(CH₃)C(OCH₃)—), or two hydrogen atoms being simultaneously substituted (e.g., a carbonyl group, a thiocarbonyl group, —C(═NH)—, —C(=N*H₂)-, or a cyclic pendant group (e.g.,

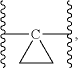 [SMILES: *C1(*)CC1]

wherein the atomic spacing is 1)).

In the present application, a compound or a group can be substituted and heterosubstituted at the same time, for example, a hydrogen atom can be replaced by a nitrophenyl group, and —CH₂—CH₂—CH₂— can be replaced by —CH₂—S—CH(CH₃)—.

In the present application, a “linking bond” without any atom is only for connection, that is, when a group is denoted as a linking bond, the group can be absent.

In the present application, “group” contains at least one atom, referring to the radical formed by a compound losing one or more atoms. With respect to a compound, the remaining group formed by removal of other groups is also denoted as “residue”. The valence of groups is not particularly limited, and examples include a monovalent group, a divalent group, a trivalent group, a tetravalent group, . . . , a hectovalent group, etc. Wherein, groups with valence being equal to or greater than two are collectively defined as linking groups. A linking group can also contain only one atom, such as an oxo group and a thio group.

In the present application, “hydrocarbon” refers to a class of compounds that contain only carbon atoms and hydrogen atoms.

In the present application, hydrocarbons are classified into aliphatic hydrocarbons and aromatic hydrocarbons in terms of the type of hydrocarbon groups. Hydrocarbons containing neither phenyl rings nor hydrocarbyl-substituted phenyl rings are defined as aliphatic hydrocarbons. Hydrocarbons containing at least one phenyl ring or hydrocarbyl-substituted phenyl ring are defined as aromatic hydrocarbons. An aromatic hydrocarbon can contain aliphatic hydrocarbyl groups, such as toluene, diphenylmethane, 2,3-dihydroindene, etc.

In the present application, hydrocarbons are classified into saturated hydrocarbons and unsaturated hydrocarbons in terms of the degree of saturation. All aromatic hydrocarbons are unsaturated hydrocarbons. Saturated aliphatic hydrocarbons are also termed alkanes. The degree of saturation of unsaturated aliphatic hydrocarbons is not particularly limited. For example, unsaturated aliphatic hydrocarbons include but are not limited to alkenes (containing carbon-carbon double bonds), alkynes (containing carbon-carbon triple bonds), dienes (containing two conjugated carbon-carbon double bonds), and the like. When the aliphatic moieties of aromatic hydrocarbons are saturated, the aromatic hydrocarbons are also termed aralkanes, such as toluene.

In the present application, the structures of hydrocarbons are not particularly limited, including linear structures without pendant groups, branched structures with pendant groups, ring-containing structures, dendritic structures, comb-like structures, hyperbranched structures, etc. Unless otherwise specified, preferable structures include linear structures without pendant groups, branched structures with pendant groups, and ring-containing structures, corresponding to linear hydrocarbons, branched hydrocarbons and cyclic hydrocarbons, respectively. Wherein, hydrocarbons that contain no rings are termed open-chain hydrocarbons, including but not limited to linear structures without pendant groups, and branched structures with pendant groups. Open-chain hydrocarbons belong to aliphatic hydrocarbons. Therefore, linear hydrocarbons are also referred to as linear aliphatic hydrocarbons, and branched hydrocarbons are also referred to as branched aliphatic hydrocarbons.

In the present application, hydrocarbons with any carbon atom replaced by heteroatom are generally referred to as heterohydrocarbons.

In the present application, aliphatic-derived heterohydrocarbon refers to the heterohydrocarbon that derived from an aliphatic hydrocarbon, including aliphatic heterocyclic hydrocarbons and aliphatic open-chain heterohydrocarbons. Saturated aliphatic heterohydrocarbons are also termed heteroalkanes.

In the present application, “hydrocarbon group” refers to the residue of a hydrocarbon molecule with at least one hydrogen atom being removed. According to the number of removed hydrogen atoms, hydrocarbon groups can be classified into monovalent hydrocarbon groups (with one hydrogen atom being removed), divalent hydrocarbon groups (with two hydrogen atoms being removed), trivalent hydrocarbon groups (with three hydrogen atoms being removed), and the like. Accordingly, when n hydrogen atoms are lost, the valence of the formed hydrocarbon group is n compared with the original molecule. Unless otherwise specified, hydrocarbon groups in the present application refer to monovalent hydrocarbon groups. Unless otherwise expressly stated in this specification, the hydrocarbon group is optionally substituted.

In the present application, the source of hydrocarbon group is not particularly limited; for example, hydrocarbon group can be derived from aliphatic hydrocarbons or aromatic hydrocarbons, from saturated hydrocarbons or unsaturated hydrocarbons, from linear hydrocarbons, branched hydrocarbons or cyclic hydrocarbons, or from hydrocarbons or heterohydrocarbons, etc. According to the degree of saturation, hydrocarbon groups can be derived from alkanes, alkenes, alkynes, dienes, etc. With respect to cyclic hydrocarbons, hydrocarbon groups can be derived from alicyclic hydrocarbons or aromatic hydrocarbons, or from monocyclic hydrocarbons or polycyclic hydrocarbons. With respect to heterocyclic hydrocarbons, hydrocarbon groups can be derived from aliphatic heterocyclic hydrocarbons or aromatic heterocyclic hydrocarbons.

In the present application, “aliphatic hydrocarbon group” refers to the residue of an aliphatic hydrocarbon molecule with at least one hydrogen atom being removed. Unless otherwise specified, aliphatic hydrocarbon groups refer to monovalent hydrocarbon groups in the present application. Aliphatic hydrocarbon group includes saturated aliphatic hydrocarbon groups and unsaturated aliphatic hydrocarbon groups. Unless otherwise expressly stated in this specification, aliphatic hydrocarbon groups are optionally substituted.

In the present application, “alkyl group” refers to a hydrocarbon group obtained from an alkane losing a hydrogen atom at any location, unless otherwise specified, the alkyl group can be linear or branched, substituted or unsubstituted. Specific examples include that a propyl group refers to either a 1-propyl group or an isopropyl group, and a propylene group can refer to a 1,3-propylene group, a 1,2-propylene group, or an isopropylidene group. Unless otherwise expressly stated in this specification, alkyl groups are optionally substituted.

In the present application, “unsaturated hydrocarbon group” refers to the hydrocarbon group obtained from an unsaturated hydrocarbon losing hydrogen atoms. The hydrocarbon groups obtained from unsaturated hydrocarbon losing hydrogen atoms bonded to unsaturated carbon atoms, can include alkenyl groups, alkynyl groups, dienyl groups, and the like, e.g., propenyl group and propynyl group. According to the type of unsaturated bond, the hydrocarbon groups formed by removing hydrogen atoms bonded to saturated carbon atoms of unsaturated hydrocarbons, include alkenyl hydrocarbon groups, alkynyl hydrocarbon groups, dienyl hydrocarbon groups, and the like, and specifically, e.g., allyl groups and propargyl groups.

In the present application, the “alkenyl” or “alkenyl group” refers to a substituted or unsubstituted alkenyl group with linear structure or branched structure, containing two or more carbon atoms (e.g., 2, 3, 4, 5, 6, 7, 8, 9, 10, 11, 12,13, 14, 15, 16, 17, 18, 19, 20 or more carbon atoms) and at least one carbon-carbon double bond. “C₂₋₁₅ alkenyl group” refers to a substituted or unsubstituted alkenyl group with linear structure or branched structure, containing 2 to 15 carbon atoms and at least one carbon-carbon double bond, that is, an alkenyl group can contain one, two, three, four, or more carbon-carbon double bonds. Unless otherwise specified, alkenyl groups include substituted and unsubstituted alkenyl groups in the present application. Unless otherwise expressly stated in this specification, alkenyl groups are optionally substituted.

In the present application, the “alkynyl” or “alkynyl group” refers to an optionally substituted hydrocarbon with linear structure or branched structure, containing two or more carbon atoms (such as 2, 3, 4, 5, 6, 7, 8, 9, 10, 11, 12,13, 14, 15, 16, 17, 18, 19, 20, or more carbon atoms) and at least one carbon-carbon triple bond. “C₂₋₁₅ alkynyl group” refers to a substituted or unsubstituted alkynyl group with linear structure or branched structure, containing 2 to 15 carbon atoms and at least one carbon-carbon triple bond, that is, an alkynyl group can contain one, two, three, four, or more carbon-carbon triple bonds. Unless otherwise specified, alkynyl groups include substituted and unsubstituted alkynyl groups in the present application. Unless otherwise expressly stated in this specification, alkynyl groups are optionally substituted.

In the present application, “hydrocarbylene group” or “hydrocarbylene chain” refers to a linear or branched divalent hydrocarbon chain that connects the remainder of a molecule to a free radical, consisting only carbon and hydrogen, being saturated or unsaturated. For example, a hydrocarbylene group with one to twenty-four carbon atoms (C₁₋₂₄ hydrocarbylene group), a hydrocarbylene group with one to twelve carbon atoms (C₁₋₁₂ hydrocarbylene group), specifically, a methylene group, an ethylene group, a propylene group, a n-butylene group, a vinylene group, a propenylene group, a n-butenylene group, a propynylene group, a n-butynylene group, etc. Unless otherwise explicitly stated in this instruction manual, hydrocarbylene groups are optionally substituted.

In the present application, “alkylene group” is also a divalent alkyl group, including an open-chain alkylene group and a divalent cycloalkyl group, an open-chain alkylene group refers to a divalent alkyl group without a cyclic structure, and a divalent cycloalkyl group refers to a divalent alkyl group containing a cyclic structure. Unless otherwise expressly stated in this specification, alkylene groups are optionally substituted.

In the present application, “molecular weight” represents the mass of a compound and “average molecular weight” represents the mass of a compound component of a general formula in macroscopic matter, and unless otherwise specified, the “average molecular weight” also refers to the “number average molecular weight” (Mₙ). The number average molecular weight can be used to describe the molecular weight of polydisperse blocks or substances, or that of monodisperse blocks or substances. The measuring unit of “molecular weight” and “average molecular weight” is Dalton (Da), unless otherwise specified. The molecular weight of polyethylene glycol chain can also be measured in “degree of polymerization” which is, specifically, the number of repeat units (oxyethylene units, i.e., EO units) in the compound molecule. Accordingly, “average degree of polymerization”, “number-average degree of polymerization” or “number of EO units” represents the average value or the number average value of the number of repeating units.

In the present application, the term “about” and “approximately” before a percentage refer to a range of ±0.5%.

In the present application, the terms “stable” (or “can remain stable”) and “degradable” (or “can be degraded”) are a pair of opposing concepts. For detailed examples of stable groups and degradable groups are given in paragraphs [0134]-[0145]in CN113402405A.

In the present application, “hydroxyl protecting group” includes all the groups that can be used as common hydroxyl protecting groups. Hydroxy protecting group is preferably selected from the group consisting of an alkanoyl group (e.g., an acetyl group, a butyryl group), an aromatic alkanoyl group (e.g., a benzoyl group), a benzyl group, a triphenylmethyl group, a trimethylsilyl group, a t-butyldimethylsilyl group, an allyl group, an acetal group, and a ketal group. Removal of acetyl groups is generally carried out under basic conditions, most commonly by the ammonolysis with NH₃/MeOH or by the methanolysis catalyzed by methanol anions. The benzyl group can be easily removed via palladium-catalyzed hydrogenolysis in neutral solution at room temperature, or via reduction reaction by metallic sodium in ethanol or liquid ammonia. The triphenylmethyl group is generally removed via catalytic hydrogenolysis. The trimethylsilyl groups are generally removed using reagents containing fluoride ions (e.g., tetrabutylammonium fluoride/anhydrous THF, etc.). The t-butyldimethylsilyl ether is relatively stable, which can withstand ester hydrolysis conditions with alcoholic potassium hydroxide and mild reduction conditions (e.g., Zn/CH₃OH and the like), so that it can be removed by fluoride ions (e.g., Bu₄N*F—) in THE solution or by aqueous acetic acid at room temperature.

In the present application, “carboxyl protecting group” refers to the protecting group which can be transformed into a carboxyl group via the hydrolysis or the deprotection reaction of the carboxyl protecting group itself. Carboxyl protecting group is preferably selected from the group consisting of an alkyl group (e.g., a methyl group, an ethyl group, and a butyl group) and an aralkyl group (e.g., a benzyl group), and more preferably selected from the group consisting of a butyl group (tBu), a methyl group (Me), and an ethyl group (Et). In the present application, “protected carboxyl group” refers to the group protected by an appropriate carboxyl protecting group, preferably selected from the group consisting of a methoxycarbonyl group, an ethoxycarbonyl group, t-butoxycarbonyl group, and a benzyloxycarbonyl group. The carboxyl protecting groups can be removed through hydrolysis catalyzed by acids or alkalis, or through pyrolysis reactions occasionally; for example, the t-butyl group can be removed under mild acidic conditions, and the benzyl group can be removed by hydrogenolysis. The reagent used for removal of carboxyl protecting groups is selected from the group consisting of TFA, H₂O, LiGH, NaOH, KOH, MeOH, EtOH, and combinations thereof, preferably selected from the group consisting of the combination of TFA and H₂O, the combination of LiGH and MeOH, and the combination of LiGH and EtOH. A protected carboxyl group can undergo deprotection and then produce the corresponding free acid; the deprotection is conducted in the presence of an alkali, and the alkali forms pharmaceutically acceptable salt with the free acid produced via the deprotection.

In the present application, “amino protecting group” includes all the groups which are used as amino protecting groups generally, such as an aryl C₁₋₆ alkyl group, a C₁₋₆ alkoxy C₁₋₆ alkyl group, a C₁₋₆ alkoxycarbonyl group, an aryloxycarbonyl group, a C₁₋₆ alkylsulfonyl group, an arylsulfonyl group, a silyl group, etc. An amino protecting group is preferably selected from the group consisting of a t-butoxycarbonyl group (Boc), ap-methoxybenzyloxycarbonyl group (Moz), and a 9-fluorene-methylene-carbonyl (Fmoc). The reagent used for removal of amino protecting groups is selected from the group consisting of TFA, H₂O, LiGH, NaOH, KOH, MeOH, EtOH, and combinations thereof, preferably selected from the group consisting of the combination of TFA and H₂O, the combination of LiGH and MeOH, and the combination of LiGH and EtOH. The reagent used for removal of the Boc protecting group can be TFA or HC/EA, preferably TFA. The reagent used for removal of the Fmoc protecting group can be the N,N-dimethylformamide (DMF) solution containing 20% piperidine.

In the present application, “cation” refers to the corresponding structure bearing a positive charge, either permanently, or non-permanently but in response to certain conditions such as pH. Therefore, the cations include permanent cations and those cationic compounds, groups, or atoms. Permanent cations refer to the corresponding compounds, groups, or atoms that bear positive charges under conditions of any pH value or hydrogen ion activity of their environment; typically, a positive charge is generated by the presence of quaternary nitrogen atom. When a compound carries multiple such positive charges, it can be termed permanent cation. A cationic substance refers to a compound, group, or atom that is positively charged at a lower pH and uncharged at a higher pH of its environment. Moreover, in non-aqueous environments where pH cannot be determined, a cationic compound, group, or atom is positively charged at high hydrogen ion concentration and uncharged at low concentration or activity of hydrogen ions, which depends on the individual properties of the cationic or polycationic compound, especially the pKa of the cationic groups or atoms which are charged or uncharged at the corresponding pH or hydrogen ion concentration. In the diluted aqueous environment, the Henderson-Hasselbalch equation can be used to estimate the fraction of positively charged cationic compounds, groups or atoms, which is well-known to those skilled in the art. For example, in some embodiments, if a compound or moiety is cationic, pH of the compound or moiety is preferably about 1 to 9, preferably 4 to 9, 5 to 8 or even 6 to 8, more preferably equal to or below 9, equal to or below 8, equal to or below 7, and most preferably at physiological pH (e.g., about 7.3 to 7.4), i.e., positively charged under physiological conditions, especially under normal saline conditions in cells in vivo. In other embodiments, it is preferably that the cationic compound or moiety is predominantly neutral at physiological pH values, e.g., about 7.0-7.4, but becomes positively charged at lower pH values. In some embodiments, pKa for the cationic compound or moiety is preferably about 5 to about 7.

In the present application, “cationic component/compound” typically refers to a charged molecule that is positively charged (cation) at a pH of about 1 to 9. In some embodiments, the cationic component/compound is preferably charged at a physiological pH (e.g., about 7.3 to 7.4) at a pH of or below 9 (e.g., 5 to 9), of or below 8 (e.g., 5 to 8), of or below 7 (e.g., 5 to 7). Thus, cationic peptides, proteins, polysaccharides, lipids or polymers according to one embodiment of the present application are positively charged under physiological conditions, especially under physiological salt conditions of cells in vivo.

In the present application, LNP, cationic peptides, proteins, polysaccharides, lipids or polymers are uncharged, having a neutral charge or being electrically neutral under physiological conditions, especially under the physiological salt conditions of the cell in vivo. Cationic peptides or proteins preferably contain a larger amount of cationic amino acids, such as a greater number of Arg, His, Lys or Orn than other amino acid residues (especially more cationic amino acids than anionic amino acid residues such as Asp or Glu) or contain components comprising mainly cationic amino acid residues. The expression “cation” can also refer to “polycationic” components/cationic components/compounds, and can also refer to cationic lipids that can be positively charged. For example, cationic lipids contain one or more amine groups with a positive charge, preferably cationic lipids are ionizable so that they may exist in a positively charged form or neutral form according to pH. The ionization of cationic lipids affects the surface charge of LNPs at different pH conditions. This charge state can affect plasma protein absorption, blood clearance and tissue distribution, as well as the ability to form non-bilayer structures that are critical for intracellular delivery of nucleic acids.

In the present application, “PEGylated lipid” (i.e., PEG-lipid) refers to the molecules containing both lipid and PEG moieties.

In the present application, “neutral lipid” refers to any lipid substance which is uncharged or exists in the form of neutral zwitterion at the chosen pH, preferably phospholipid. The neutral lipid can be synthetic or natural.

In the present application, “steroid lipid” refers to a steroid or a steroid analogue.

In the present application, “amino acid residue” is an amino acid from which, formally, a hydrogen atom has been removed from an amino group and/or from which, formally, a hydroxy group has been removed from a carboxy group and/or from which, formally, a hydrogen atom has been removed from a sulfydryl group and/or with a protected amino group and/or with a protected carboxyl group and/or with a protected sulfydryl group. Imprecisely, amino acid residue can be described as amino acid. The source of the amino acid in the present application, unless otherwise specified, is not particularly limited; that is, the amino acid can be either natural or unnatural, or a mixture thereof. The configuration of the amino acid in the present application is not particularly limited, which could be L-type or D-type, or a mixture thereof. In one embodiment of the present application, the amino acid is a hydrophobic amino acid, selected from the group consisting of tryptophan (Trp), phenylalanine (Phe), valine (Val), isoleucine (Ile), leucine (Leu) and tyrosine (Tyr). In another embodiment of the present application, amino acids are hydrophilic amino acids, selected from the group consisting of glutamic acid (Glu), aspartic acid (Asp), histidine (His), glutamine (Gln), asparagine (Asn), serine (Ser), threonine (Thr), proline (Pro), glycine (Glys), lysine (Lys) and arginine (Arg), preferably glycine or lysine, more preferably lysine.

In the present application, “variant form” refers to a structure that can be transformed into the target reactive group after any process of chemical change selected from the group consisting of oxidation, reduction, hydration, dehydration, electronic rearrangement, structural rearrangement, salt complexation and decomplexation, ionization, protonation, deprotonation, substitution, deprotection, leaving group transformation, etc.

In the present application, “variant form of reactive group” refers to a form which still has reactivity (remains reactive group) after the reactive group undergoes at least one process of chemical change selected from oxidation, reduction, hydration, dehydration, electronic rearrangement, structural rearrangement, salt complexation and decomplexation, ionization, protonation, deprotonation, substitution, deprotection, leaving group transformation, etc., or a non-reactive form of protected reactive group.

In the present application, “micro-modification” refers to a process of chemical modification that can be completed through simple chemical reactions. The simple chemical reactions mainly include deprotection, salt complexation and decomplexation, ionization, protonation, deprotonation, leaving group transformation, etc.

The “variant form with micro-modification” corresponds to the “micro-modification”, referring to a structural form that can be transformed into the target reactive group after simple chemical reactions selected from deprotection, salt complexation and decomplexation, ionization, protonation, deprotonation, leaving group transformation, etc., the leaving group transformation includes the transformation from the form of ester to the form of acyl chloride.

“Any suitable” in “any suitable linking group”, “any suitable reactive group”, etc. in the present application refers to a structure that conforms to the basic principles of chemical structure and can enable the preparation method of the present application to be successfully implemented. The chemical structure described in this way can be considered to have a clear, defined range.

When at least two structural types are enumerated, “any combination” of the enumerated structure types means a combination of any two or more of the relevant structural types listed above. And the number of structural units is not limited, the number of any structural unit can be zero, one or greater than one. When the number of structural units of the same type is greater than 1, the structural units could be the same or different chemical structures, and the total number of the structural units is at least two. For example, any arbitrary combination of an alkylene group, a divalent cycloalkyl group, a divalent cycloalkenyl group, a divalent cycloalkynyl group, a divalent cyclodienyl group, an arylene group, a carbon-carbon double bond, a carbon-carbon triple bond, a conjugated carbon-carbon double bond, a divalent aliphatic heterocyclic linking group, a divalent aromatic heterocyclic linking group, and a carbon chain linking group with heteroatom-containing pendant groups, can be, e.g., -Ph-CH₂-Ph-(arylene-alkylene-arylene), —CH₂-Ph-CH₂CH₂— (alkylene-aryllene-alkylene; wherein, the alkylene group has a quantity of 2, with different chemical structures), or a structure obtained from the phenyl ring of any aforementioned example being replaced with the ring of a hexane, diazepine, or 1-(2-pyridinyl)hexahydro-1H-1,4-diazepine. As another example, cycloalkenyl hydrocarbon group=cycloalkenyl group+hydrocarbylene group=hydrocarbon group substituted by a cycloalkenyl group, and cyclodienyl hydrocarbon group=hydrocarbon group substituted by a cyclodienyl group.

In the present application, “N/P ratio” refers to molar ratio of nitrogen atoms in cationic lipids to phosphate in nucleic acids.

In the present application, “nucleic acid” refers to DNA, RNA or their modified form, including purine or pyrimidine bases in DNA (adenine “A”, cytosine “C”, guanine “G”, thymine “T”), and purine or pyrimidine bases in RNA (adenine “A”, cytosine “C”, guanine “G”, uracil “U”).

In the present application, “RNA” refers to ribonucleic acid that may be naturally or non-naturally occurring. For example, an RNA may include modified and/or non-naturally occurring components such as one or more nucleobases, nucleosides, nucleotides, and linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA (messenger RNA), singleguiderna RNA (sgRNA), cas9 mRNA, and mixtures thereof.

In the present application, antisense oligonucleotide and small interfering RNA (siRNA) can inhibit the expression of target gene and target protein in vitro or in vivo.

In the present application, FLuc mRNA can express luciferase protein which emits bioluminescence in the presence of fluorescein substrate, so FLuc is commonly used in mammalian cell culture to measure gene expression and cell activity.

In the present application, “inhibiting expression of a target gene” refers to the ability of nucleic acids to silence, reduce or inhibit the expression of a target gene. To examine the extent of gene silencing, a test sample (e.g., a sample of cells in culture expressing the target gene) is contacted with nucleic acids that inhibit the expression of the target gene. The expression of the target gene in the test sample or test animal is compared to expression of the target gene in a control sample (e.g., a sample of cells in culture expressing the target gene) which is not contacted with or administered the nucleic acids. The expression of the target gene in the control sample may be assigned a value of 100%. In particular embodiments, inhibition of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%.

In the present application, suitable assays for determining the level of target gene expression include but are not limited to dot blots, northern blots, in situ hybridization, ELISA, immunoprecipitation, enzyme function, and phenotypic assays.

In the present application, “transfection” refers to the introduction of a species (e.g., an RNA) into a cell. Transfection may occur, for example, in vitro, ex vivo, or in vivo.

In the present application, “antigen” typically refers to a substance that can be recognized by the immune system, preferably recognized by the adaptive immune system, and trigger an antigen-specific immune response, for example, forming antibodies and/or antigen-specific T cells as a part of the adaptive immune response. Typically, the antigen may be or may contain a peptide or a protein that can be presented to T cells by MHC. In the present application, the antigen may be a translation product of the provided nucleic acid molecule (preferably mRNA as defined herein). In this context, fragments, variants, and derivatives of peptides and proteins containing at least one epitope are also defined as antigens.

In the present application, “delivery” refers to delivering an entity to the target, for example, delivering drugs and/or therapeutic agents and/or prophylactic agents to subjects, the subjects are tissues and/or cells of human and/or other animals.

In the present application, “pharmaceutically acceptable carrier” refers to a diluent, adjuvant, excipient, or vehicle administered together with the therapeutic agent, which is, within the scope of reasonable medical judgement, suitable for contacting with tissues of human and/or other animals without causing excessive toxicity, irritation, allergic reaction, or other problems or complications corresponding to reasonable benefit/risk ratio. Pharmaceutically acceptable carriers that can be used in the pharmaceutical composition in the present application include but are not limited to sterile liquids, such as water and oil, including those from petroleum, animal, vegetable, or synthesis, e.g., peanut oil, soybean oil, mineral oil, sesame oil, etc. When the pharmaceutical composition is administered intravenously, water is an exemplary carrier. Physiological saline, glucose, and aqueous glycerol solution can also be used as liquid carriers, especially as injection. Suitable pharmaceutical excipients include starch, glucose, lactose, sucrose, gelatin, maltose, chalk, silica gel, sodium stearate, glyceryl monostearate, talc, sodium chloride, defatted milk powder, glycerol, propylene glycol, water, ethanol, etc. The composition can also contain a small amount of humectant, emulsifier, or pH buffer as needed. Oral preparations can contain standard carriers, such as pharmaceutical grade mannitol, lactose, starch, magnesium stearate, sodium saccharin, cellulose, magnesium carbonate, etc. Specifically, excipients include but are not limited to anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. More specifically, excipients include but are not limited to butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (α-tocopherol), vitamin C, and xylitol.

In the present application, pharmaceutical compositions can act systematically and/or locally. For this purpose, they can be administered by appropriate routes such as injection (e.g., intravenous, intraarterial, subcutaneous, intraperitoneal, or intramuscular injection, including instillation) and transdermal delivery, and can also be administered by oral, buccal, transnasal, transmucosal, or topical routes, or in the form of ophthalmic preparation, or by inhalation. Regarding these routes of administration, the pharmaceutical compositions of the present application can be administered in suitable dosage forms. The dosage forms include but are not limited to, tablets, capsules, lozenges, hard sugar agents, powders, sprays, creams, ointments, suppositories, gels, pastes, lotions, ointments, aqueous suspensions, injectable solutions, elixirs, and syrups.

In the present application, vaccines are preventive or therapeutic materials that provide at least one antigen or antigenic function. Antigen or antigenic function can stimulate the body adaptive immune system to provide an adaptive immune response.

In the present application, treatment refers to the management and care of patients to resist diseases, obstacles, or symptoms, which is intended to delay the development of diseases, obstacles, or symptoms, reduce or alleviate symptoms and complications, and/or cure or eliminate diseases, obstacles, or symptoms. The patients to be treated are preferably mammals, especially humans.

### 1. Amino Acid-Based Cationic Lipids

An embodiment of the present application:

An amino acid-based cationic lipid, wherein, its structure is represented by the general formula (1):

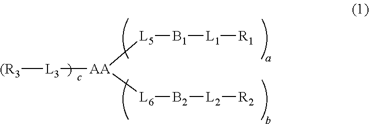

Wherein, AA is a residue of an amino acid or an amino acid derivative;
B₁ and B₂ are each independently a linking bond or a C₁₋₃₀ alkylene group at each occurrence;
L₁ and L₂ are each independently a linking bond or a divalent linking group at each occurrence;
L₅ and L₆ are each independently a linking bond or a divalent linking group at each occurrence;
L₃ is independently a linking bond or a divalent linking group at each occurrence;
R₁ and R₂ are each independently —(CH₂)ₜNRₑR_{f}, a linear C₁₋₃₀ alkylene group, a branched C₁₋₃₀ alkylene group or

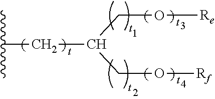

at each occurrence; wherein, t is an integer from 0 to 12; t₁ and t₂ are each independently an integer from 0 to 5; t₃ and t₄ are each independently 0 or 1; t₁, t₂, t₃ and t₄ are not 0 simultaneously; Rₑ and R_{f} are each independently a C₁₋₁₅ alkyl group, a C₂₋₁₅ alkenyl group, or a C₂₋₁₅ alkynyl group;
R₃ is independently, at each occurrence, a hydrogen atom, -R_{d}, —OR_{d}, a C₃₋₆ carbocyclic group, a nitrogen-containing heterocyclic groups, —NR_{d}R_{d}, -SR_{d}, —C(═O)R_{d}, —C(═O)OR_{d}, —OC(═O)R_{d}, —OC(═O)OR_{d} or a functional group R01 that can react with bio-related substances; wherein, R_{d} is independently a C₁₋₁₂ alkyl group at each occurrence;
a, b and c are each independently 1 or 2; when fragments -L₅-B₁-L₁-R₁ and/or -L₆-B₂-L₂-R₂ and/or -L₃-R₃ are protruded from the amino terminus of the amino acid, a, b, and c are each independently 1 or 2; when fragments -L₅-B₁-L₁-R₁ and/or -L₆-B₂-L₂-R₂ and/or -L₃-R₃ are protruded from the carboxyl terminus, hydroxyl terminus, or thiol terminus of an amino acid, a, b, and c are each independently 1; when a is 2, two -L₅-B₁-L₁-R₁ fragments are the same or different; when b is 2, two -L₆-B₂-L₂-R₂ fragments are the same or different; when c is 2, two -L₃-R₃ fragments are the same or different;
Or a salt, tautomer, stereoisomer or solvate thereof.

### 1.1. Residues of Amino Acids or Amino Acid Derivatives (AA)

In the present application, AA is the residue of amino acids or amino acid derivatives.

In a specific embodiment of the present application, the aforementioned amino acids or amino acid derivatives are preferably arginine, aspartic acid, asparagine, cysteine, glutamic acid, glutamine, histidine, lysine, methionine, phenylalanine, serine, threonine, tryptophan, tyrosine and amino acid derivatives of any of the aforementioned amino acids.

In a specific embodiment of the present application, the amino acid or amino acid derivative residue (AA) is preferably selected from the group consisting of the following structures:

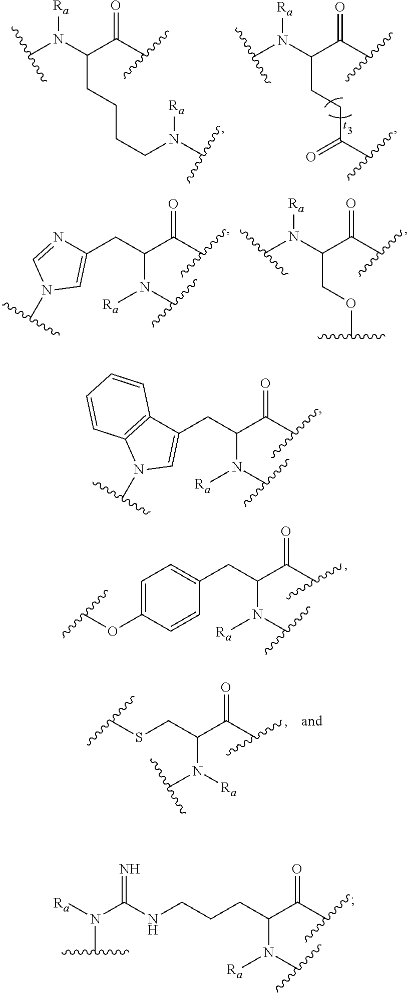

wherein, Rₐ is independently selected from the group consisting of a linking bond, H, a methyl group, an ethyl group, a propyl group, and a isopropyl group at each occurrence; or AA is the case where one or two carbonyl groups in any of the aforementioned structures are each independently capped by an oxygen atom or a secondary amine group, such as:

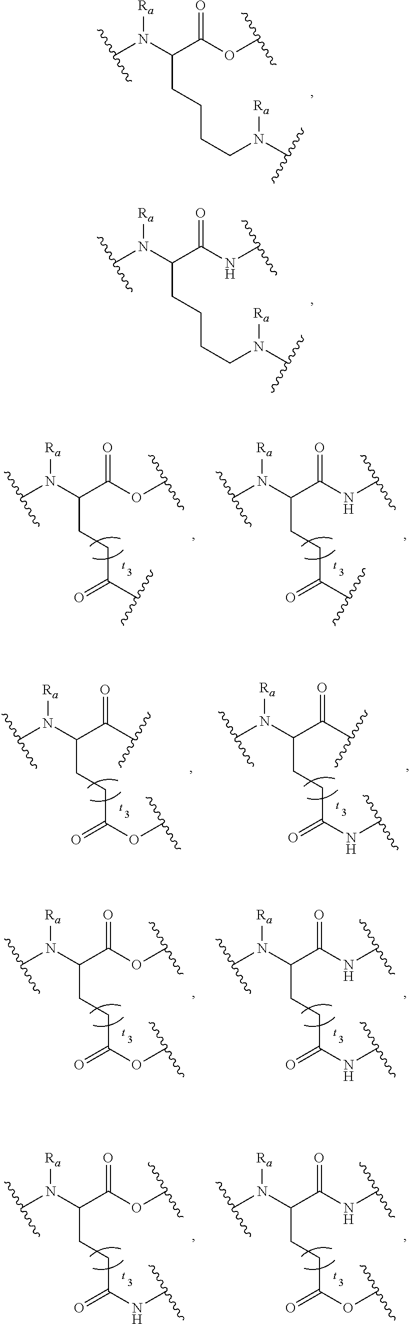

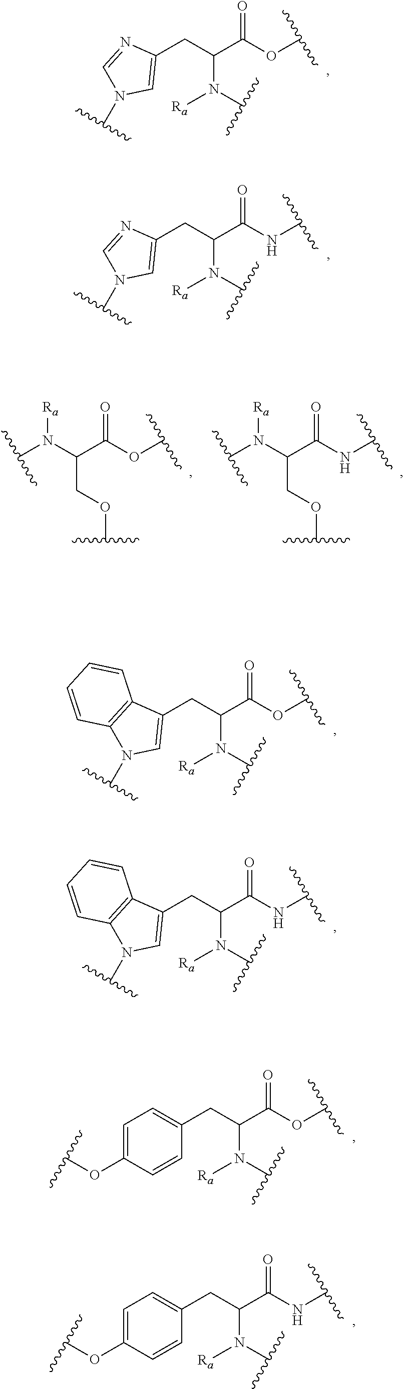

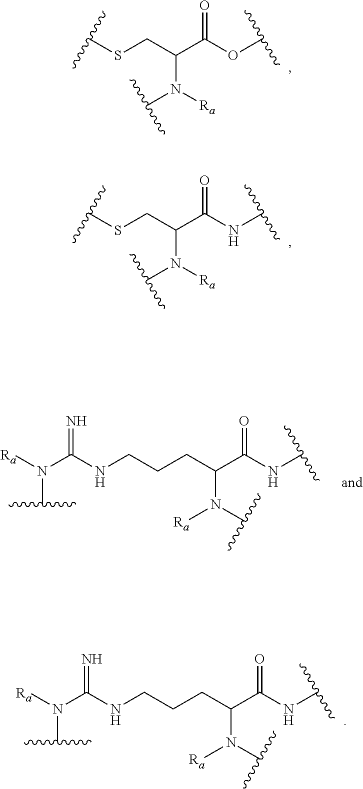

In a specific embodiment of the present application, a residue of an amino acid or an amino acid derivatives (AA) is preferably selected from the group consisting of the following structures:

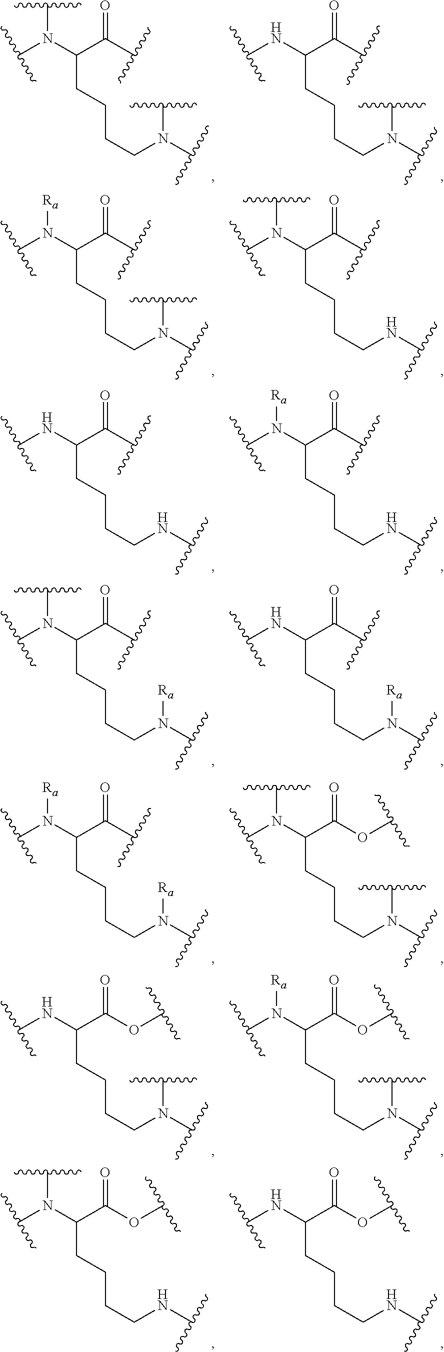

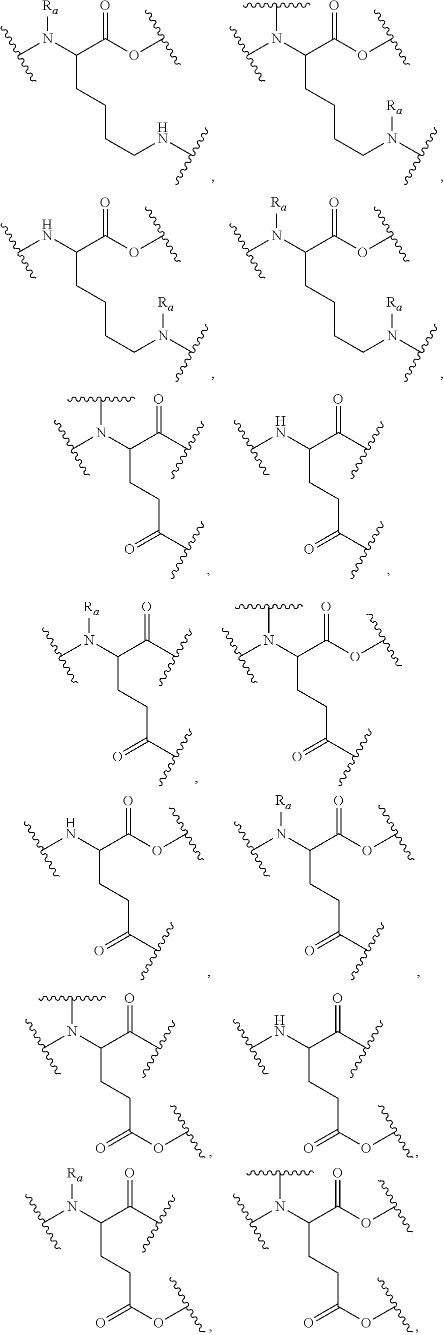

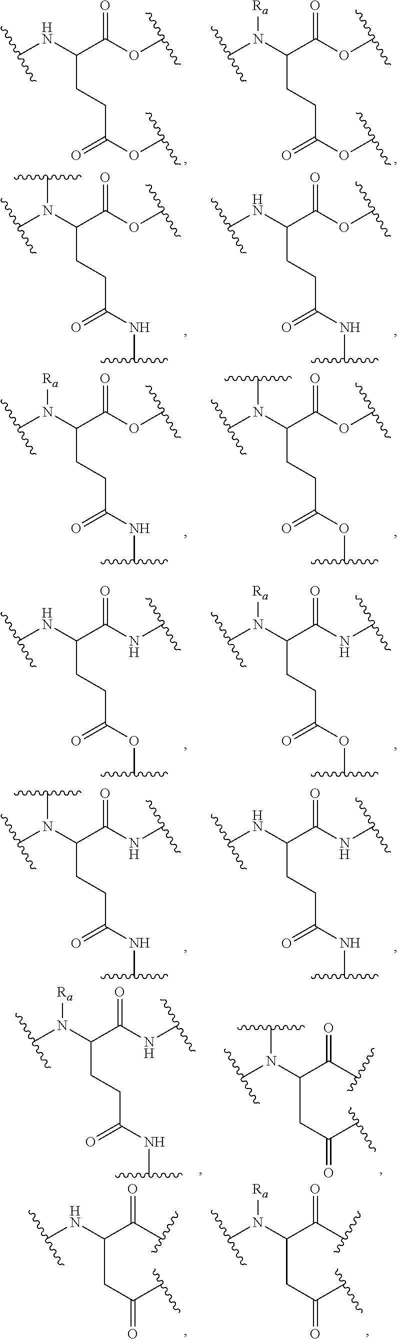

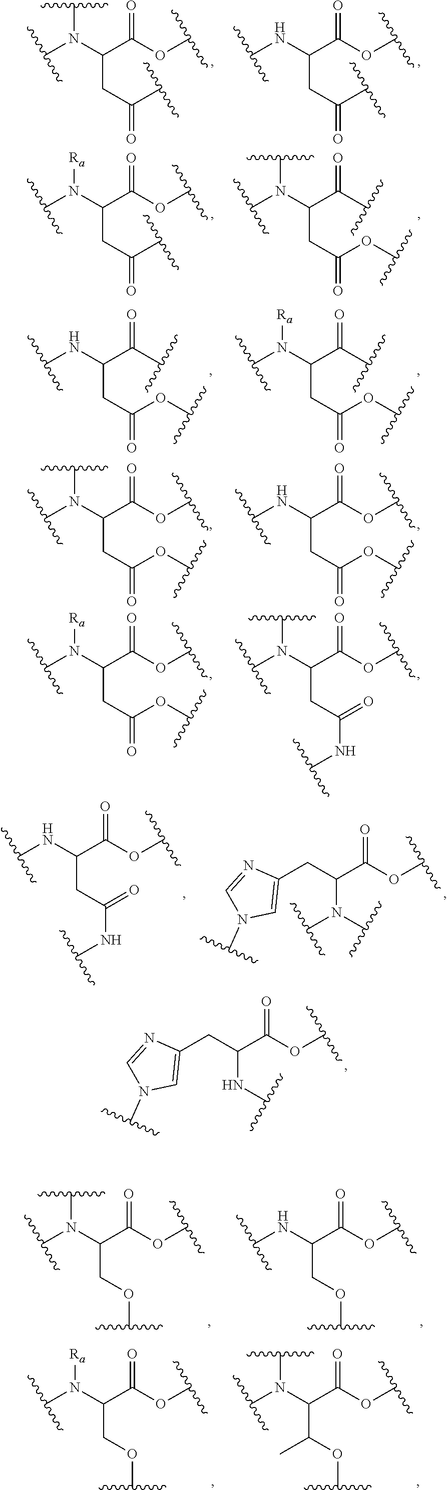

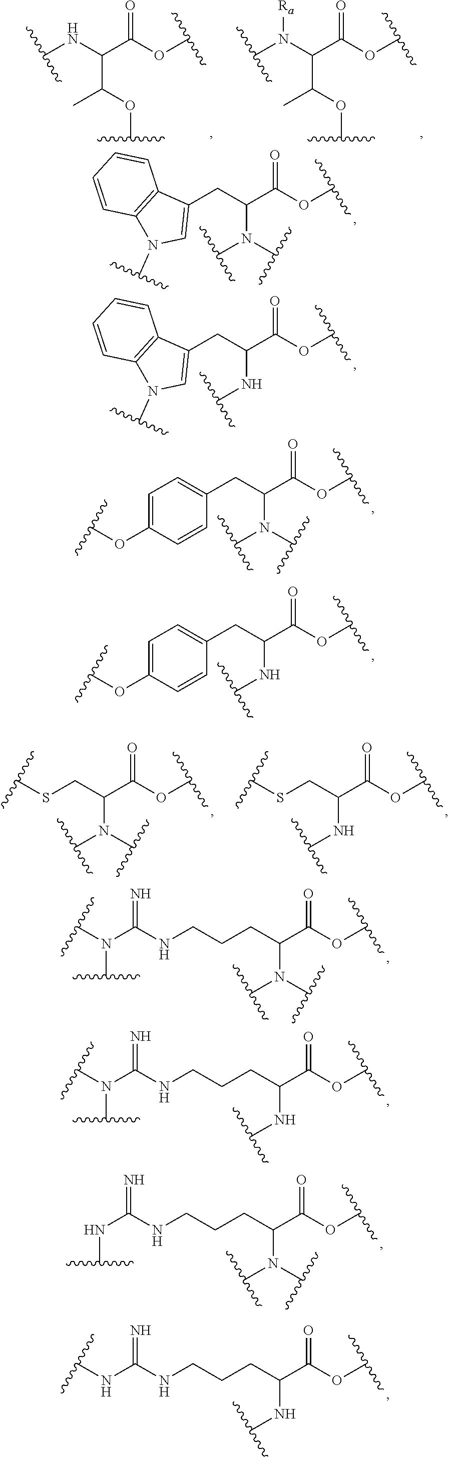

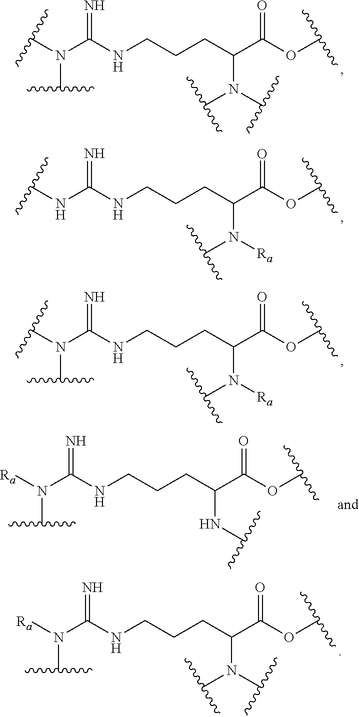

1.2. B₁, B₂

In the present application, B₁ and B₂ are each independently a linking bond or a C₁₋₃₀ alkylene group at each occurrence.

In a specific embodiment of the present application, B₁ and B₂ are each independently a linking bond or a C₁₋₂₀ alkylene group, specifically selected from any one of the following cases:
Case (1): B₁ and B₂ are each independently a C₁₋₂₀ alkylene group, and specifically B₁ and B₂ are each independently selected from the group consisting of a methylene group, an ethylene group, a propylene group, a butylene group, an pentylene group, a hexylene group, a heptylene group, an octylene group, a nonylene group, a decylene group, a undecylene group, a dodecylene group, a thirylene group, a tetradecylene group, a pentadecylene group, a hexadecylene group, a heptadecylene group, an octadecylene group, a nonadecylene group and an eicosylene group; more preferably B₁ and B₂ are each independently a C₂-10 alkylene group;
Case (2): one of B₁ and B₂ is a linking bond, and the other is a C₁₋₂₀ alkylene group;
Case (3): both B₁ and B₂ are linking bonds.
   1.3. L₁, L₂, L₃, L₅, L₆
   1.3.1. L₁, L₂

In the present application, L₁ and L₂ are each independently a linking bond or a divalent linking group at each occurrence.

In a specific embodiment of the present application, L₁ and L₂ are each independently selected from the group consisting of a linking bond, —OC(═O)—, —C(═O)O—, —OC(═O)O—, —C(═O)—, —O—, —NH—, —O(CR_{c}R_{c})ₛO—, —S—, —C(═O)S—, —SC(═O)—, —NR_{c}C(═O)—, —C(═O)NRₑ—, -NR_{c}C(═O)NR_{c}—, —OC(═O)NR_{c}—, —NR_{c}C(═O)O—, —SC(═O)NR,- and -NR_{c}C(═O)S—; wherein, R, is independently a hydrogen atom or a C₁₋₁₂ alkyl group at each occurrence, and s is 1, 2, 3 or 4; L₁ and L₂ are more preferably selected from the following cases:
Case (1): L₁ and L₂ are each independently selected from the group consisting of —OC(═O)—, —C(═O)O—, —OC(═O)O—, —C(═O)—, —O—, —O(CH₂)ₛO—, —S—, —C(═O)S—, —SC(═O)—, —NHC(═O)—, —C(═O)NH—, —NHC(═O)NH—, —OC(═O)NH—, —NHC(═O)O—, —SC(═O)NH— and —NHC(═O)S—;
Case (2): one of L₁ and L₂ is a linking bond, and the other is selected from the group consisting of —OC(═O)—, —C(═O)O—, —OC(═O)O—, —C(═O)—, —O—, —O(CH₂)ₛO—, —S—, —C(═O)S—, —SC(═O)—, —NHC(═O)—, —C(═O)NH—, —NHC(═O)NH—, —OC(═O)NH—, —NHC(═O)O—, —SC(═O)NH— and —NHC(═O)S—;
Case (3): both L₁ and L₂ are linking bonds.

In a specific embodiment of the present application, preferably L₁ and L₂ are each independently selected from the group consisting of a linking bond, —C(═O)—, —O—, —NH—, —OC(═O)—, —C(═O)O—, —OC(═O)O—, —NHC(═O)— and —C(═O)NH-.

### 1.3.2. L₃

In the present application, L₃ is independently a linking bond or a divalent linking group at each occurrence.

In a specific embodiment of the present application, L₃ is preferably a divalent linking group, selected from the group consisting of divalent linking groups L₇, L₈, Z and combinations of any two or more thereof; more preferably, L₃ is selected from the group consisting of -L₇-, -L₇-Z—, -Z-L₇-, -Z-L₇-Z—, -L₇-Z-L₈-, -Z-L₇-Z-L₈-, -L₇-Z-L₈-Z—, -Z-L₇-Z-L₈-Z— and -L₇-Z-L₈-Z-L₇-Z—, which are divalent linking groups; wherein, the L₇ and L₈ are carbon-chain linking groups, each independently represented by -(CRₐR_{b})ₜ-(CRₐR_{b})ₒ-(CRₐR_{b})ₚ—, and t, o, and p are each independently an integer of 0 to 12, and t, o, p are not 0 at the same time, Ra and R_{b} are each independently a hydrogen atom or a C₁₋₁₂ alkyl group at each occurrence; the Z is independently selected from the group consisting of —C(═O)—, —NH—, —OC(═O)—, —C(═O)O—, —OC(═O)O—, —O—, —S—, —C(═O)S—, —SC(═O)—, —NR_{c}C(═O)—, —C(═O)NR_{C}—, —NRCC(═O)NR_{C}—, —OC(═O)NR_{c}—, —NRC(═O)O—, —SC(═O)NR,- and —NR_{c}C(═O)S— at each occurrence; wherein, R, is independently H or a C₁₋₁₂ alkyl group at each occurrence.

In a specific embodiment of the present application, the aforementioned L₃ is preferably selected from the group consisting of —(CH₂)ₜ—, —(CH₂)ₜZ—, —Z(CH₂)ₜ—, —(CH₂)ₜZ(CH₂)ₜ—, —Z(CH₂)ₜZ—, —(CH₂)ₜZ(CH₂)Z—, —Z(CH₂)ₜZ(CH₂)— and —Z(CH₂)ₜZ(CH₂)ₜZ—; wherein, t is an integer of 1 to 12; more preferably L₃ is selected from the group consisting of —(CH₂)—, —(CH₂)ₜO—, —(CH₂)C(═O)—, —(CH₂)NH-, —(CH₂)C(═O)O—, —(CH₂)OC(═O)—, —(CH₂)C(═O)NH—, —(CH₂)ₜNHC(═O)—, —(CH₂)OC(═O)O—, —(CH₂)ₜNHC(═O)O—, —(CH₂)ₜOC(═O)NH—, —(CH₂)ₜNHC(═O)NH—, —O(CH₂)—, —C(═O)(CH₂)—, —C(═O)O(CH₂)—, —OC(═O)(CH₂)—, —C(═O)NH(CH₂)—, —NHC(═O)(CH₂)—, —OC(═O)O(CH₂)—, —NHC(═O)O(CH₂)—, —OC(═O)NH(CH₂)—, —NHC(═O)NH(CH₂)—, —(CH₂)O(CH₂)—, —(CH₂)ₜC(═O)(CH₂)—, —(CH₂)C(═O)O(CH₂)ₜ—, —(CH₂)OC(═O)(CH₂)ₜ—, —(CH₂)ₜC(═O)NH(CH₂)—, —O(CH₂)ₜO—, —(CH₂)NHC(═O)(CH₂)ₜ—, —(CH₂)OC(═O)O(CH₂)—, —(CH₂)NHC(═O)O(CH₂)—, —(CH₂)OC(═O)NH(CH₂)—, —(CH₂)ₜNHC(═O)NH(CH₂)ₜ—, —C(═O)(CH₂)ₜC(═O)—, —C(═O)O(CH₂)C(═O)O—, —OC(═O)(CH₂)ₜOC(═O)—, —C(═O)O(CH₂)ₜOC(═O)—, —OC(═O)(CH₂)C(═O)O—, —OC(═O)O(CH₂)ₜOC(═O)O—, —C(═O)NH(CH₂)ₜC(═O)NH—, —NHC(═O)(CH₂)ₜNHC(═O)—, —NHC(═O)(CH₂)C(═O)NH—, —C(═O)NH(CH₂)ₜNHC(═O)—, —NHC(═O)O(CH₂)ₜNHC(═O)O—, —OC(═O)NH(CH₂)OC(═O)NH—, —C(═O)(CH₂)O—, —NHC(═O)O(CH₂)OC(═O)NH—, —OC(═O)NH(CH₂)ₜNHC(═O)O—, —C(═O)(CH₂)C(═O)O—, —NHC(═O)NH(CH₂)ₜNHC(═O)NH—, —C(═O)(CH₂)ₜOC(═O)—, —C(═O)(CH₂)ₜOC(═O)O—, —C(═O)(CH₂)ₜNHC(═O)O—, —C(═O)(CH₂)ₜOC(═O)NH—, —C(═O)(CH₂)ₜNHC(═O)NH—, —C(═O)(CH₂)C(═O)O(CH₂)—, —C(═O)(CH₂)ₜOC(═O)(CH₂)—, —C(═O)(CH₂)OC(═O)O(CH₂)—, —C(═O)(CH₂)ₜNHC(═O)O(CH₂)—, —C(═O)(CH₂)OC(═O)NH(CH₂)—, —C(═O)(CH₂)ₜNHC(═O)NH(CH₂)— and —C(═O)(CH₂)ₜC(═O)(CH₂)ₜNHC(═O)O—; and most preferably L₃ is selected from the group consisting of —(CH₂)ₜ—, —(CH₂)ₜNH—, —(CH₂)ₜO—, —(CH₂)C(═O)O—, —(CH₂)OC(═O)— and —O(CH₂)ₜ—.

1.3.3. L₅, L₆

In the present application, L₅ and L₆ are each independently a linking bond or a divalent linking group at each occurrence.

In a specific embodiment of the present application, L₅ and L₆ are each independently a linking bond, —O—, —NH-, —C(═O)—, —OC(═O)- and —C(═O)O—; more preferably one of L₅ and L₆ is a linking bond, and the other is —O—, —NH—,-C(═O)—, —OC(═O)—, or —C(═O)O—; more preferably, L₅ and L₆ are simultaneously linking bonds, simultaneously —O—, simultaneously -NH-, or simultaneously -C(═O)O—.

1.4. R₁, R₂, R₃
1.4.1. R₁, R₂

In the present application, R₁ and R₂ are each independently a linear alkyl group, a branched alkyl group or

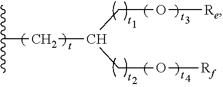

at each occurrence.

In a specific embodiment of the present application, the linear alkyl group is a C₁₋₂₅ linear alkyl group, more preferably a C₁₋₁₇ linear alkyl group which is specifically selected from the group consisting of a pentyl group, a hexyl group, a heptyl group, an octyl group, a nonyl group, a decyl group, a undecyl group, a dodecyl group, a tridecyl group, a tetradecyl group, a pentadecyl group, a hexadecyl group, or a heptadecyl group.

In one specific embodiment of the present application, the branched alkyl group is represented as

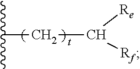

wherein, Rₑ and R_{f} are each independently a C₁₋₁₅ alkyl group; more preferably Rₑ and R_{f} are independently selected from the group consisting of a methyl group, an ethyl group, a propyl group, a butyl group, a pentyl group, a hexyl group, a heptyl group, an octyl group, a nonyl group, a decyl group; more preferably branched alkyl group is selected from the group consisting of the following structures:

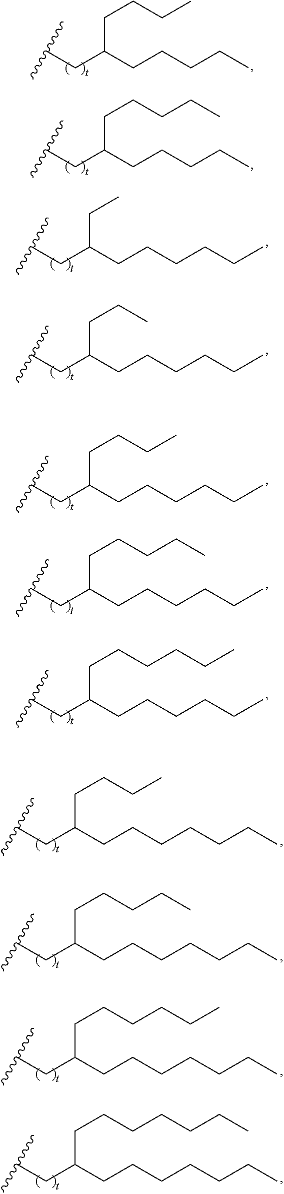

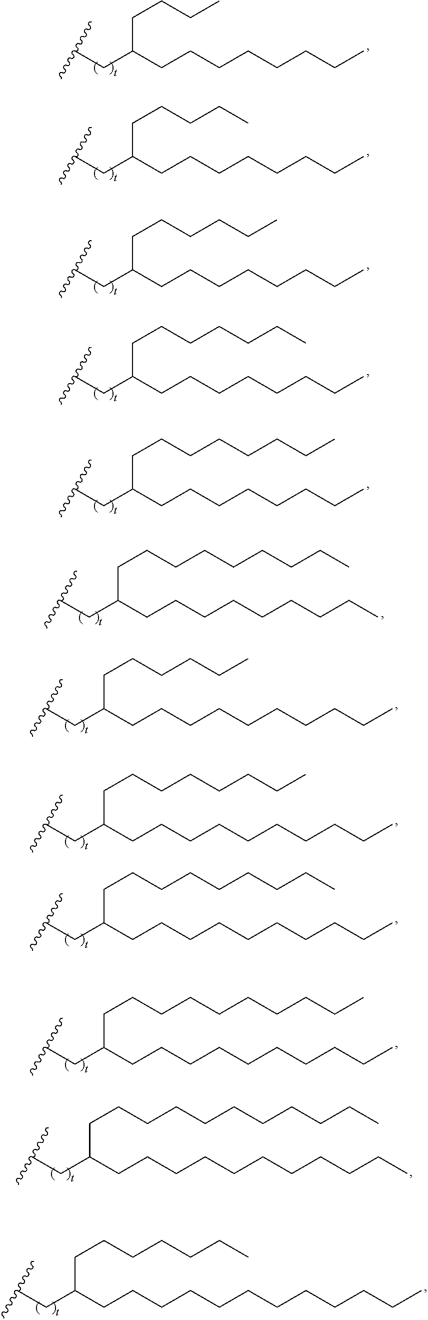

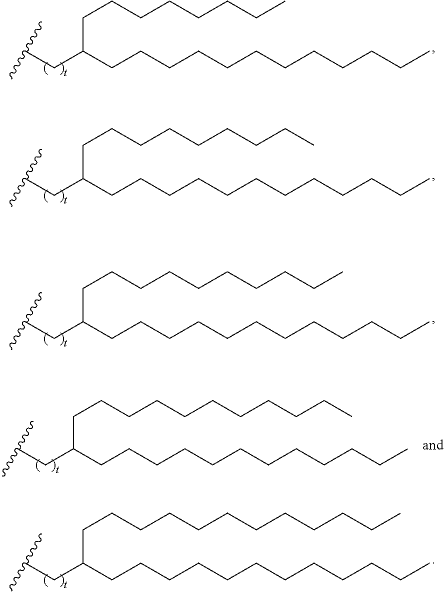

In one specific embodiment of the present application,

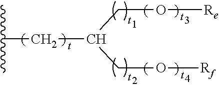

is selected from the group consisting of the following structures:

### 1.4.2. R₃

In the present application, R₃ is independently selected from the group consisting of a hydrogen atom, -R_{d}, —OR_{d}, a C₃₋₆ carbocyclic group, a nitrogen-containing heterocyclic group, —NR_{d}R_{d}, —SR_{d}, —C(═O)R_{d}, —C(═O)OR_{d}, —OC(═O)R_{d}, —OC(═O)OR_{d}, or a functional group R₀₁ capable of interacting with bio-related substances at each occurrence; wherein, R_{d} is independently a C₁₋₁₂ alkyl group at each occurrence.

In a specific embodiment of the present application, preferably R₃ is each independently selected from the group consisting of a hydrogen atom, an alkyl group, an alkoxy group, —C(═O)OR_{d}, —OC(═O)R_{d}, —OC(═O)OR_{d}, an epoxy group, a hydroxyl group, a protected hydroxyl group, a sulfhydryl group, a protected sulfhydryl group, a carboxyl group, a protected carboxyl group, an amino group, a protected amino group, an aldehyde group, a protected aldehyde group, an active ester group, a carbonate group, a urethane groups, a isocyanate group, a isothiocyanate group, a succinimidyl group, a maleimide group, a protected maleimide group, a dimethylamino group, an alkenyl group, an enoate group, an azide group, a cyano group, a dithiopyridinyl group, an α-haloacetylacetylene group, an alkyne group, a folate group, a rhodamine group, a biotinyl group, a monosaccharide group, a polysaccharide group,

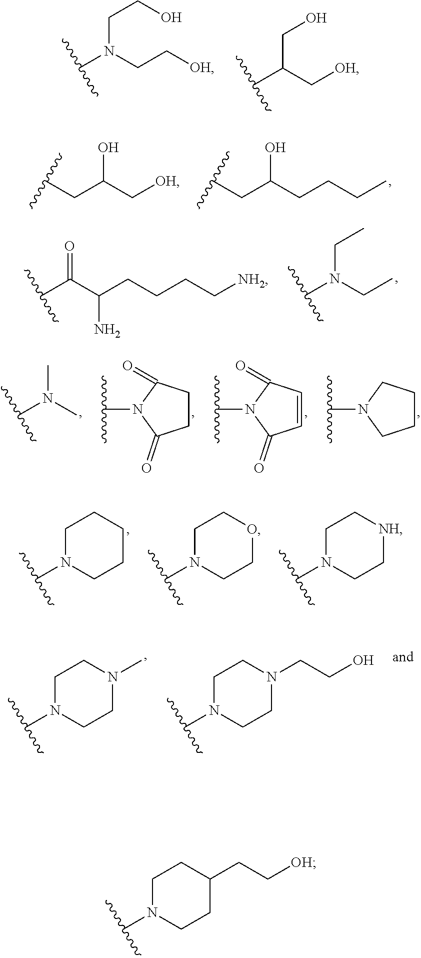

wherein, R_{d} is independently a C₁₋₁₂ alkyl group at each occurrence.

In a specific embodiment of the present application, R₃ is independently a functional group R01 that can react with a bio-related substance at each occurrence, and the R₀₁ is a functional group with therapeutic targeting properties; preferably a residue of folic acid, N-acetylgalactosamine, or a functional derivative thereof, more preferably, R₀₁ is selected from the group consisting of the following structures:

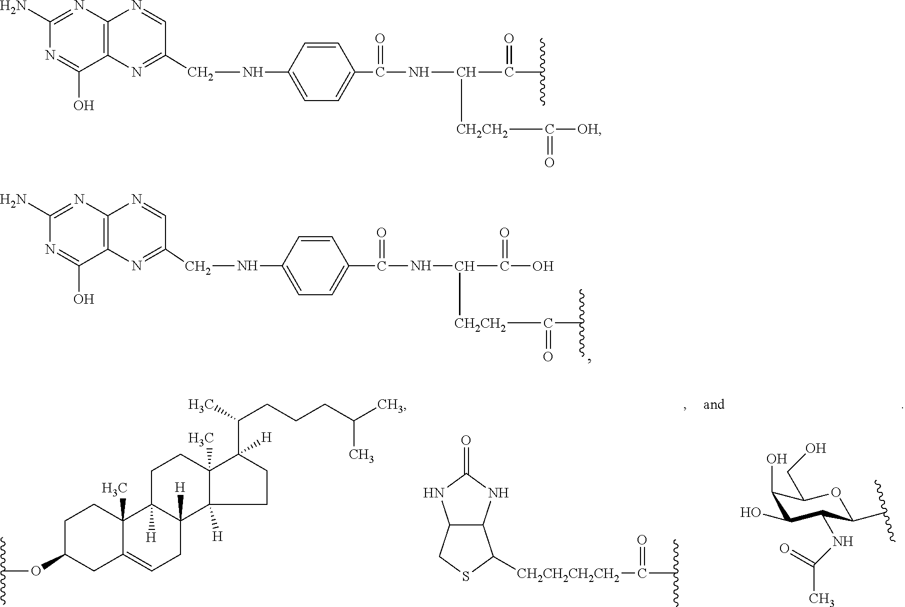

1.5. -L₃-R₃, -L₅-B₁-L₁-R₁ and -L₆-B₂-L₂-R₂ fragments
1.5.1. -L₃-R₃ fragments

In a specific embodiment of the present application, -L₃-R₃ composed of L₃ described in part 1.3.2 and R₃ described in part 1.4.2 is independently selected from any of the following structures at each occurrence:

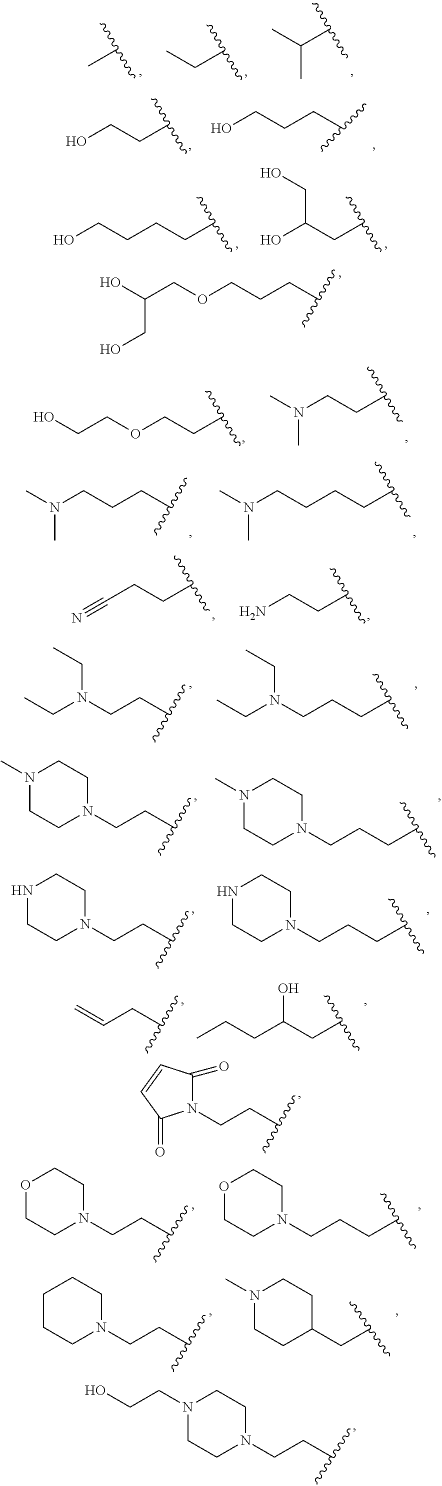

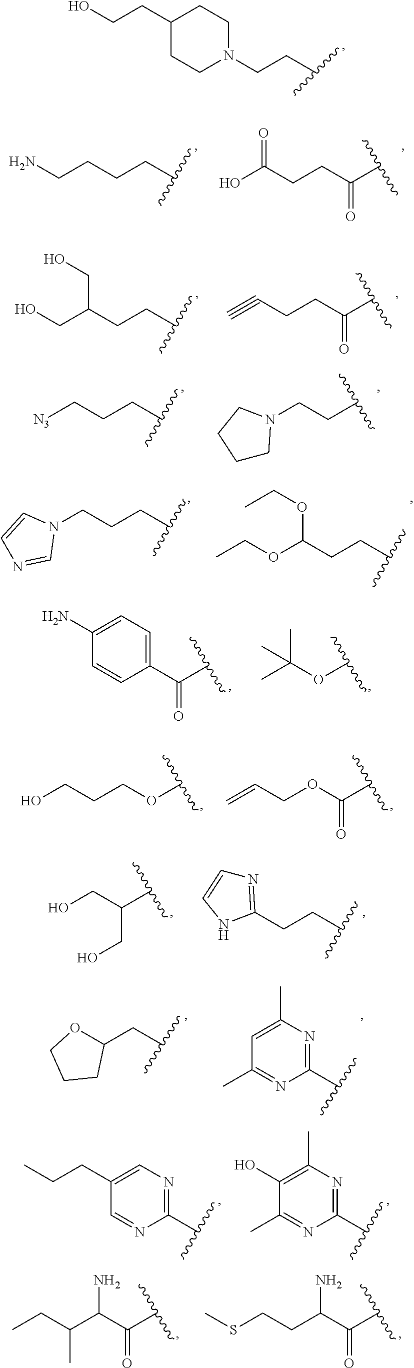

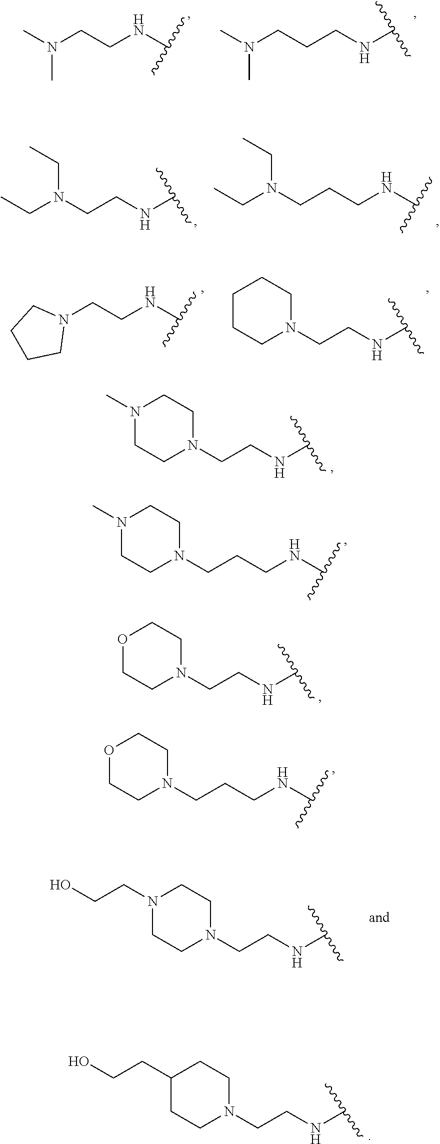

1.5.2. -L₅-B₁-L₁-R₁ and -L₆-B₂-L₂-R₂ Fragments

In a specific embodiment of the present application, -L₅-B₁-L₁-R₁ and -L₆-B₂-L₂-R₂ are each independently selected from the following structures at each occurrence:

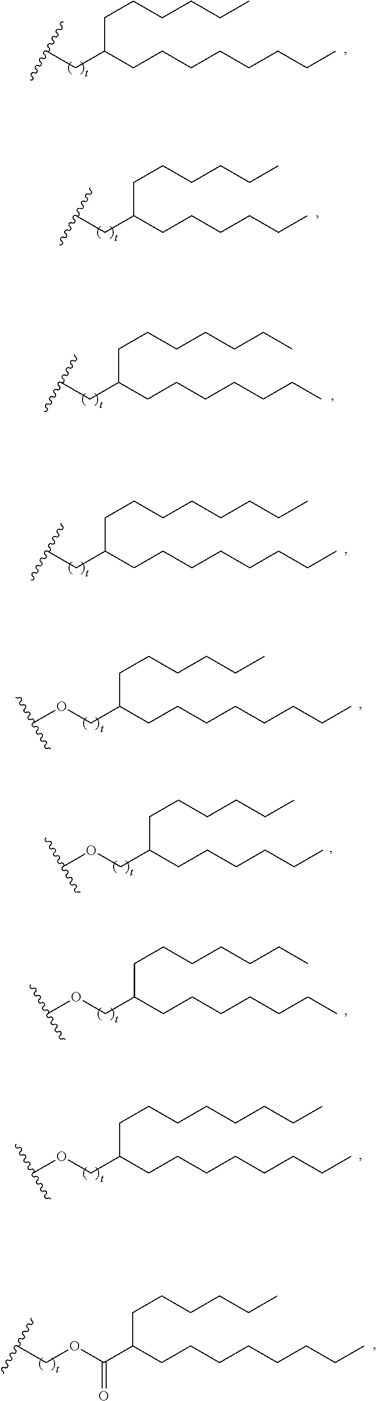

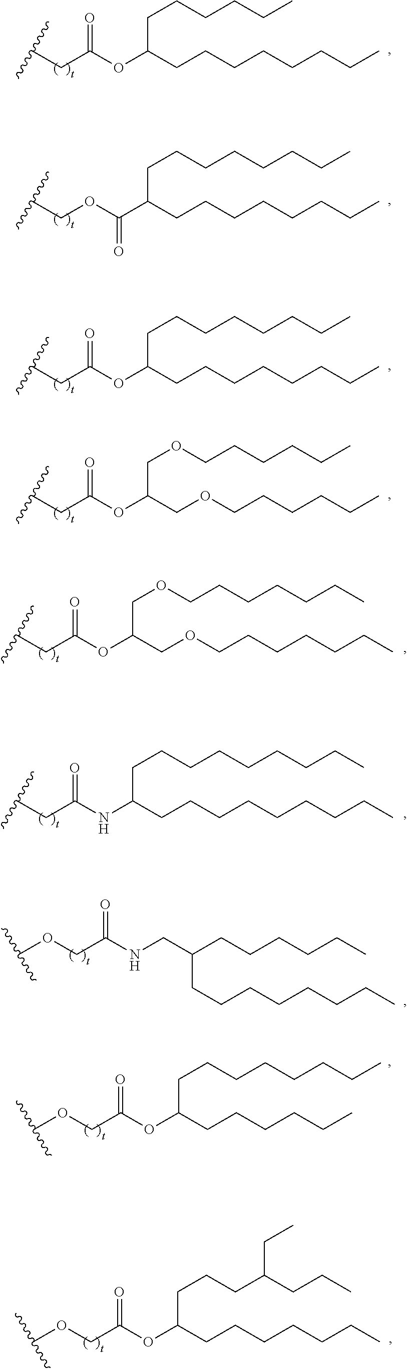

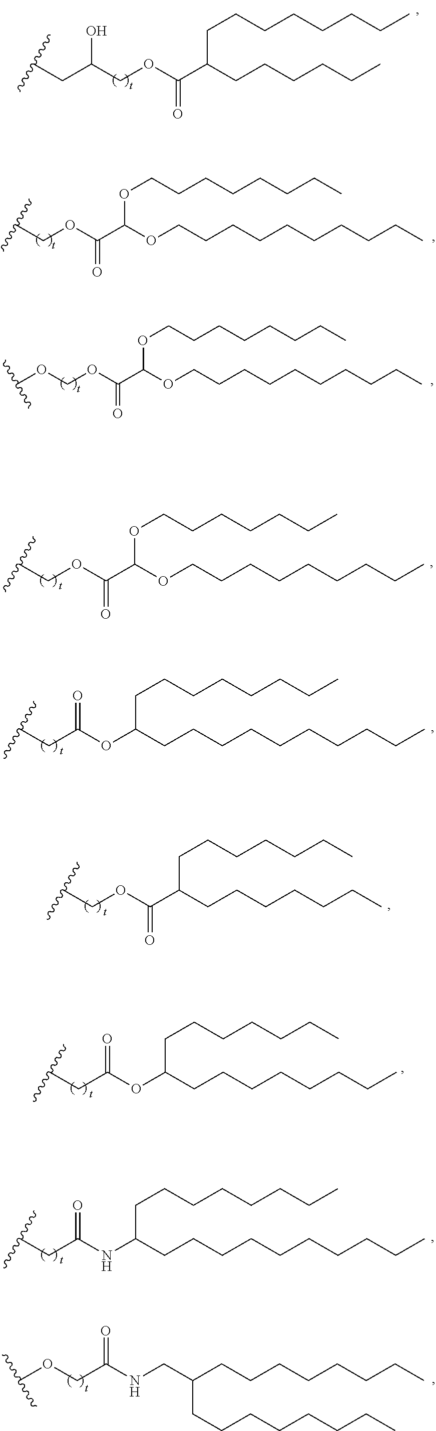

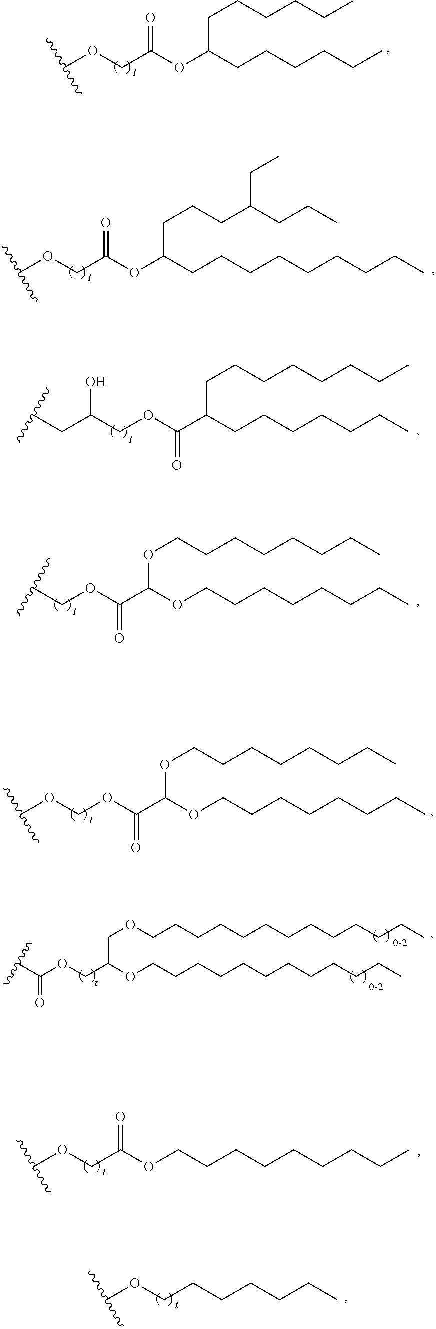

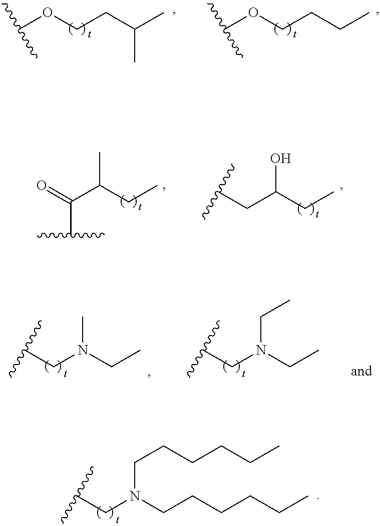

### 1.6. Examples of Structural Formulas

In a specific embodiment of the present application, according to L₁ and L₂ described in 1.3.1, the structure of amino acid-based cationic lipid of the present application is selected from the group consisting of the following general formulas:

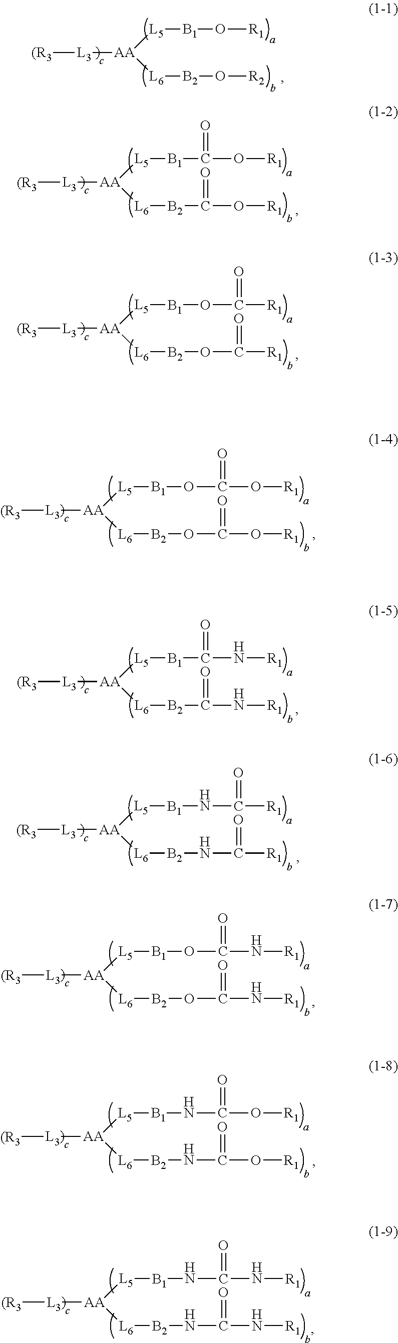

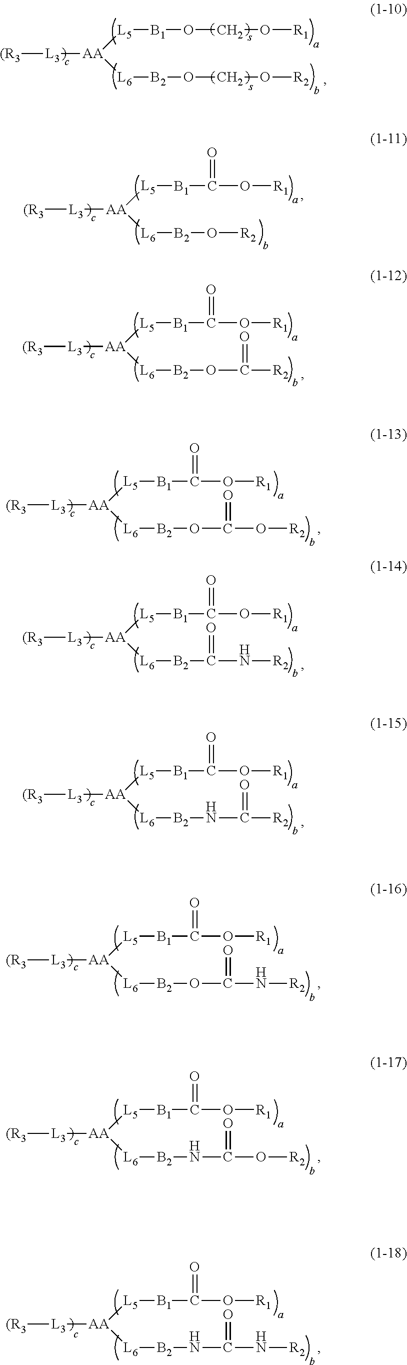

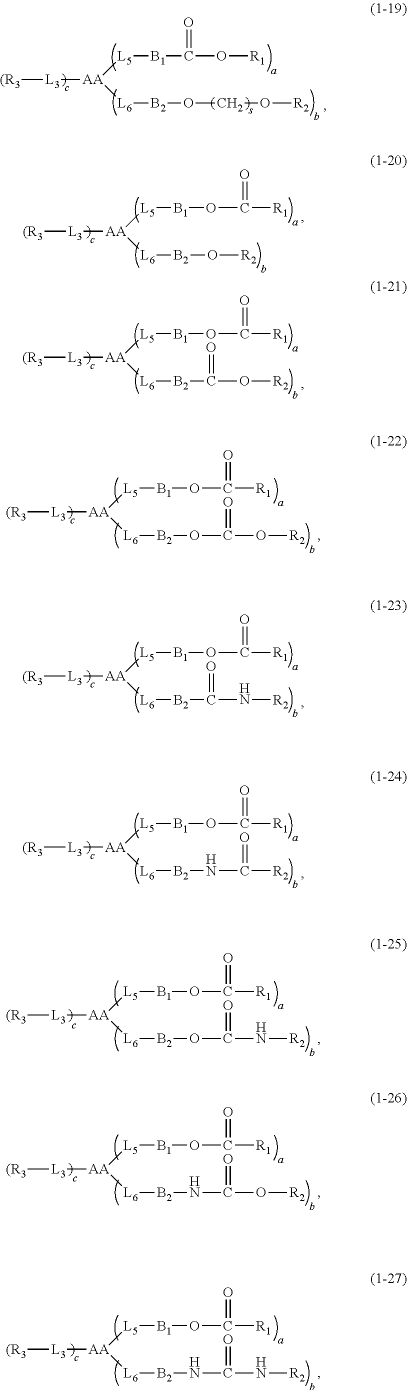

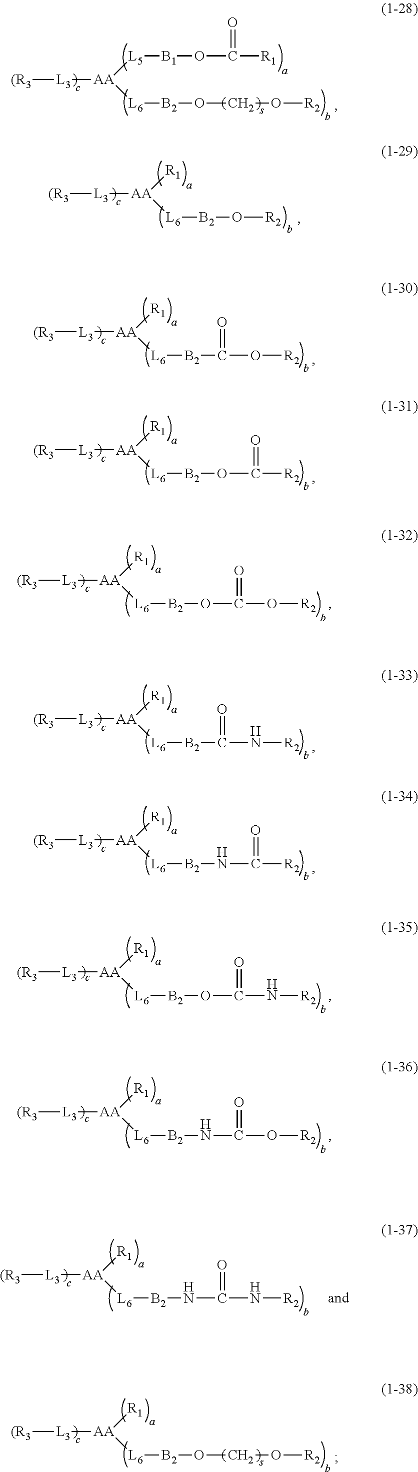

wherein, s is 1, 2, 3 or 4; the definitions of AA, B₁, B₂, L₃, L₅, B₆, R₁, R₂, R₃, a, b and c are consistent with those described in general formula (1) and will not be repeated here.

In a specific embodiment of the present application, the structure of the amino acid-based cationic lipid is preferably selected from the group consisting of the following general formulas:

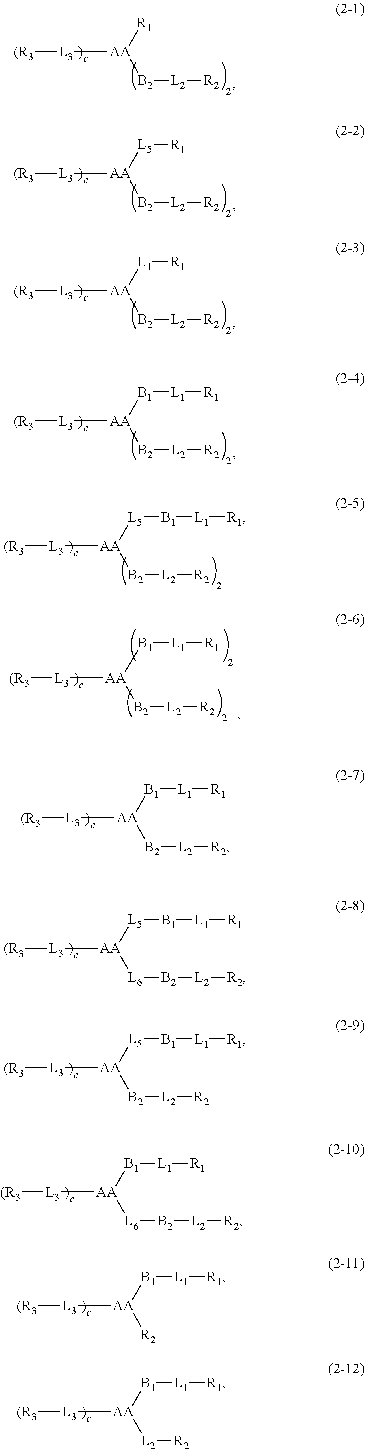

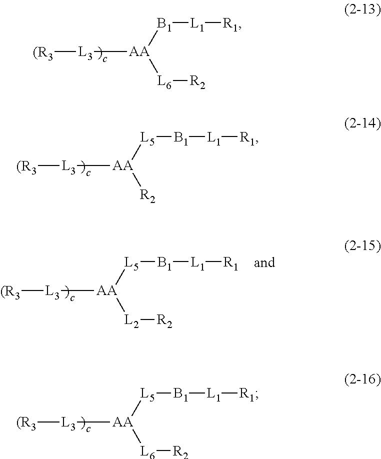

wherein, AA, B₁, B₂, L₁, L₂, L₃, L₅, B₆, R₁, R₂, R₃ and c are defined as described in the general formula (1) and will not be repeated here, and none of f₁, B₂, L₁, L₂, L₅, and L₆ is a linking bond; and more preferably L₁, L₂, L₅ and L₆ are each independently selected from the group consisting of —O—, —NH—, —C(═O)—, —C(═O)O—, —OC(═O)—, —OC(═O)O—, —C(═O)NH—, —NHC(═O)—, —OC(═O)NH—, and —NHC(═O)O— at each occurrence.

In a specific embodiment of the present application, the structure of the amino acid-based cationic lipid is selected from the group consisting of the following general formulas:

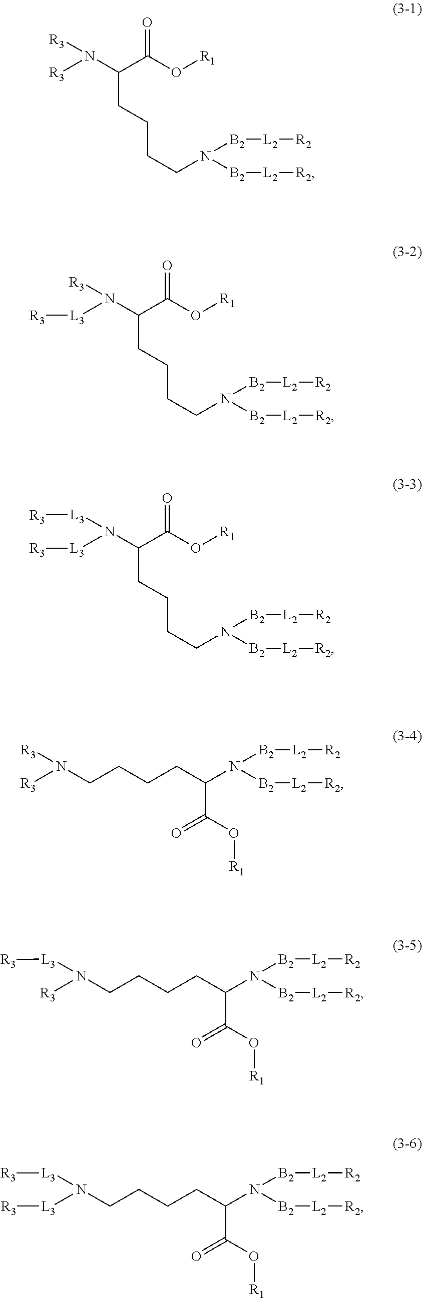

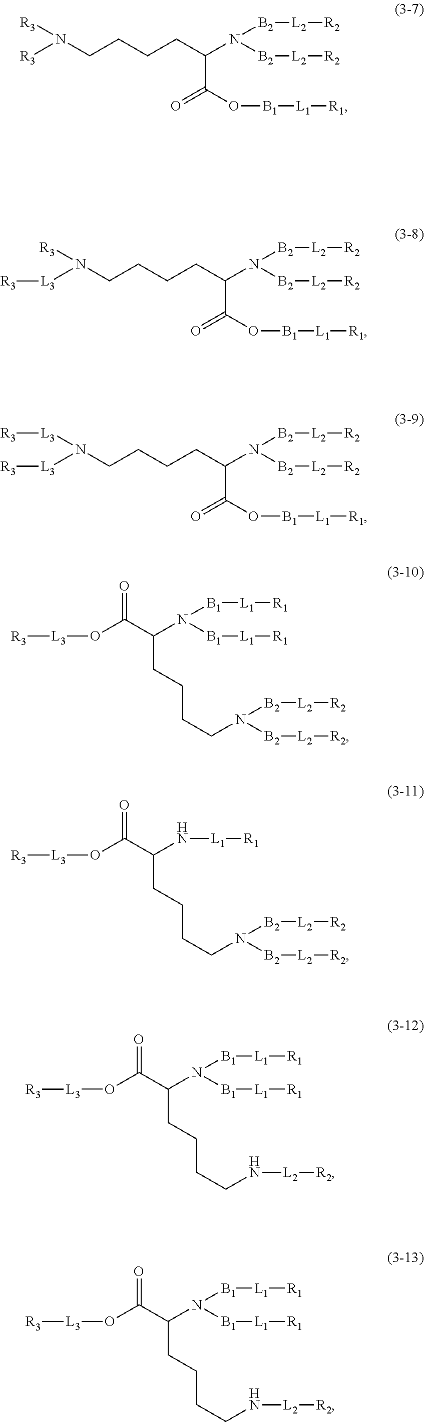

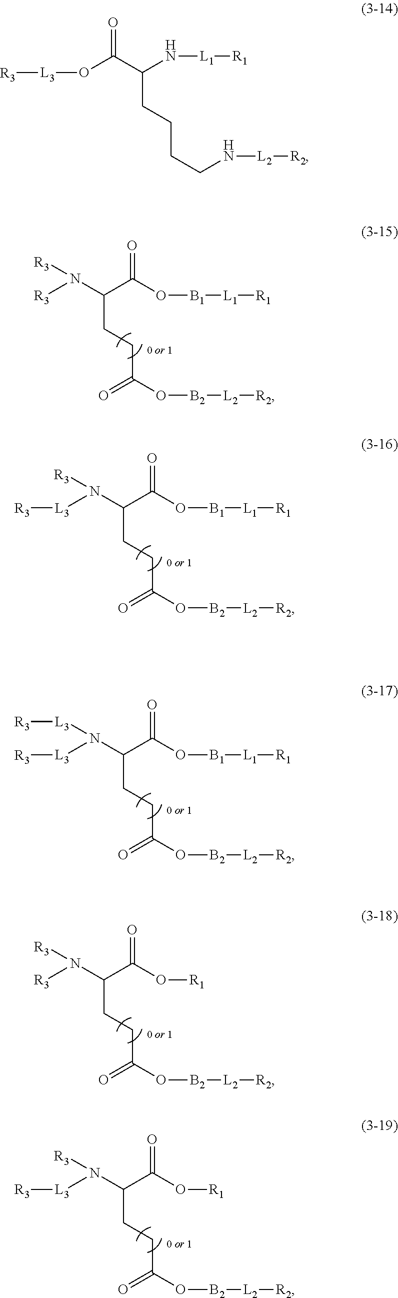

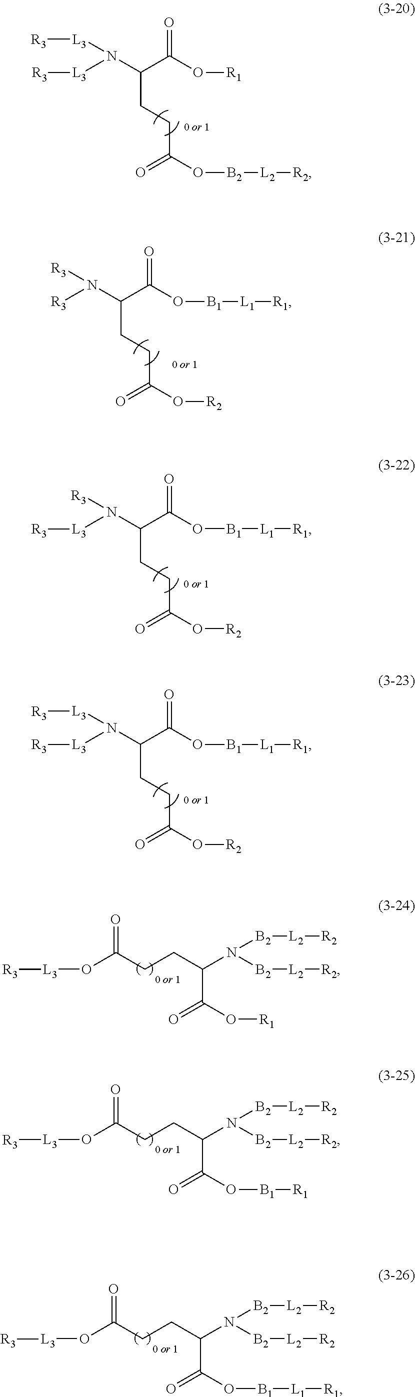

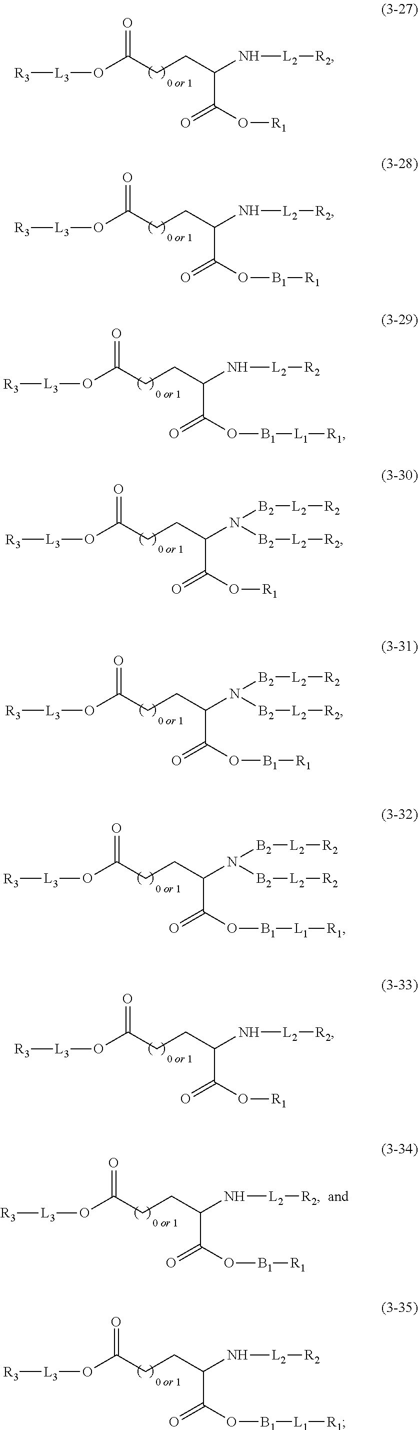

wherein, B₁, B₂, L₁, L₂, L₃, R₁, R₂ and R₃ are defined as described in the general formula (1) and will not be repeated here, and none of B₁, B₂, L₁, L₂, and L₃ is a linking bond; more preferably L₁ and L₂ are each independently selected from the group consisting of —C(═O)—, —C(═O)O—, —OC(═O)—, —OC(═O)O—, —C(═O)NH—, —NHC(═O)—, —OC(═O)NH—, and —NHC(═O)O— at each occurrence; more preferably L₁ and L₂ are each independently selected from the group consisting of —C(═O)—, —C(═O)O—, —OC(═O)— and —OC(═O)O-.

### 1.7. Examples of Specific Structures

In one embodiment of the present application, the structure of amino acid-based cationic lipid is preferably selected from the group consisting of the following structures:

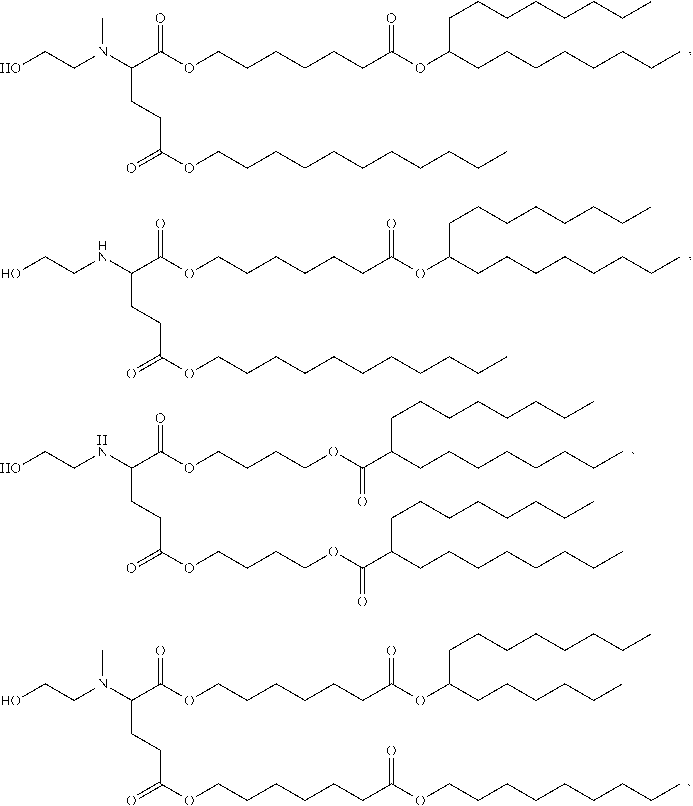

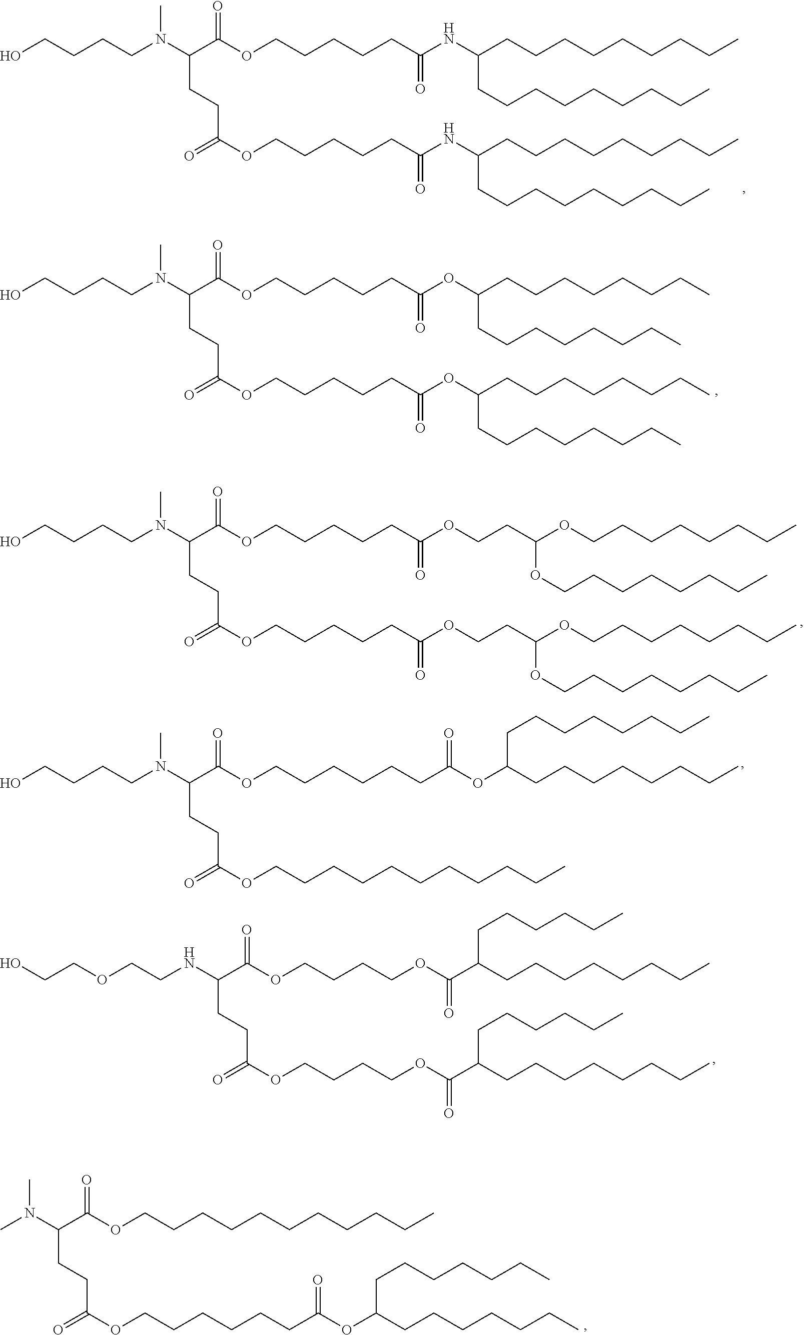

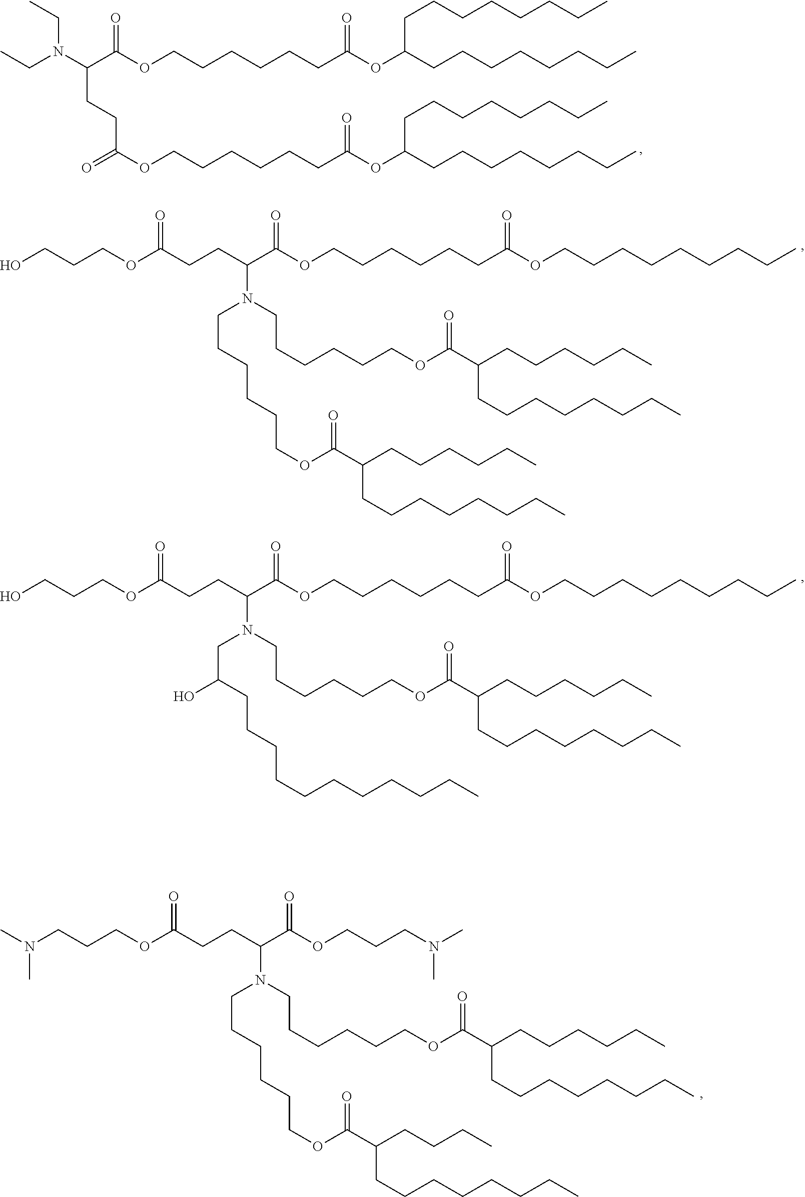

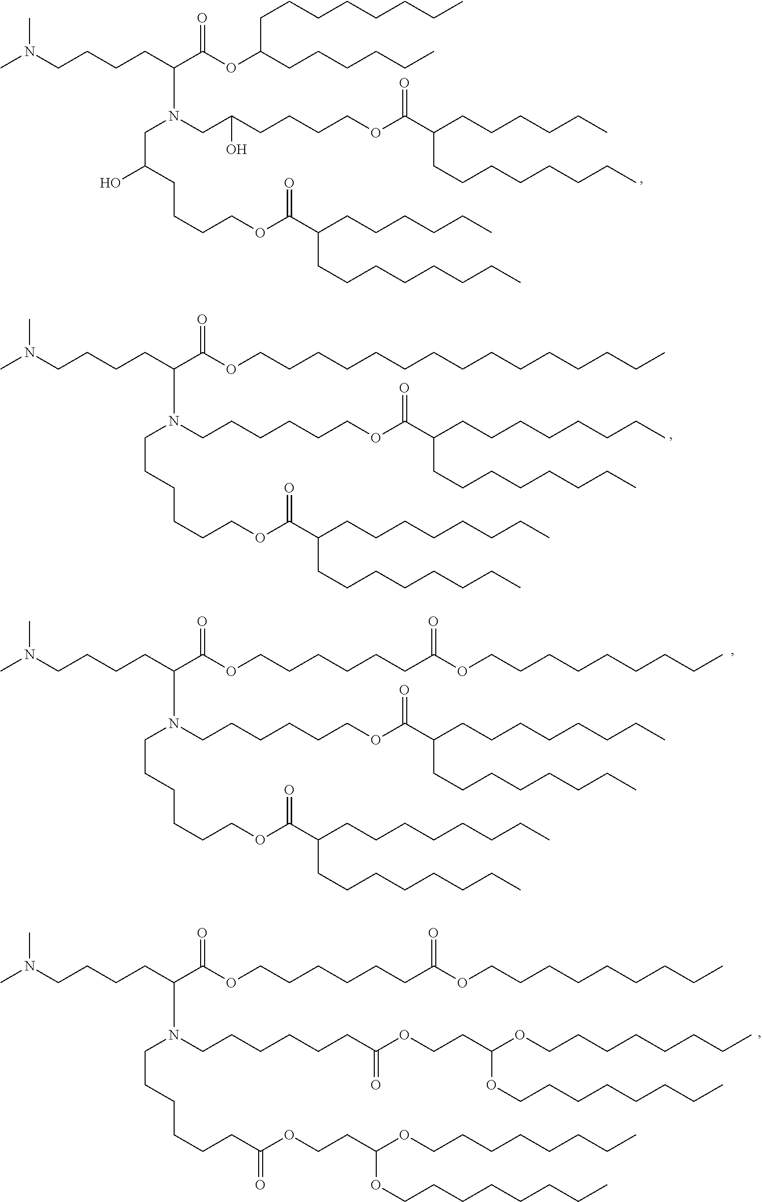

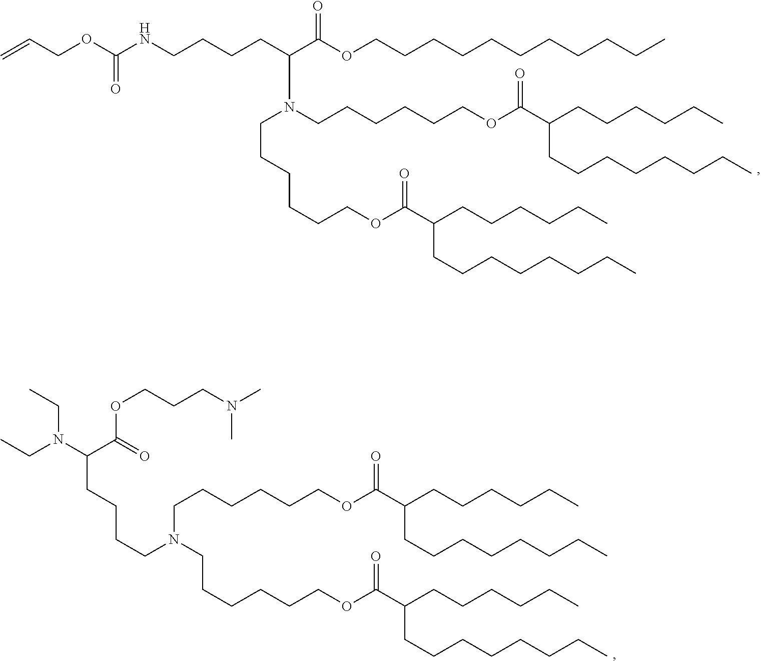

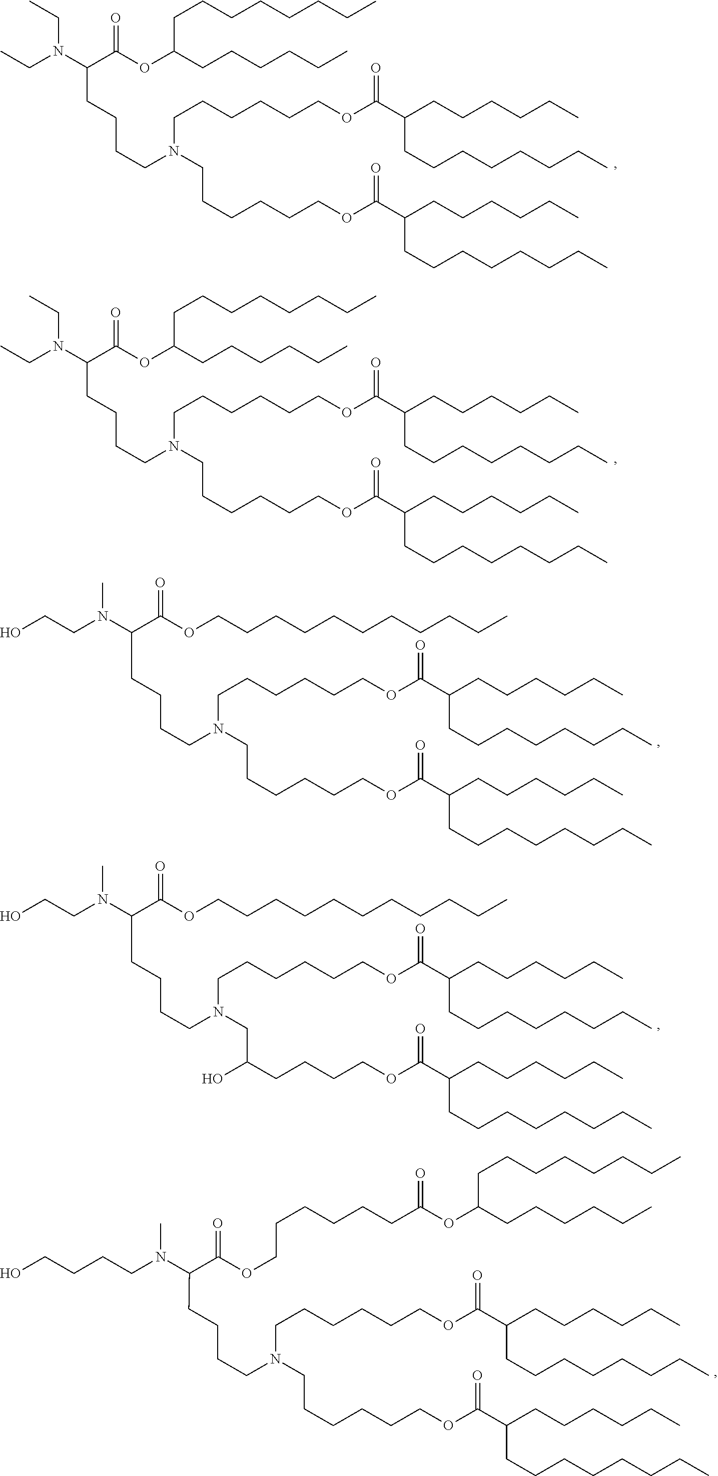

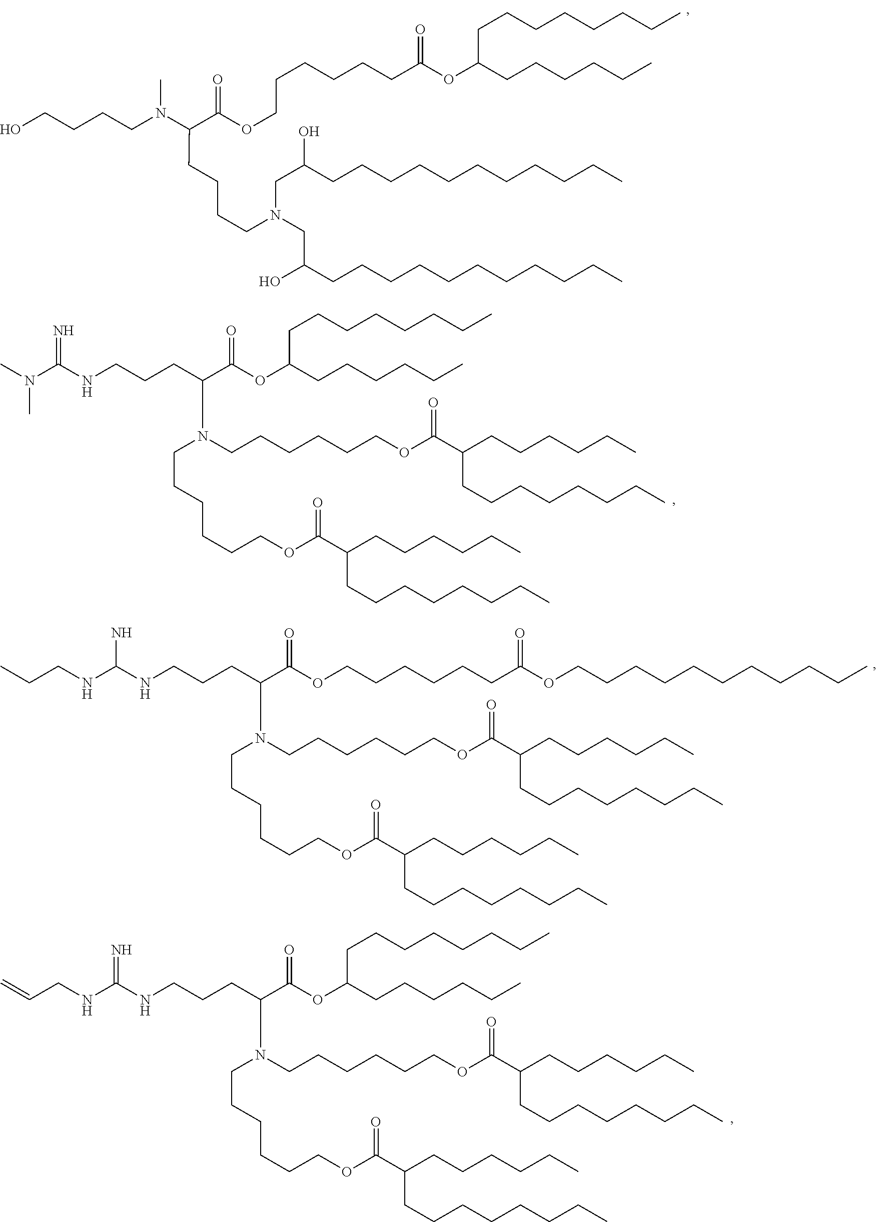

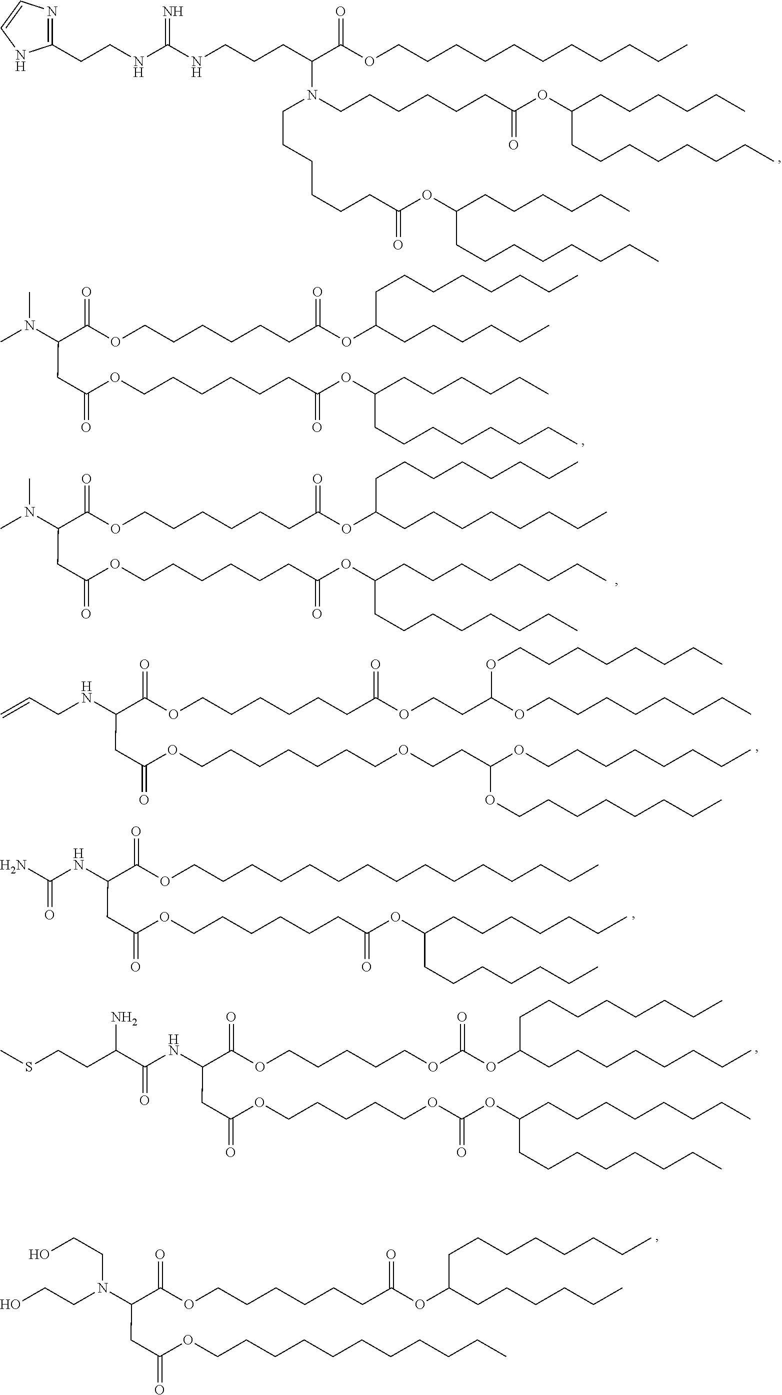

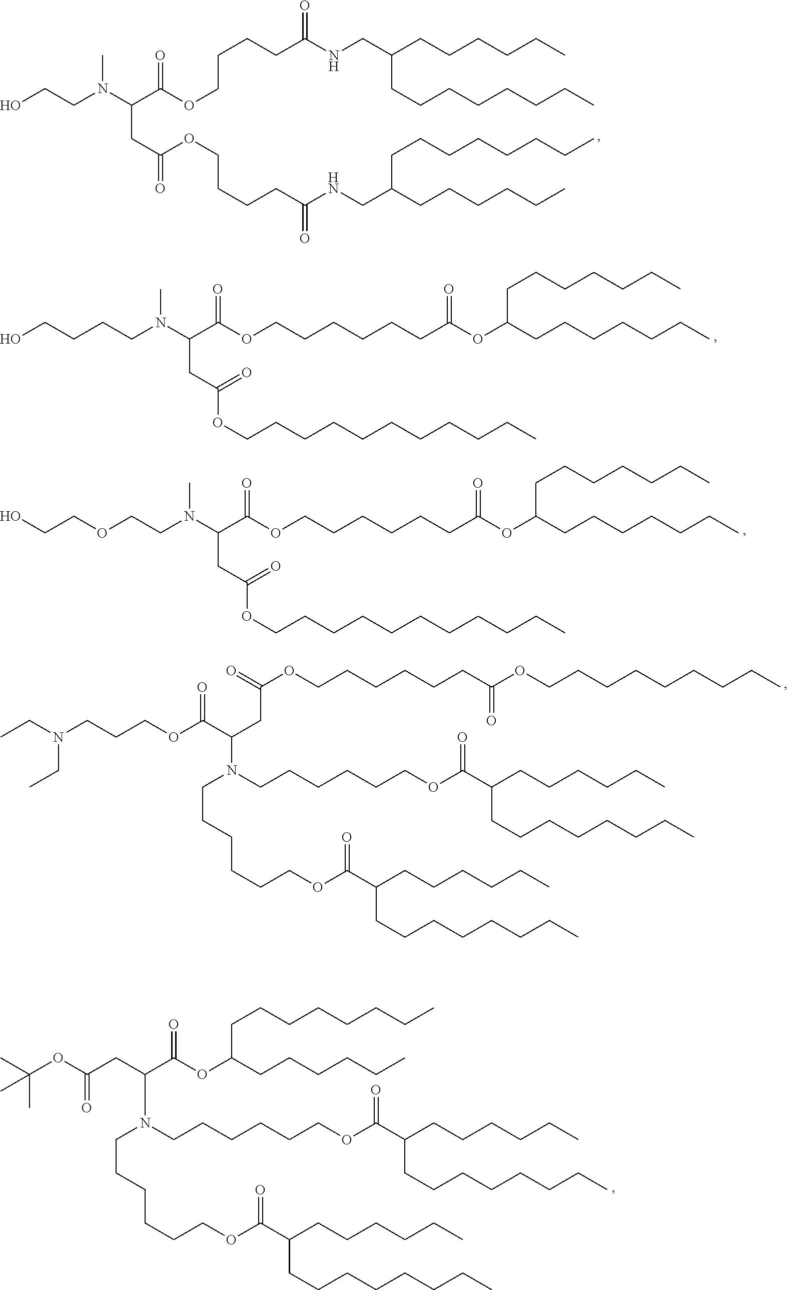

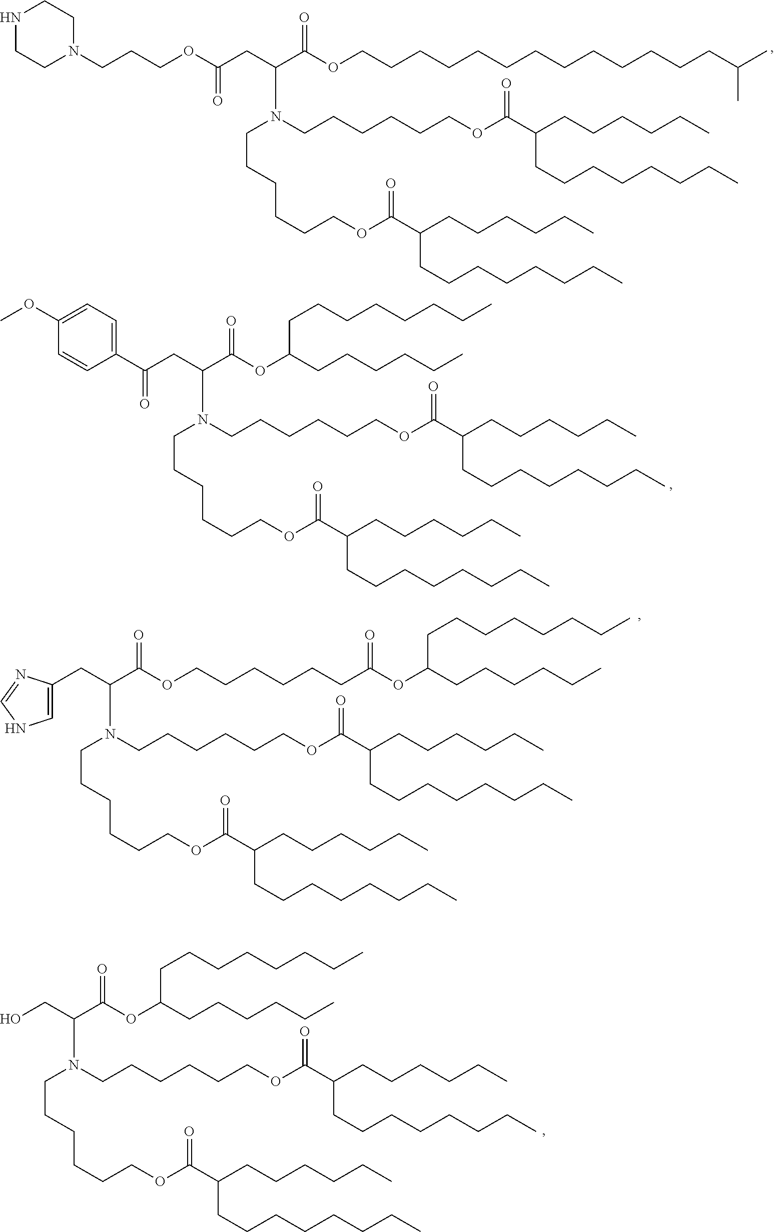

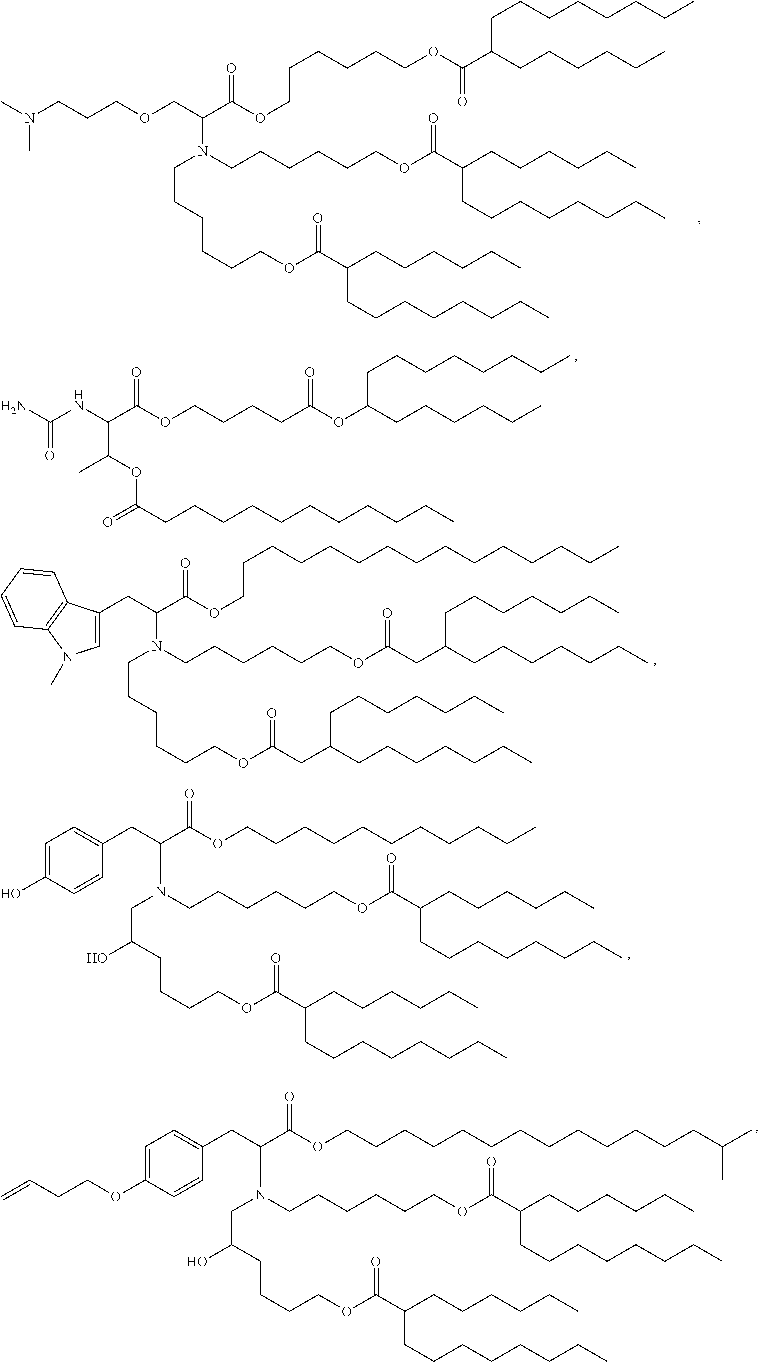

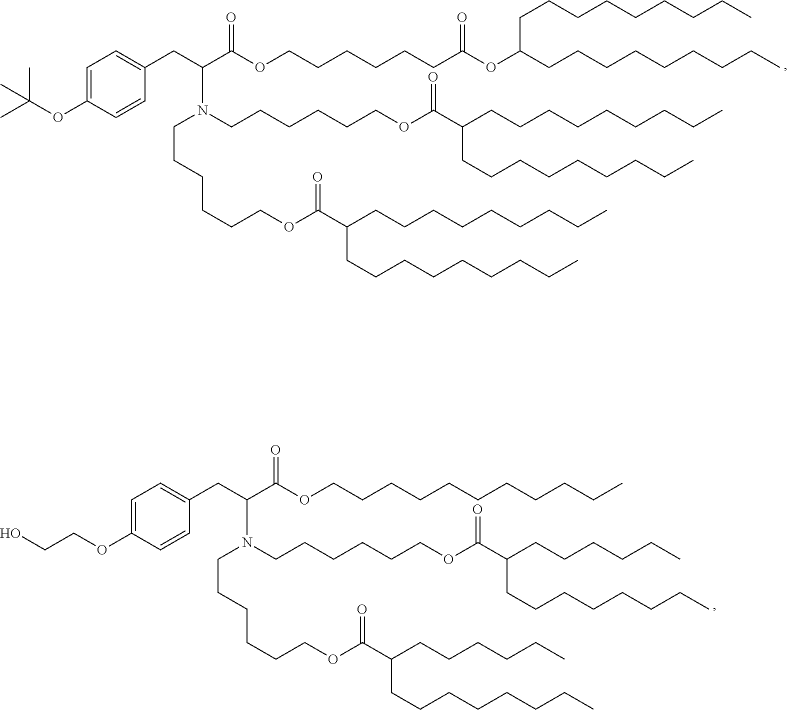 [SMILES: CCCCCCCCCC(CCCCCCCCC)OC(=O)CCCCCCOC(=O)C(Cc1ccc(OC(C)(C)C)cc1)N(CCCCCCOC(=O)C(CCCCCCCCC)CCCCCCCCC)CCCCCCOC(=O)C(CCCCCCCCC)CCCCCCCCC ; CCCCCCCCCCCOC(=O)C(Cc1ccc(OCCO)cc1)N(CCCCCCOC(=O)C(CCCCCC)CCCCCCCC)CCCCCCOC(=O)C(CCCCCC)CCCCCCCC]

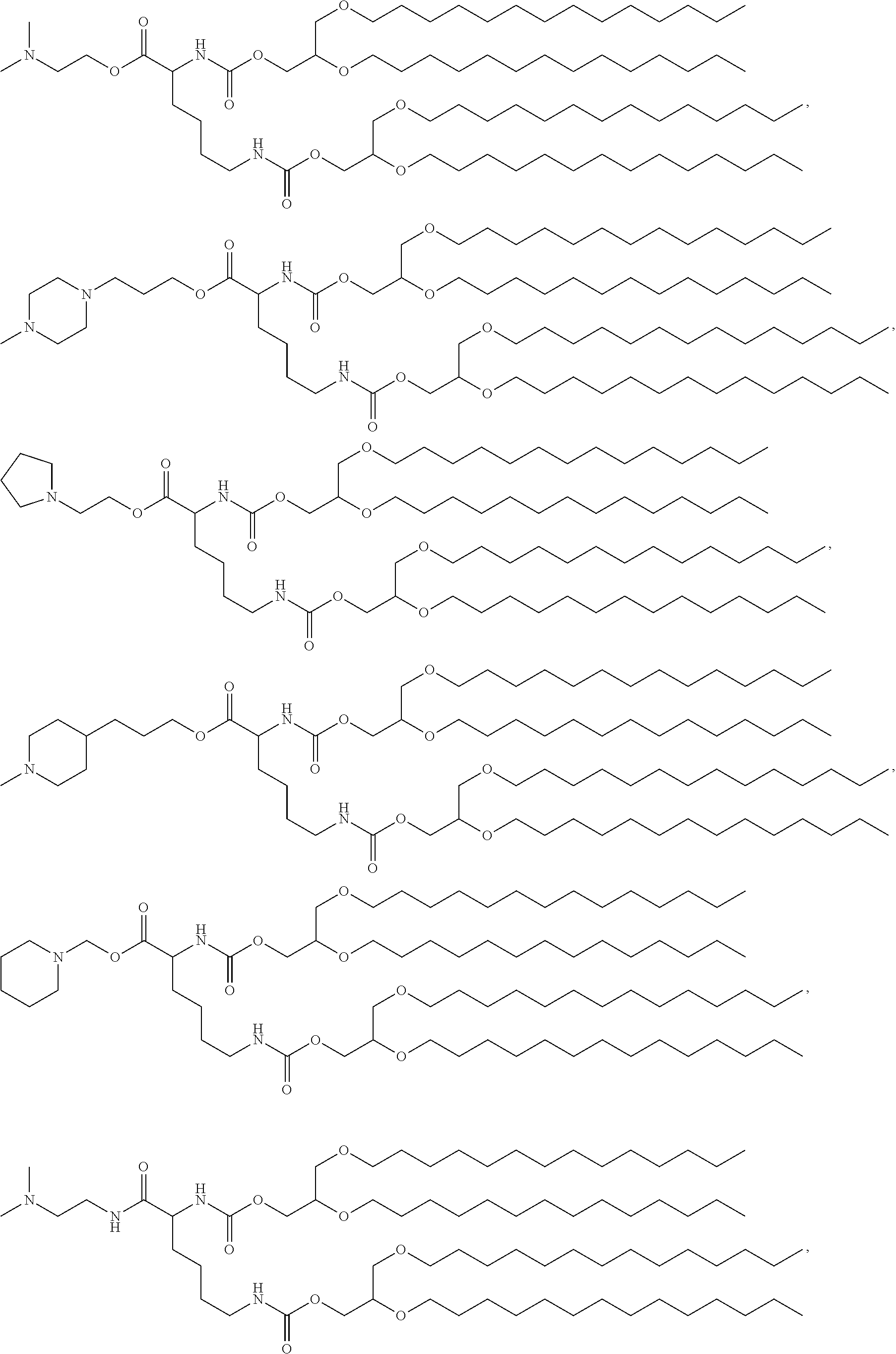

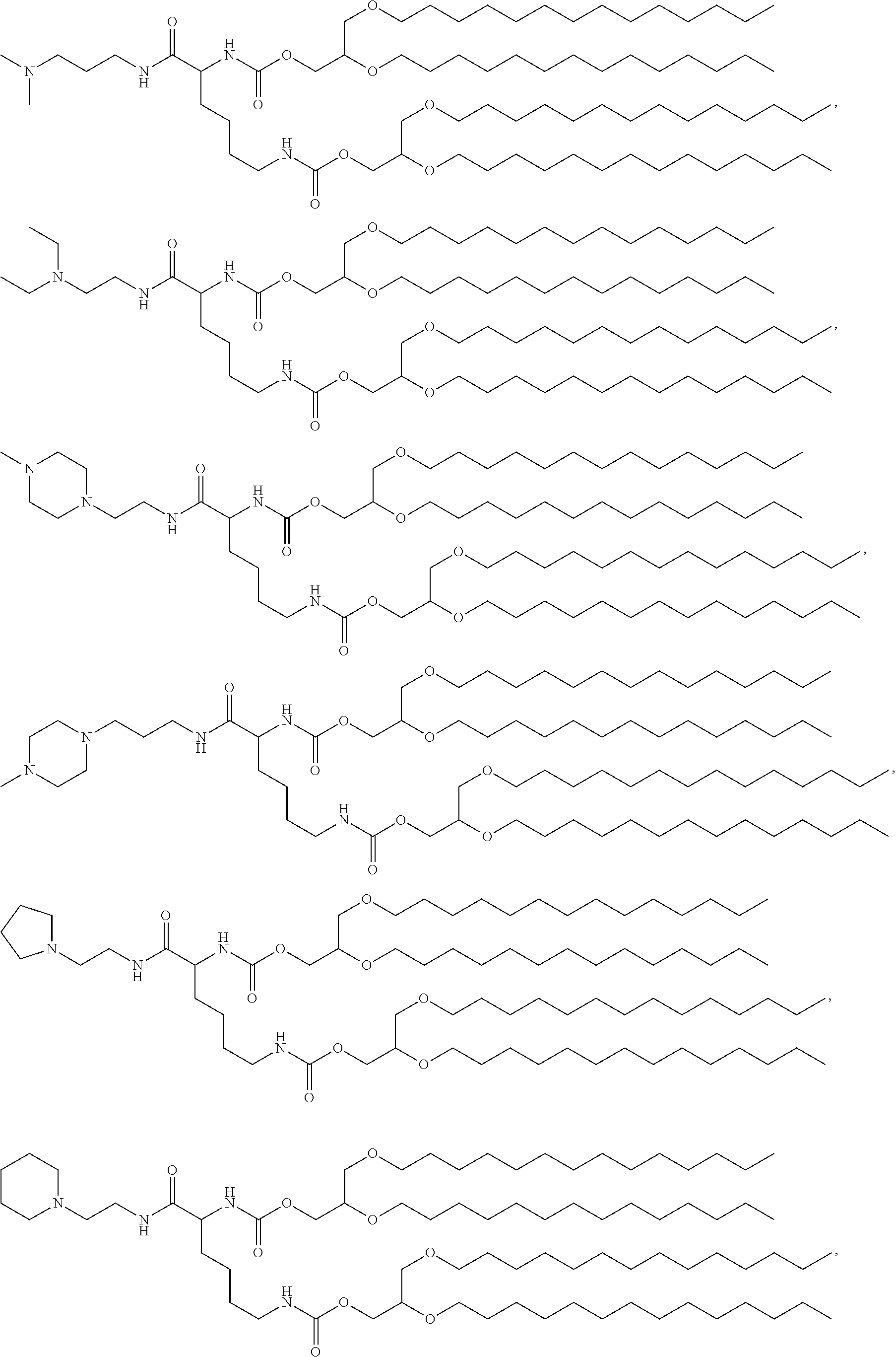

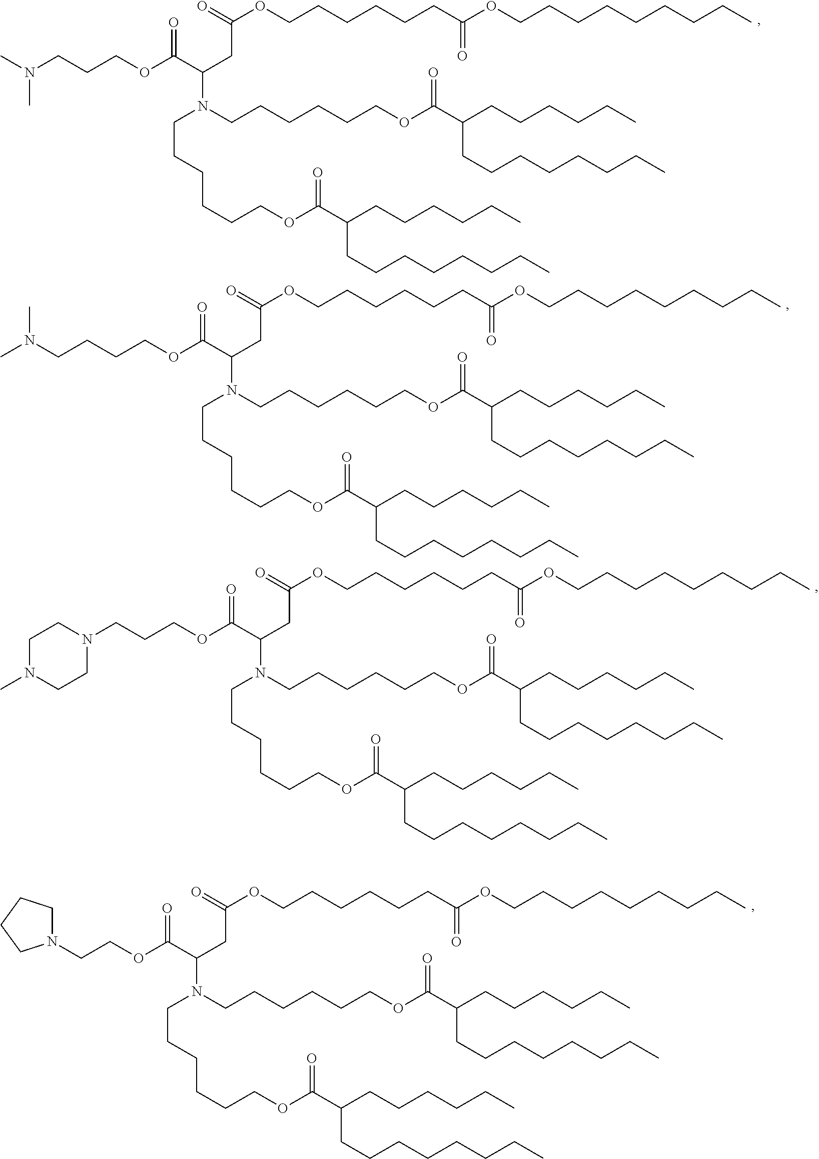

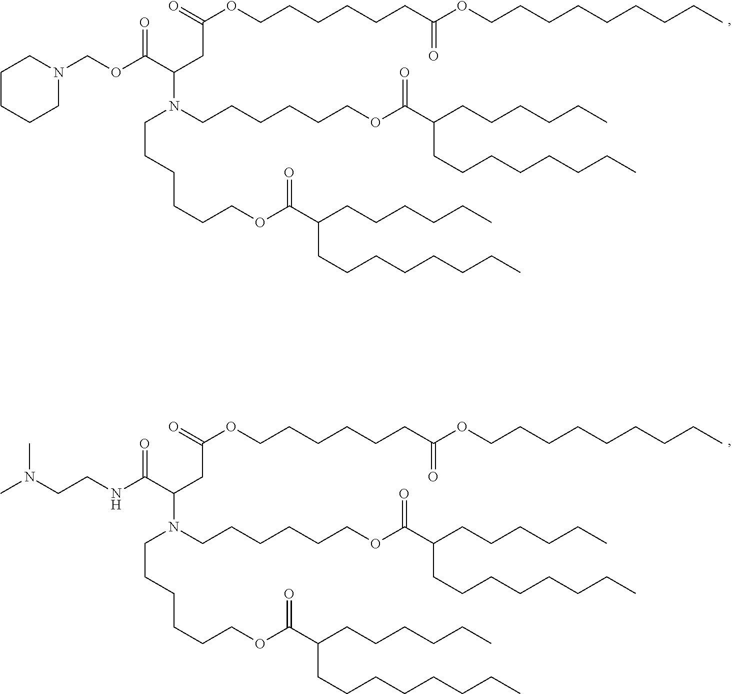

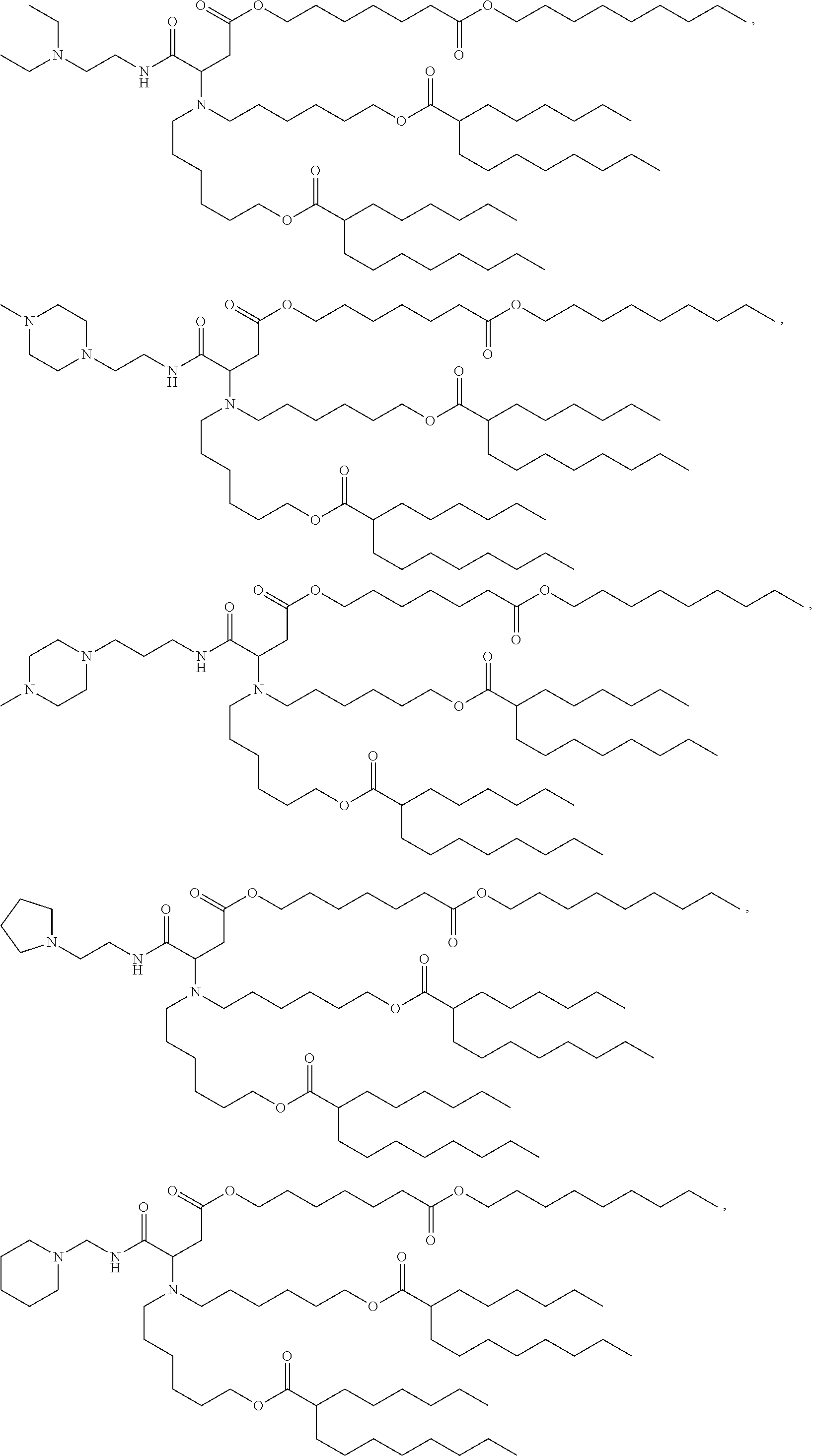

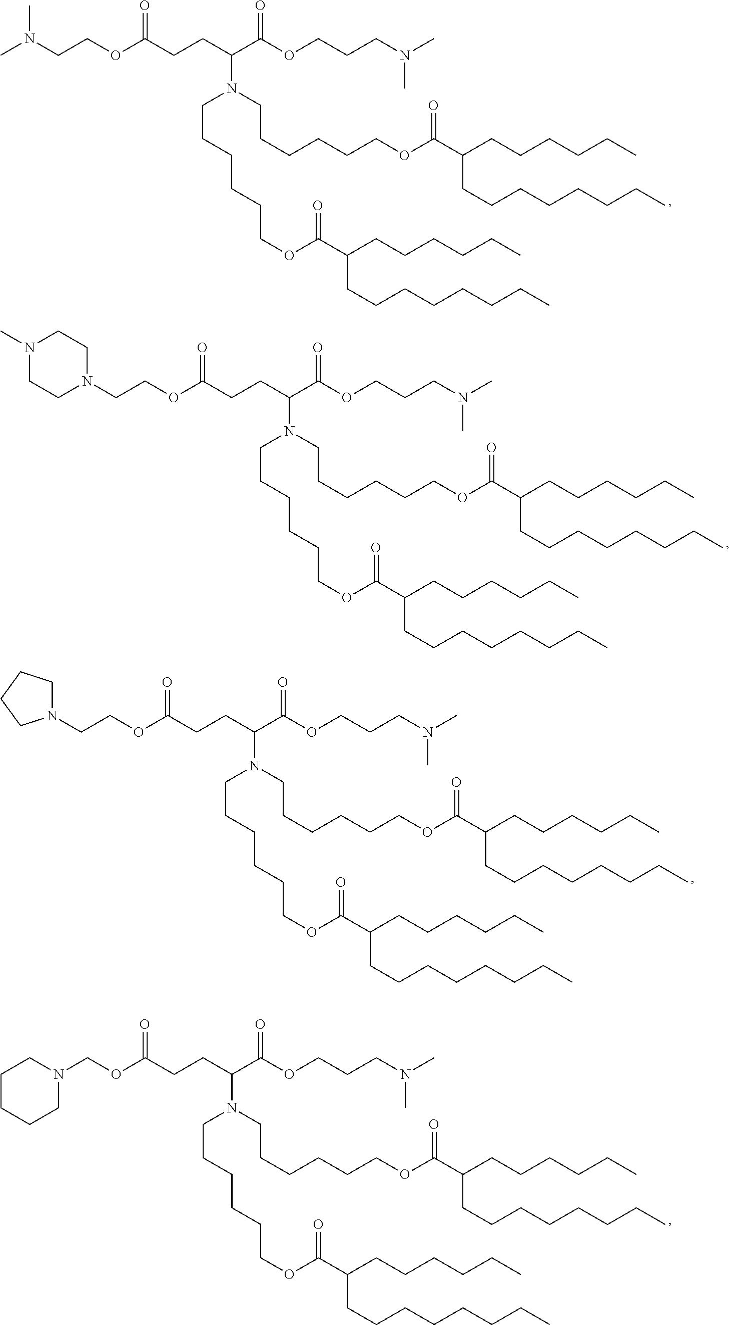

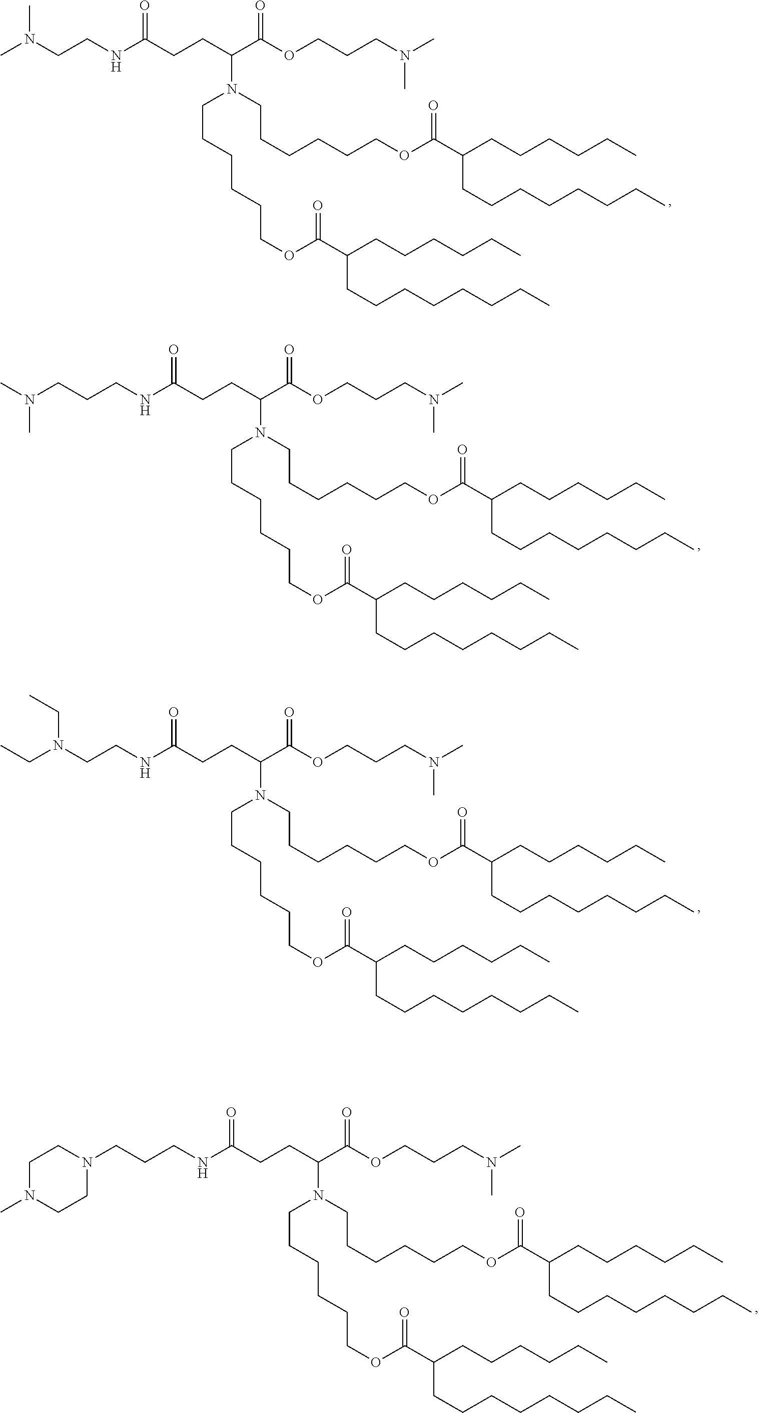

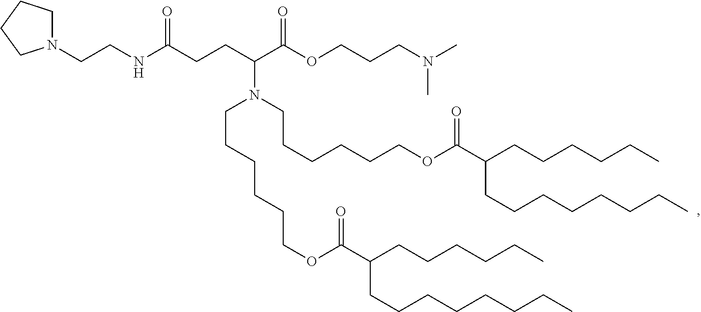 [SMILES: CCCCCCCCC(CCCCCC)C(=O)OCCCCCCN(CCCCCCOC(=O)C(CCCCCC)CCCCCCCC)C(CCC(=O)NCCN1CCCC1)C(=O)OCCCN(C)C]

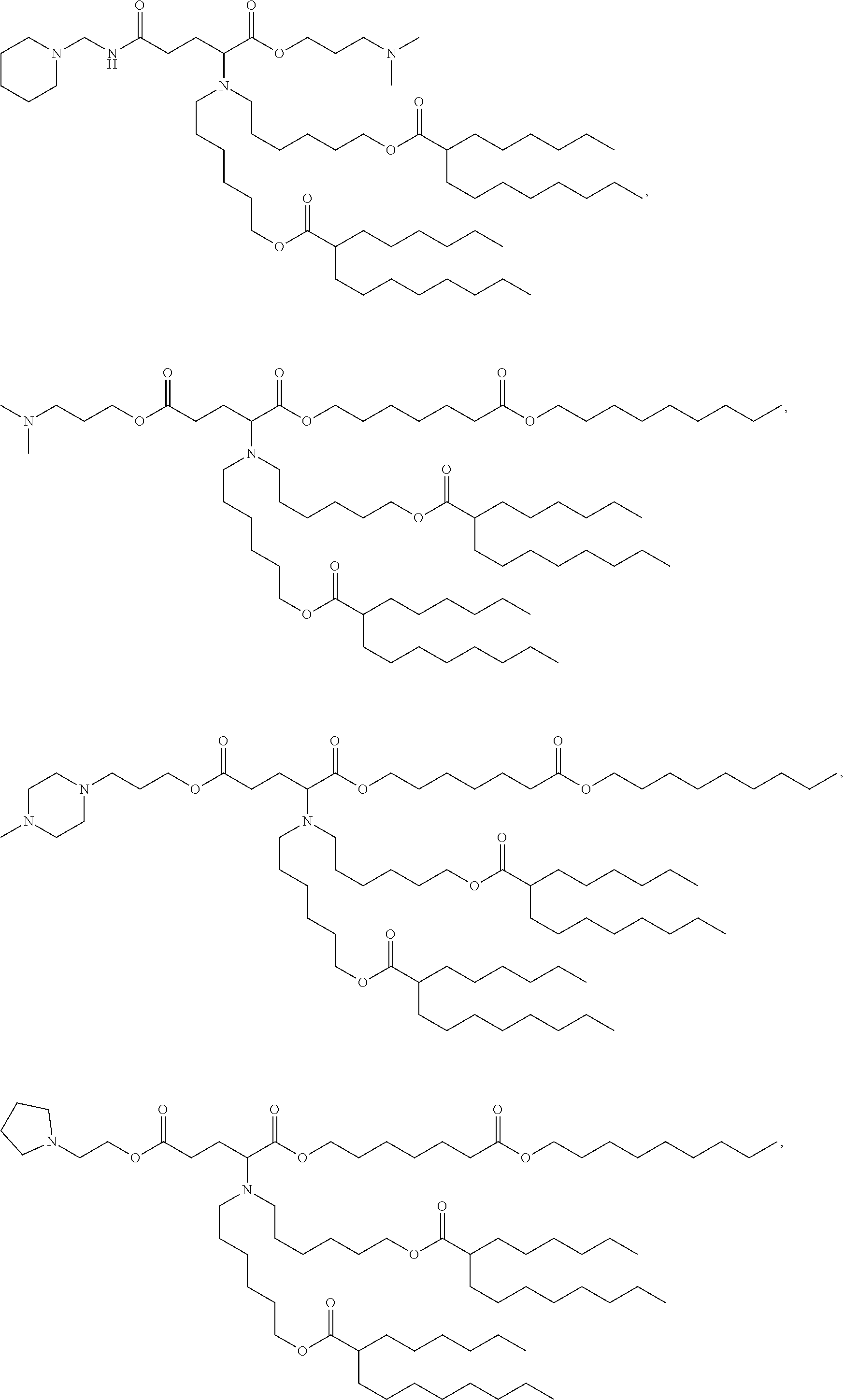

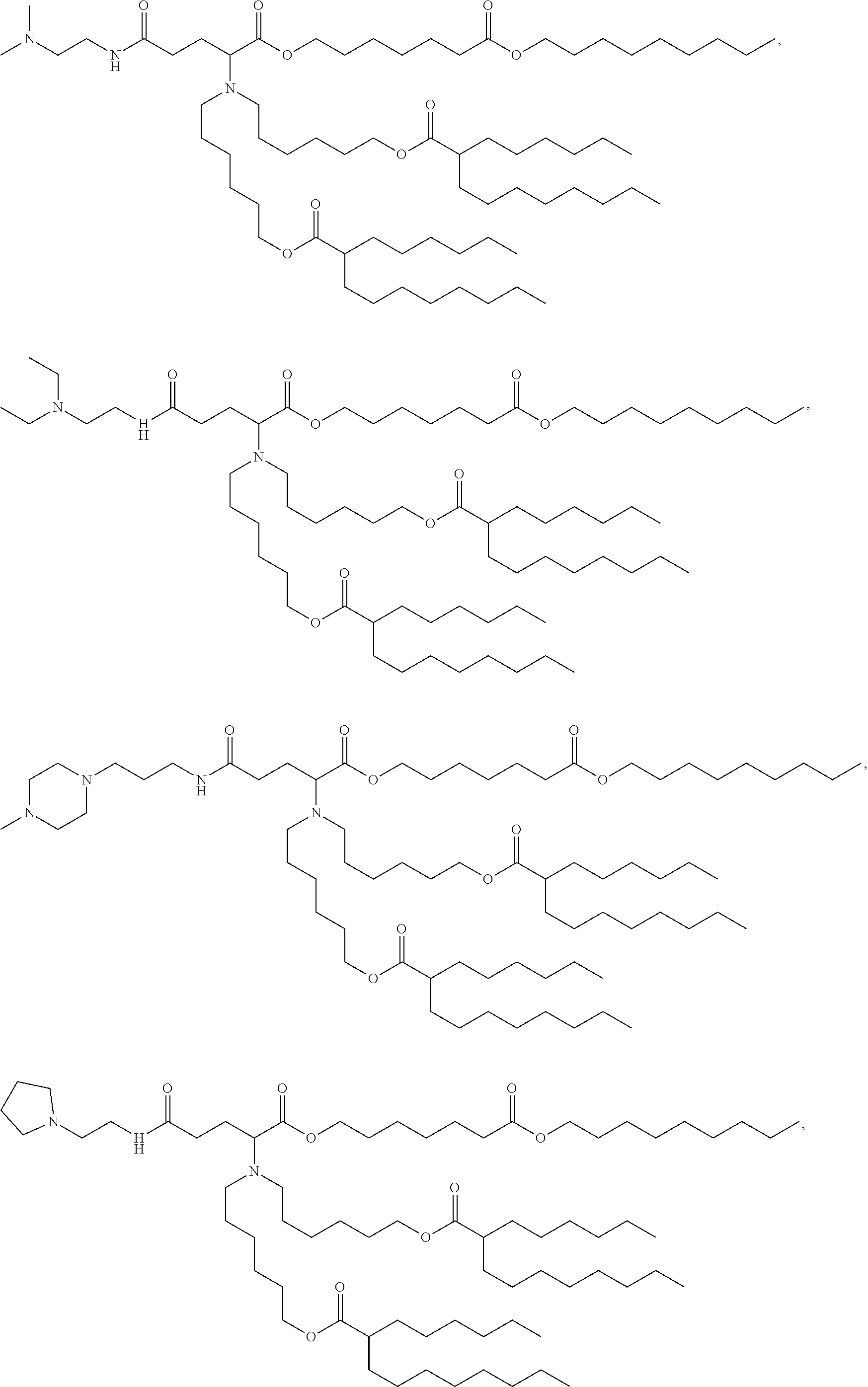

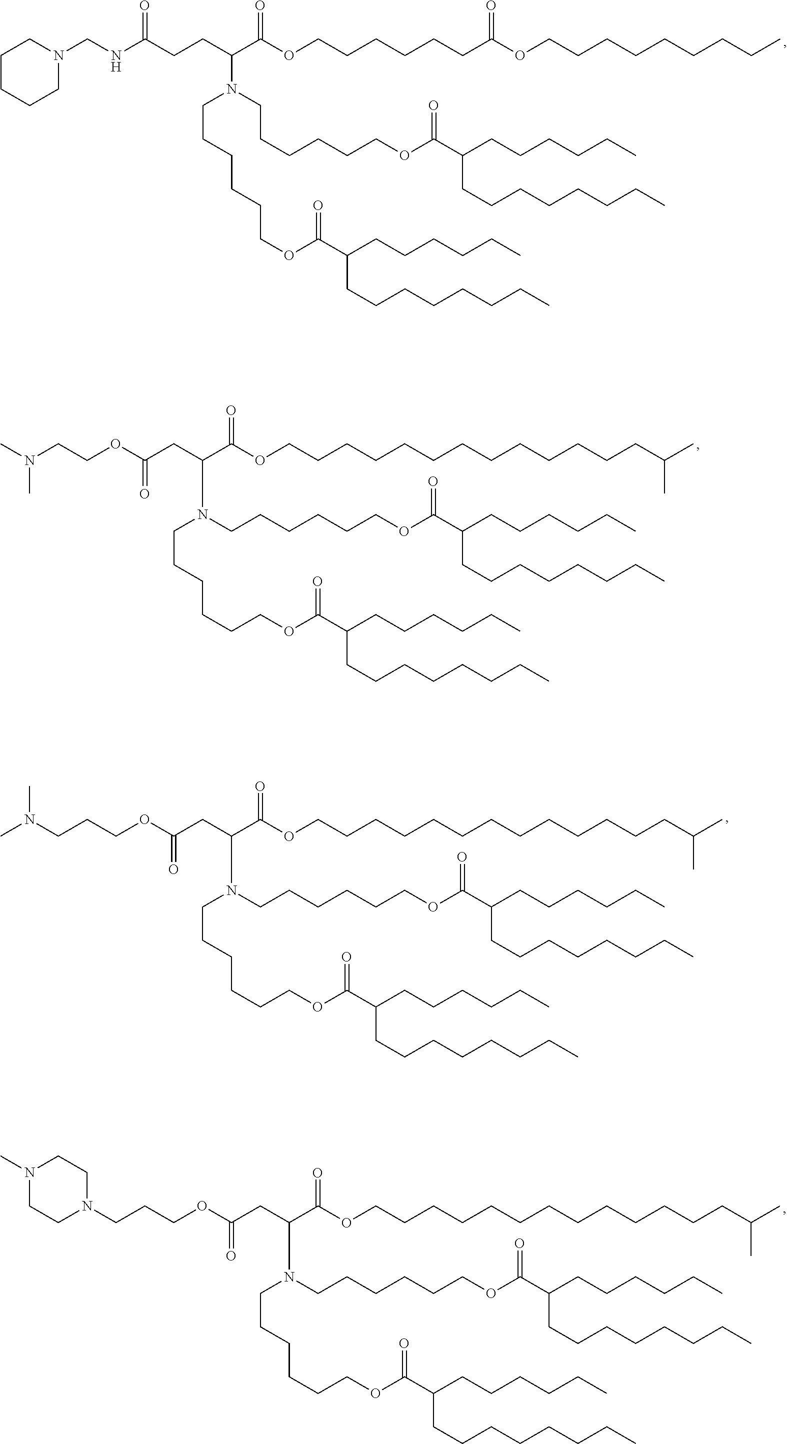

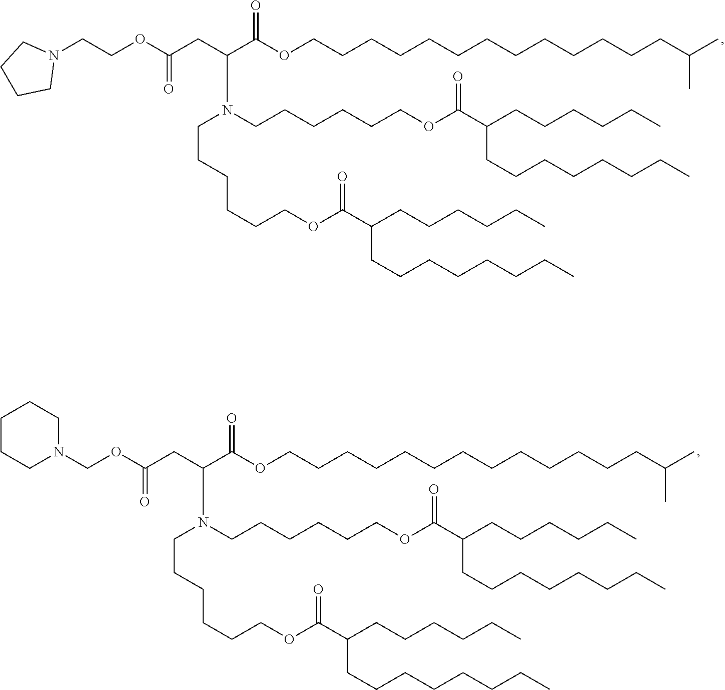 [SMILES: CCCCCCCCC(CCCCCC)C(=O)OCCCCCCN(CCCCCCOC(=O)C(CCCCCC)CCCCCCCC)C(CC(=O)OCCN1CCCC1)C(=O)OCCCCCCCCCCCCCC(C)C ; CCCCCCCCC(CCCCCC)C(=O)OCCCCCCN(CCCCCCOC(=O)C(CCCCCC)CCCCCCCC)C(CC(=O)OCN1CCCCC1)C(=O)OCCCCCCCCCCCCCC(C)C]

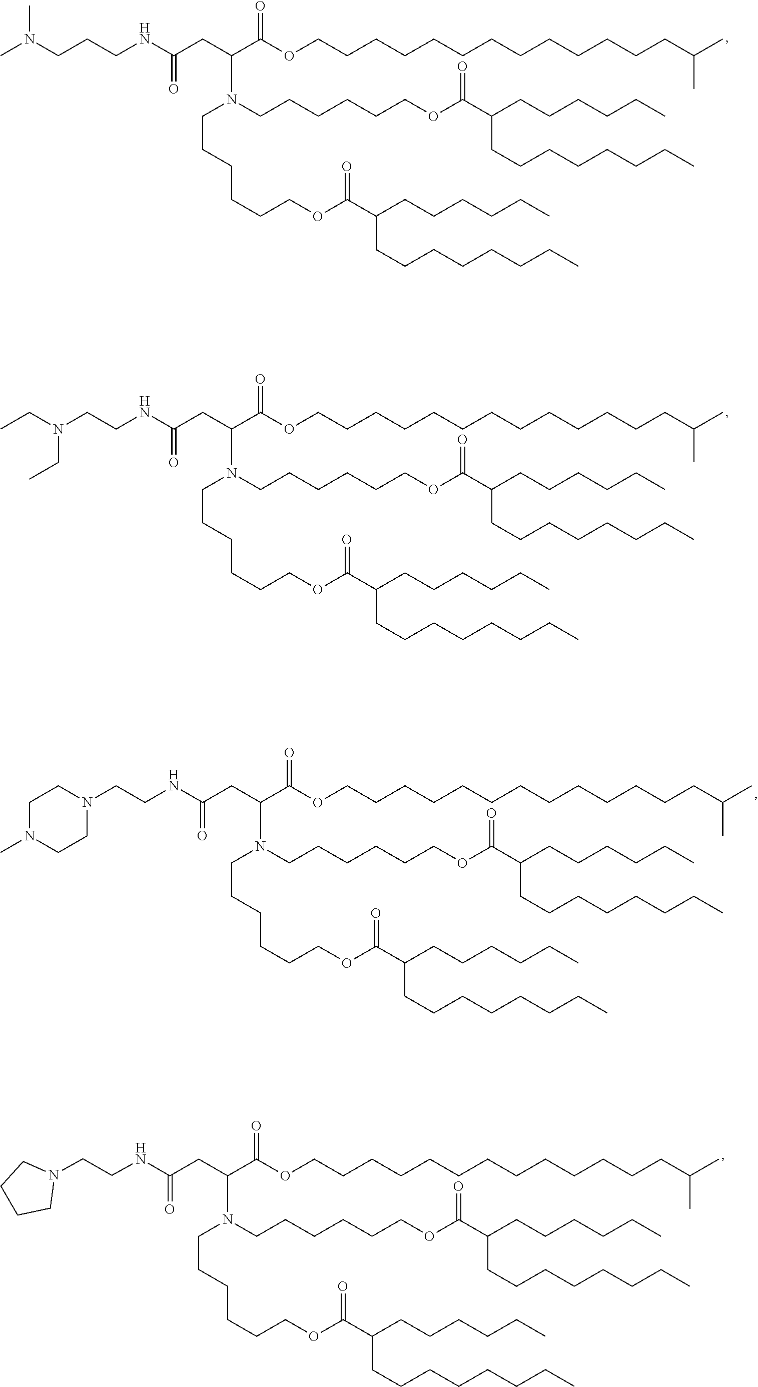

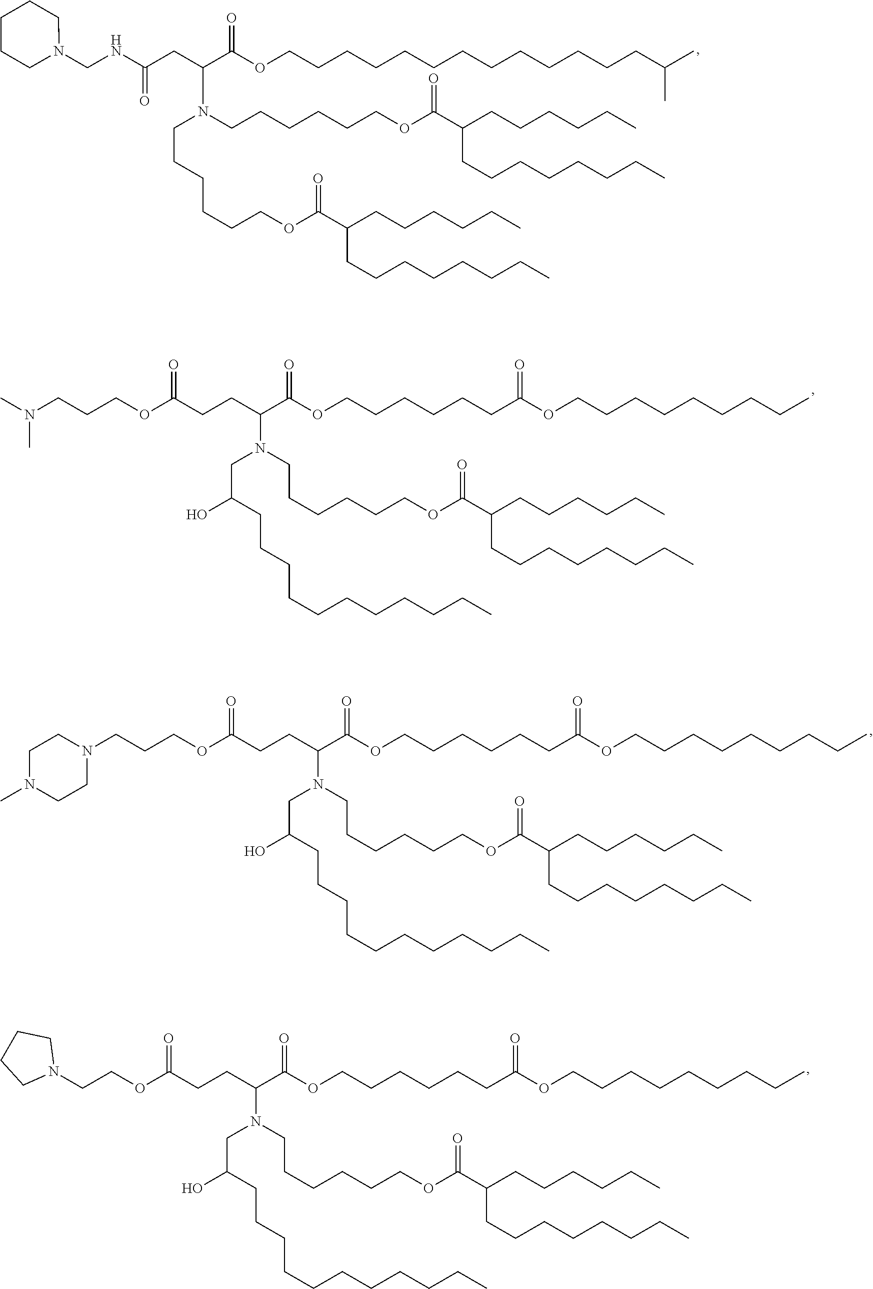

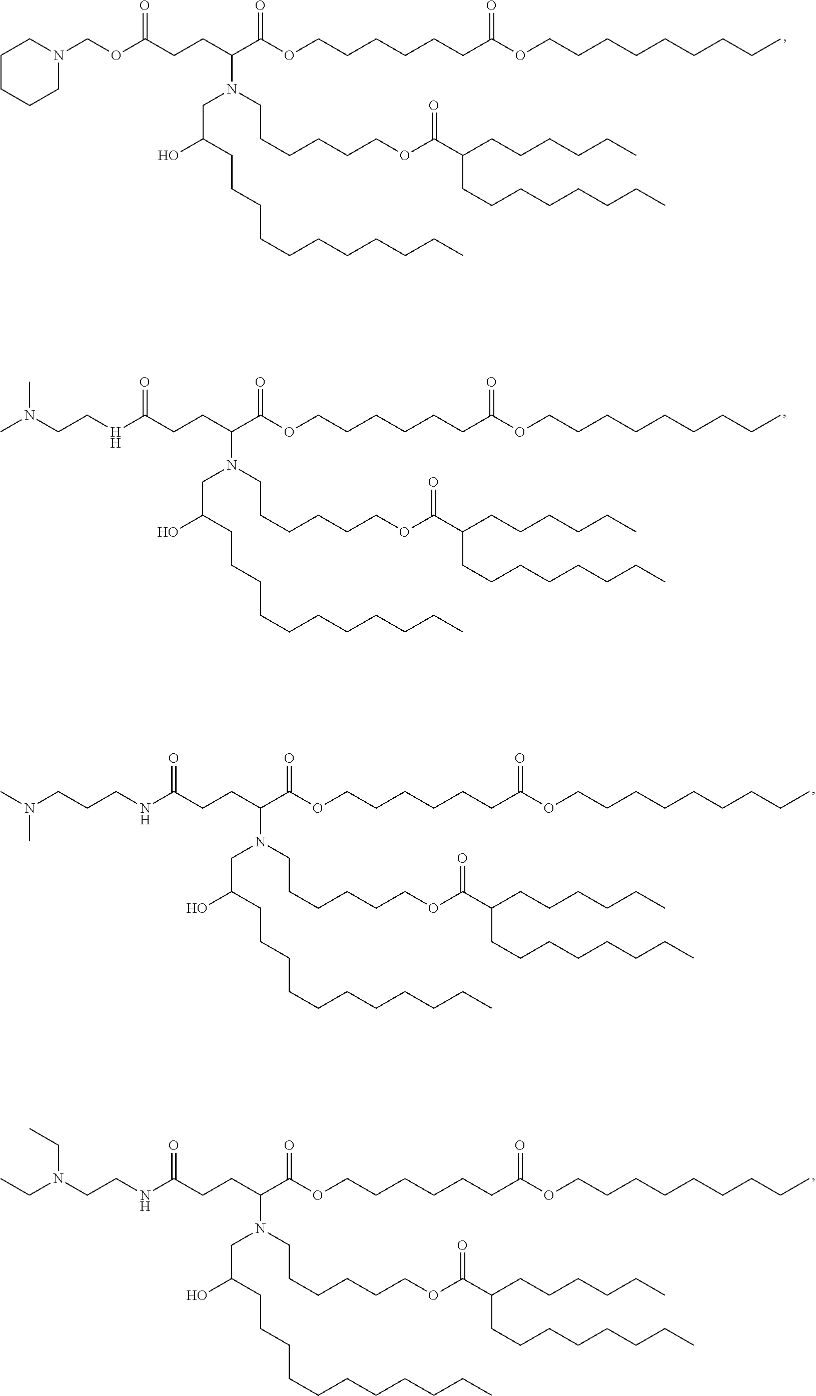

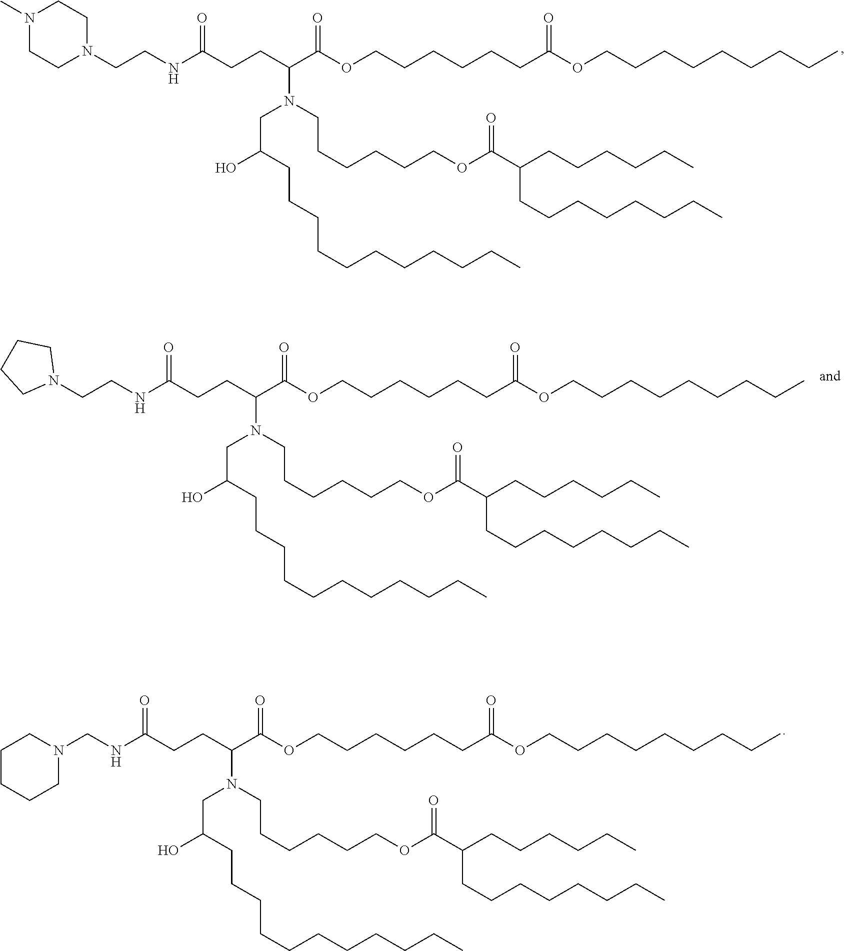 [SMILES: CCCCCCCCCCCC(O)CN(CCCCCCOC(=O)C(CCCCCC)CCCCCCCC)C(CCC(=O)NCCN1CCN(C)CC1)C(=O)OCCCCCCC(=O)OCCCCCCCCC ; CCCCCCCCCCCC(O)CN(CCCCCCOC(=O)C(CCCCCC)CCCCCCCC)C(CCC(=O)NCCN1CCCC1)C(=O)OCCCCCCC(=O)OCCCCCCCCC ; CCCCCCCCCCCC(O)CN(CCCCCCOC(=O)C(CCCCCC)CCCCCCCC)C(CCC(=O)NCN1CCCCC1)C(=O)OCCCCCCC(=O)OCCCCCCCCC]

### 2. Preparation of Amino Acid-Based Cationic Lipids

In the present application, any of the aforementioned amino acid-based cationic lipids can be prepared by methods including but not limited to the following:

### 2.1. Method-1:

Step 1: one molecule of A-1 (AA₁) is reacted with one molecule or two molecules of the same or different A-2 (R₃ [symbol] ₁) to obtain a small molecule intermediate A-3

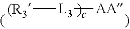

containing a divalent linking group L₃; wherein, the small molecule A-1 is an amino acid or an amino acid derivative, and A-1 contains one, two or three identical or different amino acid end groups. The small molecule A-2 contains a reactive group F₁, which can react with the amino acid end group of AA₁ to form a divalent linking group L₃, and F₁ is preferably —OH, —COOH, —NH₂, —Br, —CHO, —OMs, —COCl, etc., and R₃ [symbol] R₃ or a micro variant form containing R₀₁; AA [symbol] is an amino acid derivative residue containing two identical or different amino acid end groups;
Step 2: small molecule A-4 (R₂—F₂) is reacted with small molecule A-5 (F₃—B₂-F_{N}) to obtain a small molecule intermediate A-6 (R₂-L₂-B₂-F_{N}) containing a divalent linking group L₂, a reactive group F_{N} at one end and R₂ at the other end. Wherein, the small molecule A-4 contains the reactive group F₂, and the small molecule A-5 contains a pair of heterofunctional groups of F₃ and F_{N}; F₂ can react with F₃ to form a divalent linking group L₂, and F_{N} is a reactive group that can react with a carboxyl group, a hydroxyl group, and an amino group, and preferably —OH, —COOH, —NH₂, —F, —Cl, —Br, —COCl,

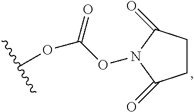 [SMILES: *OC(=O)ON1C(=O)CCC1=O]

etc.
Step 3: one molecule or two molecules of the same or different small molecule intermediate A-6 (R₂-L₂-B₂-F_{N}) reacts with small molecule intermediate A-3

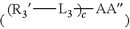

to obtain the amino acid derivative A-7

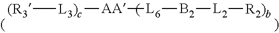

containing a divalent linking group L₆; wherein, AA [symbol] an amino acid derivative residue containing one amino acid end group;
Step 4: the amino acid derivative A-7

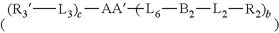

reacts with one or two molecules of small molecule intermediate A-8 (R₁-L₁-B₁-FC, synthesized according to the method of step 2 or obtained by purchase) to obtain the amino acid-based cationic lipid A-9□

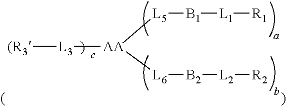

containing a divalent linking group L₅; wherein, the small molecule A-8 contains the FC group that can react with an amino acid end group (such as a carboxyl group, a hydroxyl group, a thiol group or an amino group), preferably being -OH, —COOH, —NH₂, —Br, or

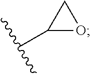 [SMILES: *C1CO1]

when R₃ □ is equal to R₃, the resulting structure A-9 corresponds to the structure represented by general formula (1);
when R₃ [symbol] s not equal to R₃, A-9 [symbol] s micromodified at the terminal group to obtain A-9, [symbol] which corresponds to the structure represented by general formula (1); the terminal micro-modification is selected from the following chemical reactions: deprotection, salt complexation and decomplexation, ionization, protonation, deprotonation, and leaving group transformation;
wherein, the definitions of AA, L₁, L₂, L₃, L₅, L₆, B₁, B₂, R₃, R₁, R₂, a, b and c are consistent with those described in general formula (1), and will not be repeated here.

The aforementioned small molecule starting materials A-1, A-2, A-3, A-4, A-5, A-8, etc. can be obtained by purchase or synthesis. For example, the small molecule A-3 in Example 1 is

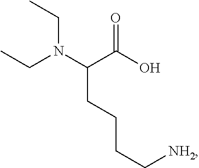

which can be independently synthesized by using lysine with a Boc protected amino group

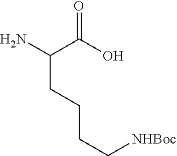 [SMILES: CC(C)(C)OC(=O)NCCCCC(N)C(=O)O]

and bromoethane

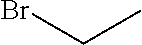

as starting materials, and then obtained after removing the Boc group; the small molecule A-3 in Example 6 is

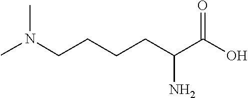

which can be obtained by purchase, so step 1 can be omitted; in Example 8, A-3 is

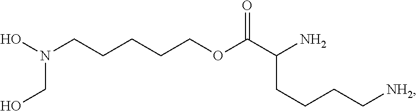 [SMILES: NCCCCC(N)C(=O)OCCCCCN(O)CO]

which can be obtained by reacting and then deprotecting with

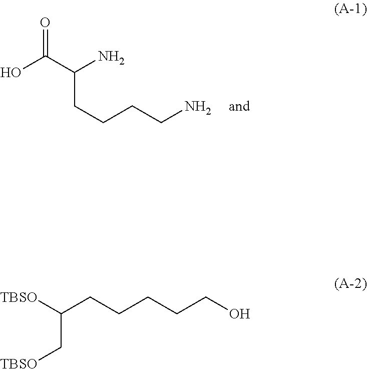 [SMILES: NCCCCC(N)C(=O)O ; CC(C)(C)[Si](C)(C)OCC(CCCCCO)O[Si](C)(C)C(C)(C)C]

as starting materials for reaction deprotection.

Step 1:

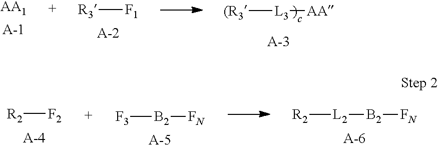

Step 3:

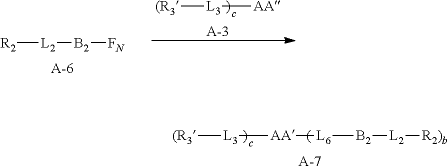

Step 4:

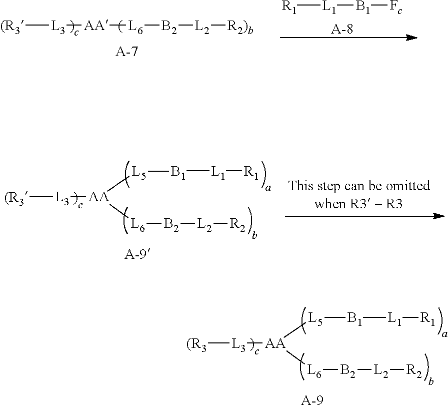

In this method, when the two amino acid end groups contained in AA [symbol] in A-3

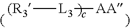

of step 3 are the same, A-3 can also react with two or four molecules of A-6 (R₂-L₂-B₂-F_{N}) at the same time to obtain A-10 [symbol] at this time, L₅ and L₆ are the same, B₁ and B₂ are the same, L₁ and L₂ are the same, R₁ and R₂ are the same, a and b are the same, L₁, L₂, L₃, L₅, L₆, B₁, B₂, R₃, R₁, R₂, a, b and c are defined as described in general formula (1), and will not be repeated here; F_{N}, AA [symbol] R₃ [symbol] s consistent with the foregoing, and will not be repeated here;
when R₃ □s equal to R₃, the resulting structure A-10□corresponds to the structure represented by general formula (1);
when R₃ [symbol] s not equal to R₃, A-10 [symbol] s micromodified at the terminal to obtain the A-10 which corresponds to the structure represented by general formula (1); the terminal micro-modification is selected from the following chemical reactions: deprotection, salt complexation and decomplexation, ionization, protonation, deprotonation, and leaving group transformation.

In aforementioned preparation steps, the reaction with the multi molecules can be carried out in a single step or in steps.

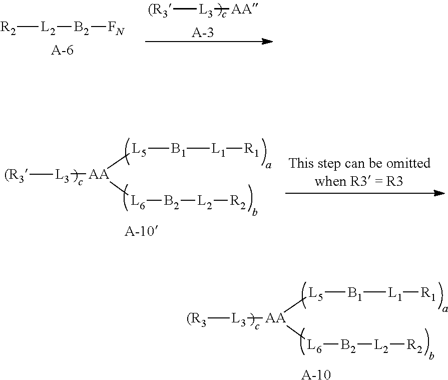

### 2.2. Method 2:

Step 1: one molecule of B-1 (AA₁) is reacted with one or two molecules of the same or different B-2 (R₂-L₂-B₂-F_{N}, synthesized according to the step 2 in 2.1. Method 1 or purchased) to obtain the intermediate B-3

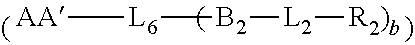

containing a divalent linking group L₆; wherein, small molecule B-1 is an amino acid or amino acid derivative containing 1, 2 or 3 identical or different amino acid end groups; small molecule B-2 contains a divalent linking group L₂ and reactive group F_{N}, which can react with the amino acid end group in B-1 to form a divalent linking group L₆, preferably being -OH, —COOH, —NH₂, —F, —Cl, —Br, —COCl,

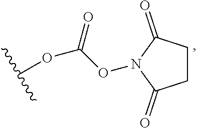 [SMILES: *OC(=O)ON1C(=O)CCC1=O]

etc.; AA [symbol] is an amino acid derivative residue containing two identical or different amino acid end groups.
Step 2: one molecule of intermediate B-3

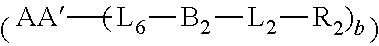

reacts with two molecules of the same or different B-4 (R₁-L₁-B₁-F_{C}, synthesized according to step 2 in 2.1.
Method 1 or purchased) to obtain intermediate B-5

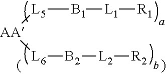

containing a divalent linking group L₅; wherein, the small molecule B-4 contains divalent linking group L₁ and reactive group F_{C}, which can react with the amino acid end group in B-3 to form a divalent linking group L₅, F_{C} is preferably —OH, —COOH, —NH₂, —F, —Cl, or —Br; AA^{□}is an amino acid derivative residue containing 1 amino acid end group.
Step 3: one molecule of intermediate B-5 reacts with one molecule or two molecules of the same or different B-6 (R₃—F₄) to obtain the amino acid-based cationic lipid B-7□

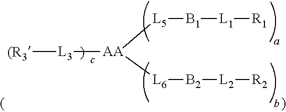

containing the divalent linking group L₃; wherein, the small molecule B-6 contains the reactive group F₄, which can react with the amino acid end group of AA^{□}to form a divalent linking group L₃, F₄ is preferably —OH, -COOH, —NH₂, —F, —Cl, —Br,

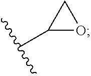 [SMILES: *C1CO1]

AA is the amino acid or amino acid derivative residue;
when R₃□is equal to R₃, the resulting structure B-7□corresponds to the structure represented by general formula (1);
when R₃ [symbol] s not equal to R₃, B-7 [symbol] s micromodified at the terminal group to obtain B-7, which corresponds to the structure represented by general formula (1); the terminal micro-modification is selected from the following chemical reactions: deprotection, salt complexation and decomplexation, ionization, protonation, deprotonation, and leaving group transformation;
wherein, the definitions of L₁, L₂, L₃, L₅, L₆, B₁, B₂, R₃, R₁, R₂, a, b and c are consistent with those described in general formula (1), and will not be repeated here.

The aforementioned small molecule starting materials B-1, B-2, B-4 and B-6 can be obtained by purchase or synthesis.

### Step 1

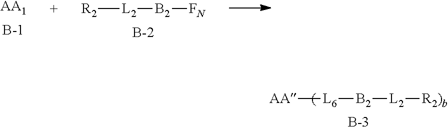

### Step 2

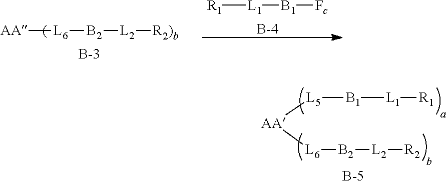

### Step 3

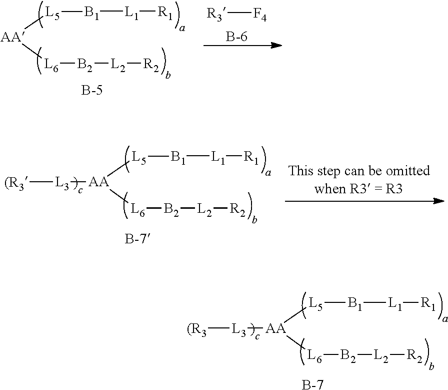

### 2.3. Method 3:

Step 1: one molecule of C-1

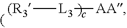

synthesized according to the step 1 in 2.1 Method 1 or purchased) is reacted with one or two molecules of the same or different C-2 (R₁-L₁-B₁-F_{c}, synthesized according to the step 2 in 2.1 Method 1 or purchased) to obtain an amino acid derivative C-3

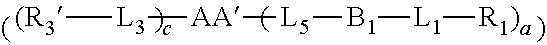

containing divalent linking groups L₃ and L₅; wherein, the small molecule C-1 is an amino acid or amino acid derivative containing two identical or different amino acid end groups; the small molecule C-2 contains a reactive group F_{c}, which can react with the amino acid end groups of C-1 to form the divalent linking group L₅, all L₅ are the same, preferably being -OH, —COOH, —NH₂, —Br, etc.; and AA ^{□}s a residue of amino acid derivative, which contains an amino acidend group.
Step 2: the amino acid derivative C-3

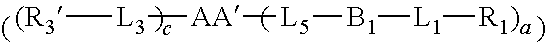

is reacted with the small molecule intermediate C-4 (R₂-L₂-B₂-F_{N}, synthesized according to the step 2 in 2.1. Method 1 or purchased) to obtain an amino acid-based cationic lipid C-5□

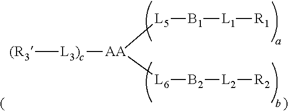

containing a divalent linking group L₆; wherein the small molecule C-4 contains a F_{N} group that can react with a carboxyl group, a hydroxyl group or an amino group, preferably being -OH, —COOH, —NH₂, —Br, —COCl,

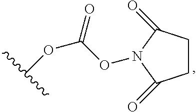 [SMILES: *OC(=O)ON1C(=O)CCC1=O]

etc.; AA is an amino acid or an amino acid derivative residue;
when R₃ □is equal to R₃, the resulting structure C-5 □corresponds to the structure represented by general formula (1);
when R₃ [symbol] s not equal to R₃, C-5 □s micromodified at the terminal group to obtain C-5, which corresponds to the structure represented by general formula (1); the terminal micro-modification is selected from the following chemical reactions: deprotection, salt complexation and decomplexation, ionization, protonation, deprotonation, and leaving group transformation;
wherein, the definitions of L₁, L₂, L₃, L₅, L₆, B₁, B₂, R₃, R₁, R₂, a, b and c are consistent with those described in general formula (1), and will not be repeated here;
The aforementioned small molecule starting materials C-1, C-2, and C-4 can be obtained by purchase or synthesis.

### Step 1

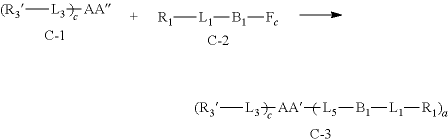

### Step 2

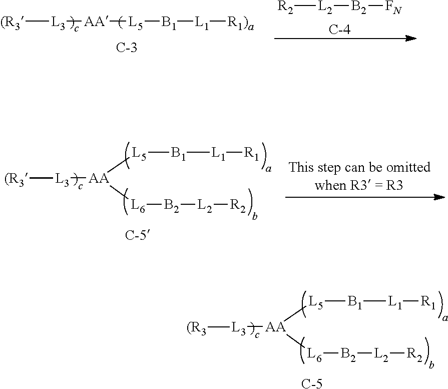

In the preceding preparation methods 1-3:

An amino acid end group is protected or unprotected, including but not limited to an amino group, a carboxyl group, a hydroxyl group, a sulfhydryl group, a protected amino group, a protected carboxyl group, a protected hydroxyl group, a protected sulfhydryl group. The variant form with micro-modification of R₀₁ refers to the structural form that can form a target reactive group after a simple chemical reaction processes such as deprotection, salt complexation and decomplexation, ionization, protonation, deprotonation, and leaving group transformation.

a, b, and c are each independently 1 or 2, depending on the type of amino acid end group linking to the fragment -L₆-B₂-L₂-R₂, -L₅-B₁-L₁-R₁, and -L₃-R₃ and the amount of starting materials. When a, b, or c is 2, the fragments can be the same or different, depending on the reaction between the amino acid derivative and the fragment, if the amino acid derivative reacts with two different fragments in sequence, the fragments will be different; if the amino acid derivative reacts with two identical fragments in a single or step-by-step manner, the fragments will be the same. For example, b in Method 1 is 1 or 2, depending on the type of end group of AA [symbol] eacting with F_{N} and the amount of R₂-L₂-B₂-F_{N}; when the end group of AA [symbol] eacting with F_{N} is a carboxyl group or a hydroxyl group, b is 1, and when it is an amino group, b is 1 or 2; and when b is 2, the two -L₆-B₂-L₂-R₂ fragments can be the same or different, that is, when F_{N}is reacted with the amino end group of AA [symbol] A-3 can react with different A-6 successively to obtain A-7 with b being 2 and two different -L₆-B₂-L₂-R₂ fragments, or one molecule of A-3 reacts with 2 molecules of the same A-6 in a single step or multiple steps to obtain A-7 with b being 2 and two identical -L₆-B₂-L₂-R₂ fragments.

R₂ in the starting material R₂-F₂ can be an etherified aliphatic hydrocarbon derivative residue

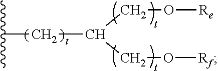

wherein, t is independently an integer of 0 to 12 at each occurrence; Rₑ and R_{f} are each independently a C₁₋₁₅ alkyl group, a C₁₋₁₅ alkenyl group or a C₂₋₁₅ alkyne group. More specifically, R₁-F₁ can be

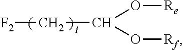

which can be obtained by purchase or synthesis, i.e., synthesized independently through aldehyde alcohol addition, for example, conducting the addiction reaction between one molecule of

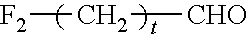

and two molecules of Rₑ-OH to obtain

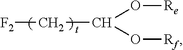

and Rₑ and R_{f} are the same; R₁-F₁ can also be

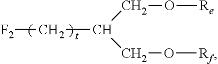

which can be obtained by purchase or synthesis, i.e., synthesized independently with the reaction between

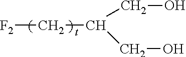

and the related alkylating reagent, the alkylating reagent preferably being a halide. For example,

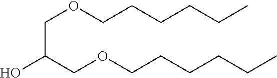 [SMILES: CCCCCCOCC(O)COCCCCCC]

can be obtained by reaction between one molecule of glycerol with TBS protecting hydroxyl group and two molecules of bromohexane and then deprotecting.
2.4. Description of Relevant Starting Materials and/or Steps in the Preparation Process

### 2.4.1. Condensing Agent, Oxidizing Agent, and Reducing Agent

In the present application, the condensing agent is not particularly limited, preferably N,N [symbol] icyclohexylcarbodiimide (DCC), 1-ethyl-(3-dimethylaminopropyl)carbodiimide hydrochloride (EDC-HCl), 2-(7-azobenzotriazole-N,N,N, [symbol] [symbol] etramethyluronium hexafluorophosphate (HATU), or 2-(1H-benzotriazol-1-yl)-1,1,3,3-tetramethyluronium hexafluorophosphate (HBTU), and most preferably DDC. Generally, the molar equivalent of the condensing agent is 1 to 20 folds of that of the carboxylic acid, preferably 5 to 10 folds, and suitable catalysts (such as 4-dimethylaminopyridine) can be added to the reaction.

In the present application, the oxidizing agent is not particularly limited as long as it is a compound or a combination of multiple compounds capable of increasing the valence of the substrate, preferably phenyliodine(III) bis(trifluoroacetate), 1,4-benzoquinone, benzyl trimethyl ammonium tribromide, pyridinium dichromate, ozone, oxygen, hydrofluoric acid, sodium hypochlorite, cobaltic acetate, cobalt acetate, manganous acetate, palladium(II) acetate, cupric acetate, monoperoxyphthalic acid, iodine, N-iodosuccinimide, iodoxybenzene, 2-iodylbenzoic acid, dimethyldioxirane, dimethyl sulfoxide-oxalyl chloride, DDQ, dichlorotris(triphenylphosphine)ruthenium, manganese dioxide, (diacetoxyiodo)benzene, periodic acid, sodium periodate, sodium periodate-osmium tetraoxide, potassium permanganate, sodium perborate, perbenzoic acid, dibenzoyl peroxide, nickel peroxide, hydrogen peroxide, cumyl hydroperoxide, 1-butyl hydroperoxide, peracetic acid, m-chloroperbenzoic acid, N-chlorosuccinimide, pyridinium chlorochromate, palladium chloride-cupric chloride, urea hydrogen peroxide adduct, triphenylcarbenium tetrafluoroborate, tributyltin oxide, cobalt trifluoride, vanadium oxytrifluoride, chromium trioxide, manganese triacetate, TEMPO, diammonium cerium nitrate, bromine, pyridine N-oxide, silver oxide, O-ethylperoxycarbonic acid, manganese acetyllacetonate, vanadyl acetylacetonate, aluminium isopropoxide, peroxymonosulfate, dichloroiodobenzene, the like, or any combination thereof, and more preferably oxygen, sodium hypochlorite, hydrogen peroxide, dichloroiodobenzene, peroxymonosulfate, the like, or any combination thereof, the molar equivalent of the oxidizing agent is 1 to 50 folds of that of the hydroxyl group of the intermediate compound, preferably 1 to 20 folds, and more preferably 5 to 10 folds.

In the present application, the reducing agent is not particularly limited as long as it can reduce the Schiff base formed by the reaction of an amine with an aldehyde or ketone to an amino group, preferably sodium borohydride, sodium cyanoborohydride, lithium aluminum hydride, borane, diborane, diisobutylaluminum hydride, diisopinocampheylborane, lithium borohydride, zinc borohydride, borane-pyridine, borane-methyl sulfide, borane-tetrahydrofuran, the like, or any combination thereof, and more preferably sodium cyanoborohydride; the molar equivalent of the reducing agent is 1 to 50 folds of that of the amino group to be modified, preferably 1 to 20 folds, and more preferably 5 to 10 folds.

In the present application, the reaction temperature is 0 to 200° C., preferably 0 to 100° C., and more preferably 0 to 25° C.; the reaction time is preferably 10 min to 48 h, and more preferably 30 min to 24 h. The obtained product can be purified by a purification means such as extraction, recrystallization, adsorption treatment, precipitation, reverse precipitation, membrane dialysis, and supercritical extraction.

In the present application, the reaction solvent can be absent or an aprotic solvent including toluene, benzene, xylene, acetonitrile, ethyl acetate, diethyl ether, tert-butyl methyl ether, tetrahydrofuran, chloroform, dichloromethane, dimethylsulfoxide, dimethylformamide, and dimethylacetamide; the aprotic solvent is preferably tetrahydrofuran, dichloromethane, dimethylsulfoxide, or dimethylformamide.

In the present application, the bases used in reactions are generally inorganic bases or organic bases, preferably organic bases (such as triethylamine, pyridine, 4-dimethylaminopyridine, imidazole or diisopropyl ethylamine); preferably Triethylamine, pyridine. The molar equivalent of the base is 1 to 50 folds of that of carboxylic acids, preferably 1 to 10 folds, and more preferably 2 to 3 folds.

### 2.4.2. “Protection” and “Deprotection” of Relevant Groups Involved in the Reaction Process

In the present application, the reaction process also involves the “protection” and “deprotection” processes of relevant groups. In order to prevent a functional group from affecting the reaction, the functional group is usually protected. In addition, when there are two or more functional groups and only the target functional group needs to react, the other functional groups should therefore be protected. The protecting group not only protects the functional group stably, but also needs to be removed easily as needed. Therefore, in organic synthesis, it is important to remove only the protecting group bonded to the specified functional group under appropriate conditions.

In the present application, the definitions of “carboxyl protecting group” and “amino protecting group” are consistent with the “Detailed description of the applicationAPPLICATION” section, and will not be repeated herein.

In the present application, the hydroxyl groups protected by hydroxyl protecting groups are not particularly limited, e.g., alcoholic hydroxyl groups, phenolic hydroxyl groups, and the like. The amino groups protected by amino protecting groups are not particularly limited, such as those from primary amines, secondary amines, hydrazines, and amides. Amino groups in the present application are not particularly limited, including but not limited to primary amino groups, secondary amino groups, tertiary amino groups, and quaternary ammonium ions.

In the present application, the deprotection of protected hydroxyl groups is related to the types of hydroxyl protecting groups. The types of hydroxyl protecting groups are not particularly limited; for example, benzyl groups, silyl ether, tert-butyl groups can be used to protect terminal hydroxyl groups, and the corresponding deprotection methods include the follows:

### A: Deprotection of Benzyl Protecting Groups

The deprotection of benzyl groups can be achieved via hydrogenation using a hydrogenative reducing agent and a hydrogen donor. As used herein, the water content should be less than 1% in order to facilitate the reaction.

The hydrogenative reduction agent is not particularly limited, preferably palladium or nickel. The agent carrier is not particularly limited, preferably alumina or carbon, and more preferably carbon. The amount of palladium is 1 to 100 wt % of that of compounds containing protected hydroxyl groups, preferably 1 to 20 wt %.

The reaction solvent is not particularly limited, as long as it allows the reagents and the products to be dissolved. Preferable solvents include methanol, ethanol, ethyl acetate, tetrahydrofuran, and acetic acid, wherein methanol is more preferable. The hydrogen donor is not particularly limited, preferably hydrogen gas, cyclohexene, 2-propanol, ammonium formate, or the like. The reaction temperature is preferably 25 to 40° C. The reaction time is not particularly limited, which is negatively correlated with the amount of catalyst used and preferably 1 to 5 hours.

### B: Deprotection of Silyl Ether Protecting Group

Compounds used for this type of hydroxyl protection include trimethylsilyl ether, triethylsilyl ether, tert-butyldimethylsilyl ether, tert-butyldiphenylsilyl ether, and the like. The deprotection of such silyl ethers uses compounds containing fluoride ions, wherein the compound is preferably tetrabutylammonium fluoride, tetraethylammonium fluoride, hydrofluoric acid, or potassium fluoride, and more preferably tetrabutylammonium fluoride or potassium fluoride. The amount of the fluorine-containing compound is 5 to 20 folds of that of the protected hydroxyl group and preferably 8 to 15 folds of that of the initiator. When the amount of the fluorine-containing compound used is less than 5 molar equivalents per molar equivalent of protected hydroxyl groups, the deprotonation might not be complete. When the amount of deprotecting reagent used exceeds 20 molar equivalents per molar equivalent of the initiator, the excess reagent tends to cause difficulty in the purification process and result in side reactions in subsequent steps. The reaction solvent is not particularly limited as long as it can dissolve the reagents and the products, preferably an aprotic solvent, and more preferably tetrahydrofuran or dichloromethane. The reaction temperature is preferably 0 to 30° C.; when it is lower than 0° C., the reaction rate is relatively slow, and the protecting group cannot be completely removed.

C: Deprotection of t-Butyl Protection Groups

The deprotection of tert-butyl groups is carried out under an acidic condition, and the pH of the solution is preferably 0 to 4. The acid is not particularly limited, but is preferably acetic acid, phosphoric acid, sulfuric acid, hydrochloric acid, or nitric acid, and more preferably hydrochloric acid. The reaction solvent is not particularly limited as long as it can dissolve the reagents and the products, preferably water. The reaction temperature is preferably 0 to 30° C.

### 2.4.3. Alkylation Reaction

In the present application, the alkylation reactions are preferably those based on hydroxyl groups, sulfhydryl groups, or amino groups, corresponding to the formation of ether bonds, thioether bonds, and secondary or tertiary amino groups, respectively. Specific examples are as follows:

2.4.3.1. Alkylation Reaction of Substrate Alcohols with Sulfonates or Halides

The amine intermediate can be obtained via the nucleophilic substitution of the substrate alcohol with a sulfonate or halide under a basic condition. Wherein, the amount of the sulfonate or halide is 1 to 50 and preferably 1 to 5 molar equivalents per molar equivalent of the substrate alcohol. When the amount of the sulfonate or halide is less than 1 molar equivalent per molar equivalent of the substrate alcohol, the substitution may not be complete, causing difficulty in the purification process. When the amount of the sulfonate or halide exceeds 50 molar equivalents per molar equivalent of the substrate alcohol, the excess sulfonate or halide tends to cause difficulty in the purification process and result in side reactions in the subsequent steps.

The resulting product is a mixture of ether intermediate and excess sulfonate or halide, and can be purified by a purification means such as anion exchange resin, osmosis treatment, ultrafiltration treatment, and the like. Wherein, the anion exchange resin is not particularly limited as long as the target product can undergo ion-exchange and adsorb on the resin, preferably the ion exchange resin of a tertiary amine or quaternary ammonia salt based on dextran, agarose, polyacrylate, polystyrene, poly(diphenylethylene), or the like. The solvents used for osmosis treatment and ultrafiltration treatment are not limited, generally water or an organic solvent. The organic solvent is not particularly limited as long as the product can be dissolved therein, preferably dichloromethane, chloroform, or the like.

The reaction solvent is not limited, preferably an aprotic solvent such as toluene, benzene, xylene, acetonitrile, ethyl acetate, tetrahydrofuran, chloroform, dichloromethane, dimethylsulfoxide, dimethylformamide, and dimethylacetamide, and more preferably dimethylformamide, dichloromethane, dimethylsulfoxide, or tetrahydrofuran.

The base can be an organic base such as triethylamine, pyridine, 4-dimethylaminopyridine, imidazole, and diisopropylethylamine, or an inorganic base such as sodium carbonate, sodium hydroxide, sodium bicarbonate, sodium acetate, potassium carbonate, and potassium hydroxide, preferably an organic base, and more preferably triethylamine or pyridine. The amount of the base is 1 to 50 and preferably 1 to 10 and more preferably 3 to 5 molar equivalents per molar equivalent of the sulfonate or halide.

2.4.3.2. Alkylation Reaction of Substrate Amines with Sulfonates or Halides

The amine intermediate can be obtained via the nucleophilic substitution of the substrate amine with a sulfonate or halide under a basic condition. Wherein, the amount of the sulfonate or halide is 1 to 50 and preferably 1 to 5 molar equivalents per molar equivalent of the substrate amine. When the amount of the sulfonate or halide is less than 1 molar equivalent per molar equivalent of the substrate amine, the substitution may not be complete, causing difficulty in the purification process. When the amount of the sulfonate or halide exceeds 50 molar equivalents per molar equivalent of the substrate amine, the excess sulfonate or halide tends to cause difficulty in the purification process and result in side reactions in the subsequent steps.

The resulting product is a mixture of amine intermediate and excess sulfonate or halide, and can be purified by a purification means such as anion exchange resin, osmosis treatment, ultrafiltration treatment, and the like. Wherein, the anion exchange resin is not particularly limited as long as the target product can undergo ion-exchange and adsorb on the resin, preferably the ion exchange resin of a tertiary amine or quaternary ammonia salt based on dextran, agarose, polyacrylate, polystyrene, poly(diphenylethylene), or the like. The solvents used for osmosis treatment and ultrafiltration treatment are not limited, generally water or an organic solvent. The organic solvent is not particularly limited as long as the product can be dissolved therein, preferably dichloromethane, chloroform, or the like.

The reaction solvent is not limited, preferably an aprotic solvent such as toluene, benzene, xylene, acetonitrile, ethyl acetate, tetrahydrofuran, chloroform, dichloromethane, dimethylsulfoxide, dimethylformamide, and dimethylacetamide, and more preferably dimethylformamide, dichloromethane, dimethylsulfoxide, or tetrahydrofuran.

The base can be an organic base such as triethylamine, pyridine, 4-dimethylaminopyridine, imidazole, and diisopropylethylamine, or an inorganic base such as sodium carbonate, sodium hydroxide, sodium bicarbonate, sodium acetate, potassium carbonate, and potassium hydroxide, preferably an organic base, and more preferably triethylamine or pyridine. The amount of the base is 1 to 50 and preferably 1 to 10 and more preferably 3 to 5 molar equivalents per molar equivalent of the sulfonate or halide.

2.4.3.3. Alkylation Reaction of Substrate Amines with Aldehyde Derivatives

The substrate amine reacts with an aldehyde derivative to obtain an imine intermediate, which is followed by obtaining an intermediate by reducing reagents. Wherein, the amount of the aldehyde derivative is 1 to 20 and preferably 1 to 2 and more preferably 1 to 1.5 molar equivalents per molar equivalent of the substrate amine. When the amount of aldehyde exceeds 20 molar equivalents per molar equivalent of the substrate amine, the excess reagent tends to cause difficulty in the purification process and result in side reactions in the subsequent steps. When the amount of aldehyde is less than 1 molar equivalent per molar equivalent of the substrate amine, the substitution may not be complete, causing the purification process to be difficult. Wherein, the resulting product can be obtained after purification by means such as cation exchange resin, osmosis treatment, ultrafiltration treatment, and the like. The cation exchange resin is not particularly limited as long as it can undergo ion-exchange with quaternary ammonium cations and realize the isolation. The solvents used for osmosis treatment and ultrafiltration treatment are not limited, generally water or an organic solvent. The organic solvent is not particularly limited as long as the product can be dissolved therein, preferably dichloromethane, chloroform, or the like.

The reaction solvent is not limited, preferably an organic solvent such as methanol, ethanol, water, toluene, benzene, xylene, acetonitrile, ethyl acetate, tetrahydrofuran, chloroform, dichloromethane, dimethylsulfoxide, dimethylformamide, dimethylacetamide, and the like, and more preferably water or methanol.

The reduction reagent is not particularly limited as long as the imine can be reduced to an amine, preferably sodium borohydride, lithium aluminum hydride, sodium cyanoborohydride, Zn/AcOH, or the like, and more preferably sodium cyanoborohydride. The molar amount of the reduction reagent is generally 0.5 to 50 folds and preferably 1 to 10 folds of that of aldehyde derivatives.

### 2.4.3.4. The Linear End-Functionalization

The method for linear end-functionalization is not particularly limited, but related to the type of the final functional group or protected form thereof. The method mainly includes the functionalization of the terminal hydroxyl group and the conversion of a reactive group into the target functional group or the protected form thereof.

The method for functionalization of the terminal hydroxyl group is described herein, which is by converting the terminal hydroxyl group of A into a group from classes A-J via end-functionalization. A specific preparation method is described in paragraphs [0960] to [1205] of the document CN104530417A. The general formula of the reaction is as follows:

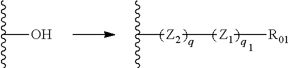

Wherein, q and q₁ are each independently 0 or 1; Z₁ and Z₂ are each independently a divalent linking group; R₀₁ is the functional group capable of reacting with bio-related substances.

Conversion of reactive groups into the target functional groups or protected forms thereof can be achieved by any of the following approaches:
Approach 1: direct modification. The target functional group or its protected form can be obtained via direct modification of a reactive group. As an example, such as the conversion of a carboxyl group to an acyl halide group, a hydrazide group, an ester group, a thioester group, or a dithioester group, the conversion of a hydroxyl group, a sulfhydryl group, an alkynyl group, an amino group, a carboxyl group, or the like to the corresponding protected structures. Another example is the modification of a hydroxyl group, an amino group, or the like by an anhydride.
Approach 2: coupling reaction between two reactive groups. The coupling reaction uses a heterofunctional reagent which contains both a reactive group and the target functional group or its protected form as the materials, the reactive group is capable of reacting with the terminal group of A to introduce the target functional group or its protected form. The modes and methods of the reaction between two reactive groups are not particularly limited, wherein the reaction conditions are related to the types of divalent linking groups formed via the reaction. The available prior art such as alkylation reaction, addition reaction of alkenes, addition reaction of alkynes, combination of Schiff-base reaction and reduction reaction, condensation reaction, and the like, can be used herein. Wherein, the alkylation reaction is preferably based on a sulfhydryl group or an amino group, corresponding to the formation of a thioether bond, and a secondary or tertiary amino group, respectively. Wherein, the condensation reaction includes but is not limited to those forming an ester bond, a thioester bond, an amide bond, an imine bond (—C═N-), a hydrazone bond, a carbamate bond, or the like. The target functional group or its protected form can also be introduced via click reactions using materials such as a heterofunctional reagent containing both the target functional group or its protected form and a reactive group selected from the group consisting of an azido group, an alkynyl group, an alkenyl group, a trithioester group, a sulfhydryl group, a dienyl group, a furyl group, a 1,2,4,5-tetrazinyl group, a cyanate group, and the like. The reaction between two reactive groups is accompanied by the formation of new bonds. Typical representatives of the newly formed divalent linking groups include amide bonds, urethane bonds, ester bonds, secondary amino bonds, thioether bonds, triazole groups, and the like.
Approach 3: combination of direct modification and coupling reaction; wherein, the target functional group or its protected form can be obtained via the combination.

In the present application, starting materials in every preparation method can be synthesized or purchased.

The intermediates and end-products prepared in the present application can be purified by the purification method including but not limited to extraction, recrystallization, adsorption treatment, precipitation, reverse precipitation, membrane dialysis, supercritical extraction, and the like. The characterization of the structure and the molecular weight of end-products can use methods including but not limited to NMR, electrophoresis, UV-visible spectrophotometer, FTIR, AFM, GPC, HPLC, MALDI-TOF, circular dichroism spectroscopy, and the like.

### 3.1. Lipid Compositions

In the present application, provided herein is a lipid composition containing any amino acid-based cationic lipid whose structure is represented by the general formula (1).

One specific embodiment of the present application, the lipid composition preferably contains, in addition to a cationic lipid with a structure represented by general formula (1), one or more types of lipids selected from the group consisting of phospholipid, steroidal lipid and PEGylated lipid, selected from any one of the following cases:
Case (1): contains a phospholipid;
Case (2): contains a steroid lipid;
Case (3): contains a PEGylated lipid;
Case (4): contains a phospholipid and a steroid lipid;
Case (5): contains a phospholipid and a PEGylated lipid;
Case (6): contains a steroid lipid and a PEGylated lipid;
Case (7): contains a phospholipid, a steroid lipid and a PEGylated lipid;
more preferably, it also contains a neutral lipid, a steroid lipid and a PEGylated lipid, simultaneously.

In a specific embodiment of the present application, the phospholipid in the lipid composition is preferably 1,2-dilinoleoyl-sn-glycero-3-phosphocholine (DLPC),
1,2-dimyristoleoyl-sn-glycero-3-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphatidylcholine (DSPC),
1,2-diundecanoyl-sn-glycero-3-phosphatidylcholine (DUPC),
1-plamitoyl-2-oleoyl-sn-glycero-3-phosphocholine (POPC),
1,2-di-O-octadecenyl-sn-glycero-3-phosphatidylcholine (18:0 Diether PC),
1-oleoyl-2-cholesterylhemisuccinyl-sn-glycero-3-phosphocholine (OChemsPC),
1-O-hexadecyl-sn-glycero-3-phosphatidylcholine (C16 Lyso PC),
1,2-dilinolenoyl-sn-glycero-3-phosphatidylcholine,
1,2-diarachidonoyl-sn-glycero-3-phosphatidylcholine,
1,2-didecosahexaenoyl-sn-glycero-3-phosphocholine,
1,2-dioleoyl-sn-glycero-3-phosphoethanolamine (DOPE),
1,2-diphytanyl-sn-glycero-3-phosphoethanolamine (ME 16.0 PE),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didecosahexaenoyl-sn-glycero-3-phosphoethanolamine,
   1,2-dioleoyl-sn-glycero-3-phospho-rac-(1-glycerol) sodium salt (DOPG), dioleoyl phosphatidylserine (DOPS), dipalmitoyl phosphatidylglycerol (DPPG), palmitoyloleoyl phosphatidylethanolamine (POPE), distearoyl phosphatidylethanolamine (DSPE), dipalmitoyl phosphatidylethanolamine (DPPE), dimyristoleoyl phosphoethanolamine (DMPE), 1-stearoyl-2-oleoyl-stearoylethanolamine (SOPE), 1-stearoyl-2-oleoyl-phosphatidylcholine (SOPC), sphingomyelin, phosphatidylcholine, phosphatidylethnolamine, phosphatidylserine, phosphatidylinositol, phosphatidic acid, palmitoyloleoyl phosphatidylcholine, lysophosphatidylcholine, lysophosphatidylethanolamine (LPE), and combinations thereof.

In a specific embodiment of the present application, the steroid lipid in the lipid compositions is preferably selected from the group consisting of cholesterol, fecal sterol, sitosterol, ergosterol, campesterol, stigmasterol, rapeseed sterol, lycopene, ursolic acid, α-tocopherol, and combinations thereof.

In a specific embodiment of the present application, the PEGylated lipid in the lipid composition is preferably any one of 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-cholesterol, PEG-diacylglycamide (PEG-DAG), and PEG-dialkyloxypropyl (PEG-DAA), specifically including PEG500-dipalmitoylphosphatidylcholine, PEG2000-dipalmitoylphosphatidylcholine, PEG500-stearylphosphatidylethanolamine PEG2000-distearylphosphatidylethanolamine, PEG500-1,2-oleoylphosphatidylethanolamine, PEG2000-1,2-oleoylphosphatidylethanolamine and PEG2000-2,3-dimyristoylglycerol, and combinations thereof.

In a specific embodiment of the present application, the PEGylated lipid in the lipid composition is preferably selected from the group consisting of the following structures and combinations thereof:

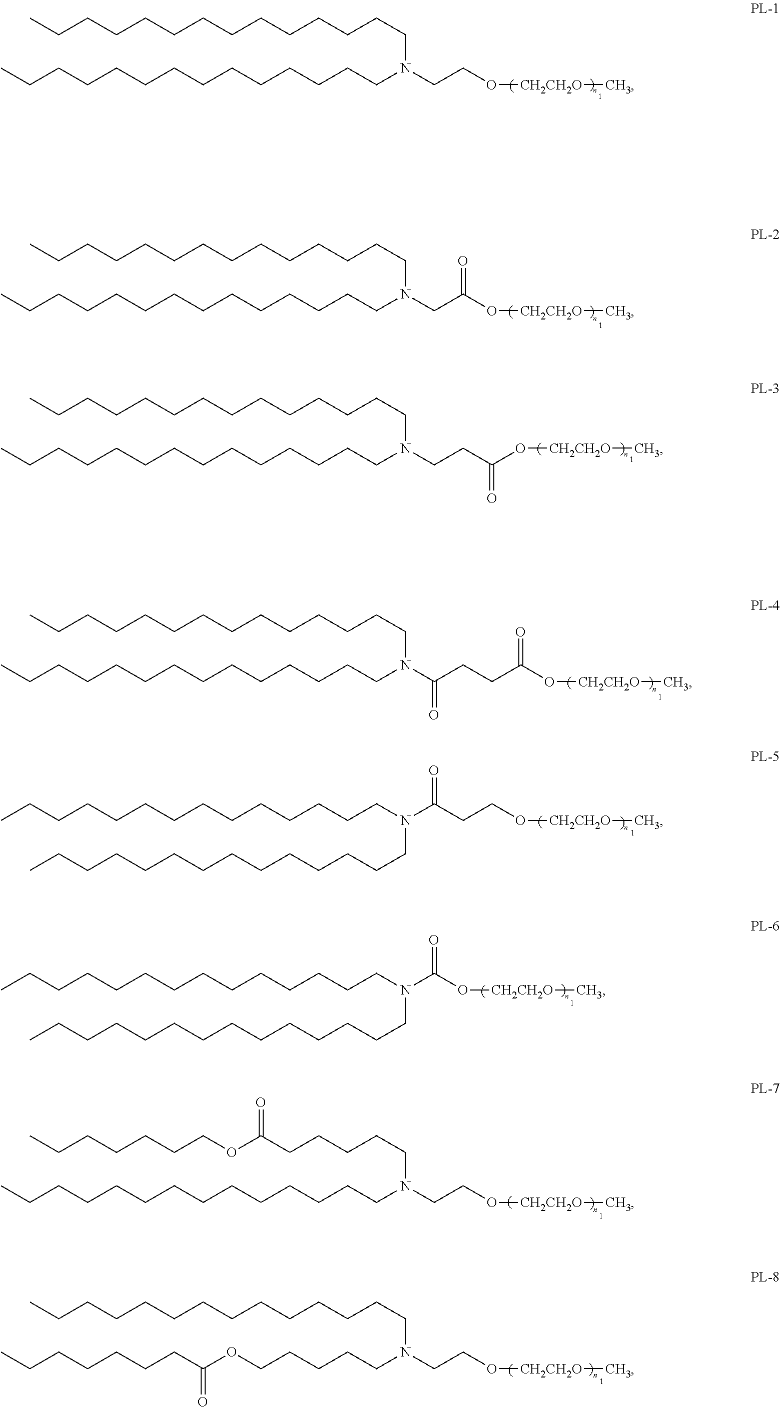

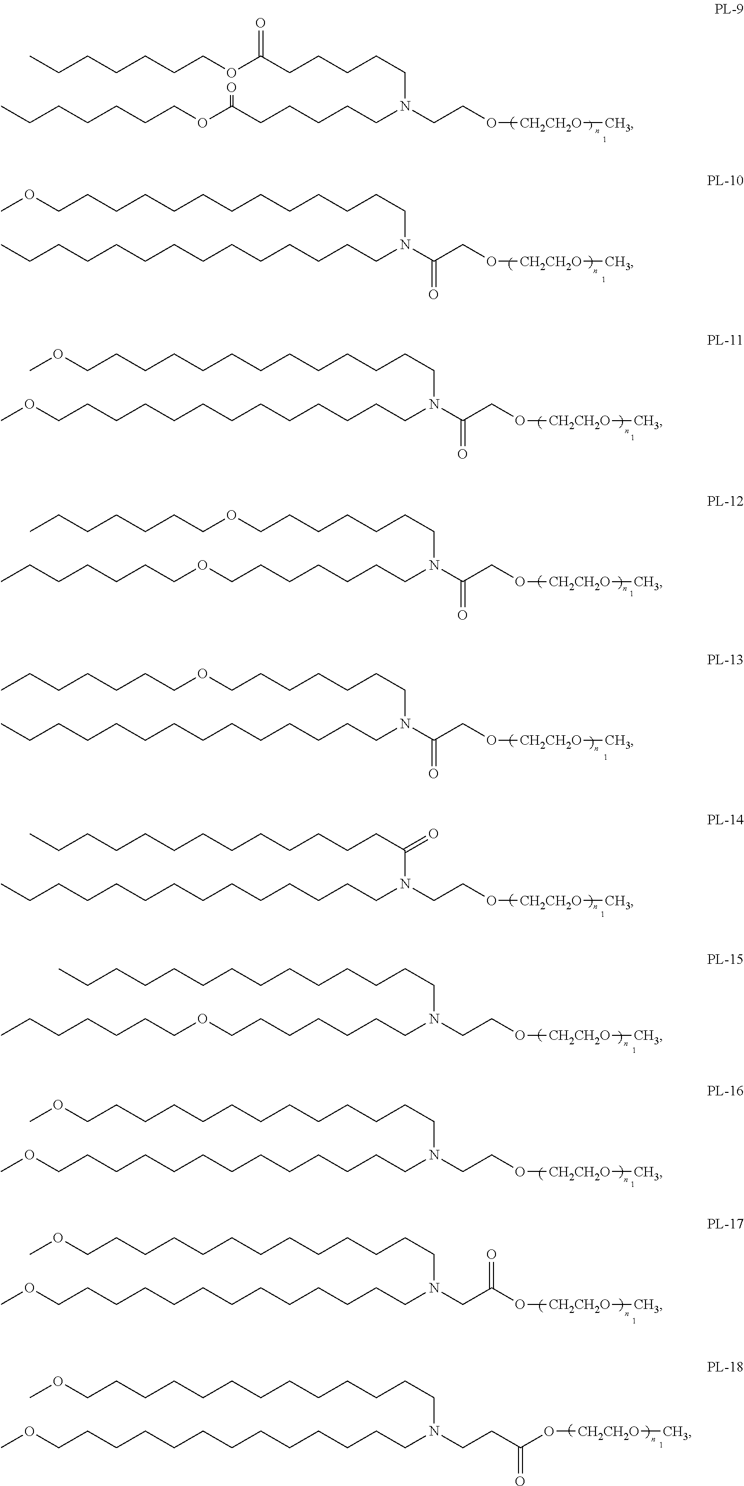

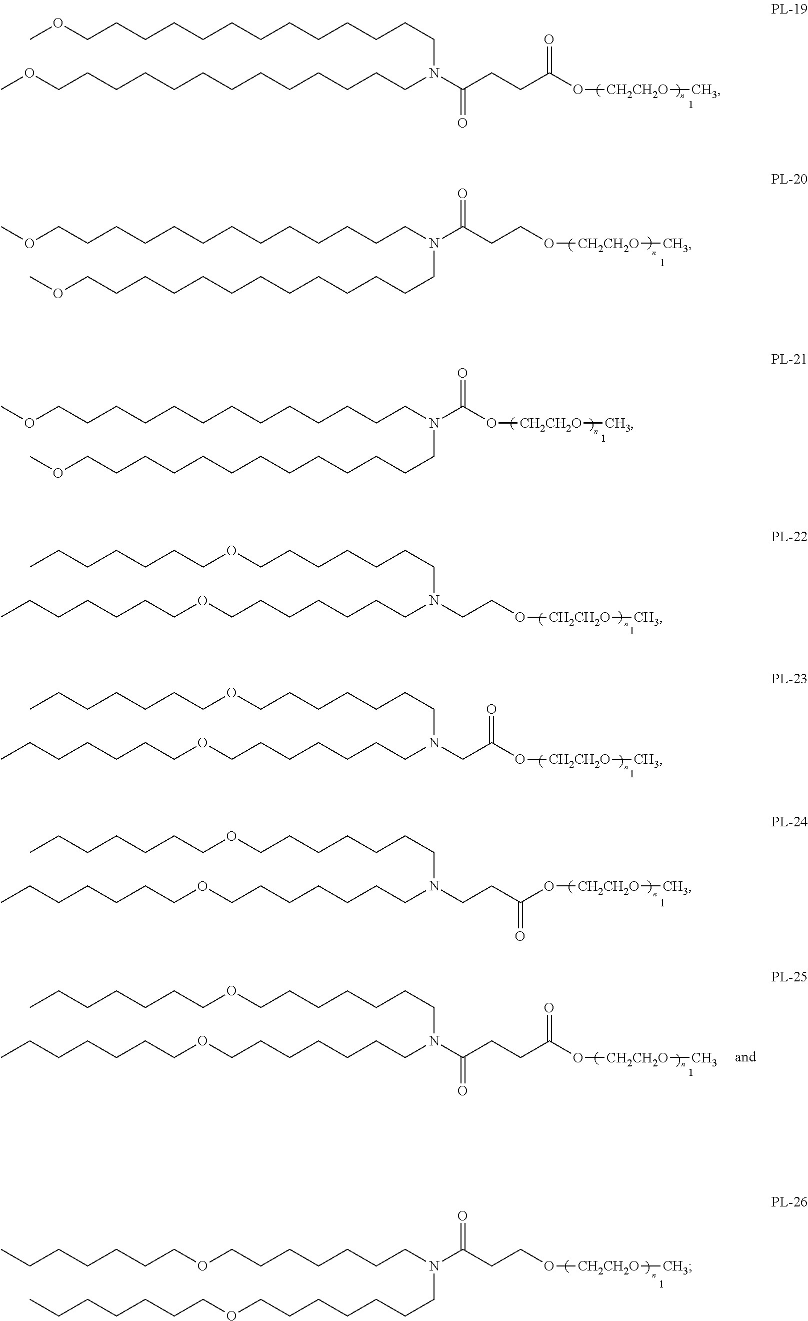

wherein, n₁ is an integer of 25 to 300. More preferably n₁ is selected from the group consisting of 44, 45, 46, 47, and 48.

In one specific embodiment of the present application, any of the aforementioned lipid compositions preferably contain 20-80% amino acid-based cationic lipid represented by formula (1), 5-15% phospholipid, 25-55% steroid lipid, and 0.5-10% PEGylated lipid, the percentage is the molar percentage of each lipid in the total lipids in the solution containing solvent.

In a specific embodiment of the present application, in any of the aforementioned lipid compositions, the molar percentage of cationic lipids in the total lipids in a solution containing solvent is preferably 30-65%; more preferably about 35%, 40%, 45%, 46%, 47%, 48%, 49%, 50%, or 55%.

In a specific embodiment of the present application, in any of the aforementioned lipid compositions, the molar percentage of phospholipids in the total lipids in a solution containing solvent is preferably about 7.5-13%; more preferably about 8%, 9%, 10%, 11%, or 12%.

In a specific embodiment of the present application, in any of the aforementioned lipid compositions, the molar percentage of steroid lipids in the total lipids in a solution containing solvent is preferably 35-50%, more preferably about 40%, 41%, 42%, 43%, 44%, 45%, 46%, 47%, 48%, 49%, or 50%.

In a specific embodiment of the present application, in any of the aforementioned lipid compositions, the molar percentage of PEGylated lipids in the total lipids in a solution containing solvent is 0.5-5%; preferably 1-3%; more preferably about 1.5%, 1.6%, 1.7%, 1.8%, or 1.9%.

### 3.2. Preparation of Lipid Compositions

In the present application, the lipid composition can be prepared by the following methods, including but not limited to ethanol injection, microfluidic method, T-tube mixing method, periplasmic extrusion method, preferably ethanol injection and microfluidic method.

### 4. Lipid Pharmaceutical Compositions and Formulations Thereof

### 4.1. Lipid Pharmaceutical Compositions

In one embodiment of the present application, a lipid pharmaceutical composition contains any lipid composition and drug described above; wherein, the lipid composition contains any of the previously described amino acid-based cationic lipids with the structure represented by formula (1), and the drug is selected from the group consisting of a nucleic acid drug, a gene vaccine, an antitumor drug, a small molecule drug, a peptide drug or a protein drug.

In a specific embodiment of the present application, in a lipid pharmaceutical composition, the nucleic acid drug is selected from the group consisting of RNA, DNA, antisense nucleic acid, plasmid, interfering nucleic acid, aptamer, antagomir and ribozyme, the RNA is selected from the group consisting of mRNA, saRNA, circRNA, miRNA and siRNA; nucleic acid drug is preferably selected from the group consisting of DNA, mRNA, miRNA or siRNA.

In a specific embodiment of the present application, the lipid pharmaceutical composition is preferably used as a drug, selected from any of the following drugs: an antineoplastic agent, an antiviral agent, an antifungal agent and a vaccine.

In one specific embodiment of the present application, the drug preferably includes, but is not limited to doxorubicin, mitoxantrone, camptothecin, cisplatin, bleomycin, cyclophosphamide, streptozotocin, actinomycin D, vincristine, vinblastine, cytosine arabinoside, anthracycline, nitrogen mustard, tioteppa, chlorambucil, rachelmycin, melphalan, carmustine, romustine, busulfan, dibromannitol, mitomycin C, cis-diammineplatinum(II) dichloride, methotrexate, 6-mercaptopurine, 6-thioguanine, cytosine arabinoside, 5-fluorouracil dacarbazine, dibucaine, chlorpromazine, propranolol, demorol, labetalol, clonidine, hydralazine, imipramine, amitriptyline, doxepin, phenytoin, diphenhydramine, chlorpheniramine, promethazine, gentamicin, ciprofloxacin, cefoxitin, miconazole, terconazole, econazole, isoconazole, butoconazole, clotrimazole, itraconazole, nystatin, naftifine, amphotericin B, antiparasitic agents, hormones, hormone antagonists, immunomodulators, neurotransmitter antagonists, glaucoma drugs, vitamins, tranquilizers, imaging agents, taxol, cytochalasin B, gramicidin D, ethidium bromide, emetine, mitomycin, etoposide, teniposide, colchicine, daunorubicin, quinizarin, mithramycin, 1-dihydrotestosterone, glucocorticoid, procaine, tetracaine, lidocaine, puromycin, and maytansinoid.

In a specific embodiment of the present application, the N/P ratio of the lipid composition to the nucleic acid is preferably (0.1 ~100): 1, more preferably (0.2~30): 1, and most preferably (0.5~20): 1.

### 4.2. Formulations of Lipid Pharmaceutical Compositions

In a specific embodiment of the present application, the drug in the lipid pharmaceutical composition is the nucleic acid drug, and the working solution is preferably deionized water, ultrapure water, phosphate buffer or normal saline, and is more preferably phosphate buffer or normal saline, and most preferably normal saline; preferred lipid composition: working solution=(0.05-20) g: 100 mL, more preferably (0.1~10) g: 100 mL, most preferably (0.2~5) g: 100 mL.

In a specific embodiment of the application, a formulation of lipid pharmaceutical composition contains the aforementioned lipid pharmaceutical composition and pharmaceutically acceptable diluents or excipients; the diluent or excipient is preferably deionized water, ultrapure water, phosphate buffer or normal saline, more preferably phosphate buffer or normal saline, most preferably normal saline.

In the present application, the preparation of formulation of lipid pharmaceutical composition includes the following steps:
(1) equilibrate the lipid composition in the diluent or excipient;
(2) nucleic acid drugs are added to the mixture of the balanced lipid composition and the diluent or excipient for compounding;
wherein, the equilibrium time is preferably 0.1-12 h, preferably 0.2-6 h, and more preferably 0.5-3 h; the composite time is preferably 0.1-12 h, preferably 0.2-5 h, and more preferably 0.5-2 h.

### 5. Liposomes or Lipid Nanoparticles and the Preparation Thereof

### 5.1. Liposomes or Lipid Nanoparticles

In a specific embodiment of the present application, a liposome or lipid nanoparticle contains any lipid pharmaceutical composition described above.

In a specific embodiment of the present application, the aforementioned lipid nanoparticle is preferably an LNP pharmaceutical composition, an LPP pharmaceutical composition or a PNP pharmaceutical composition; preferably an LNP pharmaceutical composition; more preferably an LNP-nucleic acid pharmaceutical composition; more preferably an LNP-mRNA pharmaceutical composition.

### 5.2. Preparation of Liposomes or Lipid Nanoparticles

In a specific embodiment of the present application, liposomes can be prepared by the following methods, including but not limited to thin-film dispersion method, ultrasonic dispersion method, reverse-phase evaporation method, freeze-drying method, freeze-thaw method, double emulsion method and injection method, preferably by thin-film dispersion method, ultrasonic dispersion method and/or reverse-phase evaporation method.

In a specific embodiment of the present application, lipid nanoparticles can be prepared by the following methods, including but not limited to microemulsion method, double emulsion method, high shear homogenization and ultrasonication, thin film hydration extrusion method, and microfluidic method.

In a specific embodiment of the present application, liposomes are prepared by thin-film dispersion methods which includes the following steps:
Step (1): weigh cationic lipids, steroid lipids, neutral lipids, and PEGylated lipids, fully dissolve them in organic solvents, shake them well, remove the organic solvents by vacuum rotary evaporation to obtain an oil film, and furtherly dry with a vacuum pump to remove the organic solvents;
Step (2): add phosphate buffer with dissolved cryoprotectant, and use water-bath ultrasonication to obtain a translucent emulsion;
Step (3): add the emulsion to a high-pressure homogenizer for overpressure, and then add the over pressurized homogenized emulsion to the liposome extruder for film-passing to obtain cationic liposomes;
Step (4): optionally, dry the liposomes in a freeze dryer to obtain a liposome powder;
wherein, the organic solvent is preferably dichloromethane, chloroform, methanol, or a combination thereof, more preferably chloroform, methanol, or the combination thereof; wherein, the rotational speed of vacuum rotary evaporation is preferably 30 to 300 rpm, more preferably 50 to 200 rpm, and most preferably 100 to 170 rpm; and wherein, the temperature of vacuum rotary evaporation is preferably 10 to 200° C., more preferably 20 to 200° C., and most preferably 40 to 80° C.;
the time of the drying with a vacuum pump is preferably 1 to 72 h, more preferably 5 to 48 h, and most preferably 15 to 36 h;
the mass concentration of the cryoprotectant dissolved in phosphate buffer is preferably 0.1 to 80%, preferably 1 to 50%, and more preferably 5 to 20%;
the frequency of the water-bath ultrasonication is preferably 10 to 300 kHz, more preferably 30 to 200 kHz, and most preferably 60 to 150 kHz;
the time of the water-bath ultrasonication is preferably 0.1 to 5 h, more preferably 0.2 to 2 h, and most preferably 0.25 to 1 h;
the pressure of the high-pressure homogenizer is preferably 50 to 240 MPa, more preferably 80 to 200 MPa, and most preferably 100 to 150 MPa;
the times of the overpressure of high-pressure homogenizer is preferably any integer from 1 to 50, more preferably any integer from 3 to 20, and most preferably any integer from 5 to 10;
the pressure of the liposome extruder is preferably 50 to 300 MPa, more preferably 80 to 250 MPa, and most preferably 120 to 200 MPa;
the times of the film-passing of liposome extruder is preferably any integer from 1 to 50, more preferably any integer from 3 to 30, and most preferably any integer from 5 to 20;
the time of the drying in a freeze dryer is preferably 1 to 120 h, more preferably 5 to 72 h, and most preferably 10 to 36 h.

In a specific embodiment of the present application, in the preparation methods of liposomes, the ratio of liposome to phosphate buffer with dissolved cryoprotectant could be 1 mg: (0.1~100) mL, preferably 1 mg: (0.3~50) mL, and more preferably 1 mg: (0.5~5) mL.

In a specific embodiment of the present application, the lipid nanoparticles are preferably prepared by the microfluidic method, the steps are as follows:
Step (1): dissolve each lipid component in the organic solvent to obtain lipid compositions dissolved in the organic phase; the organic phase is preferably ethanol;
Step (2): add nucleic acid drugs to buffer to obtain aqueous solution; the aqueous phase is preferably a citrate buffer salt or sodium acetate buffer;
Step (3): the organic phase solution and aqueous phase solution are mixed by microfluidic equipment to obtain lipid nanoparticle compositions, and purified by ultrafiltration and the like to remove organic solvents and free nucleic acid molecules.

The following specific examples are further descriptions of the preparation methods of amino acid-based cationic lipids, lipid compositions, and formulations of lipid pharmaceutical compositions, and the biological activity assays for lipid pharmaceutical compositions; the specific examples are disclosed to further illustrate the application, but should not be regarded as a limitation of the scope of present application. Wherein, in the embodiments of preparing cationic lipids, the structures of end products are characterized by NMR, and the molecular weight is confirmed by mass spectrometry.

### Example-1.1: Cationic Lipid (E1-1)

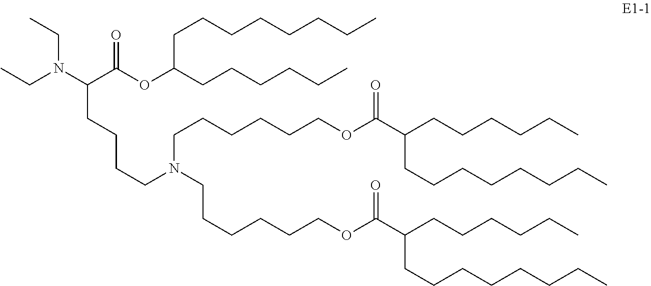 [SMILES: CCCCCCCCC(CCCCCC)OC(=O)C(CCCCN(CCCCCCOC(=O)C(CCCCCC)CCCCCCCC)CCCCCCOC(=O)C(CCCCCC)CCCCCCCC)N(CC)CC]

The preparation process is as follows:
Step a: under nitrogen atmosphere, N,N [symbol] icyclohexylcarbodiimide (DCC, 3.63 g, 17.6 mmol) was added to a round-bottom flask containing 2-hexyldecanoic acid (S1-1, 2.05 g, 8.0 mmol), 6-bromo-1-hexanol (S1-2, 1.74 g, 9.6 mmol) and 4-(dimethylamino)pyridine (DMAP, 0.24 g, 2.0 mmol) dissolved in dichloromethane (50 mL) and reacted for 16 h at room temperature. After the end of the reaction, the precipitate was removed by filtration, the filtrate was concentrated, and the resulting residue was purified by column chromatography to obtain 6-bromohexyl-2-hexylcaprate (S1-3, 2.77 g).
Step b: S1-3 (2.30 g, 5.5 mmol) was dissolved in 30 mL of DMF, NN-diethyllysine (S1-4, 0.51 g, 2.5 mmol, S1-4 was obtained by the reaction between lysine with a Boc protected amino group

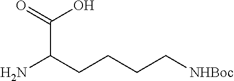 [SMILES: CC(C)(C)OC(=O)NCCCCC(N)C(=O)O]

and bromoethane

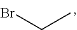

and then removing the Boc group) and potassium carbonate (K₂CO₃, 0.91 g, 6.6 mmol) were added, the reaction solution was stirred overnight at room temperature. After the reaction, the reaction mixture was concentrated under reduced pressure and 30 mL of ethyl acetate was poured into the concentrate. After washing with 10% citric acid (20 mL) and saline (20 mL) successively, the organic phase was dried with anhydrous sodium sulfate, filtered and concentrated, and the crude product was purified by column chromatography to obtain S1-5 (1.67 g).
Step c: DCC (0.68 g, 3.3 mmol) was added to a round-bottom flask containing compounds S1-5 (1.32 g, 1.5 mmol), 2-hexylnonanol (S1-6, 0.41 g, 1.8 mmol) and DMAP (45.00 mg, 0.4 mmol) dissolved in dichloromethane (20 mL) under nitrogen atmosphere, and the reaction solution was reacted for 16 h at room temperature. After the reaction, the precipitate was removed by filtration, the filtrate was concentrated, and the resulting residue was purified by column chromatography to obtain cationic lipid E1-1 (1.33 g). ¹H NMR (400 MHz, CDCl₃) δ: 4.93-4.81 (m, 1H), 4.09 (t, 4H), 3.46 (t, 1H), 3.18-2.99 (m, 6H), 2.49 (t, 4H), 2.33-2.25 (m, 2H), 2.01-1.82 (m, 4H), 1.68-1.26 (m, 96H), 0.88 (t, 18H). MS (ESI): m/z=1090.03 ([M+H] ⁺).

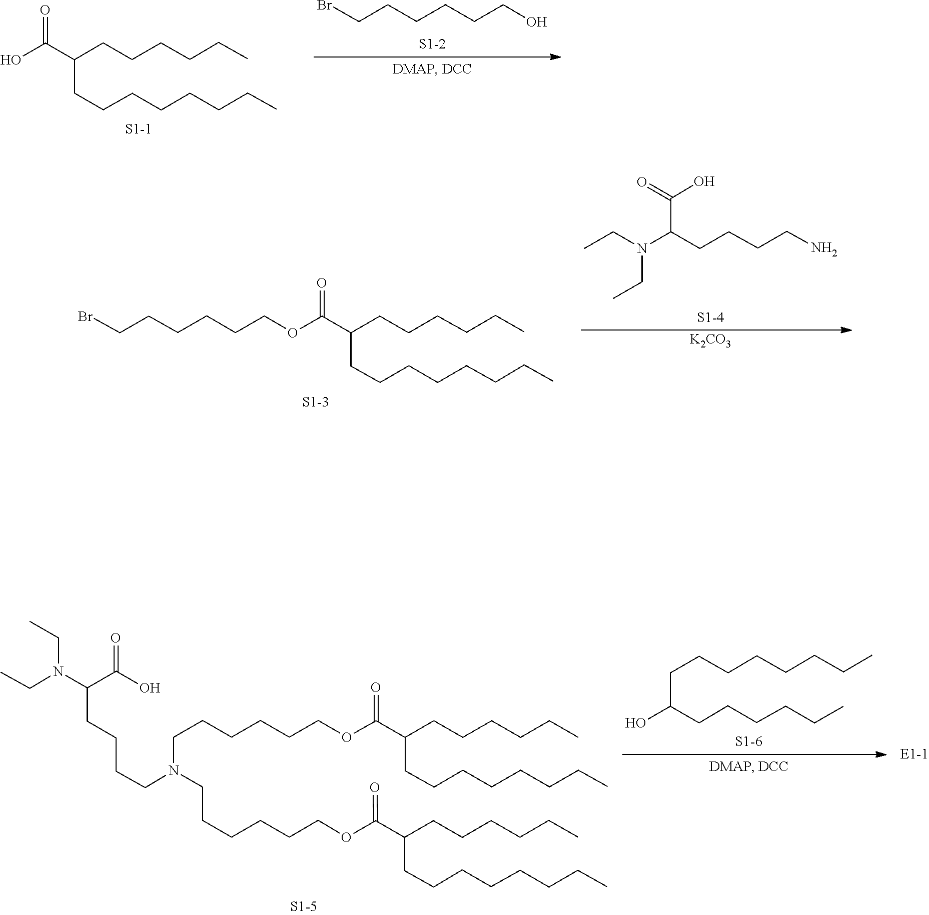

### Example 1.2: Cationic Lipid (E-2)

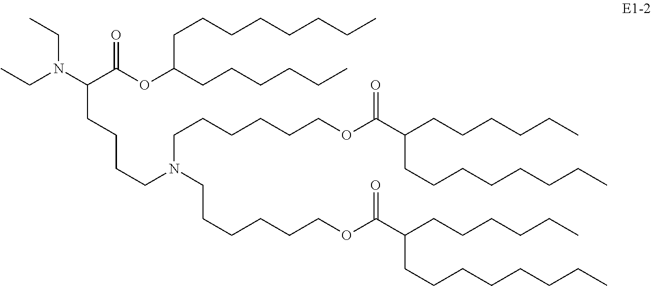 [SMILES: CCCCCCCCC(CCCCCC)OC(=O)C(CCCCN(CCCCCCOC(=O)C(CCCCCC)CCCCCCCC)CCCCCCOC(=O)C(CCCCCC)CCCCCCCC)N(CC)CC]

The starting material S1-6 in Example 1.1 was replaced with 2-octylnonanol

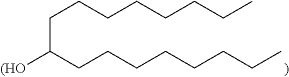

and prepared according to the same reaction steps to obtain cationic lipid E1-2. The structure of EI-2 was also verified by NMR and mass spectrometry.

### Example 2: Cationic Lipid (E2-2)

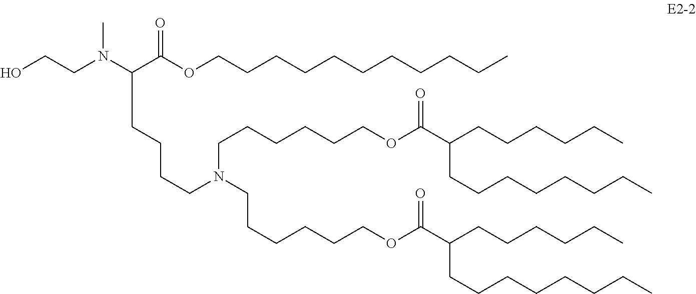 [SMILES: CCCCCCCCCCCOC(=O)C(CCCCN(CCCCCCOC(=O)C(CCCCCC)CCCCCCCC)CCCCCCOC(=O)C(CCCCCC)CCCCCCCC)N(C)CCO]

The preparation process is as follows:
Step a: S1-3 (2.77 g, 6.6 mmol) was dissolved in 50 mL of DMF, TBS-N-hydroxyethyl-N-methyllysine (S2-1, 0.96 g, 3.0 mmol, S2-1 was obtained by the reaction between N-methyllysine with a Boc protected amino group

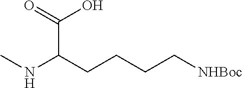 [SMILES: CNC(CCCCNC(=O)OC(C)(C)C)C(=O)O]

and 2-bromoethanol with a TBS protected hydroxyl group

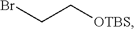 [SMILES: CC(C)(C)[Si](C)(C)OCCBr]

and then removing the Boc group) and K₂CO₃ (1.09 g, 7.9 mmol) were added and stirred overnight at room temperature. At the end of the reaction, the reaction mixture was concentrated under reduced pressure and 50 mL of ethyl acetate was poured into the concentrate. After washing with 10% citric acid (20 mL) and saline (20 mL) successively, the organic phase was dried with anhydrous sodium sulfate, filtered and concentrated, and the crude product was purified by column chromatography to obtain S2-2 (2.36 g).
Step b: under nitrogen atmosphere, DCC (0.91 g, 4.4 mmol) was added to a round-bottom flask containing compounds S2-2 (1.99 g, 2.0 mmol), undecanol (S2-3, 0.4 g, 2.4 mmol) and DMAP (61.00 mg, 0.5 mmol) dissolved in dichloromethane (30 mL) and reacted for 16 h at room temperature. After the reaction, the precipitate was removed by filtration, the filtrate was concentrated, and the resulting residue was purified by column chromatography to obtain compound E2-1 (1.91 g).
Step c: the above compound E2-1 (1.72 g, 1.5 mmol) was dissolved in THE (20 mL) and added into a nitrogen-protected flask, a solution of tetrabutyl ammonium fluoride (TBAF, 20 mL, 1 M) was added, and the reaction was conducted overnight to remove TBS protection. After the reaction, the reaction solution was concentrated, extracted, the organic phases were combined, dried with anhydrous sodium sulfate, filtered and concentrated, and purified by column chromatography to obtain cationic lipid E2-2 (1.36 g, 87.3%). ¹H NMR (400 MHz, CDCl₃) δ: 4.08 (t, 6H), 3.54 (t, 2H), 3.46 (t, 1H), 3.18-2.99 (m, 2H), 2.56 (s, 3H), 2.52 (t, 2H), 2.49 (t, 4H), 2.32-2.25 (m, 2H), 2.01-1.82 (m, 4H), 1.70-1.23 (m, 84H), 0.88 (t, 15H). MS (ESI): m/z=1035.96 ([M+H] ⁺).

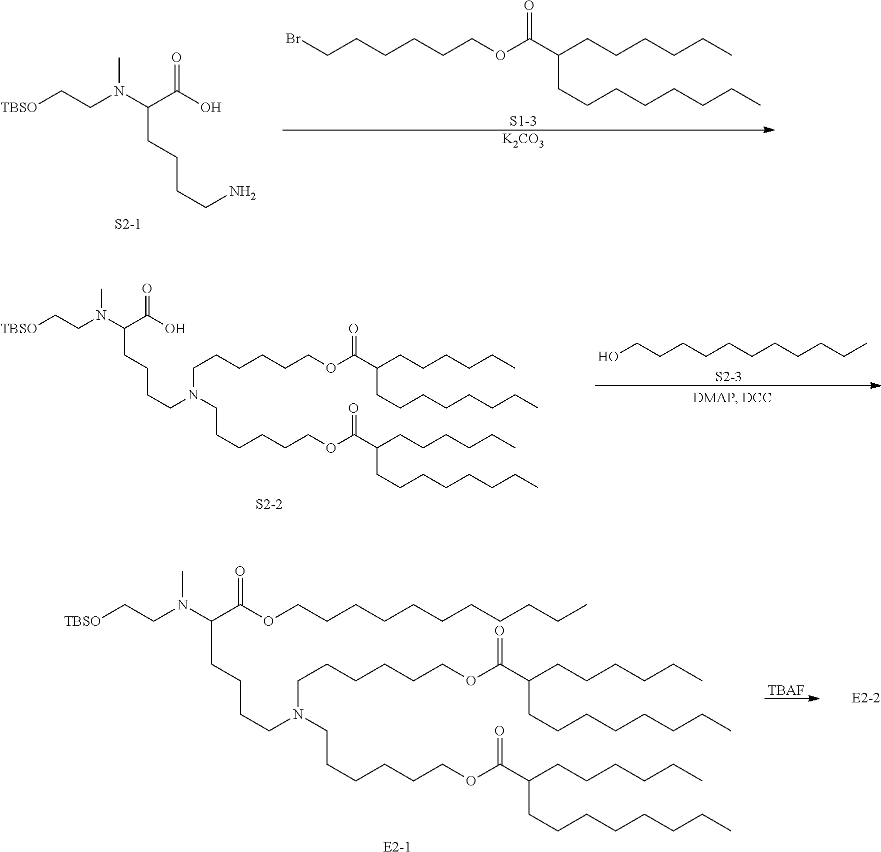

### Example 1.2: Cationic Lipid (E3-2)

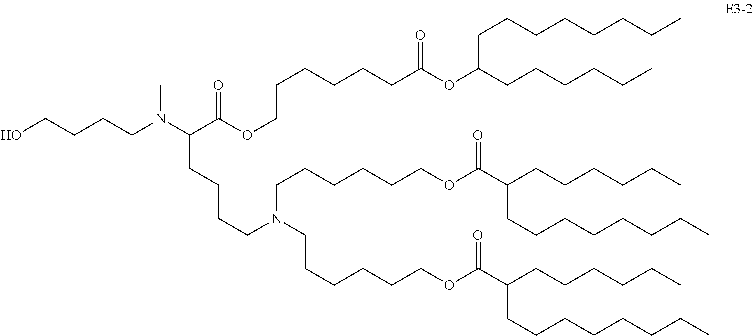 [SMILES: CCCCCCCCC(CCCCCC)OC(=O)CCCCCCOC(=O)C(CCCCN(CCCCCCOC(=O)C(CCCCCC)CCCCCCCC)CCCCCCOC(=O)C(CCCCCC)CCCCCCCC)N(C)CCCCO]

The preparation process is as follows:
Step a: S1-3 (2.77 g, 6.6 mmol) was dissolved in 50 mL of DMF, TBS-N-hydroxybutyl-N-methyllysine (S3-1, 1.04 g, 3.0 mmol, S3-1 was obtained by the reaction between N-methyllysine

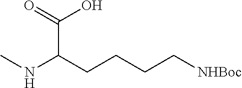 [SMILES: CNC(CCCCNC(=O)OC(C)(C)C)C(=O)O]

with a Boc protected amino group and 4-bromobutanol with a TBS protected hydroxyl group

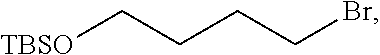 [SMILES: CC(C)(C)[Si](C)(C)OCCCCBr]

and then removing the Boc group) and K₂CO₃ (1.09 g, 7.9 mmol) were added and the reaction was stirred overnight at room temperature. At the end of the reaction, the reaction mixture was concentrated under reduced pressure and 50 mL of ethyl acetate is poured into the concentrate. After washing with 10% citric acid (20 mL) and saline (20 mL) successively, the organic phase was dried with anhydrous sodium sulfate, filtered and concentrated, and the crude product was purified by column chromatography to obtain S3-2 (2.37 g).
Step b: under nitrogen atmosphere, DCC (0.91 g, 4.4 mmol) was added to a round-bottom flask containing compounds S3-2 (2.05 g, 2.0 mmol), 1-hexylnonyl 7-hydroxyheptanoic acid (S3-3, 0.86 g, 2.4 mmol, S3-3 was prepared by the reaction between 7-hydroxyheptanoic acid and pentadecane-7-o1) and DMAP (61.00 mg, 0.5 mmol) dissolved in dichloromethane (30 mL), and the reaction was conducted for 16 h at room temperature. After the reaction, the precipitate was removed by filtration, the filtrate was concentrated, and the resulting residue was purified by column chromatography to obtain compound E3-1 (2.26 g).
Step c: the above compound E3-1 (2.04 g, 1.5 mmol) was dissolved in THE (20 mL), and added into a nitrogen-protected flask, then TBAF solution (20 mL, 1 M) was added to react overnight and remove TBS protection. After the reaction, the reaction solution was concentrated and extracted, the organic phase was combined, dried with anhydrous sodium sulfate, filtered, and the filtrate was concentrated and purified by column chromatography to obtain cationic lipid E3-2 (1.66 g, 88.9%). ¹H NMR (400 MHz, CDCl₃) δ: 4.93-4.81 (m, 1H), 4.08 (t, 6H), 3.68-3.60 (t, 2H), 3.46 (t, 1H), 3.18-2.99 (m, 2H), 2.56 (s, 3H), 2.52 (t, 2H), 2.49 (t, 4H), 2.33-2.25 (m, 4H), 2.01-1.82 (m, 4H), 1.69-1.22 (m, 102H), 0.88 (t, 18H). MS (ESI): m/z=1048.13 ([M+H] ⁺).

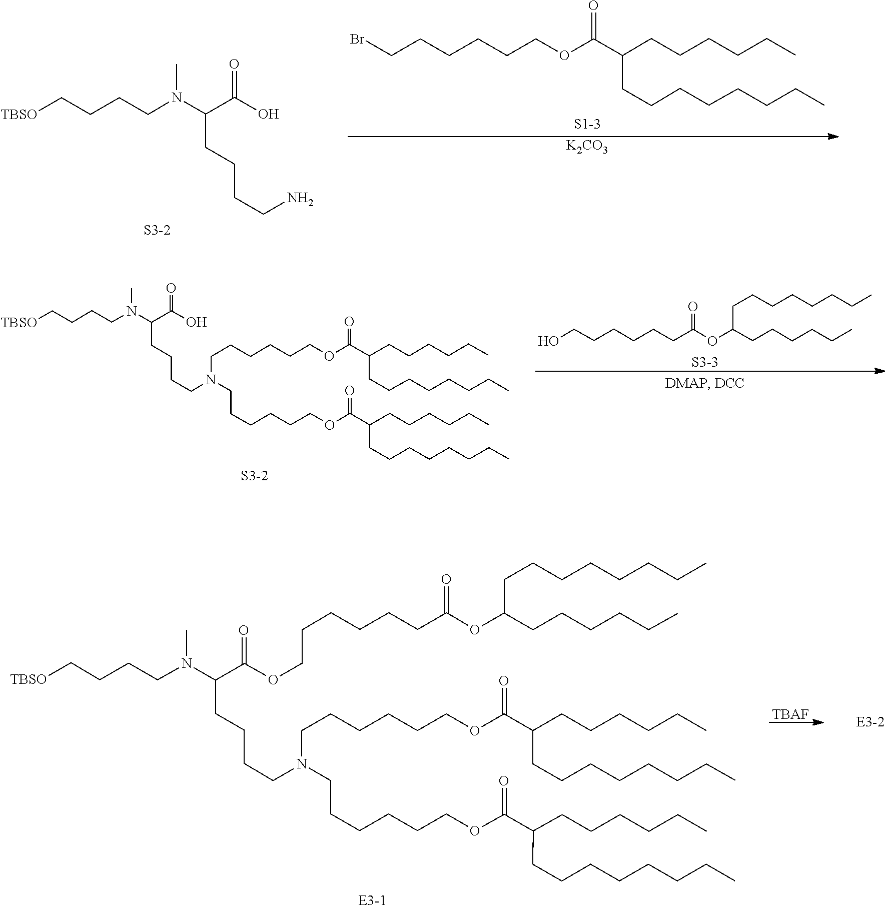

### Example 4: Cationic Lipid (E4-2)

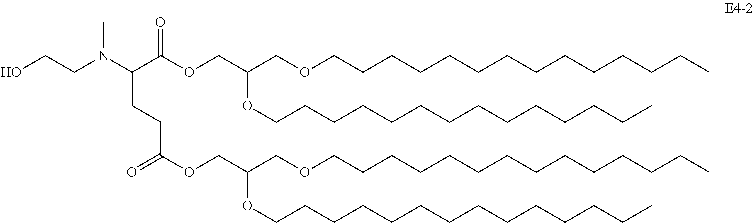 [SMILES: CCCCCCCCCCCCCCOCC(COC(=O)CCC(C(=O)OCC(COCCCCCCCCCCCCCC)OCCCCCCCCCCCCCC)N(C)CCO)OCCCCCCCCCCCCCC]

The preparation process is as follows:
Step a: under nitrogen atmosphere, glycerol (S4-1, 2.06 g, 10.0 mmol) containing a TBS protected hydroxyl group, K₂CO₃ (4.14 g, 30.0 mmol), and bromotetradecane (S4-2, 3.05 g, 11.0 mmol) were dissolved in 60 mL of DMF, the mixture was stirred at 110° C. for 16 h. After the completion of reaction was confirmed by thin layer chromatography, the reaction solution was poured into water (60 mL) for precipitation and filtration, and compound S4-3 (5.35 g, 89.3%) was obtained by further separation and purification with column chromatography.
Step b: the above product S4-3 (4.79 g, 8.0 mmol) was dissolved in THE (50 mL) and added into a nitrogen-protected flask, TBAF (50 mL, 1 M) was added to react overnight and remove TBS protection. After the reaction, the reaction solution was concentrated and extracted, the organic phases were combined, dried with anhydrous sodium sulfate, filtered and concentrated, and purified by column chromatography to obtain 1,2-di-O-tetradecyl-5H-glyceride (S4-4, 3.41 g, 87.9%).
Step c: S4-4 (3.03 g, 6.3 mmol) and S4-5 (0.80 g, 2.5 mmol) were dissolved in anhydrous dichloromethane, respectively, after stirring well, the two component solutions were combined, DMAP (30.50 mg, 0.3 mmol), EDCI (1.20 g, 6.3 mmol) and DIPEA (1.13 g, 12.5 mmol) were added into the mixture sequentially, and the reaction was stirred for 9 h at room temperature. After the end of the reaction, the reaction solution was diluted with dichloromethane (50 mL), then washed twice with saturated sodium carbonate solution (20 mL*2), washed twice with 20 mL aqueous solution, and washed once with saturated saline, the organic phase was dried with anhydrous sodium sulfate, and the solvent was concentrated to obtain a crude product. The crude product was separated and purified by column chromatography, the target eluate was collected, and concentrated to obtain the product E4-1 (1.66 g).
Step d: the above compound E4-1 (1.25 g, 1.0 mmol) was dissolved in THE (20 mL), and added into a nitrogen-protected flask. TBAF solution (20 mL, 1 M) was added and the reaction was conducted overnight to remove TBS protection. After the reaction, the reaction solution was concentrated and extracted, the organic phases were combined, dried with anhydrous sodium sulfate, filtered and concentrated, and purified by column chromatography to obtain cationic lipid E4-2 (1.00 g, 88.1%). ¹H NMR (400 MHz, CDCl₃) δ: 4.51 (t, 1H), 4.13-4.01 (m, 4H), 3.59-3.33 (m, 16H), 2.56 (s, 3H), 2.52 (m, 2H), 2.34 (t, 2H), 2.13-1.92 (m, 2H), 1.55-1.40 (m, 8H), 1.25-1.17 (m, 88H), 0.88 (t, 12H). MS (ESI): m/z=1139.04 ([M+H] ⁺).

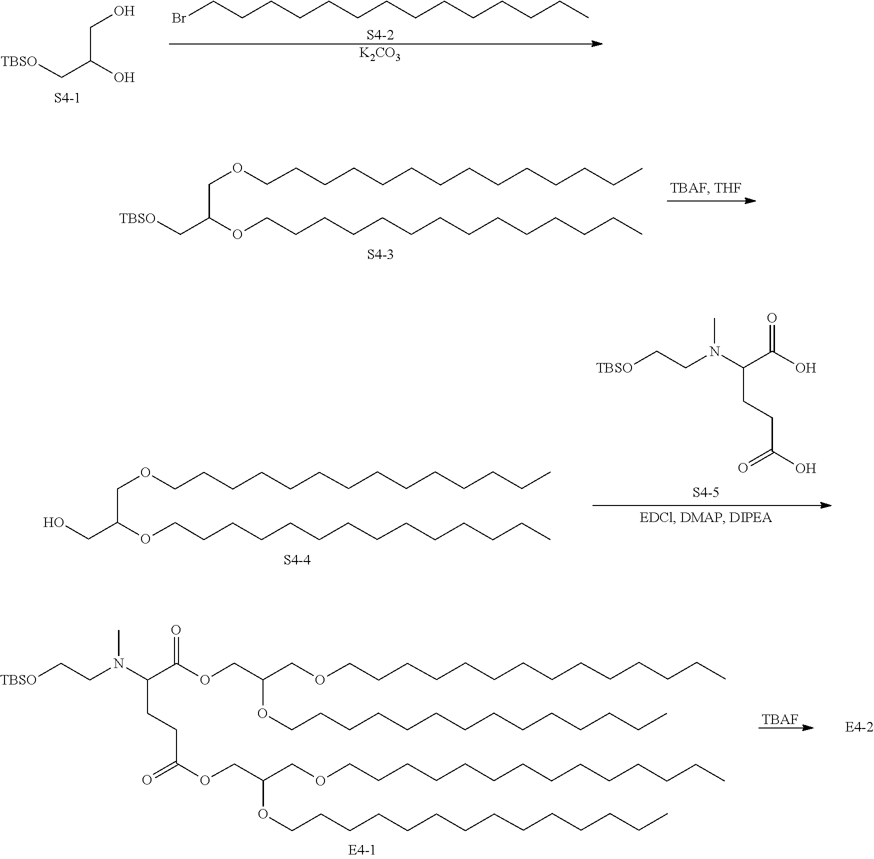

### Example 5: Cationic Lipid (E5-2)

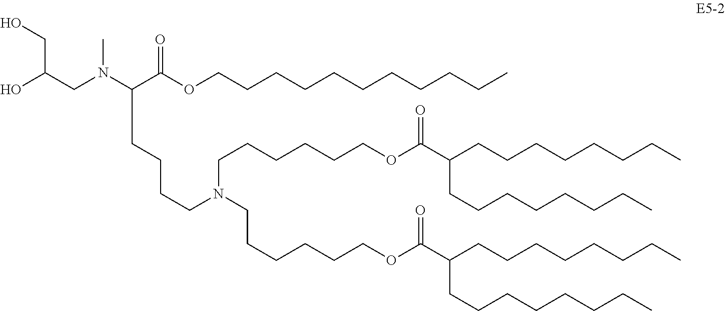 [SMILES: CCCCCCCCCCCOC(=O)C(CCCCN(CCCCCCOC(=O)C(CCCCCCCC)CCCCCCCC)CCCCCCOC(=O)C(CCCCCCCC)CCCCCCCC)N(C)CC(O)CO]

The preparation process is as follows:
Step a: DCC (4.08 g, 19.8 mmol) was added into a round-bottom flask containing 1,16-heptadecandiene-9-carboxylic acid (S5-1, 2.56 g, 9.0 mmol), S1-2 (1.95 g, 10.8 mmol) and DMAP (0.27 g, 2.3 mmol) dissolved in dichloromethane (50 mL). The reaction was carried out at room temperature for 16 h. After the reaction, the precipitate was removed by filtration, the filtrate was concentrated, and the resulting residue was purified by column chromatography to obtain compound S5-2 (3.35 g).
Step b: S5-2 (2.96 g, 6.6 mmol) was dissolved in 50 mL of DMF, then S5-3 (1.39 g, 3.0 mmol, S5-3 was obtained by the reaction between N-methyllysine with a Boc protected amino group

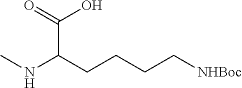 [SMILES: CNC(CCCCNC(=O)OC(C)(C)C)C(=O)O]

and 3-bromo-1,2-propanediol with two TBS protected hydroxyl groups

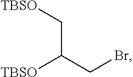 [SMILES: CC(C)(C)[Si](C)(C)OCC(CBr)O[Si](C)(C)C(C)(C)C]

followed by removing the Boc) and K₂CO₃ (1.09 g, 7.9 mmol) were added and stirred overnight at room temperature. At the end of the reaction, the reaction mixture was concentrated under reduced pressure and 50 mL of ethyl acetate was poured into the reaction. After washing with 10% citric acid (20 mL) and saline (20 mL) successively, the organic phase was dried with anhydrous sodium sulfate, filtered and concentrated, and the crude product was purified by column chromatography to obtain S5-4 (2.48 g).
Step c: DCC (0.82 g, 4.0 mmol) was added into a round-bottom flask containing compounds S5-4 (2.15 g, 1.8 mmol), S2-3 (0.82 g, 2.2 mmol) and DMAP (54.90 mg, 0.45 mmol) dissolved in dichloromethane under nitrogen atmosphere, the reaction was conducted for 16 h at room temperature. After the reaction, the precipitate was removed by filtration, the filtrate was concentrated, and the resulting residue was purified by column chromatography to obtain the target compound E5-1 (2.00 g).
Step d: the above compound E5-1 (1.76 g, 1.3 mmol) was dissolved in THE (20 mL), and added into a nitrogen-protected flask, TBAF solution (20 mL, 1 M) was added to react overnight to remove TBS protection. After the reaction, the reaction solution was concentrated and extracted, the organic phases were combined, dried with anhydrous sodium sulfate, filtered and concentrated, and purified by column chromatography to obtain cationic lipid E5-2 (1.28 g, 87.5%). ¹H NMR (400 MHz, CDCl₃) δ: 4.09 (t, 6H), 3.67-3.54 (m, 2H), 3.46-3.38 (m, 2H), 3.18-2.99 (m, 2H), 2.56 (s, 3H), 2.49 (t, 4H), 2.40-2.37 (m, 2H), 2.33-2.25 (m, 2H), 2.01-1.82 (m, 4H), 1.71-1.22 (m, 92H), 0.88 (t, 15H). MS (ESI): m/z=1122.03 ([M+H] ⁺).

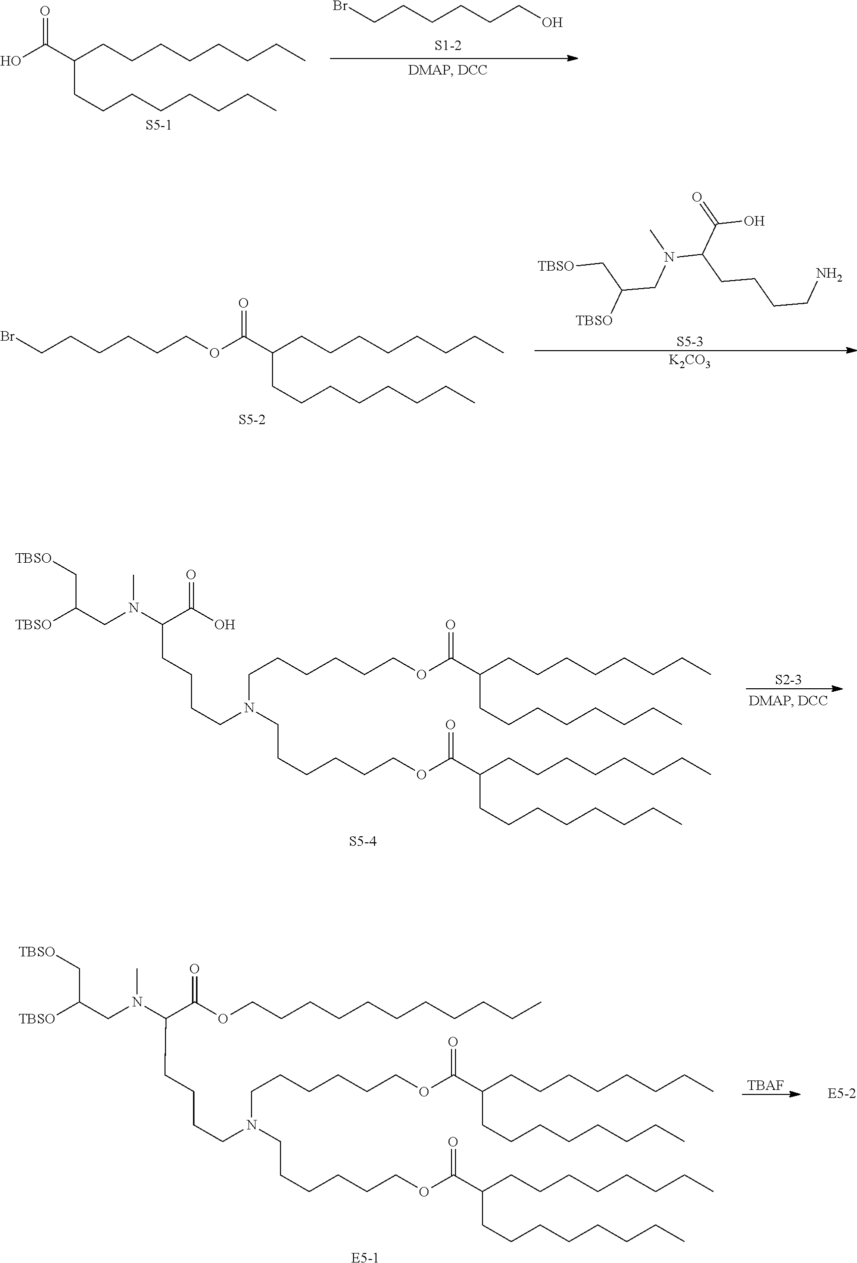

### Example 6: Cationic Lipid (E6-1)

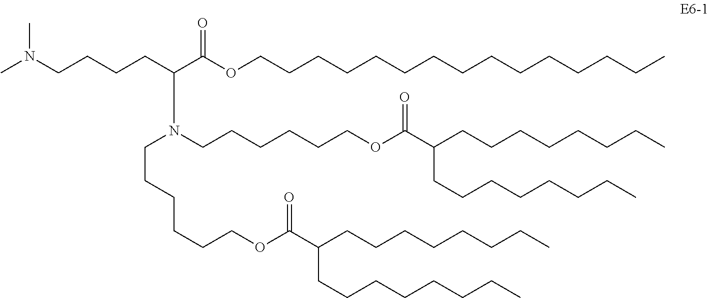 [SMILES: CCCCCCCCCCCCCCCOC(=O)C(CCCCN(C)C)N(CCCCCCOC(=O)C(CCCCCCCC)CCCCCCCC)CCCCCCOC(=O)C(CCCCCCCC)CCCCCCCC]

The preparation process is as follows:
Step a: the above compounds S1-3 (1.84 g, 4.4 mmol) was dissolved in 30 mL of DMF, N,N-dimethyllysine (S6-1, 0.35 g, 3.0 mmol) and K₂CO₃ (0.73 g, 5.3 mmol) were added and stirred overnight at room temperature. At the end of the reaction, the reaction mixture was concentrated under reduced pressure and 30 mL of ethyl acetate was poured into the reaction solution. After washing with 10% citric acid (20 mL) and saline (20 mL) successively, the organic phase was dried with anhydrous sodium sulfate, filtered and concentrated, and the crude product was purified by column chromatography to obtain compound S6-2 (1.38 g).
Step b: DCC (0.45 g, 2.2 mmol) was added into a round-bottom flask containing compounds S6-2 (0.85 g, 1.0 mmol), 1-pentadecanol (S6-3, 0.27 g, 1.2 mmol) and DMAP (30.50 mg, 0.3 mmol) dissolved in dichloromethane (10 mL) under nitrogen atmosphere, and the reaction was conducted for 16 h at room temperature. After the reaction, the precipitate was removed by filtration, the filtrate was concentrated, and the resulting residue was purified by column chromatography to obtain cationic lipid E6-1 (0.93 g). ¹H NMR (400 MHz, CDCl₃) δ: 4.07 (t, 6H), 3.27 (t, 1H), 2.64-2.53 (m, 2H), 2.45-2.37 (m, 2H), 2.33-2.31 (m, 2H), 2.29-2.26 (m, 6H), 2.25-2.19 (m, 2H), 2.07-1.95 (m, 2H), 1.66-1.51 (m, 14H), 1.29-1.23 (m, 88H), 0.88 (t, 15H). MS (ESI): m/z=1118.07 ([M+H] ⁺).

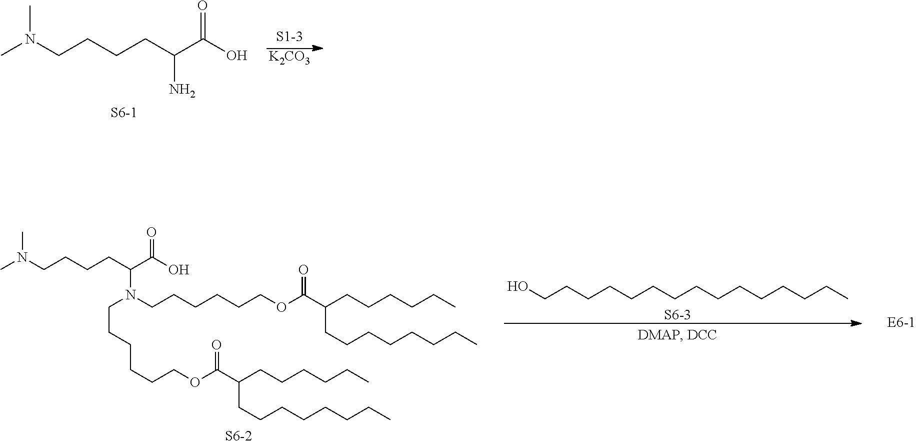

### Example 7: Cationic Lipid (E7-1)

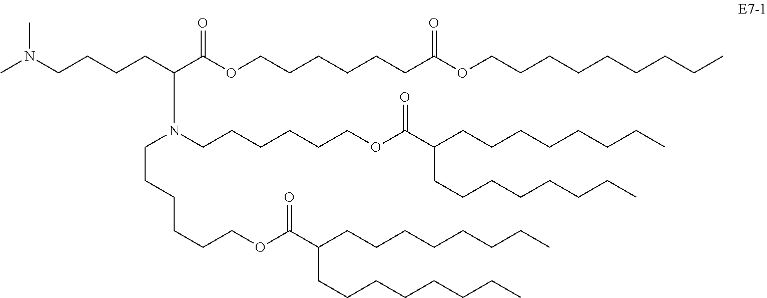 [SMILES: CCCCCCCCCOC(=O)CCCCCCOC(=O)C(CCCCN(C)C)N(CCCCCCOC(=O)C(CCCCCCCC)CCCCCCCC)CCCCCCOC(=O)C(CCCCCCCC)CCCCCCCC]

The preparation process is as follows:
Step a: DCC (3.63 g, 17.6 mmol) was added to a round-bottom flask containing TBS-7-hydroxyheptanoic acid (S7-1, 2.08 g, 8.0 mmol), 1-nonanol (S7-2, 1.38 g, 9.6 mmol) and DMAP (0.24 g, 2.0 mmol) dissolved in dichloromethane (50 mL), and the reaction was conducted for 16 h at room temperature. After the reaction, the precipitate was removed by filtration, the filtrate was concentrated, and the resulting residue was purified by column chromatography to obtain the compound TBS-7-hydroxyheptanoate nonyl ester (S7-3, 2.56 g).
Step b: the above compound S7-3 (1.94 g, 5.0 mmol) was dissolved in THE (20 mL), TBAF solution (20 mL, 1 M) was added under nitrogen protection to react overnight to remove TBS protection. After the reaction, the reaction solution was concentrated and extracted, the organic phases were combined, dried with anhydrous sodium sulfate, filtered and concentrated, and purified by column chromatography to obtain nonyl 7-hydroxyheptanoate (S7-4, 1.20 g, 88.4%).
Step c: DCC (0.91 g, 4.4 mmol) was added into a round-bottom flask containing compounds S6-2 (1.70 g, 2.0 mmol), S7-4 (0.65 g, 2.4 mmol) and DMAP (61.00 mg, 0.5 mmol) dissolved in dichloromethane (20 mL) under nitrogen atmosphere and the reaction was conducted for 16 h at room temperature. After the reaction, the precipitate was removed by filtration, the filtrate was concentrated, and the resulting residue was purified by column chromatography to obtain cationic lipid E7-1 (1.93 g). ¹H NMR (400 MHz, CDCl₃) δ: 4.09 (t, 8H), 3.46 (t, 1H), 3.18-2.99 (m, 2H), 2.49 (t, 4H), 2.32-2.25 (m, 4H), 2.23 (s, 6H), 2.01-1.82 (m, 4H), 1.71-1.22 (m, 96H), 0.88 (t, 15H). MS (ESI): m/z=1162.06 ([M+H] ⁺).

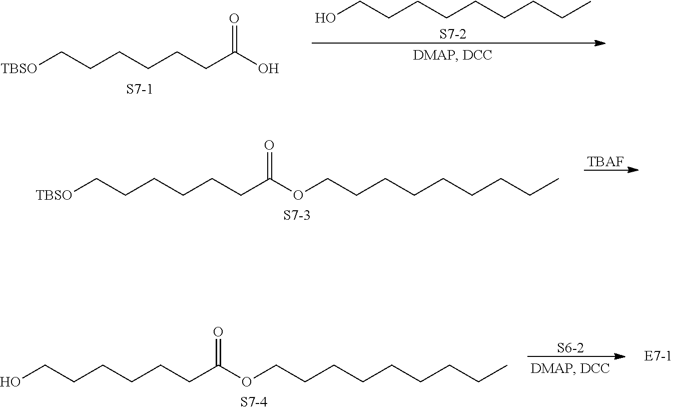

### Example 8: Cationic Lipid (E8-2)

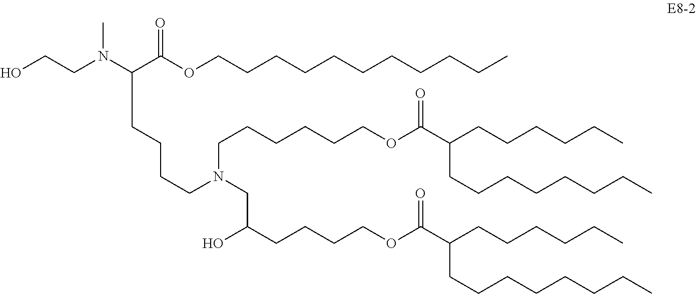 [SMILES: CCCCCCCCCCCOC(=O)C(CCCCN(CCCCCCOC(=O)C(CCCCCC)CCCCCCCC)CC(O)CCCCOC(=O)C(CCCCCC)CCCCCCCC)N(C)CCO]

The preparation process is as follows:
Step a: DCC (5.44 g, 26.4 mmol) was added into a round-bottom flask containing compounds S1-1 (3.07 g, 12.0 mmol), 5-hexen-1-ol (S8-1, 1.44 g, 14.4 mmol) and DMAP (0.37 g, 3.0 mmol) dissolved in dichloromethane (50 mL) under nitrogen atmosphere, and the reaction was conducted for 16 h at room temperature. After the reaction, the precipitate was removed by filtration, the filtrate was concentrated, and the resulting residue was purified by column chromatography to obtain the target compound 5-hexene-2-hexylcaprate (S8-2, 3.61 g).
Step b: m-chloroperoxybenzoic acid (3.11 g, 18.0 mmol) was added to a solution of dichloromethane (50 mL) in which S8-2 (3.18 g, 9.0 mmol) was dissolved, and the reaction was stirred for 10 h at room temperature. After the reaction was completed, the reaction mixture was poured into NaHCO₃ (20 mL) solution and extracted with dichloromethane (20 mL*3). The combined organic layer was washed with Na₂S₂O₃ (aqueous solution) and saline, dried with anhydrous sodium sulfate, and concentrated under vacuum. The crude product was purified by column chromatography to obtain 5,6-epoxyhexane-2-hexylcapanoate (S8-3, 2.63 g).
Step c: compound S2-1 (3.34 g, 10.5 mmol) was added to an anhydrous ethanol (30 mL) solution in which S8-3 (2.48 g, 7.0 mmol) was dissolved, and the reaction was stirred for 10 h at room temperature. After the reaction, the reaction solution was concentrated and extracted, the organic phases were combined, dried with anhydrous sodium sulfate, filtered and concentrated, and purified by column chromatography to obtain compound S8-4 (2.17 g).
Step d: compound S8-4 (1.88 g, 2.8 mmol) was dissolved in 30 mL of anhydrous THE under nitrogen protection, and the solution was cooled to 0° C. t-butyldimethylchlorosilane (TBSCl, 0.85 g, 5.6 mmol) was slowly added into the above reaction solution, followed by imidazole (0.76 g, 11.2 mmol), and then the reaction was stirred for 18 h at room temperature.

After the reaction, the reaction was quenched with ice water (10 mL), extracted with ethyl acetate (10 mL*3), the organic phases were combined, washed with saturated NaHCO₃ solution to remove acidic impurities, dried with anhydrous sodium sulfate, evaporated under reduced pressure to concentrate to obtain the crude product and then purified by column chromatography to obtain compound S8-5 (1.87 g).
Step e: S1-3 (1.00 g, 2.4 mmol) was dissolved in 30 mL of DMF, then S8-5 (1.57 g, 2.0 mmol) and K₂CO₃ (0.73 g, 5.3 mmol) were added and the reaction was stirred overnight at room temperature. At the end of the reaction, the reaction mixture was concentrated under reduced pressure and 30 mL of ethyl acetate was poured into the reaction solution. After washing with 10% citric acid (20 mL) and saline (20 mL) successively, the organic phase was dried with anhydrous sodium sulfate, filtered and concentrated, and the crude product was purified by column chromatography to obtain S8-6 (1.82 g).
Step f: DCC (0.59 g, 2.9 mmol) was added into a round-bottom flask containing compounds S8-6 (1.46 g, 1.3 mmol), S2-3 (0.27 g, 1.6 mmol) and DMAP (39.65 mg, 0.33 mmol) dissolved in dichloromethane (20 mL) under nitrogen atmosphere and the reaction was conducted for 16 h at room temperature. After the reaction, the precipitate was removed by filtration, the filtrate was concentrated, and the resulting residue was purified by column chromatography to obtain the target compound E8-1 (1.42 g).
Step g: the above compound E8-1 (1.28 g, 1.0 mmol) was dissolved in THE (20 mL) and added into a nitrogen-protected flask, TBAF solution (20 mL, 1 M) was added to react overnight and remove TBS protection. After the reaction, the reaction solution was concentrated, extracted, the organic phases were combined, dried with anhydrous sodium sulfate, filtered and concentrated, and purified by column chromatography to obtain cationic lipid E8-2 (0.92 g, 87.5%). ¹NMR (400 MHz, CDCl₃) δ: 4.07 (t, 6H), 3.55-3.53 (m, 3H), 3.46 (t, 1H), 3.18-2.99 (m, 2H), 2.56 (s, 3H), 2.52 (t, 2H), 2.49 (t, 2H), 2.33-2.25 (m, 4H), 2.01-1.82 (m, 4H), 1.71-1.22 (m, 82H), 0.88 (t, 15H). MS (ESI): m/z=1051.95 ([M+H] ⁺).

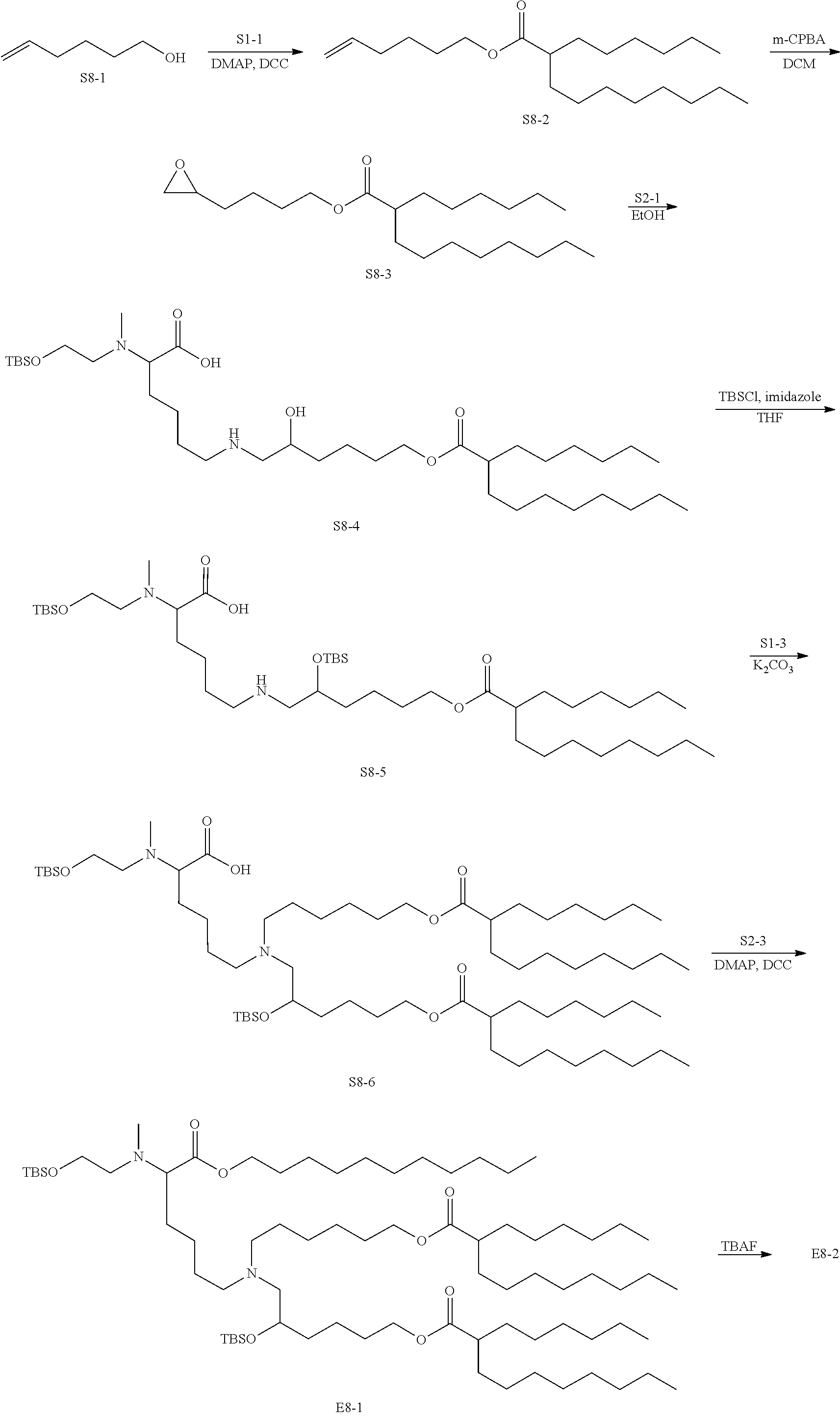

### Example 9: Cationic Lipid (E9-3)

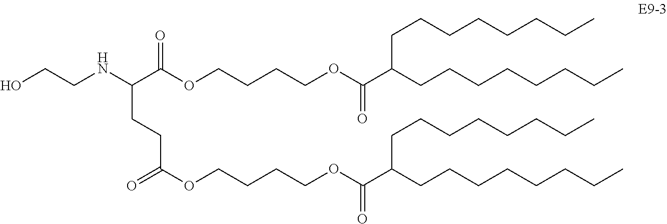 [SMILES: CCCCCCCCC(CCCCCCCC)C(=O)OCCCCOC(=O)CCC(NCCO)C(=O)OCCCCOC(=O)C(CCCCCCCC)CCCCCCCC]

The preparation process is as follows:
Step a: DCC (4.53 g, 22.0 mmol) was added to a round-bottom flask containing compounds 2-octylcapric acid (S9-2, 2.84 g, 10.0 mmol), 1,4-butanediol (S9-1, 4.53 g, 12.0 mmol) and DMAP (0.31 g, 2.5 mmol) dissolved in dichloromethane (60 mL) under nitrogen atmosphere, and the reaction was conducted for 16 h at room temperature. At the end of the reaction, the precipitate was removed by filtration, the filtrate was concentrated, and the resulting residue was purified by column chromatography to obtain a small molecule intermediate alcohol derivative (S9-3, 2.98 g).
Step b: N-hydroxyethyl glutamic acid (S9-4, 1.22 g, 3.0 mmol, S9-4 was obtained by the reaction between glutamic acid with a Boc protected amino group

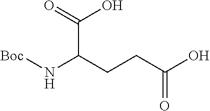 [SMILES: CC(C)(C)OC(=O)NC(CCC(=O)O)C(=O)O]

and 2-bromoethanol with TBS-protected hydroxyl groups

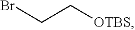 [SMILES: CC(C)(C)[Si](C)(C)OCCBr]

followed by removing the Boc group) and S9-3 (2.68 g, 7.5 mmol) were dissolved in anhydrous dichloromethane separately, stirred well and combined with a two-component solution, and DMAP (36.60 mg, 0.3 mmol), 1-ethyl-(3-dimethylaminopropyl)carbodiimide hydrochloride (EDCI, 1.44 g, 7.5 mmol) and N,N-diisopropylethylamine (DIPEA, 1.35 g, 15.0 mmol) were added sequentially to the mixture and stirred for 48 h at room temperature. After the end of the reaction, the reaction solution was diluted with dichloromethane (50 mL), then washed twice with saturated sodium carbonate solution (30 mL*2), washed twice with 30 mL aqueous solution, and then washed once with saturated saline, and the organic phase was dried with anhydrous sodium sulfate, and the solvent was concentrated to obtain a crude product. The crude product was separated and purified by column chromatography, the target eluate was collected and concentrated to obtain the product E9-1 (1.72 g).
Step c: removing the Boc protecting group. A solution of trifluoroacetic acid/dichloromethane (1:2, v/v) was prepared in a dry and clean round-bottom flask, dichloromethane solution of E9-1 (1.30 g, 1.2 mmol) was slowly added dropwise under ice bath conditions, and the reaction was conducted for 2 h at room temperature. At the end of the reaction, the reaction solution was concentrated, diluted with purified water, and extracted with dichloromethane, the organic phase was dried with anhydrous magnesium sulfate and filtered, and the filtrate was concentrated and recrystallized to obtain compound E9-2 (1.07 g, 90.5%).
Step d: the above compound E9-2 (0.79 g, 0.8 mmol) was dissolved in THE (20 mL), and added into a nitrogen-protected flask, TBAF solution (20 mL, 1 M) was added and reacted overnight to remove TBS protection. After the reaction, the reaction solution was concentrated and extracted, the organic phases were combined, dried with anhydrous sodium sulfate, filtered and concentrated, and purified by column chromatography to obtain the cationic lipid E9-3 (0.61 g, 88.4%). ¹H NMR (400 MHz, CDCl3) δ: 4.51 (t, 1H), 4.09 (t, 8H), 3.54 (t, 2H), 2.74 (t, 2H), 2.34-2.26 (m, 4H), 2.13-1.92 (m, 2H), 1.71-1.22 (m, 64H), 0.88 (t, 12H). MS (ESI): m/z=868.72 ([M+H] ⁺).

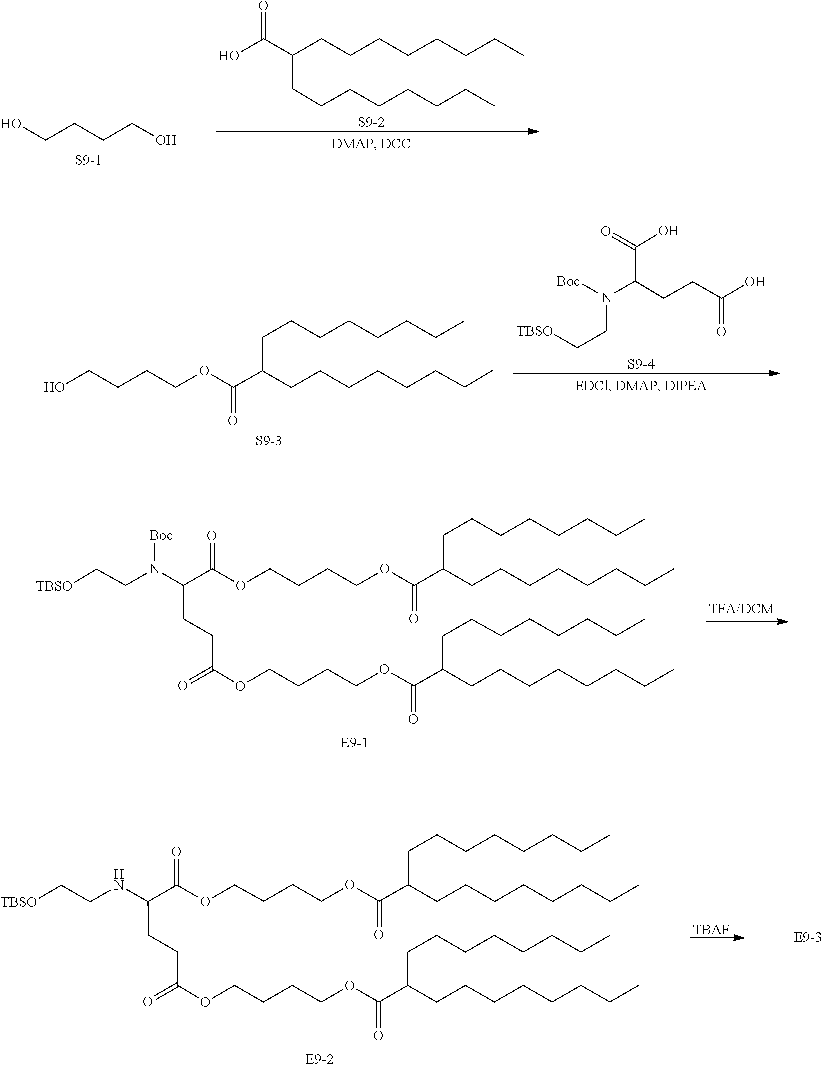

### Example 10: Cationic Lipid (E10-3)

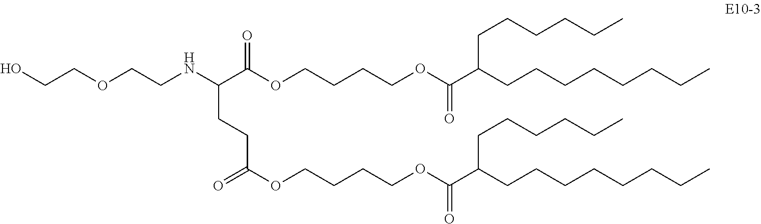 [SMILES: CCCCCCCCC(CCCCCC)C(=O)OCCCCOC(=O)CCC(NCCOCCO)C(=O)OCCCCOC(=O)C(CCCCCC)CCCCCCCC]

The preparation process is as follows:
Step a: DCC (5.44 g, 26.4 mmol) was added into a round-bottom flask containing compounds S1-1 (3.07 g, 12.0 mmol), S9-1 (1.30 g, 14.4 mmol) and DMAP (0.37 g, 3.0 mmol) dissolved in dichloromethane (50 mL) under nitrogen atmosphere and the reaction was conducted for 16 h at room temperature. After the reaction, the precipitate was removed by filtration, the filtrate was concentrated, and the resulting residue was purified by column chromatography to obtain small molecule intermediate alcohol derivatives (S10-1, 3.30 g).
Step b: S10-1 (2.96 g, 9.0 mmol) and S10-2 (1.62 g, 3.6 mmol, S10-2 was obtained by the reaction between glutamic acid with a Boc-protected amino group

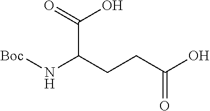 [SMILES: CC(C)(C)OC(=O)NC(CCC(=O)O)C(=O)O]

and 2-(2-bromoethoxy)ethanol with TBS-protected hydroxyl groups

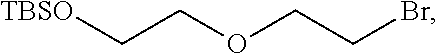 [SMILES: CC(C)(C)[Si](C)(C)OCCOCCBr]

followed by removing the Boc group) were dissolved in anhydrous dichloromethane respectively. After stirring well, the two component solutions were combined, and DMAP (43.92 mg, 0.36 mmol), EDCI (1.73 g, 9.0 mmol) and DIPEA (1.62 g, 18.0 mmol) were added into the mixture sequentially, and the reaction was stirred for 48 h at room temperature. After the end of the reaction, the reaction solution was diluted with dichloromethane (50 mL), then washed twice with saturated sodium carbonate solution (30 mL*2), washed twice with 30 mL aqueous solution, and then washed once with saturated saline, and the organic phase was dried with anhydrous sodium sulfate, and the solvent was concentrated to obtain a crude product. The crude product was separated and purified by column chromatography, the target eluate was collected and concentrated to obtain the product E10-1 (1.98 g).
Step c: removing the Boc protecting group. A solution of trifluoroacetic acid/dichloromethane (1:2, v/v) was prepared in a dry and clean round-bottom flask. A dichloromethane solution of E10-1 (1.61 g, 1.5 mmol) was slowly added dropwise under ice bath conditions, and the reaction was conducted for 2 h at room temperature. At the end of the reaction, the reaction solution was concentrated, diluted with purified water, and extracted with dichloromethane, the organic phases was dried with anhydrous magnesium sulfate and filtered, the filtrate was concentrated and recrystallized to obtain compound E10-2 (1.31 g, 90.1%).
Step d: The above compound E10-2 (0.97 g, 1.0 mmol) was dissolved in THE (20 mL) and added into a nitrogen-protected flask. TBAF solution (20 mL, 1 M) was added to react overnight and remove TBS protection. After the reaction, the reaction solution was concentrated and extracted, the organic phases were combined, dried with anhydrous sodium sulfate, filtered and concentrated, and purified by column chromatography to obtain cationic lipid E10-3 (0.76 g, 89.0%). ¹H NMR (400 MHz, CDCl3) δ: 1H NMR (400 MHz, CDCl3) δ: 4.51 (t, 1H), 4.07 (t, 8H), 3.70 (t, 2H), 3.63 (t, 4H), 2.74 (t, 2H), 2.33-2.25 (m, 4H), 2.13-1.92 (m, 2H), 1.68-1.22 (m, 56H), 0.88 (t, 12H). MS (ESI): m/z=856.68 ([M+H] ⁺).

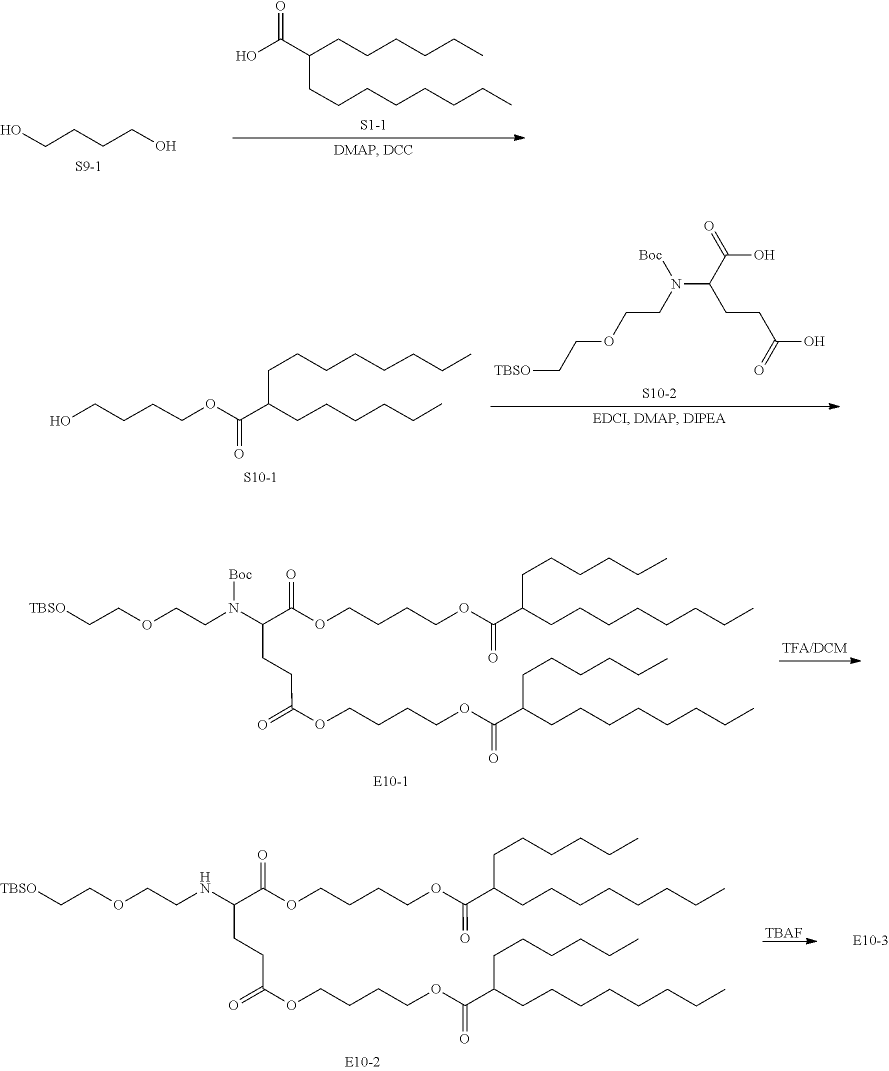

### Example 11: Cationic Lipid (E11-2)

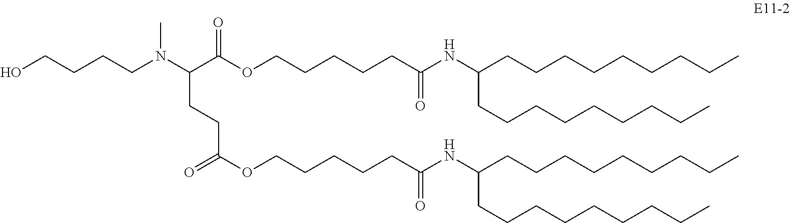 [SMILES: CCCCCCCCCC(CCCCCCCCC)NC(=O)CCCCCOC(=O)CCC(C(=O)OCCCCCC(=O)NC(CCCCCCCCC)CCCCCCCCC)N(C)CCCCO]

The preparation process is as follows:
Step a: TBS-6-hydroxyhexanoic acid (S11-1, 3.69 g, 15.0 mmol), EDCI (3.46 g, 18.0 mmol) and N-hydroxysuccinimide (NHS, 1.88 g, 16.5 mmol) were dissolved in dichloromethane (50 mL) and stirred overnight at room temperature. After the reaction, the reaction solution was backwashed twice (20 mL*2) with 0.1 mol/L HCl, backwashed once with saturated sodium chloride (20 mL), the dichloromethane phase was collected, dried with anhydrous magnesium sulfate, and filtered, and the filtrate was concentrated to obtain compound S11-2 (5.07 g, 98.6%).
Step b: the above compound S11-2 (4.12 g, 12.0 mmol) was dissolved in dichloromethane (50 mL), 10-nonadecamine (S11-3, 3.75 g, 13.2 mmol) and triethylamine (TEA, 2.5 mL, 18.0 mmol) were added and the reaction was conducted overnight at room temperature to precipitate a large amount of white solid. The solids were filtered, slurried with methanol (20 mL), filtered, and rinsed twice with methanol, and the solids were collected and dried to obtain TBS-6-hydroxy-N-(10-nonadecalaminyl)hexacamide (S11-4, 5.15 g, 83.8%).
Step c: the above compound S11-4 (4.61 g, 9.0 mmol) was dissolved in THE (50 mL), and added into a nitrogen-protected flask. TBAF solution (50 mL, 1 M) was added to react overnight to remove TBS protection. After the reaction, the reaction solution was concentrated and extracted, the organic phases were combined, dried with anhydrous sodium sulfate, filtered, concentrated, and purified by column chromatography to obtain the compound S11-5 (3.15 g, 87.9%).
Step d: S11-5 (2.48 g, 6.3 mmol) and TBS-N-hydroxybutyl-N-methylglutamic acid (S11-6, 0.87 g, 2.5 mmol, S11-6 was obtained by the reaction between glutamic acid with a Boc protected amino group

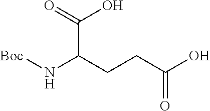 [SMILES: CC(C)(C)OC(=O)NC(CCC(=O)O)C(=O)O]

and 4-bromobutanol with TBS protected hydroxyl groups

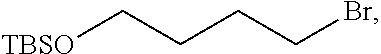 [SMILES: CC(C)(C)[Si](C)(C)OCCCCBr]

followed by removing the Boc group) were dissolved in anhydrous dichloromethane, respectively. After stirring well, the two component solutions were combined. DMAP (30.50 mg, 0.25 mmol), EDCI (1.20 g, 6.3 mmol) and DIPEA (1.13 g, 12.5 mmol) were added to the mixture, then stirred for 48 h at room temperature. At the end of the reaction, the reaction solution was diluted with dichloromethane (30 mL), then washed twice with saturated sodium carbonate solution (20 mL*2), washed twice with 20 mL aqueous solution, and then washed once with saturated saline, the organic phase was dried with anhydrous sodium sulfate, and the solvent was concentrated to obtain a crude product. The crude product was separated and purified by column chromatography, the target eluate was collected and concentrated to obtain the product E11-1 (1.43 g).
Step e: the above compound E11-1 (1.11 g, 1.0 mmol) was dissolved in THE (20 mL), and added into a nitrogen-protected flask. TBAF solution (20 mL, 1 M) was added to react overnight and remove TBS protection. After the reaction, the reaction solution was concentrated and extracted, the organic phases were combined, dried with anhydrous sodium sulfate, filtered and concentrated, and purified by column chromatography to obtain cationic lipid E11-2 (0.88 g, 88.3%). ¹H NMR (400 MHz, CDCl₃) δ: 4.51 (t, 1H), 4.07 (t, 4H), 3.96-3.82 (m, 2H), 3.68-3.60 (t, 2H), 2.56 (s, 3H), 2.52 (t, 2H), 2.34 (t, 2H), 2.17 (t, 4H), 2.13-1.92 (m, 2H), 1.69-1.21 (m, 80H), 0.88 (t, 12H). MS (ESI): m/z=992.89 ([M+H] ⁺).

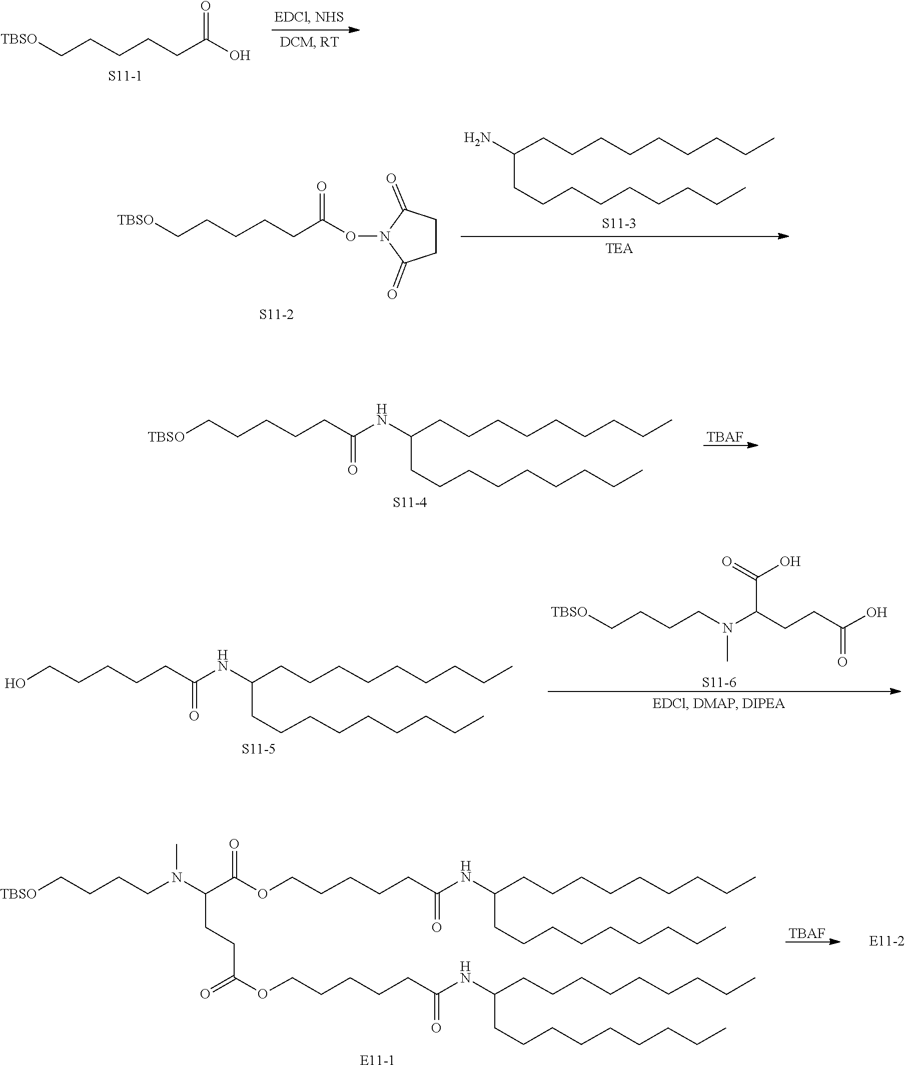

### Example 12: Cationic Lipid (E12-2)

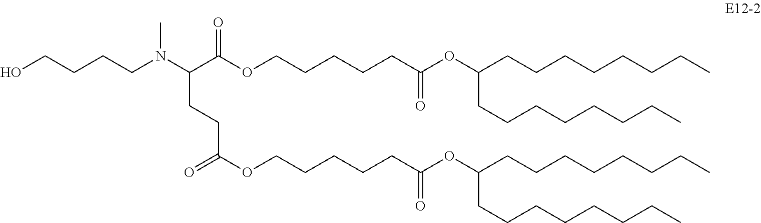 [SMILES: CCCCCCCCC(CCCCCCCC)OC(=O)CCCCCOC(=O)CCC(C(=O)OCCCCCC(=O)OC(CCCCCCCC)CCCCCCCC)N(C)CCCCO]

The preparation process is as follows:
Step a: DCC (3.63 g, 17.6 mmol) was added into a round-bottom flask containing compounds 9-hepattacanol (S12-1, 2.46 g, 9.6 mmol), S11-1 (1.97 g, 8.0 mmol) and DMAP (0.24 g, 2.0 mmol) dissolved in dichloromethane (50 mL) under nitrogen atmosphere, and the reaction was conducted for 16 h at room temperature. After the reaction, the precipitate was removed by filtration, the filtrate was concentrated, and the resulting residue was purified by column chromatography to obtain compound 6-hydroxycaproate-1-octylnonyl ester (S12-2, 3.22 g).
Step b: the above compound S12-2 (2.91 g, 6.0 mmol) was dissolved in THE (30 mL), and added into a nitrogen-protected flask. TBAF solution (30 mL, 1 M) was added and reacted overnight to remove TBS protection. After the reaction, the reaction solution was concentrated and extracted, the organic phases were combined, dried with anhydrous sodium sulfate, filtered and concentrated, and purified by column chromatography to obtain the compound S12-3 (1.96 g, 88.2%).
Step c: S12-3 (1.86 g, 5.0 mmol) and S11-6 (0.70 g, 2.0 mmol) were dissolved in anhydrous dichloromethane, respectively. After stirring well, two component solutions were combined, and then DMAP (24.40 mg, 0.2 mmol), EDCI (0.96 g, 5.0 mmol) and DIPEA (0.90 g, 10.0 mmol) were added sequentially into the mixture, and the reaction was stirred at room temperature for 48 h. After the end of the reaction, the reaction solution was diluted with dichloromethane (30 mL), then washed twice with saturated sodium carbonate solution (20 mL*2), washed twice with 20 mL aqueous solution, and then washed once with saturated saline, the organic phase was dried with anhydrous sodium sulfate, and the solvent was concentrated to obtain a crude product. The crude product was separated and purified by column chromatography, the target eluate was collected and concentrated to obtain compound E12-1 (1.18 g).
Step d: the above compound E12-1 (0.84 g, 0.8 mmol) was dissolved in THE (10 mL), and added into a nitrogen-protected flask. TBAF solution (10 mL, 1 M) was added to react overnight and remove TBS protection. After the reaction, the reaction solution was concentrated and extracted, the organic phases were combined, dried with anhydrous sodium sulfate, filtered and concentrated, and purified by column chromatography to obtain the cationic lipid E12-2 (0.66 g, 88.1%). ¹H NMR (400 MHz, CDCl₃) δ: 4.93-4.81 (m, 2H), 4.51 (t, 1H), 4.07 (t, 4H), 3.68-3.60 (t, 2H), 2.56 (s, 3H), 2.52 (t, 2H), 2.34 (t, 2H), 2.32 (t, 4H), 2.13-1.92 (m, 2H), 1.70-1.21 (m, 72H), 0.88 (t, 12H). MS (ESI): m/z=938.79 ([M+H] ⁺).

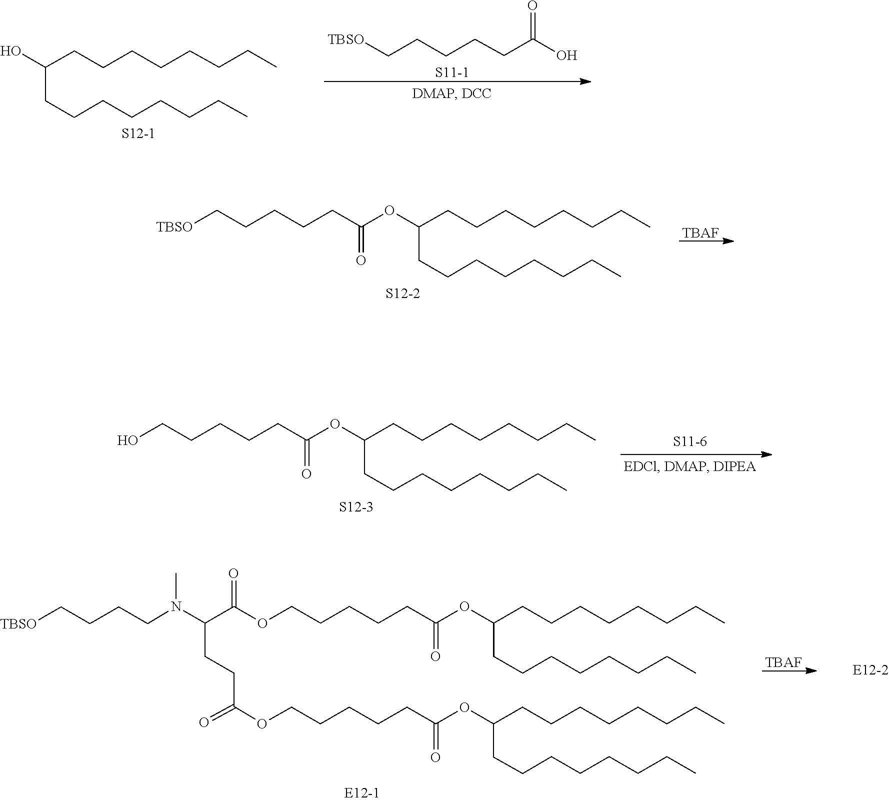

### Example 13: Cationic Lipid (E13-1)

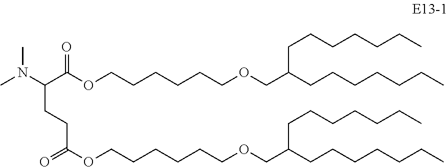 [SMILES: CCCCCCCC(CCCCCCC)COCCCCCCOC(=O)CCC(C(=O)OCCCCCCOCC(CCCCCCC)CCCCCCC)N(C)C]

The preparation process is as follows:
Step a: 2-heptylnonanol (S13-1, 3.63 g, 15.0 mmol) was dissolved in dried THF (50 mL). Under nitrogen protection and ice bath stirring, NaH (60%, 0.60 g, 15.0 mmol) was slowly added and then reacted under ice bath for 1 h. TBS-6-bromohexanol (S13-2, 5.31 g, 18.0 mmol, the hydroxyl group of S13-2 was protected by TBS) was then added and reacted overnight at room temperature. After the reaction, the reaction was placed under an ice bath, 5 mL of water was slowly added to quench the reaction, the reaction solution was poured into 20 mL of water, extracted twice with ethyl acetate (50 mL*2), the organic phases were combined, and backwashed once with saturated sodium chloride aqueous solution (50 mL), dried with anhydrous sodium sulfate, and filtered, and the filtrate was concentrated to obtain an oily crude product. The small molecule intermediate S13-3 (3.57 g) was obtained by column chromatography purification, concentration, and oil pump drying.
Step b: the above compound S13-3 (3.20 g, 7.0 mmol) was dissolved in THE (50 mL), and added into a nitrogen-protected flask. TBAF solution (50 mL, 1 M) was added to react overnight and remove TBS protection. After the reaction, the reaction solution was concentrated and extracted, the organic phases were combined, dried with anhydrous sodium sulfate, filtered and concentrated, and purified by column chromatography to obtain the the compound S13-4 (2.12 g, 88.3%).
Step c: S13-4 (1.72 g, 5.0 mmol) and S13-5 (0.35 g, 2.0 mmol) were dissolved in anhydrous dichloromethane, respectively. After stirring well, two component solutions were combined, and then DMAP (24.40 mg, 0.2 mmol), EDCI (0.96 g, 5.0 mmol), and DIPEA (0.90 g, 10.0 mmol) were added sequentially into the mixture, and the reaction was stirred at room temperature for 48 h. After the reaction, the reaction solution was diluted with dichloromethane (30 mL), then washed twice with saturated sodium carbonate solution (20 mL*2), washed twice with 20 mL aqueous solution, and then washed once with saturated saline, the organic phase was dried with anhydrous sodium sulfate, and the solvent was concentrated to obtain a crude product. The crude product was separated and purified by column chromatography, the target eluate was collected and concentrated to obtain cationic lipid E13-1 (0.87 g). ¹H NMR (400 MHz, CDCl₃) δ: 4.51 (t, 1H), 4.08 (t, 4H), 3.37 (t, 4H), 3.28-3.24 (m, 4H), 2.56 (s, 6H), 2.34 (t, 2H), 2.13-1.92 (m, 2H), 1.72-1.21 (m, 66H), 0.88 (t, 12H). MS (ESI): m/z=824.76 ([M+H] ⁺).

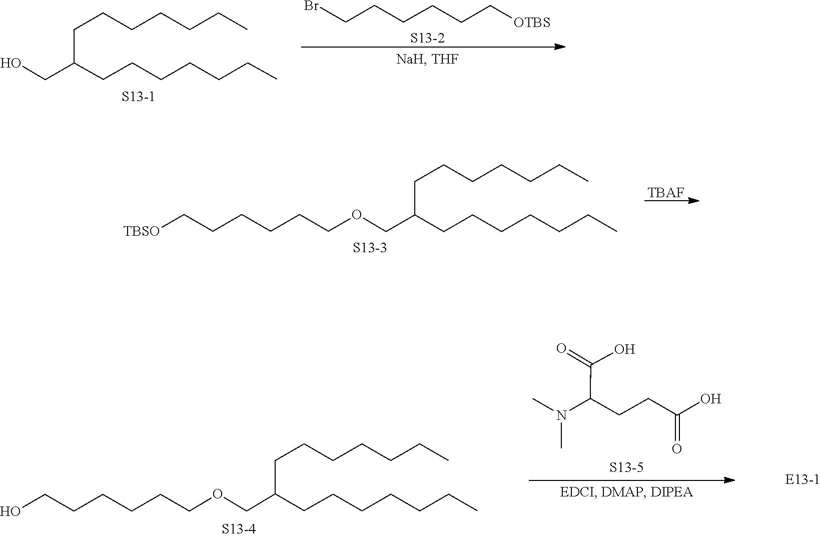

### Example 14: Cationic Lipid (E14-1)

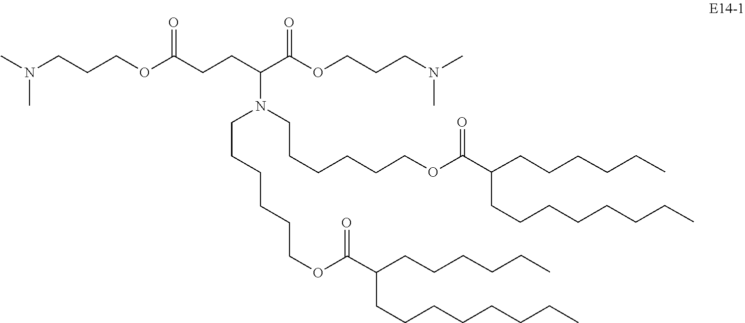 [SMILES: CCCCCCCCC(CCCCCC)C(=O)OCCCCCCN(CCCCCCOC(=O)C(CCCCCC)CCCCCCCC)C(CCC(=O)OCCCN(C)C)C(=O)OCCCN(C)C]

The preparation process is as follows:
Step a: glutamic acid (S14-1, 1.48 g, 6.0 mmol, the amino group of S14-1 was protected by Boc) and 3-dimethylamino-1-propanol (S14-2, 1.55 g, 15.0 mmol) were dissolved in anhydrous dichloromethane, respectively. After stirring well, two component solutions were combined. DMAP (73.20 mg, 0.6 mmol), EDCI (2.88 g, 15.0 mmol) and DIPEA (2.70 g, 30.0 mmol) were added into the mixture and the reaction was stirred at room temperature for 48 h. After the reaction, the reaction solution was diluted with dichloromethane (50 mL), then washed twice with saturated sodium carbonate solution (20 mL*2), washed twice with 20 mL aqueous solution, and washed once with saturated saline, the organic phase was dried with anhydrous sodium sulfate, and the solvent was concentrated to obtain a crude product. The crude product was separated and purified by column chromatography, the target eluate was collected and concentrated to obtain the product S14-3 (1.31 g).
Step b: removing the Boc protecting group. A solution of trifluoroacetic acid/dichloromethane (1:2, v/v) was prepared in a dry and clean round-bottom flask. A dichloromethane solution of S14-3 (1.04 g, 2.5 mmol) was slowly added dropwise under ice bath conditions and the reaction was carried out for 2 h at room temperature. After the reaction, the reaction solution was concentrated, diluted with purified water, and extracted with dichloromethane, the organic phase was dried with anhydrous magnesium sulfate and filtered, and the filtrate was concentrated and recrystallized to obtain compound S14-4 (0.73 g, 92.1%).
Step c: S14-4 (0.57 g, 1.8 mmol) was dissolved in 30 mL of DMF, then S1-3 (1.66 g, 4.0 mmol) and K₂CO₃ (0.66 g, 4.8 mmol) were added and stirred at room temperature overnight. At the end of the reaction, the reaction mixture was concentrated under reduced pressure and 30 mL of ethyl acetate was poured into the reaction solution. After washing with 10% citric acid (20 mL) and saline (20 mL) successively, the organic phase was dried with anhydrous sodium sulfate, filtered and concentrated, and the crude product was purified by column chromatography to obtain cationic lipid E14-1 (1.47 g). ¹H NMR (400 MHz, CDCl₃) δ: 4.52 (t, 1H), 4.09 (t, 8H), 2.87-2.80 (m, 4H), 2.49 (t, 4H), 2.34-2.31 (m, 4H), 2.23 (s, 12H), 2.13-1.98 (m, 4H), 1.69-1.21 (m, 66H), 0.88 (t, 12H). MS (ESI): m/z=994.87 ([M+H] ⁺).

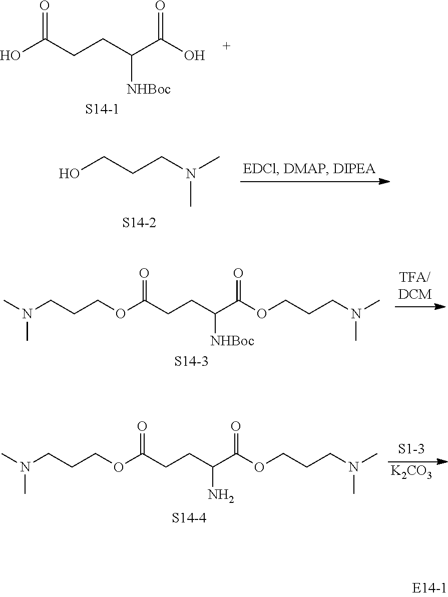

### Example 15: Cationic Lipid (E15-1)

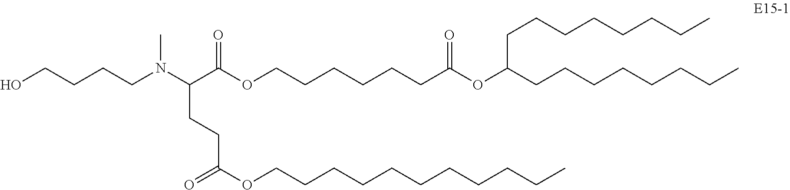 [SMILES: CCCCCCCCCCCOC(=O)CCC(C(=O)OCCCCCCC(=O)OC(CCCCCCCC)CCCCCCCC)N(C)CCCCO]

The preparation process is as follows
Step a: DCC (3.63 g, 17.6 mmol) was added into a round-bottom flask containing compounds S12-1 (2.46 g, 9.6 mmol), S7-1 (2.08 g, 8.0 mmol) and DMAP (0.24 g, 2.0 mmol) dissolved in dichloromethane (50 mL) under nitrogen atmosphere and reacted for 16 h at room temperature. After the reaction, the precipitate was removed by filtration, the filtrate was concentrated, and the resulting residue was purified by column chromatography to obtain TBS-7-hydroxyheptanoic acid-1-octylnonyl ester (S15-1, 3.26 g).
Step b: the above compound S15-1 (2.99 g, 6.0 mmol) was dissolved in THE (50 mL) in a nitrogen protected flask. TBAF solution (50 mL, 1 M) was added and the reaction was carried out overnight to remove the TBS protection. After the reaction, the reaction solution was concentrated and extracted, the organic phases were combined, dried with anhydrous sodium sulfate, filtered and concentrated, and purified by column chromatography to obtain the compound 7-hydroxyheptanoic acid-1-octylnonyl ester (S15-2, 2.05 g, 88.6%).
Step c: under nitrogen atmosphere, DCC (2.72 g, 13.2 mmol) was added into a round-bottom flask containing compounds S15-2 (0.97 g, 2.5 mmol), N-methylglutamic acid (S15-3, 2.11 g, 6.0 mmol, the secondary amino group and carboxyl group of S15-3 were protected by Boc and a benzyl group, respectively) and DMAP (0.18 g, 1.5 mmol) dissolved in dichloromethane (50 mL) and the reaction was conducted for 16 h at room temperature. After the reaction, the precipitate was removed by filtration, the filtrate was concentrated, and the resulting residue was purified by column chromatography to obtain compound S15-4 (3.48 g).
Step d: compound S15-4 (2.87 g, 4.0 mmol) was dissolved in methanol (50 mL), nickel chloride hexahydrate (NiCl₂·6H₂O, 2.86 g, 12.0 mmol) was added, and after stirring well, sodium borohydride (NaBH₄, 1.37 g, 36.0 mmol) was added slowly. The reaction was stirred at room temperature until TLC showed that the reaction was complete, then the reaction was quenched by slowly adding 20 mL of methanol to the reaction solution, after stirring for 30 min, the reaction solution was filtered with the diatomite and washed with methanol (50 mL). 20 mL of water was added into the reaction solution, after mixing well, the reaction solution was extracted with ethyl acetate (20 mL*3) for three times, the organic phase was dried with anhydrous magnesium sulfate, filtered, the filtrate was concentrated, then purified by column chromatography, concentrated, and dried by oil pump to obtain the compound S15-5 (2.24 g, 89.3%).
Step e: DCC (1.36 g, 6.6 mmol) was added into a round-bottom flask containing compounds S2-3 (0.62 g, 3.6 mmol), S15-5 (1.88 g, 3.0 mmol) and DMAP (91.50 mg, 0.8 mmol) dissolved in dichloromethane (30 mL) under nitrogen atmosphere and the reaction was conducted for 16 h at room temperature. After the reaction, the precipitate was removed by filtration, the filtrate was concentrated, and the resulting residue was purified by column chromatography to obtain compound S15-6 (1.96 g).
Step f: removing the Boc protecting group. A solution of trifluoroacetic acid/dichloromethane (1:2, v/v) was prepared in a dry and clean round-bottom flask. And a dichloromethane solution of S15-6 (1.51 g, 2.0 mmol) was added slowly and dropwise under an ice bath, and the reaction was carried out for 2 h at room temperature. At the end of the reaction, the reaction solution was concentrated, diluted with purified water, and extracted with dichloromethane, and the organic phase was dried with anhydrous magnesium sulfate and filtered, and the filtrate was concentrated and recrystallized to obtain compound S15-7 (1.23 g, 90.3%)
Step g: the above compound S15-7 (1.02 g, 1.5 mmol) was dissolved in dried THE (20 mL), NaH (60%, 0.06 g, 1.5 mmol) was slowly added under an ice bath, and the reaction was conducted under an ice bath for 1 h. After the reaction, compound 4-bromo-1-butanol (S15-8, 0.28 g, 1.8 mmol) was added, and after stirring the reaction under an ice bath for 1 h, the reaction solution was slowly cooled to room temperature and the reaction was continued overnight. After the reaction, the reaction was placed under an ice bath, 2 mL of water was slowly added to quench the reaction, after stirring for 30 minutes, water (10 mL) was added to mix. The reaction solution was extracted twice with dichloromethane (20 mL*2), the organic phases were collected and combined, backwashed once with saturated sodium chloride (20 mL), dried with anhydrous sodium sulfate, and filtered, and the filtrate was concentrated to obtain the crude product E15-1. Cationic lipid E15-1 (0.58 g) was obtained by column chromatography purification, concentration, and oil pump drying. ¹H NMR (400 MHz, CDCl₃) δ: 4.93-4.82 (m, 1H), 4.52 (t, 1H), 4.09 (t, 4H), 3.68-3.60 (t, 2H), 2.57 (s, 3H), 2.52 (t, 2H), 2.34-2.30 (m, 4H), 2.13-1.92 (m, 2H), 1.70-1.21 (m, 58H), 0.88 (t, 9H). MS (ESI): m/z=754.66 ([M+H] ⁺).

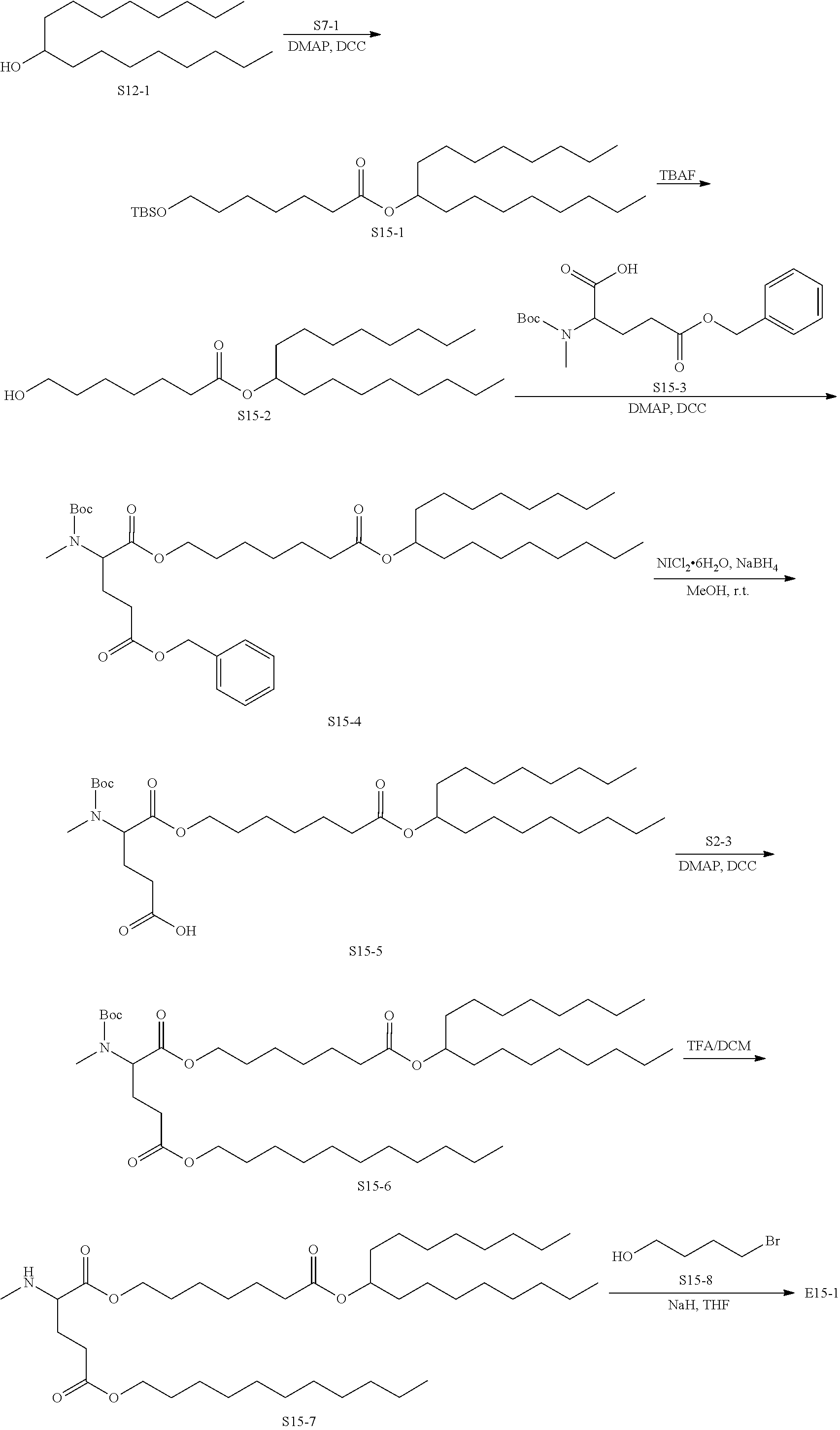

### Example 16.1: Cationic Lipid (E16-1)

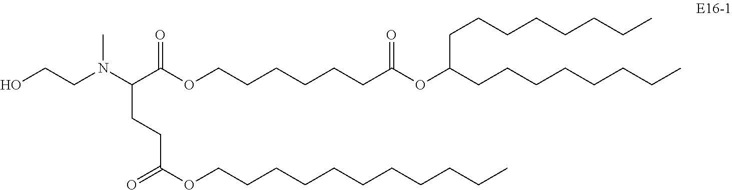 [SMILES: CCCCCCCCCCCOC(=O)CCC(C(=O)OCCCCCCC(=O)OC(CCCCCCCC)CCCCCCCC)N(C)CCO]

The preparation process is as follows:
the above compound S15-7 (3.41 g, 5.0 mmol) was dissolved in dried THE (50 mL), NaH (60%, 0.20 g, 5.0 mmol) was slowly added under an ice bath, and the reaction was carried out in an ice bath for 1 h. After the end of the reaction, compound 2-bromoethanol (S16-1, 0.75 g, 6.0 mmol) was added, and after 1 hour of stirring in an ice bath, the reaction solution was slowly returned to room temperature and the reaction was continued overnight. At the end of the reaction, the reaction was placed under an ice bath, 5 mL of water is slowly added to quench the reaction, the reaction was stirred for 30 minutes, and then water (20 mL) was added to mix the solution. The reaction solution was extracted twice with dichloromethane (50 mL*2), the organic phase was collected and combined, backwashed once with saturated sodium chloride (50 mL), dried with anhydrous sodium sulfate, and filtered, and the filtrate was concentrated to obtain the crude product E16-1. Cationic lipid E16-1 (1.93 g) was obtained by column chromatography purification, concentration, and oil pump drying. ¹H NMR (400 MHz, CDCl₃) δ: 4.91-4.81 (m, 1H), 4.51 (t, 1H), 4.08 (t, 4H), 3.54 (t, 2H), 2.57 (s, 3H), 2.52 (t, 2H), 2.34-2.32 (m, 4H), 2.13-1.92 (m, 2H), 1.68-1.21 (m, 54H), 0.88 (t, 9H). MS (ESI): m/z=726.62 ([M+H] ⁺).

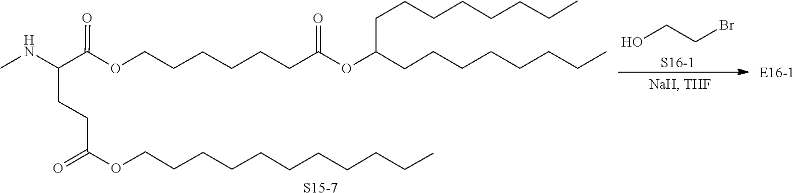

### Example 16.2: Cationic Lipid (E16-2)

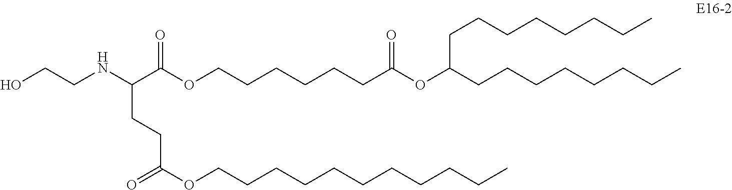 [SMILES: CCCCCCCCCCCOC(=O)CCC(NCCO)C(=O)OCCCCCCC(=O)OC(CCCCCCCC)CCCCCCCC]

The preparation process is as follows:
Step a: DCC (2.72 g, 13.2 mmol) was added into a round-bottom flask containing the compounds glutamic acid (S16-2, 2.02 g, 6.0 mmol, a carboxyl group and a primary amino group of S16-2 were protected by a benzyl group and Boc, respectively), S15-2 (2.78 g, 7.2 mmol) and DMAP (0.18 g, 1.5 mmol) dissolved in dichloromethane (40 mL) under nitrogen atmosphere and the reaction was conducted for 16 h at room temperature. After the reaction, the precipitate was removed by filtration, the filtrate was concentrated, and the resulting residue was purified by column chromatography to obtain compound S16-3 (3.45 g).
Step b: compound S16-3 (2.82 g, 4.0 mmol) was dissolved in methanol (50 mL), NiCl₂·6H₂O (2.86 g, 12.0 mmol) was added and stirred well, then NaBH₄ (1.37 g, 36.0 mmol) was added slowly. The reaction was stirred at room temperature until the reaction was complete according to the TLC, then the reaction was quenched by slowly adding 20 mL of methanol, and after stirring for 30 min, the reaction solution was filtered with diatomite and washed with methanol (50 mL). 20 mL of water was added to the reaction solution, after mixing well, the reaction solution was extracted with ethyl acetate (20 mL*3) for three times, the organic phase was dried with anhydrous magnesium sulfate and filtered, the filtrate was concentrated and purified by column chromatography, the filtrate was concentrated and dried by oil pump to obtain the compound S16-4 (2.19 g, 89.3%).
Step c: DCC (1.36 g, 6.6 mmol) was added to a round-bottom flask containing compounds S2-3 (0.62 g, 3.6 mmol), S16-4 (1.84 g, 3.0 mmol) and DMAP (91.50 mg, 0.8 mmol) dissolved in dichloromethane (30 mL) under nitrogen atmosphere and the reaction was carried out at room temperature for 16 h. At the end of the reaction, the precipitate was removed by filtration, the filtrate was concentrated and the residue obtained was purified by column chromatography to obtain compound S16-5 (1.89 g).
Step d: removing the Boc protecting group. A solution of trifluoroacetic acid/dichloromethane (1:2, v/v) was prepared in a dry and clean round-bottomed flask. A dichloromethane solution of S16-5 (1.54 g, 2.0 mmol) was slowly added dropwise under an ice bath, and the reaction was carried out for 2 h at room temperature. At the end of the reaction, the reaction solution was concentrated, diluted by adding purified water, extracted with dichloromethane, the organic phase was dried with anhydrous magnesium sulphate and filtered, and the filtrate was concentrated and recrystallized to obtain compound S16-6 (1.21 g, 90.2%).
Step e: S16-6 (1.00 g, 1.5 mmol) was dissolved in 20 mL of DMF, then S16-1 (0.23 g, 1.8 mmol) and K₂CO₃ (0.29 g, 2.1 mmol) were added and stirred at room temperature overnight. At the end of the reaction, the reaction mixture was concentrated under reduced pressure and 20 mL of ethyl acetate was poured into the reaction solution. After washing with 10% citric acid (10 mL) and saline (10 mL) successively, the organic phase was dried with anhydrous sodium sulfate, filtered and concentrated, and the crude product was purified by column chromatography to obtain cationic lipid E16-2 (0.87 g). ¹H NMR (400 MHz, CDCl₃) δ: 4.93-4.81 (m, 1H), 4.51 (t, 1H), 4.08 (t, 4H), 3.54 (t, 2H), 2.74 (t, 2H), 2.34-2.31 (m, 4H), 2.13-1.92 (m, 2H), 1.69-1.21 (m, 54H), 0.88 (t, 9H). MS (ESI): m/z=712.60 ([M+H] ⁺).

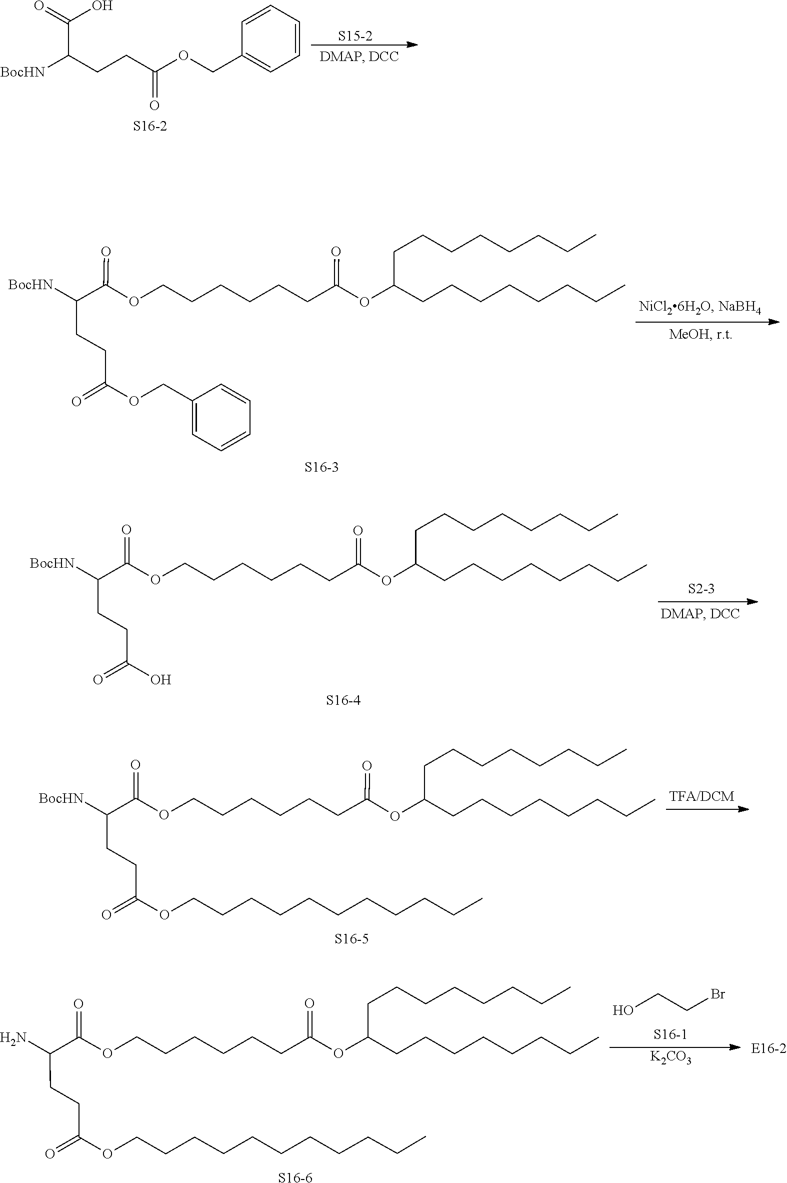

### Example 17: Cationic Lipid (E17-1)

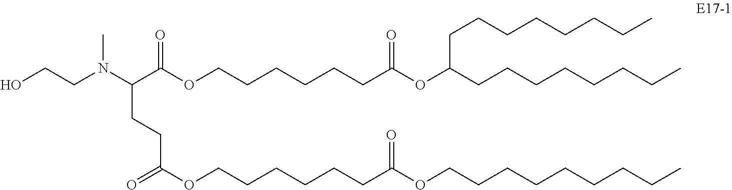 [SMILES: CCCCCCCCCOC(=O)CCCCCCOC(=O)CCC(C(=O)OCCCCCCC(=O)OC(CCCCCCCC)CCCCCCCC)N(C)CCO]

The preparation process is as follows:
Step a: DCC (2.27 g, 11.0 mmol) was added to a round-bottom flask containing compounds S15-5 (3.14 g, 5.0 mmol), S7-4 (1.63 g, 6.0 mmol) and DMAP (0.15 g, 1.3 mmol) dissolved in dichloromethane (50 mL) under nitrogen atmosphere and the reaction was conducted for 16 h at room temperature. At the end of the reaction, the precipitate was removed by filtration, the filtrate was concentrated, and the resulting residue was purified by column chromatography to obtain compound S17-1 (3.68 g).
Step b: removing the Boc protecting group. In a dry and clean round-bottomed flask, a solution of trifluoroacetic acid/dichloromethane (1:2, v/v) was prepared. A dichloromethane solution of S17-1 (2.65 g, 3.0 mmol) was slowly added dropwise under an ice bath, and the reaction was carried out for 2 h at room temperature. At the end of the reaction, the reaction solution was concentrated, diluted by adding purified water, extracted with dichloromethane, the organic phase was dried with anhydrous magnesium sulphate and filtered, and the filtrate was concentrated and recrystallized to obtain compound S17-2 (2.12 g, 90.4%).
Step c: the above compound S17-2 (1.56 g, 2.0 mmol) was dissolved in dried THE (30 mL), and NaH (60%, 0.08 g, 2.0 mmol) was slowly added under ice bath and the reaction was conducted for 1 h. At the end of the reaction, compound S16-1 (0.30 g, 2.4 mmol) was added and stirred under an ice bath for 1 h, and the reaction solution was slowly returned to room temperature and stirred overnight. After the end of the reaction, the reaction was quenched by slowly adding 3 mL of water under an ice bath, and after 30 min, water (15 mL) was added to mix the solution. The reaction solution was extracted twice with dichloromethane (30 mL*2), the organic phases were collected and combined, backwashed once with saturated sodium chloride (30 mL), dried with anhydrous sodium sulfate, and filtered, and the filtrate was concentrated to obtain the crude product E17-1. Cationic lipid E17-1 (0.87 g) was obtained by column chromatography purification, concentration, and oil pump drying. ¹H NMR (400 MHz, CDCl₃) δ: 4.92-4.81 (m, 1H), 4.51 (t, 1H), 4.08 (t, 6H), 3.54 (t, 2H), 2.56 (s, 3H), 2.52 (t, 2H), 2.34-2.32 (m, 6H), 2.13-1.92 (m, 2H), 1.70-1.21 (m, 58H), 0.88 (t, 9H). MS (ESI): m/z=826.67 ([M+H] ⁺).

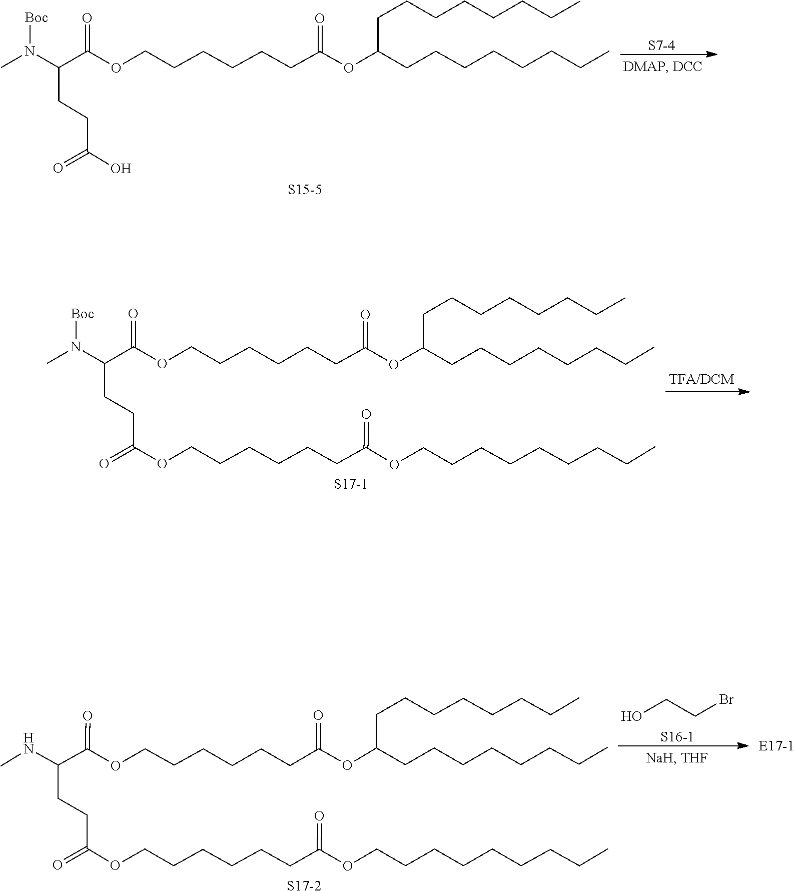

### Example 18: Cationic Lipid (E18-2)

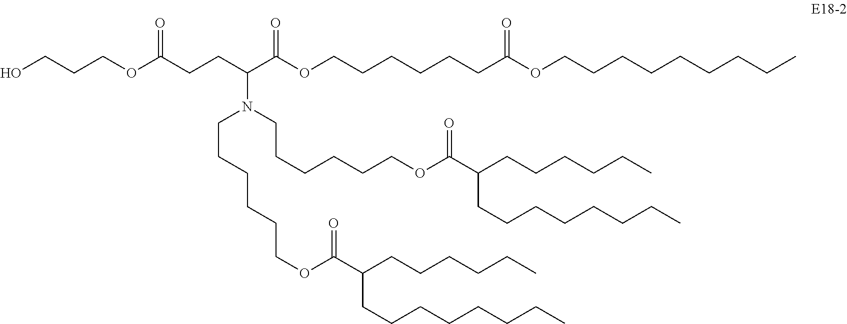 [SMILES: CCCCCCCCCOC(=O)CCCCCCOC(=O)C(CCC(=O)OCCCO)N(CCCCCCOC(=O)C(CCCCCC)CCCCCCCC)CCCCCCOC(=O)C(CCCCCC)CCCCCCCC]

The preparation process is as follows:
Step a: under nitrogen atmosphere, DCC (2.27 g, 11.0 mmol) was added to a round-bottom flask containing compound S18-1 (2.10 g, 5.0 mmol, S18-1 was obtained by the reaction between

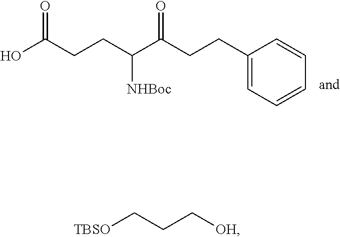 [SMILES: CC(C)(C)OC(=O)NC(CCC(=O)O)C(=O)CCc1ccccc1 ; CC(C)(C)[Si](C)(C)OCCCO]

followed by carboxyl deprotection), S7-4 (1.63 g, 6.0 mmol) and DMAP (0.15 g, 1.3 mmol) dissolved in dichloromethane (30 mL) and the reaction was conducted for 16 h at room temperature. After the reaction, the precipitate was removed by filtration, the filtrate was concentrated, and the resulting residue was purified by column chromatography to obtain compound S18-2 (2.75 g).
Step B: removing the Boc protecting group. In a dry and clean round-bottom flask, a solution of trifluoroacetic acid/dichloromethane (1:2, v/v) was prepared. And a dichloromethane solution of S18-2 (2.02 g, 3.0 mmol) was slowly added dropwise at room temperature for 2 h under an ice bath condition. After the reaction, the reaction solution was concentrated, then diluted with purified water, and extracted with dichloromethane, the organic phase was dried with anhydrous magnesium sulfate and filtered, and the filtrate was concentrated and recrystallized to obtain compound S18-3 (1.55 g, 90.2%).
Step c: S18-3 (1.15 g, 2.0 mmol) was dissolved in 30 mL of DMF, S1-3 (1.84 g, 4.4 mmol) and K₂CO₃ (0.73 g, 5.3 mmol) were added and stirred at room temperature overnight. At the end of the reaction, the reaction mixture was concentrated under reduced pressure and poured into 30 mL of ethyl acetate. After washed with 10% citric acid (20 mL) and saline (20 mL) sequentially, the organic phase was dried with anhydrous sodium sulfate, filtered and concentrated, and the crude product was purified by column chromatography to obtain E18-1 (2.03 g).
Step d: the above compound E18-1 (1.25 g, 1.0 mmol) was dissolved in THE (20 mL) in a nitrogen-protected flask, and TBS protection was removed by adding TBAF solution (20 mL, 1 M) and reacting overnight. At the end of the reaction, the reaction solution was concentrated and extracted, and the organic phases were combined, dried with anhydrous sodium sulfate, filtered and concentrated, and purified by column chromatography to obtain cationic lipid E18-2 (1.01 g, 88.6%). ¹H NMR (400 MHz, CDCl₃) δ: 4.51 (t, 1H), 4.09 (t, 8H), 3.70-3.69 (t, 2H), 4.23 (t, 2H), 2.49 (m, 4H), 2.34-2.32 (m, 4H), 2.30 (t, 2H), 2.13-1.92 (m, 2H), 1.87 (m, 2H), 1.71-1.21 (m, 86H), 0.88 (t, 15H). MS (ESI): m/z=1136.95 ([M+H] ⁺).

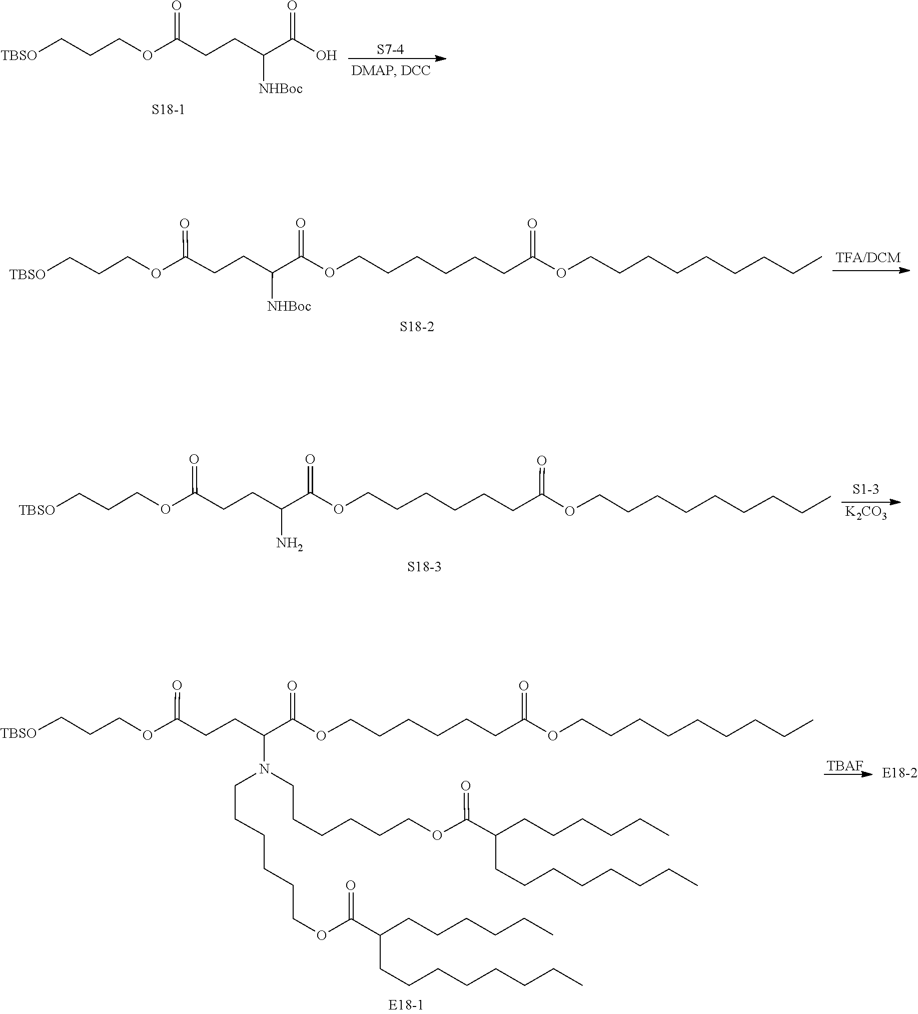

### Example 19.1: Cationic Lipid (E19-1)

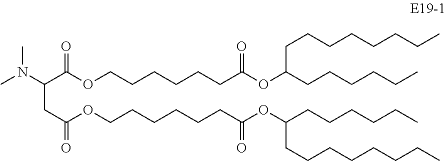 [SMILES: CCCCCCCCC(CCCCCC)OC(=O)CCCCCCOC(=O)CC(C(=O)OCCCCCCC(=O)OC(CCCCCC)CCCCCCCC)N(C)C]

The preparation process is as follows:
N,N-dimethylaspartic acid (S19-1, 0.32 g, 2.0 mmol) and S3-3 (1.79 g, 5.0 mmol) were dissolved in anhydrous dichloromethane respectively, stirred well and then the two component solutions were combined, and DMAP (24.40 mg, 0.2 mmol), EDCI (0.96 g, 5.0 mmol) and DIPEA (0.90 g, 10.0 mmol) were added to the mixture sequentially. The reaction was stirred at room temperature for 48 h. At the end of the reaction, the reaction solution was diluted with dichloromethane (30 mL), and then washed twice with saturated sodium carbonate solution (30 mL*2), washed twice with 30 mL of aqueous solution, and washed once with saturated saline, and then the organic phase was dried with anhydrous sodium sulfate, concentrated to obtain the crude product. The crude product was separated and purified by column chromatography, and the target eluent was collected and concentrated to obtain the cationic lipid E19-1 (0.88 g). ¹H NMR (400 MHz, CDCl₃) δ: 4.93-4.81 (m, 2H), 4.08 (t, 4H), 3.40 (t, 1H), 2.56 (s, 6H), 2.52-2.48 (m, 2H), 2.30 (t, 4H). 2.30 (t, 4H), 1.70-1.22 (m, 64H), 0.88 (t, 12H). MS (ESI): m/z=838.71 ([M+H] ⁺).

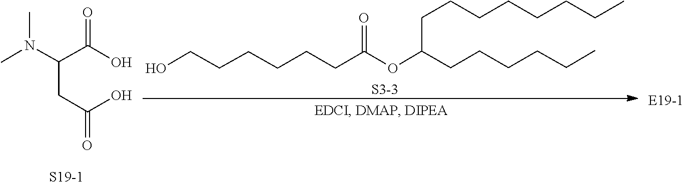

### Example 19.2: Cationic Lipid (E19-2)

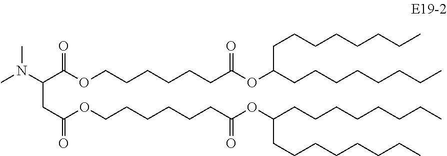 [SMILES: CCCCCCCCC(CCCCCCCC)OC(=O)CCCCCCOC(=O)CC(C(=O)OCCCCCCC(=O)OC(CCCCCCCC)CCCCCCCC)N(C)C]

Starting material S3-3 in Example 19.1 was replaced with starting material

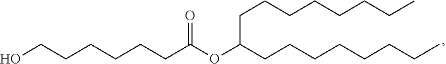 [SMILES: CCCCCCCCC(CCCCCCCC)OC(=O)CCCCCCO]

and prepared according to the same reaction steps to obtain cationic lipid E19-2. The structure of E19-2 was also verified by NMR and mass spectrometry.

### Example 20: Cationic Lipid (E20-1)

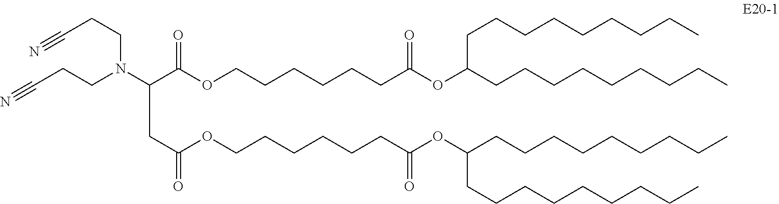 [SMILES: CCCCCCCCCC(CCCCCCCCC)OC(=O)CCCCCCOC(=O)CC(C(=O)OCCCCCCC(=O)OC(CCCCCCCCC)CCCCCCCCC)N(CCC#N)CCC#N]

The preparation process is as follows:
Step a: DCC (3.63 g, 17.6 mmol) was added to a round-bottom flask containing compounds S7-1 (2.08 g, 8.0 mmol), 10-nonadecanol (S20-1, 2.74 g, 9.6 mmol) and DMAP (0.24 g, 2.0 mmol) dissolved in dichloromethane (50 mL) under nitrogen atmosphere and reacted for 16 h at room temperature. After the reaction, the precipitate was removed by filtration, the filtrate was concentrated, and the resulting residue was purified by column chromatography to obtain compound S20-2 (3.47 g).
Step b: the above compound S20-2 (2.64 g, 5.0 mmol) was dissolved in THE (50 mL), and added in a nitrogen-protected flask. Then TBAF solution (50 mL, 1 M) was added to react overnight to remove TBS protection. After the reaction, the reaction solution was concentrated and extracted, the organic phases were combined, dried with anhydrous sodium sulfate, filtered and concentrated, and purified by column chromatography to obtain the compound S20-3 (1.82 g, 87.9%).
Step c: S20-3 (1.55 g, 3.8 mmol), S20-4 (0.36 g, 1.5 mmol) were dissolved in anhydrous dichloromethane respectively, stirred well and then the two solutions were combined. And then DMAP (18.30 mg, 0.15 mmol), EDCI (0.72 g, 3.8 mmol) and DIPEA (0.68 g, 7.5 mmol) were added to the mixture and the reaction was stirred for 48 h at room temperature. After the reaction, the reaction solution was diluted with dichloromethane (20 mL), then washed twice with saturated sodium carbonate solution (10 mL*2), washed twice with 10 mL aqueous solution, and then washed once with saturated saline, the organic phase was dried with anhydrous sodium sulfate and concentrated to obtain a crude product. The crude product was separated and purified by column chromatography, the target eluate was collected and concentrated to obtain cationic lipid E20-1 (0.82 g). ¹H NMR (400 MHz, CDCl₃) δ: 4.93-4.81 (m, 2H), 4.08 (t, 4H), 3.40 (t, 1H), 2.49-2.51 (m, 6H), 2.33-2.25 (m, 8H), 1.71-1.22 (m, 80H), 0.87 (t, 12H). MS (ESI): m/z=1028.85 ([M+H] ⁺).

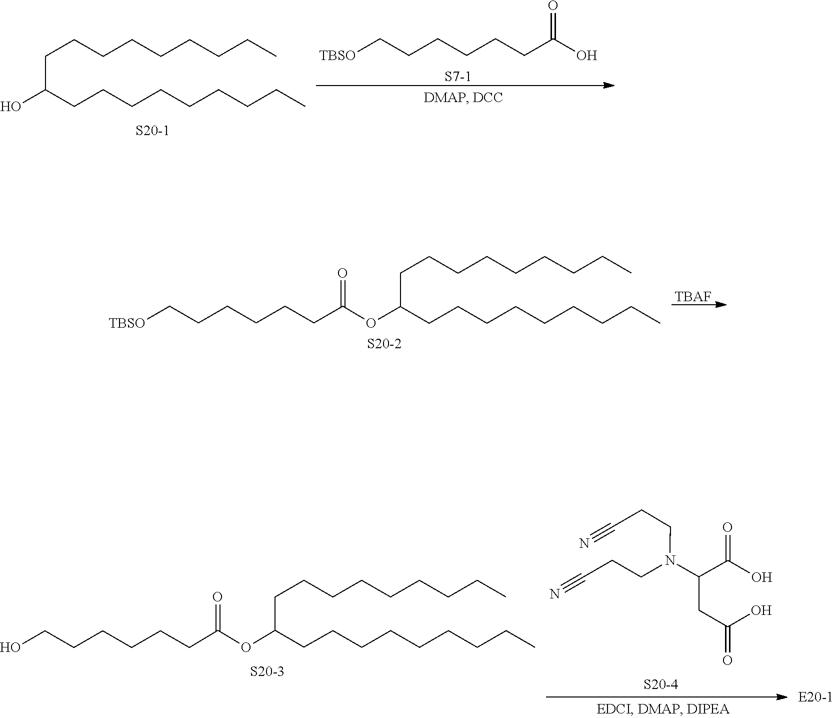

### Example 21: Cationic Lipid (E21-2)

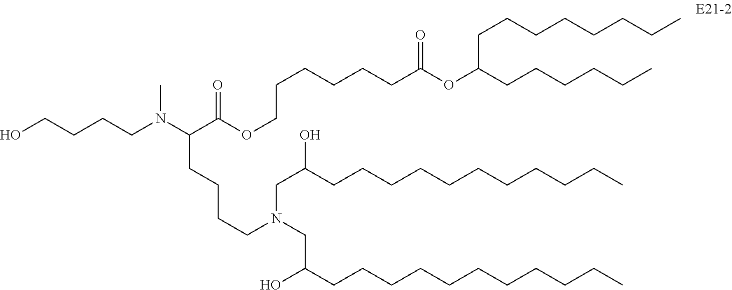 [SMILES: CCCCCCCCCCCC(O)CN(CCCCC(C(=O)OCCCCCCC(=O)OC(CCCCCC)CCCCCCCC)N(C)CCCCO)CC(O)CCCCCCCCCCC]

The preparation process is as follows:
Step a: DCC (2.72 g, 13.2 mmol) was added to a round-bottom flask containing compounds S3-1 (2.08 g, 6.0 mmol), S3-3 (2.56 g, 7.2 mmol) and DMAP (0.18 g, 1.5 mmol) dissolved in dichloromethane (50 mL) under nitrogen atmosphere and reacted for 16 h at room temperature. After the reaction, the precipitate was removed by filtration, the filtrate was concentrated, and the resulting residue was purified by column chromatography to obtain the target compound S21-1 (3.42 g).
Step b: 1,2-epoxytridecane (S21-2, 3.17 g, 16.0 mmol) was added to a solution of anhydrous ethanol (50 mL) in which S21-1 (2.74 g, 4.0 mmol) was dissolved, and the reaction was stirred at room temperature for 10 h. After the reaction, the reaction solution was concentrated and extracted, the organic phases were combined, dried with anhydrous sodium sulfate, filtered and concentrated, and purified by column chromatography to obtain the compound E21-1 (2.00 g).
Step c: the above compound E21-1 (1.62 g, 1.5 mmol) was dissolved in THE (20 mL) in a flask protected by nitrogen, and TBAF solution (20 mL, 1 M) was added and reacted overnight to remove the TBS protection. At the end of the reaction, the reaction solution was concentrated and extracted, the organic phases were combined, dried with anhydrous sodium sulfate, filtered and concentrated, and purified by column chromatography to obtain the cationic lipid E21-2 (1.21 g, 83.6%). 1H NMR (400 MHz, CDCl₃) δ: 4.93-4.81 (m, 1H), 3.68-3.60 (t, 2H), 3.55-3.53 (m, 2H), 3.45 (t, 1H), 3.18-2.98 (m, 2H), 2.56 (s, 3H), 2.52 (t, 2H), 2.36-2.33 (m, 4H), 2.31 (t, 4H), 2.01-1.82 (m, 4H), 1.71-1.22 (m, 78H), 0.89 (t, 12H). MS (ESI): m/z=967.89 ([M+H] ⁺).

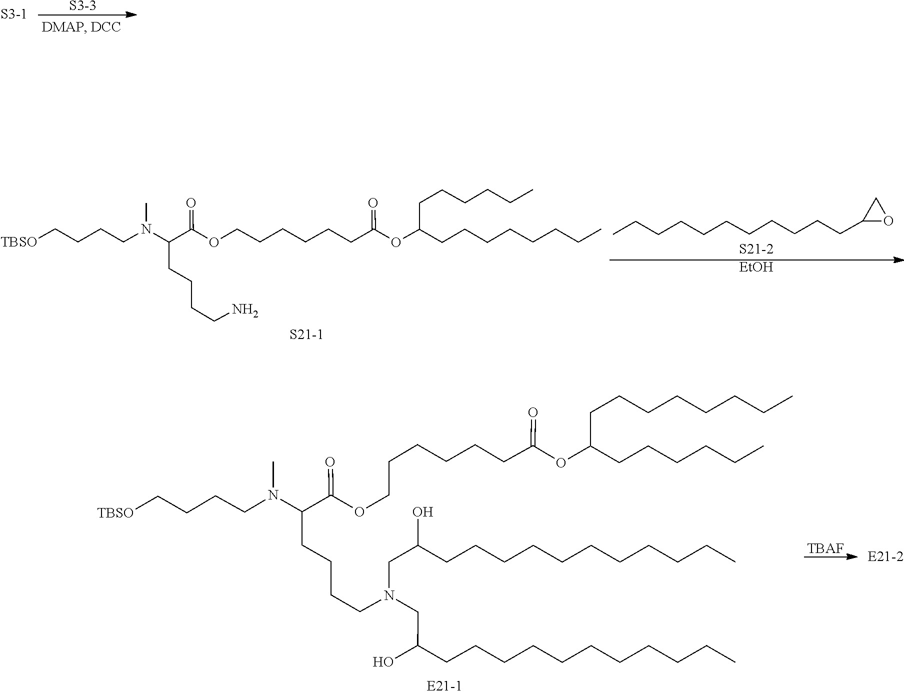

### Example 22: Cationic Lipid (E22-2)

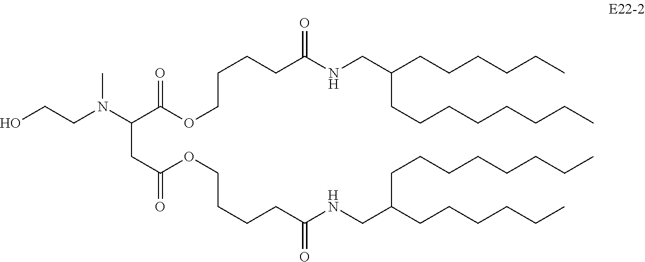 [SMILES: CCCCCCCCC(CCCCCC)CNC(=O)CCCCOC(=O)CC(C(=O)OCCCCC(=O)NCC(CCCCCC)CCCCCCCC)N(C)CCO]

The preparation process is as follows:
Step a: 5-hydroxyvaleric acid (S22-1, 2.32 g, 10.0 mmol, the hydroxyl group of S22-1 is protected by TBS), EDCI (2.30 g, 12.0 mmol) and NHS (1.25 g, 11.0 mmol) were dissolved in dichloromethane (50 mL), and the reaction was stirred at room temperature overnight. After the reaction, the reaction solution was backwashed twice (20 mL*2) with 0.1 mol/L HCl, backwashed once with saturated sodium chloride (50 mL), the dichloromethane phases were collected, dried with anhydrous magnesium sulfate, and filtered, and the filtrate was concentrated to obtain compound S22-2 (3.22 g, 97.8%).
Step b: the above compound S22-2 (2.96 g, 9.0 mmol) was dissolved in dichloromethane (50 mL), 2-hexyldecyldeamine (S22-3, 2.39 g, 9.9 mmol) and TEA (2.8 mL, 18.0 mmol) were added and reacted overnight at room temperature to precipitate a large amount of white solid. The solids were obtained by filtration, slurried with methanol (20 mL), filtered, and rinsed twice with methanol, and the solids were collected and dried to obtain compound S22-4 (3.41 g, 83.3%).
Step c: the above compound S22-4 (3.19 g, 7.0 mmol) was dissolved in THE (30 mL) and added to a nitrogen-protected flask. TBAF solution (30 mL, 1 M) was added and the reaction was conducted overnight to remove TBS protection. After the reaction, the reaction solution was concentrated and extracted, the organic phases were combined, dried with anhydrous sodium sulfate, filtered and concentrated, and purified by column chromatography to obtain the compound S22-5 (2.13 g, 89.2%).
Step d: S22-5 (1.88 g, 5.5 mmol) and S22-6 (0.67 g, 2.2 mmol, the hydroxyl group of S22-6 was protected by TBS) were dissolved in anhydrous dichloromethane, respectively. After stirring well, the two components of the solution were combined. DMAP (26.84 mg, 0.22 mmol), EDCI (1.06 g, 5.5 mmol) and DIPEA (0.99 g, 11.0 mmol) were added to the mixture and stirred for 48 h at room temperature. After the reaction, the reaction solution was diluted with dichloromethane (30 mL), then washed twice with saturated sodium carbonate solution (20 mL*2), washed twice with 30 mL aqueous solution, and washed once with saturated saline, the organic phase was dried with anhydrous sodium sulfate, concentrated to obtain a crude product. The crude product was separated and purified by column chromatography, the target eluent was collected and concentrated to obtain the product E22-1 (1.11 g).
Step e: the above compound E22-1 (0.95 g, 1.0 mmol) was dissolved in THF (10 mL) and added in a flask protected by nitrogen, and TBAF solution (10 mL, 1 M) was added and reacted overnight to remove the TBS protection. At the end of the reaction, the reaction solution was concentrated and extracted, the organic phases were combined, dried with anhydrous sodium sulfate, filtered and concentrated, and purified by column chromatography to obtain the cationic lipid E22-2 (0.74 g, 88.3%). ¹H NMR (400 MHz, CDCl₃) δ: 4.09 (t, 4H), 3.54 (t, 2H), 3.40 (t, 1H), 3.19-3.13 (m, 4H), 2.74 (t, 2H), 2.56 (s, 3H), 2.52-2.48 (m, 2H), 2.19-2.15 (t, 4H), 2.01-1.98 (m, 2H), 1.71-1.22 (m, 56H), 0.87 (t, 12H). MS (ESI): m/z=838.72 ([M+H] ⁺).

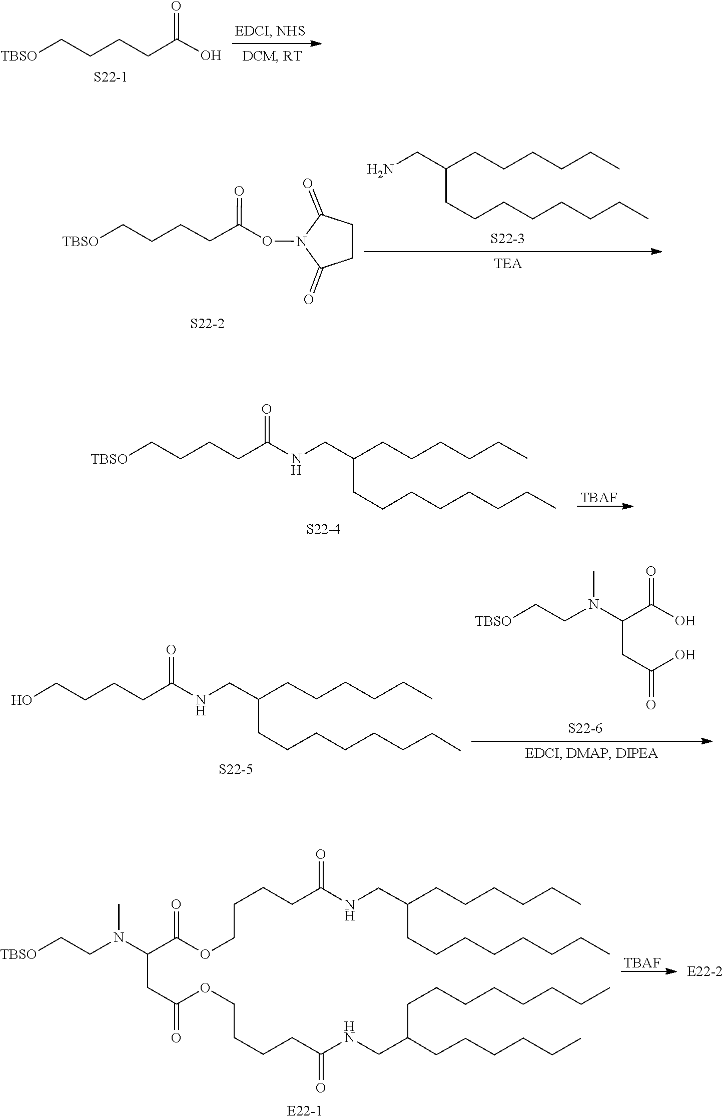

### Example 23: Cationic Lipid (E23-1)

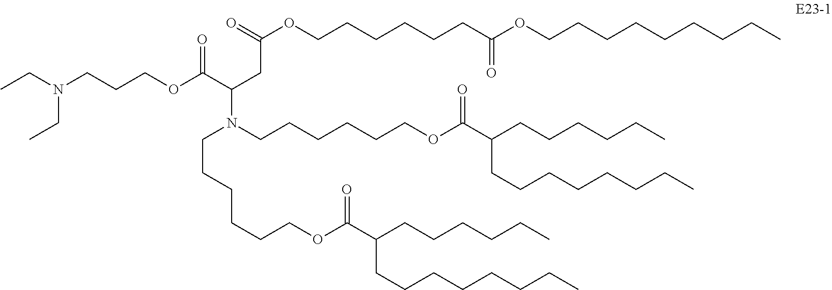 [SMILES: CCCCCCCCCOC(=O)CCCCCCOC(=O)CC(C(=O)OCCCN(CC)CC)N(CCCCCCOC(=O)C(CCCCCC)CCCCCCCC)CCCCCCOC(=O)C(CCCCCC)CCCCCCCC]

The preparation process is as follows:
Step a: DCC (2.27 g, 11.0 mmol) was added to a round-bottom flask containing aspartic acid 4-benzyl ester (S23-1, 1.62 g, 5.0 mmol, the amino group of S23-1 was protected by Boc), 3-diethylamino-1-propanol (S23-2, 0.79 g, 6.0 mmol) and DMAP (0.15 g, 1.3 mmol) dissolved in dichloromethane (30 mL) under nitrogen atmosphere, and the reaction was conducted at room temperature for 16 h. After the reaction, the precipitate was removed by filtration, the filtrate was concentrated, and the resulting residue was purified by column chromatography to obtain compound S23-3 (1.82 g).
Step b: removing the Boc protecting group. In a dry and clean round-bottom flask, a solution of trifluoroacetic acid/dichloromethane (1:2, v/v) was prepared. S23-3 (1.31 g, 3.0 mmol) dichloromethane solution was slowly added dropwise under ice bath condition and the reaction was conducted for 2 h at room temperature. After the end of the reaction, the reaction solution was concentrated, diluted with purified water, and extracted with dichloromethane, the organic phase was dried with anhydrous magnesium sulfate and filtered, the filtrate was concentrated and recrystallized to obtain compound S23-4 (0.91 g, 90.0%).
Step c: S1-3 (1.84 g, 4.4 mmol) was dissolved in 30 mL of DMF, then S23-4 (0.67 g, 2.0 mmol) and K₂CO₃ (0.73 g, 5.3 mmol) were added and stirred overnight at room temperature. At the end of the reaction, the reaction mixture was concentrated under reduced pressure and 30 mL of ethyl acetate was poured into the solution. After washing with 10% citric acid (20 mL) and saline (20 mL) successively, the organic phase was dried with anhydrous sodium sulfate, filtered and concentrated, and the crude product was purified by column chromatography to obtain S23-5 (1.64 g).
Step d: compound S23-5 (1.22 g, 1.2 mmol) was dissolved in methanol (20 mL) solution, NiCl₂·6H₂O (0.86 g, 3.6 mmol) was added and stirred well, then NaBH₄ (0.41 g, 10.8 mmol) was added slowly and the reaction was stirred at room temperature until the reaction was complete as indicated by the TLC. 10 mL of methanol was added to the reaction solution to quench the reaction. After stirring for 30 min, the reaction solution was filtered with diatomite, and washed with methanol (20 mL). Into the reaction solution, 20 mL of water was added, after mixing well, the reaction solution was extracted three times with ethyl acetate (10 mL*3), the organic phase was dried with anhydrous magnesium sulfate and filtered, the filtrate was concentrated, then purified by column chromatography, concentrated, and dried by oil pump to obtain the compound S23-6 (0.98 g).
Step e: DCC (0.36 g, 1.8 mmol) was added to a round-bottom flask containing compounds S23-6 (0.74 g, 0.8 mmol), S7-4 (0.23 g, 1.0 mmol) and DMAP (24.40 mg, 0.2 mmol) dissolved in dichloromethane (10 mL) under nitrogen atmosphere and reacted for 16 h at room temperature. After the reaction, the precipitate was removed by filtration, the filtrate was concentrated, and the resulting residue was purified by column chromatography to obtain cationic lipid E23-1 (0.81 g, 86.5%). ¹H NMR (400 MHz, CDCl₃) δ: 4.09 (t, 10H), 3.40 (t, 1H), 2.87-2.80 (t, 2H), 2.57 (q, 4H), 2.51-2.49 (m, 6H), 2.33-2.27 (m, 4H), 1.98-1.15 (m, 94H), 0.89 (m, 15H). MS (ESI): m/z=1178.02 ([M+H] ⁺).

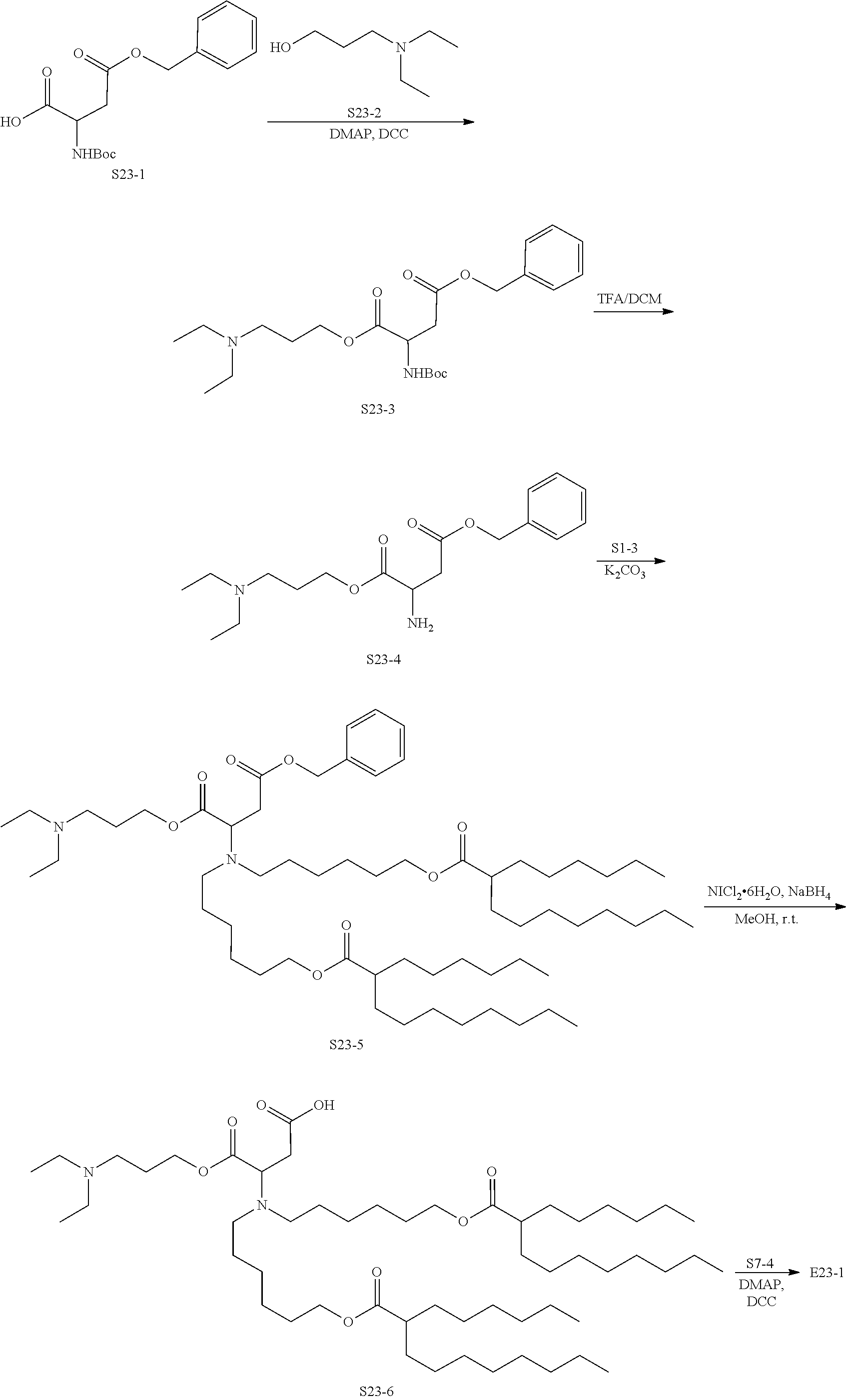

### Example 24: Cationic Lipid (E24-1)

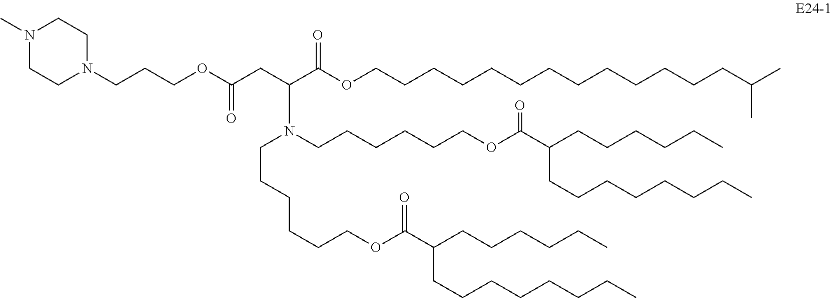 [SMILES: CCCCCCCCC(CCCCCC)C(=O)OCCCCCCN(CCCCCCOC(=O)C(CCCCCC)CCCCCCCC)C(CC(=O)OCCCN1CCN(C)CC1)C(=O)OCCCCCCCCCCCCCC(C)C]

The starting material S23-2 in Example 23 was replaced with isopentadecanol (S24-1,

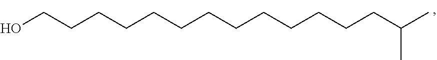 [SMILES: CC(C)CCCCCCCCCCCCCO]

1.31 g, 5.4 mmol), and the starting material S7-4 was replaced with 1-(3-hydroxypropyl)-4-methylpiperazine (S24-6,

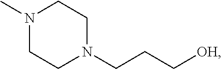

0.37 g, 2.3 mmol), Cationic lipid E24-1 (1.02 g, 87.2%) was prepared by following the same reaction steps. ¹H NMR (400 MHz, CDCl₃) δ: 4.09 (t, 8H), 3.40 (t, 1H), 2.85 (t, 4H), 2.50 (m, 6H), 2.35 (t, 6H), 2.30 (s, 3H), 2.25 (m, 2H), 1.80-1.70 (m, 2H), 1.67-1.15 (m, 89H), 0.92-0.89 (m, 18H). MS (ESI): m/z=1175.07 ([M+H] ⁺).

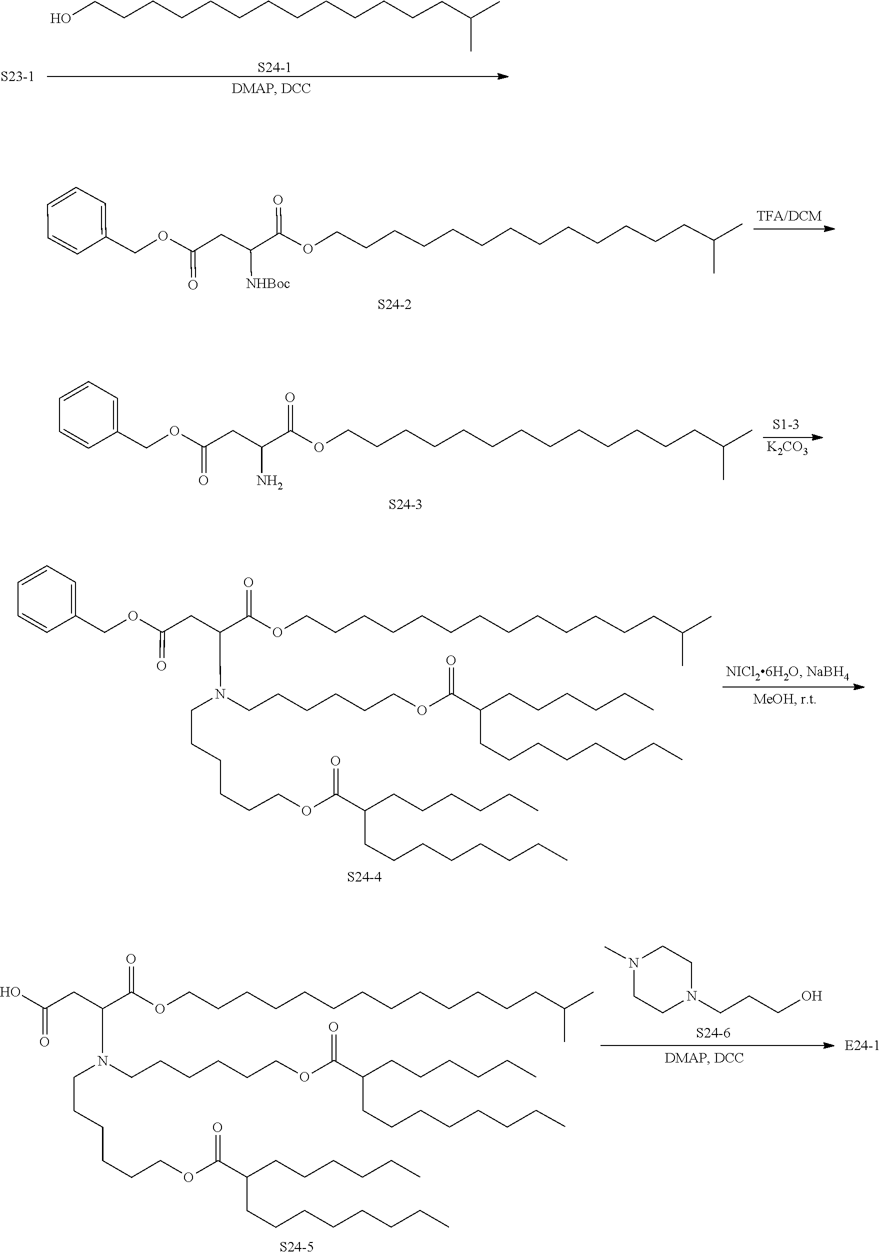

### Example 25: Cationic Lipid (E25-1)

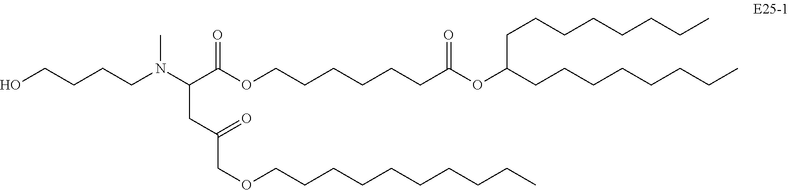 [SMILES: CCCCCCCCCCOCC(=O)CC(C(=O)OCCCCCCC(=O)OC(CCCCCCCC)CCCCCCCC)N(C)CCCCO]

The preparation process is as follows:
Step a: DCC (2.27 g, 11.0 mmol) was added to a round-bottom flask containing N-methyl-N-tert-butyl ester-aspartic acid-4-benzyloxycarbonyl (S25-1, 1.69 g, 5.0 mmol), S15-2 (2.30 g, 6.0 mmol) and DMAP (0.15 g, 1.3 mmol) dissolved in dichloromethane (30 mL) for 16 h at room temperature. After the reaction, the precipitate was removed by filtration, the filtrate was concentrated, and the resulting residue was purified by column chromatography to obtain compound S25-2 (2.94 g).
Step b: compound S25-2 (2.46 g, 3.5 mmol) was dissolved in methanol (50 mL), NiCl₂·6H₂O (2.50 g, 10.5 mmol) was added and stirred well, then NaBH₄ (1.20 g, 31.5 mmol) was added and the reaction was stirred at room temperature until the reaction was complete as shown by the TLC. 20 mL of methanol was added to the reaction solution slowly to quench the reaction. After stirring for 30 min, the reaction solution was filtered with diatomite and washed with methanol (50 mL). Into the reaction solution, 50 mL of water was added, after mixing well, the reaction solution was extracted three times with ethyl acetate (20 mL*3), the organic phase was dried with anhydrous magnesium sulfate and filtered, the filtrate was concentrated, and then purified by column chromatography, concentrated, and dried by oil pump to obtain the compound S25-3 (1.92 g, 89.5%).
Step c: DCC (1.22 g, 5.9 mmol) was added to a round-bottom flask containing compounds S25-3 (1.66 g, 2.7 mmol), S2-3 (0.56 g, 3.2 mmol) and DMAP (82.35 mg, 0.68 mmol) dissolved in dichloromethane (20 mL) under nitrogen atmosphere and reacted for 16 h at room temperature. At the end of the reaction, the precipitate was removed by filtration, the filtrate was concentrated and the residue obtained was purified by column chromatography to obtain compound S25-4 (1.73 g).
Step d: removing the Boc protecting group. In a dry and clean round-bottom flask, a solution of trifluoroacetic acid/dichloromethane (1:2, v/v) was prepared. A dichloromethane solution of S25-4 (1.53 g, 2.0 mmol) was slowly dropwise added under ice bath condition and the reaction was conducted for 2 h at room temperature. After the reaction, the reaction solution was concentrated, diluted with purified water, and extracted with dichloromethane, the organic phase was dried with anhydrous magnesium sulfate and filtered, the filtrate was concentrated and recrystallized to obtain compound S25-5 (1.25 g, 92.5%).
Step e: compound S25-5 (1.00 g, 1.5 mmol) was dissolved in dried THE (20 mL), then NaH (60%, 0.60 g, 15.0 mmol) was slowly added under an ice bath and reacted under an ice bath for 1 h. After the reaction, compound S15-8 (0.27 g, 1.8 mmol) was added and stirred for 1 hour under an ice bath, and the reaction solution was slowly returned to room temperature and reacted overnight. After the reaction, the reaction was put under an ice bath, 2 mL of methanol was slowly added to quench the reaction, after stirring for 30 minutes, water (10 mL) was added to mix the solution well. The reaction solution was extracted twice with dichloromethane (20 mL*2), the organic phases were collected and combined, backwashed once with saturated sodium chloride (20 mL), dried with anhydrous sodium sulfate, and filtered, and the filtrate was concentrated to obtain the crude product E25-1. Cationic lipid E25-1 (0.83 g) was obtained by column chromatography purification, concentration, and oil pump drying. ¹H NMR (400 MHz, CDCl₃) δ: 4.93-4.81 (m, 1H), 4.07 (t, 4H), 3.68-3.60 (t, 2H), 3.40 (t, 1H), 2.56 (s, 3H), 2.52-2.49 (m, 4H), 2.32 (t, 2H), 1.65-1.25 (m, 58H), 0.89 (t, 9H). MS (ESI): m/z=740.63 ([M+H] ⁺).

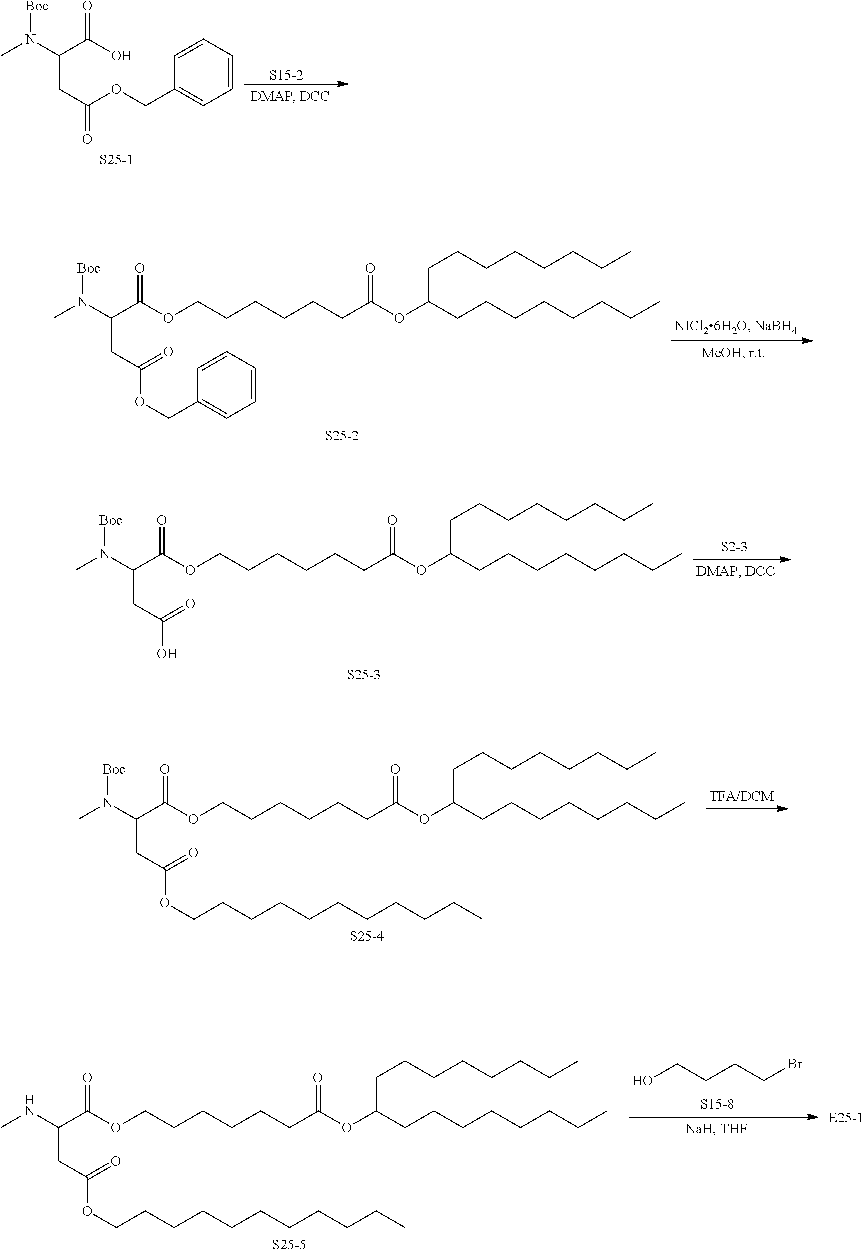

### Example 26: Cationic Lipid (E26-1)

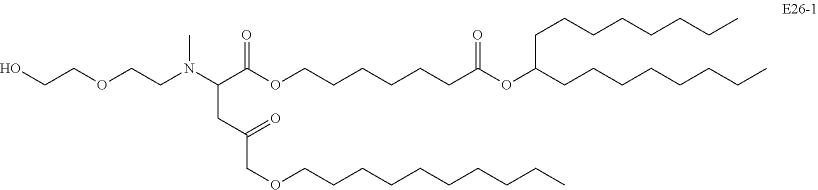 [SMILES: CCCCCCCCCCOCC(=O)CC(C(=O)OCCCCCCC(=O)OC(CCCCCCCC)CCCCCCCC)N(C)CCOCCO]

The preparation process is as follows:

The compound S25-5 (2.67 g, 4.0 mmol) was dissolved in dried THF (40 mL), then NaH (60%, 1.60 g, 40.0 mmol) was slowly added under an ice bath and reacted under an ice bath for 1 h. After the reaction, 2-(2-bromoethoxy)ethanol (S26-1, 0.81 g, 4.8 mmol) was added and stirred for 1 h under an ice bath, and the reaction solution was slowly returned to room temperature and reacted overnight. After the reaction, the reaction was put under an ice bath, 4 mL of methanol was slowly added to quench the reaction, after stirring for 30 min, water (15 mL) was added to mix the solution well. The reaction solution was extracted twice with dichloromethane (40 mL*2), the organic phases were collected and combined, backwashed once with saturated sodium chloride (40 mL), dried with anhydrous sodium sulfate, and filtered, and the filtrate was concentrated to obtain the crude product E26-1. Cationic lipid E26-1 (2.26 g) was obtained by column chromatography purification, concentration, and oil pump drying.

¹H NMR (400 MHz, CDCl₃) δ: 4.93-4.81 (m, 1H), 4.07 (t, 4H), 3.70 (t, 2H), 3.63 (t, 4H), 3.40 (t, 1H), 2.56 (s, 3H), 2.52 (t, 2H), 2.48 (t, 2H), 2.32 (t, 2H), 1.65-1.25 (m, 54H), 0.89 (t, 9H).

MS (ESI): m/z=756.63 ([M+H] ⁺).

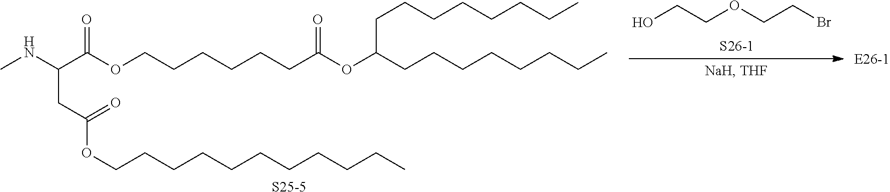

### Example 27: Cationic Lipid (E27-2)

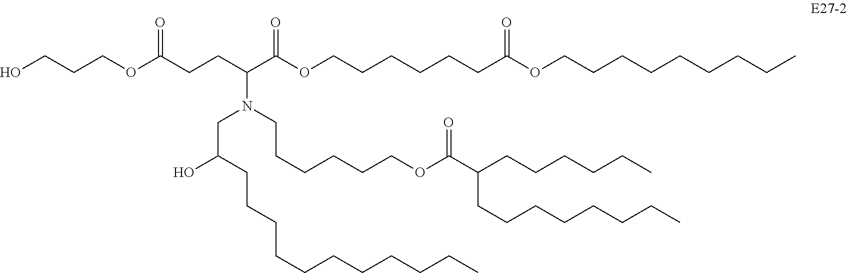 [SMILES: CCCCCCCCCCCC(O)CN(CCCCCCOC(=O)C(CCCCCC)CCCCCCCC)C(CCC(=O)OCCCO)C(=O)OCCCCCCC(=O)OCCCCCCCCC]

The preparation process is as follows:
Step a: compound S18-3 (5.15 g, 9.0 mmol) was added to anhydrous ethanol solution (100 mL) of compound S21-2 (5.80 g, 6.0 mmol) and the reaction was stirred for 10 h at room temperature. After the reaction, the reaction solution was concentrated, diluted with purified water, and extracted with dichloromethane, the organic phase was dried with anhydrous magnesium sulfate and filtered, the filtrate was concentrated and recrystallized to obtain compound S27-1 (2.15 g).
Step b: S1-3 (1.00 g, 2.4 mmol) was dissolved in 20 mL of DMF, then S27-1 (1.54 g, 2.0 mmol) and K₂CO₃ (0.73 g, 5.3 mmol) were added and the reaction was stirred overnight at room temperature. At the end of the reaction, the reaction mixture was concentrated under reduced pressure and 20 mL of ethyl acetate was poured into the solution. After washing with 10% citric acid (10 mL) and saline (10 mL) successively, the organic phase was dried with anhydrous sodium sulfate, filtered and concentrated, and the crude product was purified by column chromatography to obtain E27-1 (1.84 g).
Step c: the above compound E27-1 (1.11 g, 1.0 mmol) was dissolved in THE (10 mL), and added in a nitrogen-protected flask. TBAF solution (10 mL, 1 M) was added and the reaction was conducted overnight to remove TBS protection. After the reaction, the reaction solution was concentrated, diluted with purified water, and extracted with dichloromethane, the organic phase was dried with anhydrous magnesium sulfate and filtered, the filtrate was concentrated and recrystallized to obtain cationic lipid E27-2 (0.83 g, 83.6%). ¹H NMR (400 MHz, CDCl₃) δ: 4.51 (t, 1H), 4.07 (t, 8H), 3.70 (t, 2H), 3.55-3.53 (m, 1H), 2.50 (t, 2H), 2.35-2.25 (m, 7H), 2.13-1.92 (m, 2H), 1.88 (q, 2H), 1.70-1.25 (m, 74H), 0.89 (t, 12H). MS (ESI): m/z=996.84 ([M+H] ⁺).

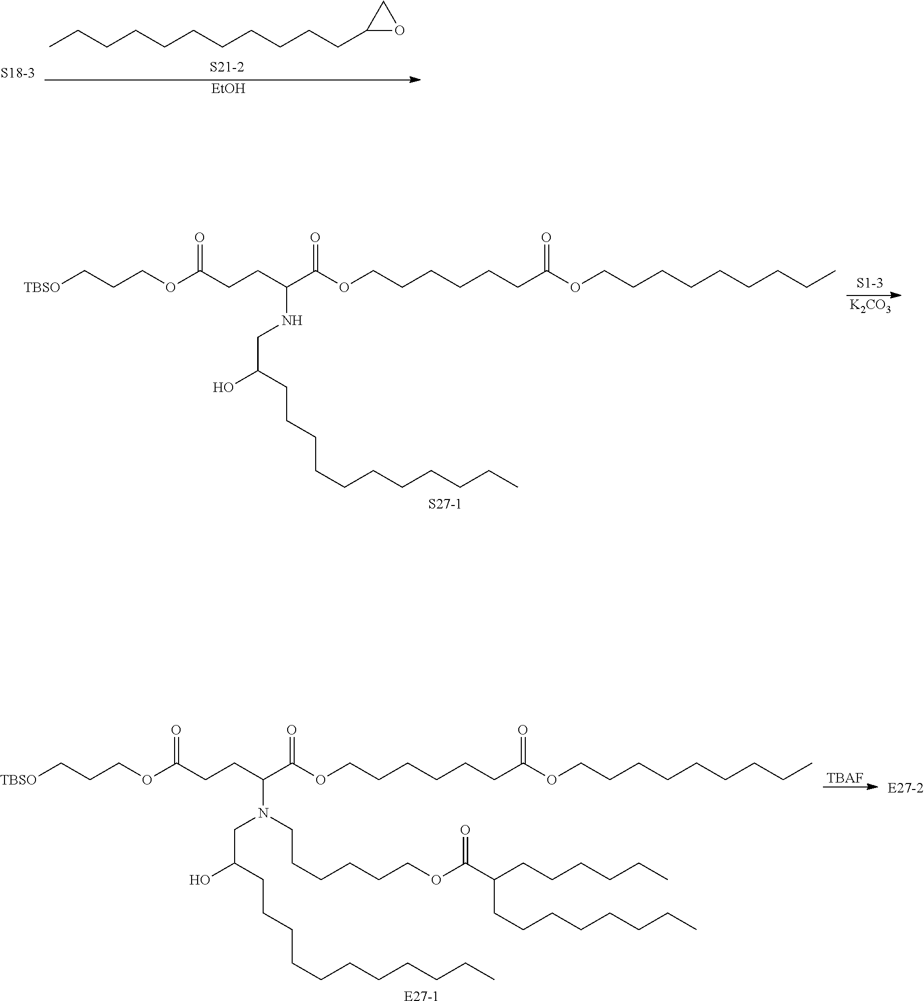

### Example 28: Cationic Lipid (E28-2)

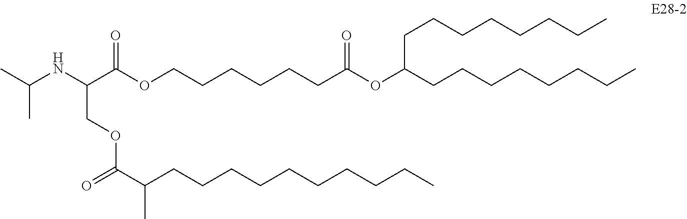 [SMILES: CCCCCCCCCCC(C)C(=O)OCC(NC(C)C)C(=O)OCCCCCCC(=O)OC(CCCCCCCC)CCCCCCCC]

The preparation process is as follows:
Step a: DCC (1.81 g, 8.8 mmol) was added to a round-bottom flask containing compounds S15-2 (1.54 g, 4.0 mmol), S28-1 (1.73 g, 4.8 mmol, the secondary amino group and hydroxyl group of S28-1 are protected by Boc and TBS, respectively) and DMAP (0.12 g, 1.0 mmol) dissolved in dichloromethane (30 mL) under nitrogen atmosphere, and the reaction was conducted for 16 h at room temperature. At the end of the reaction, the precipitate was removed by filtration, the filtrate was concentrated and the residue obtained was purified by column chromatography to obtain compound E28-2 (2.37 g).
Step b: the above compound S28-2 (2.18 g, 3.0 mmol) was dissolved in THE (20 mL), and added in a nitrogen-protected flask, and then TBAF solution (20 mL, 1 M) was added to react overnight to remove TBS protection. After the reaction, the reaction solution was concentrated, diluted with purified water, and extracted with dichloromethane, the organic phase was dried with anhydrous magnesium sulfate and filtered, the filtrate was concentrated and recrystallized to obtain compound S28-3 (1.56 g, 85.0%).
Step c: DCC (0.91 g, 4.4 mmol) was added to a round-bottom flask containing compounds S28-3 (1.23 g, 2.0 mmol), 2-methyldodecanoic acid (S28-4, 0.51 g, 2.4 mmol) and DMAP (61.00 mg, 0.5 mmol) dissolved in dichloromethane (20 mL) under nitrogen atmosphere and the reaction was conducted for 16 h at room temperature. After the reaction, the precipitate was removed by filtration, the filtrate was concentrated, and the resulting residue was purified by column chromatography to obtain compound E28-1 (1.34 g).
Step d: removing the Boc protecting group. A solution of trifluoroacetic acid/dichloromethane (1:2, v/v) was prepared in a dry and clean round-bottom flask, and a dichloromethane solution of E28-1 (1.05 g, 1.3 mmol) was slowly dropwise added under ice bath condition for 2 h at room temperature. After the reaction, the reaction solution was concentrated, diluted with purified water, extracted with dichloromethane, the organic phase was dried with anhydrous magnesium sulfate and filtered, the filtrate was concentrated and recrystallized to obtain cationic lipid E28-2 (0.85 g, 91.7%). ¹H NMR (400 MHz, CDCl₃) δ: 5.00 (t, 1H), 4.93-4.81 (m, 1H), 4.61-4.51 (m, 2H), 4.09 (t, 2H), 4.04 (m, 1H), 2.42-2.26 (m, 3H), 1.65-1.15 (m, 63H), 0.89 (t, 9H). MS (ESI): m/z=710.63 ([M+H] ⁺).

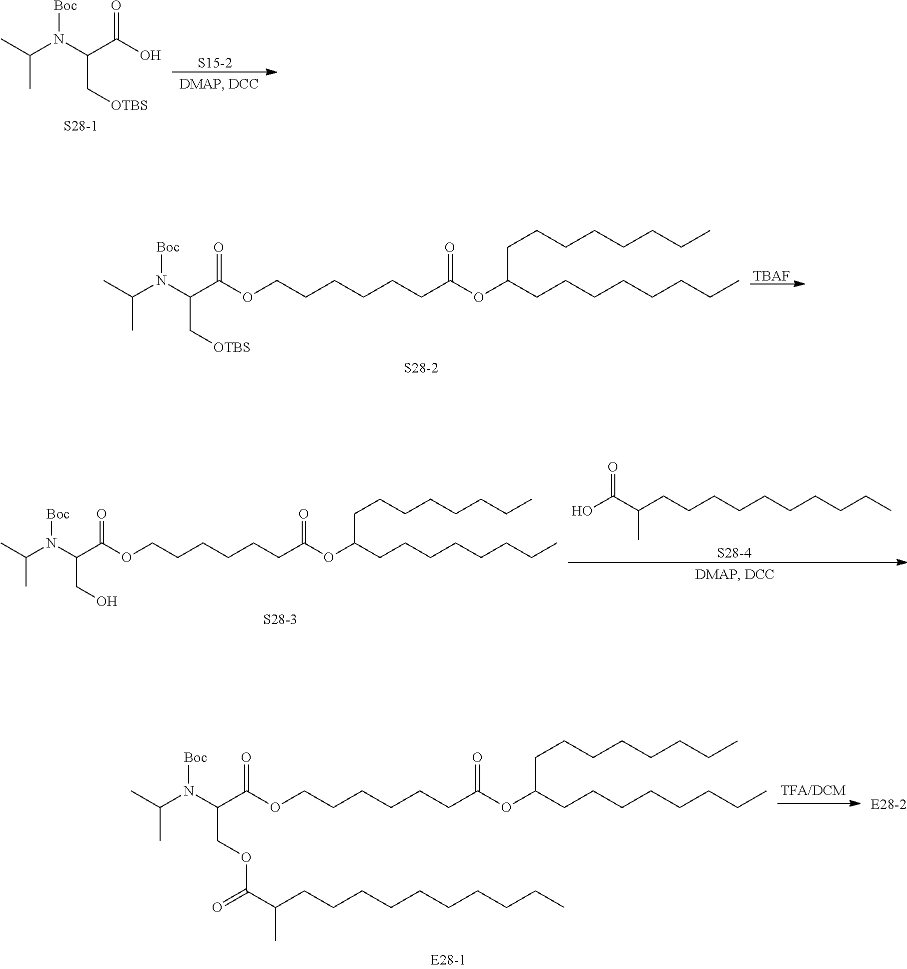

### Example 29: Cationic Lipid (E29-1)

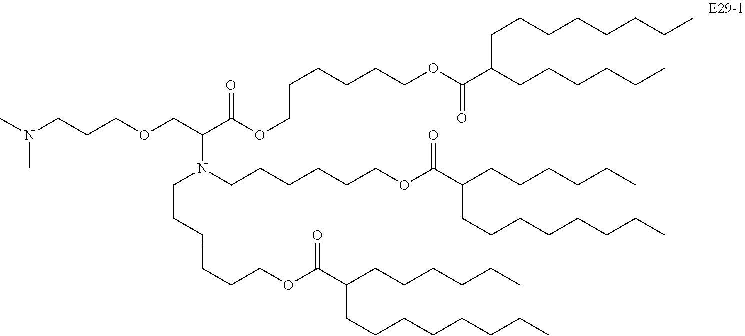 [SMILES: CCCCCCCCC(CCCCCC)C(=O)OCCCCCCOC(=O)C(COCCCN(C)C)N(CCCCCCOC(=O)C(CCCCCC)CCCCCCCC)CCCCCCOC(=O)C(CCCCCC)CCCCCCCC]

The preparation process is as follows:
Step a: S1-3 (1.84 g, 4.4 mmol) was dissolved in 30 mL of DMF, then S29-1 (0.38 g, 2.0 mmol, S29-1 was prepared by the reaction between serine

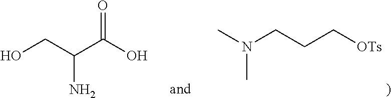 [SMILES: NC(CO)C(=O)O ; Cc1ccc(S(=O)(=O)OCCCN(C)C)cc1]

and K₂CO₃ (0.73 g, 5.3 mmol) were added and stirred overnight at room temperature. At the end of the reaction, the reaction mixture was concentrated under reduced pressure and 30 mL of ethyl acetate was poured into the solution. After washing with 10% citric acid (20 mL) and saline (20 mL) successively, the organic phase was dried with anhydrous sodium sulfate, filtered, and concentrated, and the crude product was purified by column chromatography to obtain S29-2 (1.38 g).
Step b: DCC (0.63 g, 3.1 mmol) was added to a round-bottom flask containing compounds S29-2 (1.21 g, 1.4 mmol), S29-3 (0.60 g, 1.7 mmol, S29-3 was prepared by the reaction between 1,6-hexanediol and S1-1) and DMAP (42.70 mg, 0.4 mmol) dissolved in dichloromethane under nitrogen atmosphere and the reaction was conducted for 16 h at room temperature. After the reaction, the precipitate was removed by filtration, the filtrate was concentrated, and the resulting residue was purified by column chromatography to obtain cationic lipid E29-1 (1.36 g). ¹H NMR (400 MHz, CDCl₃) δ: 4.08 (t, 8H), 3.63 (t, 2H), 3.53 (t, 1H), 3.17-3.12 (m, 2H), 2.87-2.80 (t, 2H), 2.51 (t, 4H), 2.25-2.23 (m, 9H), 1.65-1.23 (m, 98H), 0.88 (t, 18H). MS (ESI): m/z=1206.09 ([M+H] ⁺).

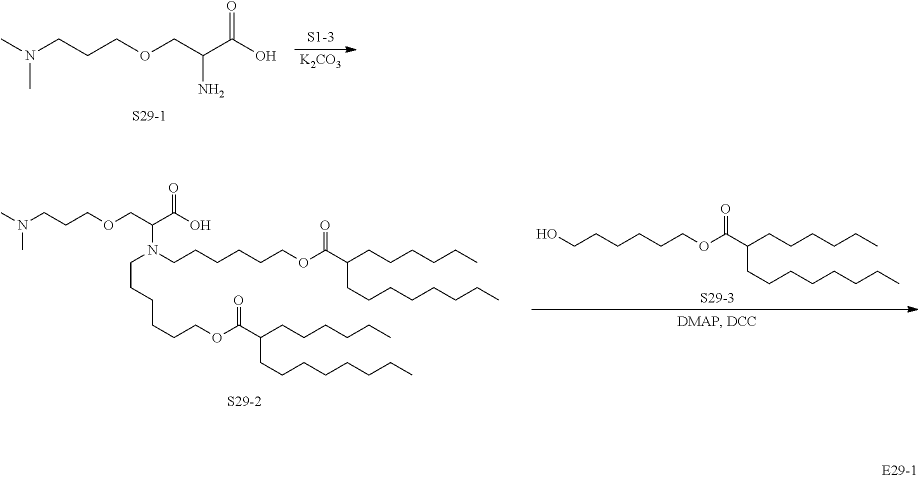

### Example 30: Cationic Lipid (E30-2)

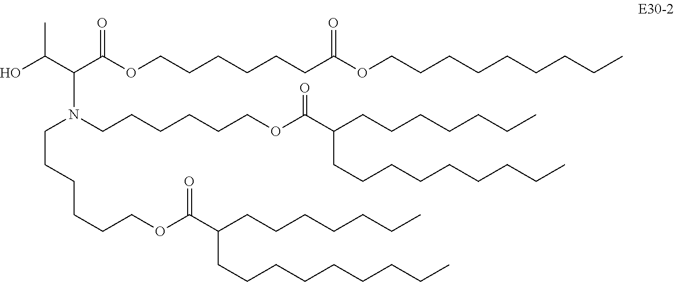 [SMILES: CCCCCCCCCOC(=O)CCCCCCOC(=O)C(C(C)O)N(CCCCCCOC(=O)C(CCCCCCC)CCCCCCCCC)CCCCCCOC(=O)C(CCCCCCC)CCCCCCCCC]

The preparation process is as follows:
Step a: DCC (2.72 g, 13.2 mmol) was added to a round-bottom flask containing isostearic acid (S30-1, 1.70 g, 6.0 mmol), S1-2 (1.30 g, 7.2 mmol) and DMAP (0.18 g, 1.5 mmol) dissolved in dichloromethane (30 mL) under nitrogen atmosphere and the reaction was conducted for 16 h at room temperature. After the end of the reaction, the precipitate was removed by filtration, the filtrate was concentrated, and the resulting residue was purified by column chromatography to obtain compound 6-bromohexyl-2-heptyl undecanoate (S30-2, 2.23 g).
Step b: S30-2 (1.96 g, 4.4 mmol) was dissolved in 30 mL of DMF, then OTBS-threonine (S30-3, 0.47 g, 2.0 mmol) and K₂CO₃ (0.73 g, 5.3 mmol) were added and stirred overnight at room temperature. At the end of the reaction, the reaction mixture was concentrated under reduced pressure and 30 mL of ethyl acetate was poured into the solution. After washing with 10% citric acid (20 mL) and saline (20 mL) successively, the organic phase was dried with anhydrous sodium sulfate, filtered, and concentrated, and the crude product was purified by column chromatography to obtain S30-4 (1.54 g).
Step c: DCC (0.63 g, 3.1 mmol) was added to a round-bottom flask containing compounds S30-4 (1.35 g, 1.4 mmol), S7-4 (0.46 g, 1.7 mmol) and DMAP (42.30 mg, 0.4 mmol) dissolved in dichloromethane (30 mL) under nitrogen atmosphere and reacted for 16 h at room temperature. After the reaction, the precipitate was removed by filtration, the filtrate was concentrated, and the resulting residue was purified by column chromatography to obtain the target compound E30-1 (1.41 g).
Step d: the above compound E30-1 (1.22 g, 1.0 mmol) was dissolved in THE (20 mL) in a nitrogen-protected flask, then TBAF solution (20 mL, 1 M) was added and reacted overnight to remove the TBS protection. At the end of the reaction, the reaction solution was concentrated and extracted, the organic phases were combined, dried with anhydrous sodium sulfate, filtered and concentrated, and purified by column chromatography to obtain cationic lipid E30-2 (0.95 g, 85.7%). ¹H NMR (400 MHz, CDCl₃) δ: 4.09 (t, 8H), 4.05-3.90 (m, 1H), 3.61-3.53 (m, 1H), 2.50 (t, 4H), 2.32 (t, 2H), 2.25 (m, 2H), 1.70-1.15 (m, 97H), 0.88 (t, 15H). MS (ESI): m/z=1106.98 ([M+H] ⁺).

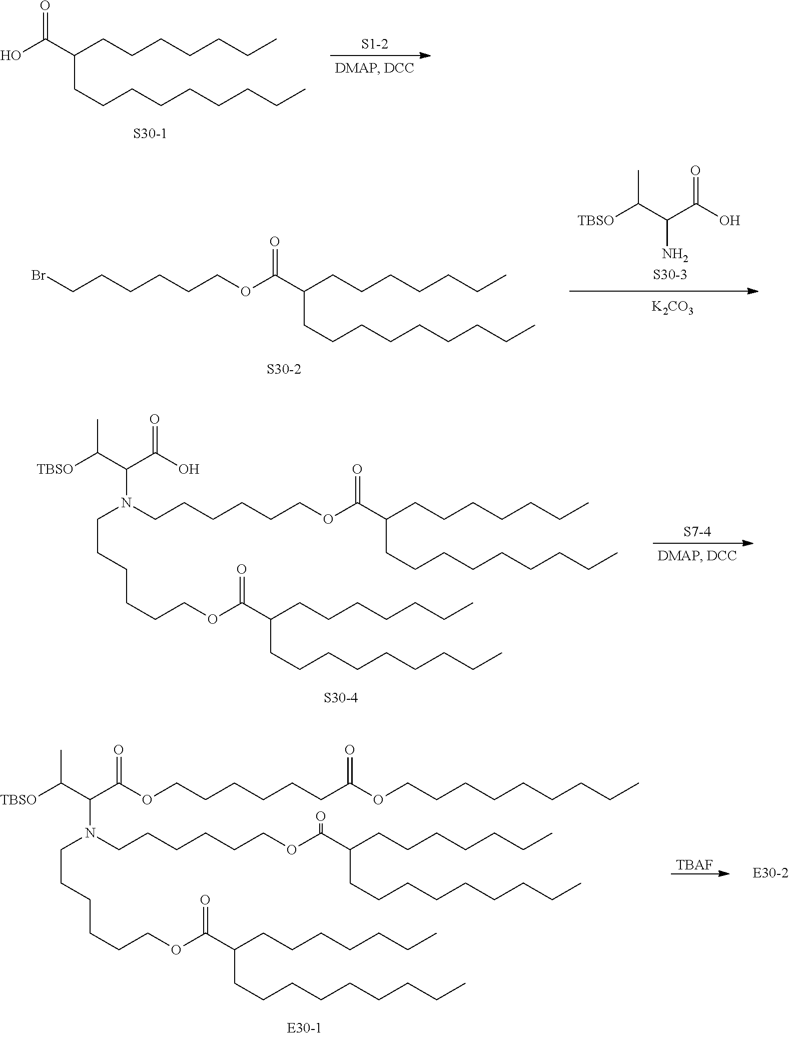

### Example 31: Cationic Lipid (E31-1)

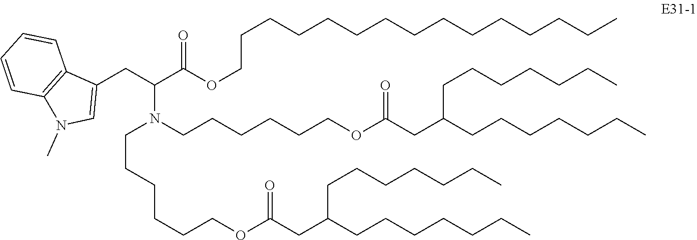 [SMILES: CCCCCCCCCCCCCCCOC(=O)C(Cc1cn(C)c2ccccc12)N(CCCCCCOC(=O)CC(CCCCCCC)CCCCCCC)CCCCCCOC(=O)CC(CCCCCCC)CCCCCCC]

The preparation is shown below:
Step a: DCC (2.72 g, 13.2 mmol) was added to a round-bottom flask containing compounds 3-heptyl-decanoic acid (S31-1, 1.62 g, 6.0 mmol), S1-2 (1.30 g, 7.2 mmol) and DMAP (0.18 g, 1.5 mmol) dissolved in dichloromethane (40 mL) and reacted for 16 h at room temperature. After the end of the reaction, the precipitate was removed by filtration, the filtrate was concentrated, and the resulting residue was purified by column chromatography to obtain 6-bromohexyl-3-heptyl caprate (S31-2, 2.13 g).
Step b: S31-2 (1.90 g, 4.4 mmol) was dissolved in 30 mL of DMF, then 1-methyltryptophan (S31-3, 0.44 g, 2.0 mmol) and K₂CO₃ (0.73 g, 5.3 mmol) were added and stirred at room temperature overnight. At the end of the reaction, the reaction mixture was concentrated under reduced pressure and 30 mL of ethyl acetate was poured into the solution. After washing with 10% citric acid (20 mL) and saline (20 mL) successively, the organic phase was dried with anhydrous sodium sulfate, filtered, and concentrated, and the crude product was purified by column chromatography to obtain S31-4 (1.49 g).
Step c: DCC (0.63 g, 3.1 mmol) was added to a round-bottom flask containing compounds S31-4 (1.29 g, 1.4 mmol), S6-3 (0.38 g, 1.7 mmol) and DMAP (42.70 mg, 0.4 mmol) dissolved in dichloromethane (20 mL) under nitrogen atmosphere and reacted for 16 h at room temperature. After the reaction, the precipitate was removed by filtration, the filtrate was concentrated, and the resulting residue was purified by column chromatography to obtain cationic lipid E31-1 (1.31 g). ¹H NMR (400 MHz, CDCl₃) δ: 7.72-7.70 (m, 1H), 7.28 (d, 1H), 7.19 (m, 1H), 7.05 (d, 1H), 6.82 (s, 1H), 4.64 (t, 1H), 4.09 (t, 6H), 3.71 (s, 3H), 3.60-3.38 (d, 2H), 2.51 (t, 4H), 2.20 (t, 4H), 1.70-1.15 (m, 92H), 0.88 (t, 15H). MS (ESI): m/z=1134.01 ([M+H] ⁺).

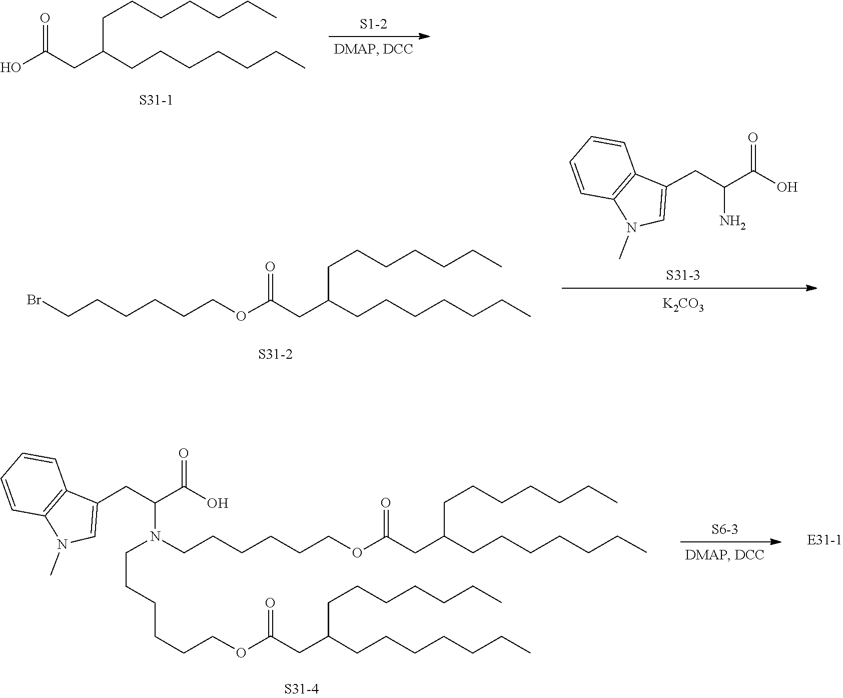

### Example 32: Cationic Lipid (E32-2)

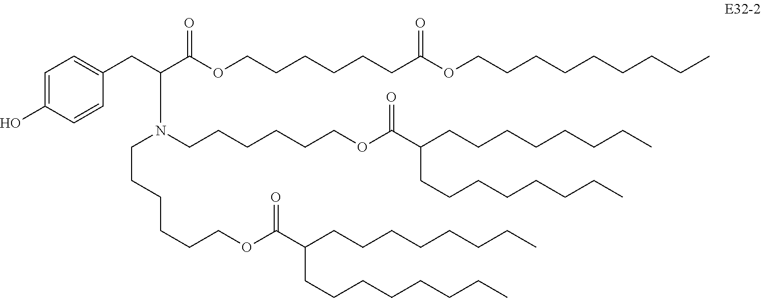 [SMILES: CCCCCCCCCOC(=O)CCCCCCOC(=O)C(Cc1ccc(O)cc1)N(CCCCCCOC(=O)C(CCCCCCCC)CCCCCCCC)CCCCCCOC(=O)C(CCCCCCCC)CCCCCCCC]

The preparation process is as follows:
Step a: compound S5-2 (1.97 g, 4.4 mmol) was dissolved in 30 mL of DMF, then OTBS-tyrosine (S32-1, 0.59 g, 2.0 mmol) and K₂CO₃ (0.73 g, 5.3 mmol) were added and stirred overnight at room temperature. At the end of the reaction, the reaction mixture was concentrated under reduced pressure and 30 mL of ethyl acetate was poured into the solution. After washing with 10% citric acid (20 mL) and saline (20 mL) successively, the organic phase was dried with anhydrous sodium sulfate, filtered and concentrated, and the crude product was purified by column chromatography to obtain S32-2 (1.63 g).
Step b: DCC (0.63 g, 3.1 mmol) was added to a round-bottom flask containing compounds S32-2 (1.44 g, 1.4 mmol), S7-4 (0.46 g, 1.7 mmol) and DMAP (42.70 mg, 0.4 mmol) dissolved in dichloromethane (20 mL) under nitrogen atmosphere and the reaction was conducted for 16 h at room temperature. After the end of the reaction, the precipitate was removed by filtration, the filtrate was concentrated, and the resulting residue was purified by column chromatography to obtain compound E32-1 (1.48 g).
Step c: the above compound E32-1 (1.28 g, 1.0 mmol) was dissolved in THE (20 mL), and added to a nitrogen-protected flask, and then TBAF solution (20 mL, 1 M) was added to react overnight to remove TBS protection. After the reaction, the reaction solution was concentrated and extracted, the organic phases were combined, dried, filtered, and concentrated by anhydrous sodium sulfate, and the cationic lipid E32-2 (1.01 g, 86.1%) was obtained by column chromatography purification. ¹H NMR (400 MHz, CDCl₃) δ: 7.28-6.85 (m, 4H), 4.08 (t, 8H), 3.84 (t, 1H), 3.33-2.90 (m, 2H), 2.51 (t, 4H), 2.33-2.25 (m, 4H), 1.66-1.25 (m, 94H), 0.88 (t, 15H). MS (ESI): m/z=1169.00 ([M+H] ⁺).

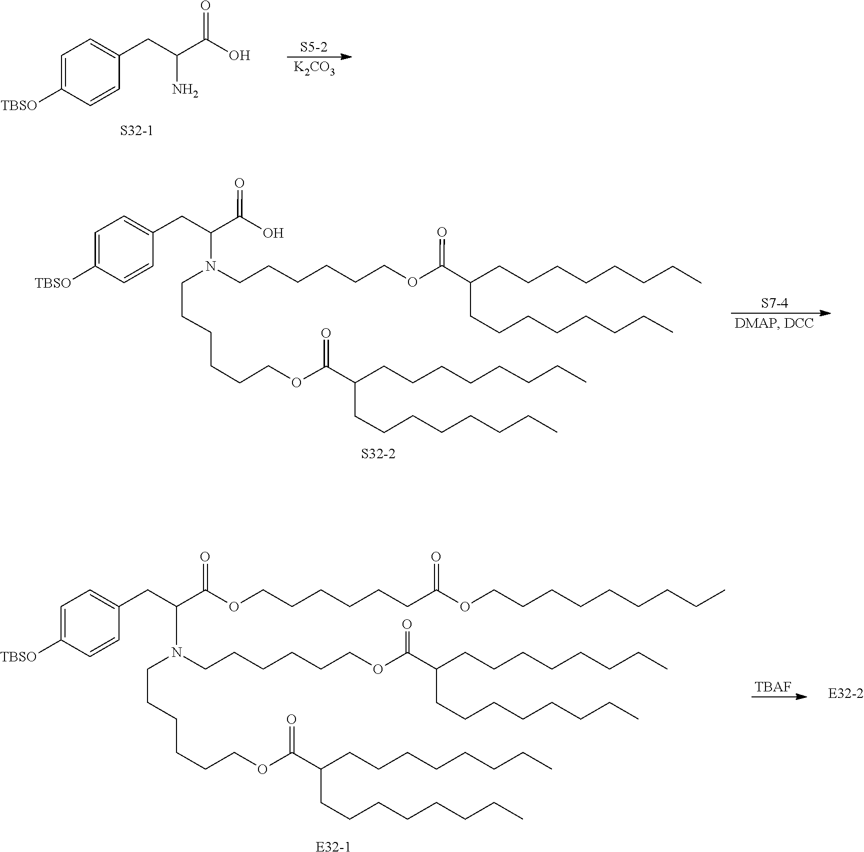

### Example 33: Cationic Lipid (E33-1)

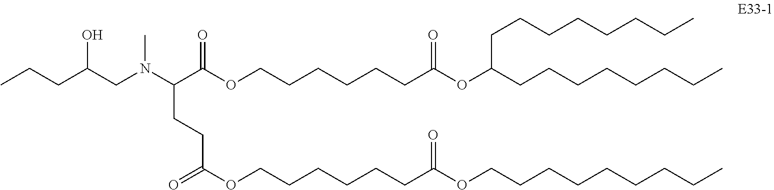 [SMILES: CCCCCCCCCOC(=O)CCCCCCOC(=O)CCC(C(=O)OCCCCCCC(=O)OC(CCCCCCCC)CCCCCCCC)N(C)CC(O)CCC]

The preparation process is as follows:

1,2-epoxypentane (S33-1, 0.17 g, 2.0 mmol) and S17-2 (1.56 g, 2.0 mmol) were dissolved in 20 mL of acetonitrile, followed by the addition of calcium trifluoromethanesulfonate (Ca(OTf)₂, 0.34 g, 1.0 mmol). After stirring the reaction mixture at room temperature until the reaction was complete showed by TLC, acetonitrile was evaporated, water (10 mL) was added, and the solution was extracted with dichloromethane (20 mL*3). The organic layer was combined, dried with anhydrous sodium sulfate, filtered, and evaporated. The crude product was separated and purified by column chromatography, the target eluent was collected and concentrated to obtain cationic lipid E33-1 (1.65 g, 95.1%). ¹H NMR (400 MHz, CDCl₃) δ: 4.93-4.81 (m, 1H), 4.51 (t, 1H), 4.07 (t, 6H), 3.65-3.58 (m, 1H), 2.56 (s, 3H), 2.34-2.32 (m, 6H), 2.30-2.28 (m, 2H), 2.13-1.92 (m, 2H), 1.70-1.25 (m, 62H), 0.89 (t, 12H). MS (ESI): m/z=868.72 ([M+H] ⁺).

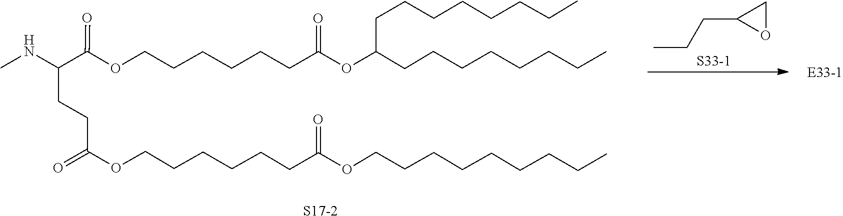

### Example 34: Cationic Lipid (E34-1)

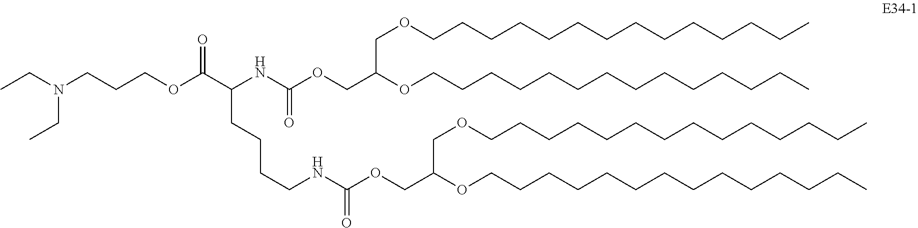 [SMILES: CCCCCCCCCCCCCCOCC(COC(=O)NCCCCC(NC(=O)OCC(COCCCCCCCCCCCCCC)OCCCCCCCCCCCCCC)C(=O)OCCCN(CC)CC)OCCCCCCCCCCCCCC]

The preparation process is as follows:
Step a: S4-4 (2.43 g, 5.0 mmol) and N,N [symbol] uccinimide carbonate (DSC, S34-1, 1.92 g, 7.5 mmol) were dissolved in dichloromethane (40 mL) and stirred under an ice bath. TEA (2.10 mL, 15.0 mmol) was added to the stirred solution, the reaction mixture was stirred overnight at room temperature, and the reaction progress was detected using TLC. After the end of the reaction, the reaction mixture was diluted with dichloromethane, the organic layer was washed with water (40 mL) and sodium bicarbonate aqueous solution (40 mL) successively, concentrated, further purified by column chromatography, concentrated, and vacuum dried to obtain compound S34-2 (2.67 g).
Step b: S34-3 (1.33 g, 6.6 mmol) was dissolved in dichloromethane (40 mL), then S34-2 (1.88 g, 3.0 mmol) and TEA (0.75 mL, 5.4 mmol) were added sequentially and the reaction was stirred at room temperature overnight. At the end of the reaction, the reaction solution was concentrated and the crude product was purified by column chromatography, concentrated and dried by oil pump to obtain compound S34-4 (2.86 g)
Step c: removing the tBu protecting group. A solution of trifluoroacetic acid/dichloromethane (1:2, v/v) was prepared in a dry and clean round-bottomed flask. A dichloromethane solution of S34-4 (2.45 g, 2.0 mmol) was slowly added dropwise under the condition of ice bath, and the reaction was carried out for 2 hours at room temperature. After the end of the reaction, the reaction solution was concentrated, purified water was added, the reaction was extracted with dichloromethane, the extract was dried with anhydrous magnesium sulfate and filtered, and the filtrate was concentrated and recrystallized to obtain compound S34-5 (2.15 g, 92.2%).
Step d: DCC (0.45 g, 2.2 mmol) was added into a round-bottom flask containing S23-2 (0.16 g, 1.2 mmol), S34-4 (1.17 g, 1.0 mmol) and DMAP (30.50 mg, 0.3 mmol) dissolved in dichloromethane (20 mL) under nitrogen atmosphere, and the reaction was conducted for 16 h at room temperature. After the reaction, the precipitate was removed by filtration, the filtrate was concentrated, and the resulting residue was purified by column chromatography to obtain cationic lipid E34-1 (1.11 g). ¹H NMR (400 MHz, CDCl₃) δ: 4.34-4.29 (m, 1H), 4.13-4.01 (m, 6H), 3.59-3.33 (m, 14H), 3.17-3.00 (m, 6H), 2.87-2.80 (m, 2H), 2.01-1.82 (m, 2H), 1.98-1.21 (m, 108H), 0.88 (t, 12H). MS (ESI): m/z=1281.15 ([M+H] ⁺).

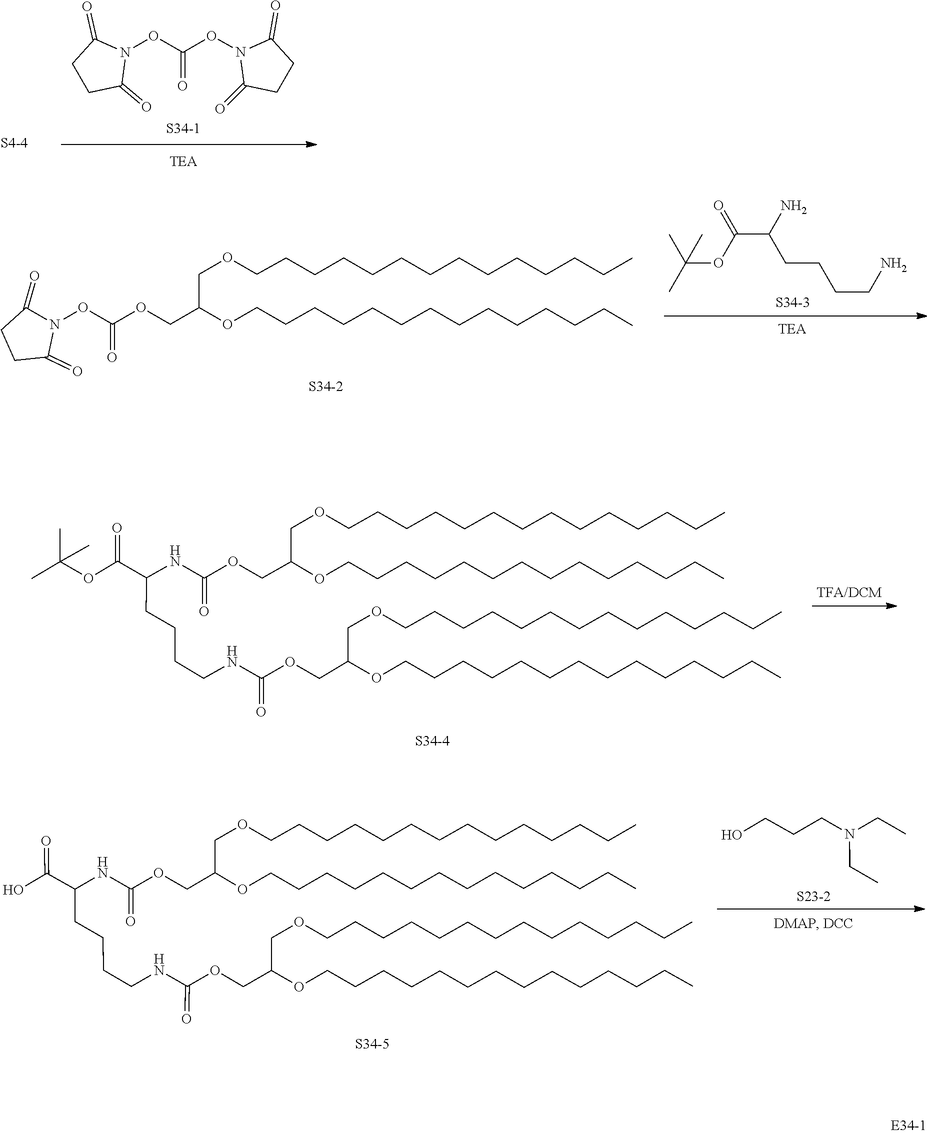

### Example 35: Cationic Lipid (E35-1)

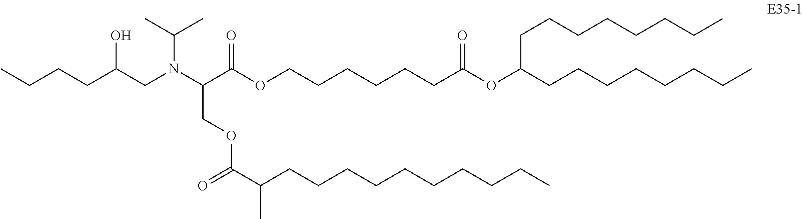 [SMILES: CCCCCCCCCCC(C)C(=O)OCC(C(=O)OCCCCCCC(=O)OC(CCCCCCCC)CCCCCCCC)N(CC(O)CCCC)C(C)C]

The preparation process is as follows:

1,2-Epoxyhexane (S35-1, 0.20 g, 2.0 mmol) and E28-2 (1.12 g, 2.0 mmol) were dissolved in 20 mL of acetonitrile and then Ca(OTf)₂ (0.34 g, 1.0 mmol) was added. The reaction mixture was stirred at room temperature until TLC showed that the reaction was complete. Then the acetonitrile was evaporated, water (10 mL) was added and the reaction solution was extracted with dichloromethane (10 mL*3). The organic layers were combined, dried with anhydrous sodium sulfate, filtered, and evaporated. The crude product was separated and purified by column chromatography, and the target eluent was collected and concentrated to obtain the cationic lipid E35-1 (1.51 g, 93.2%). ¹H NMR (400 MHz, CDCl₃) δ: 5.00 (t, 1H), 4.93-4.80 (m, 1H), 4.61-4.51 (m, 2H), 4.09 (t, 2H), 4.04 (t, 1H), 3.64-3.57 (m, 1H), 2.42-2.26 (m, 5H), 1.65-1.10 (m, 69H), 0.89 (t, 12H). MS (ESI): m/z=810.71 ([M+H] ⁺).

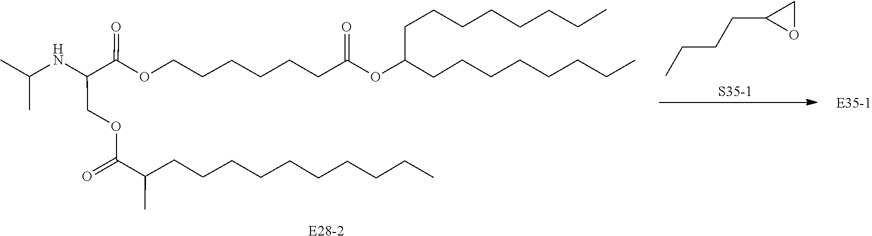

### Example 36: Cationic Lipid (E36-1)

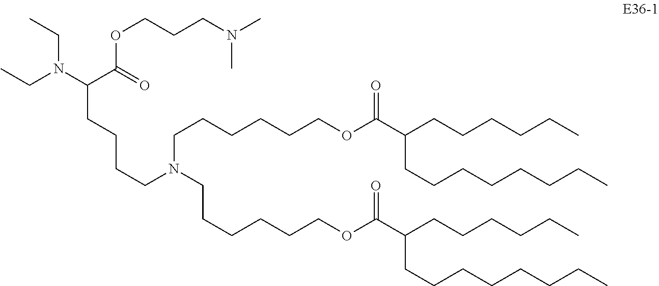 [SMILES: CCCCCCCCC(CCCCCC)C(=O)OCCCCCCN(CCCCCCOC(=O)C(CCCCCC)CCCCCCCC)CCCCC(C(=O)OCCCN(C)C)N(CC)CC]

The preparation process is as follows:

DCC (0.63 g, 3.1 mmol) was added to a round-bottom flask containing compounds S1-5 (1.23 g, 1.4 mmol), S14-2 (0.17 g, 1.7 mmol) and DMAP (42.70 mg, 0.4 mmol) dissolved in dichloromethane (20 mL) under nitrogen atmosphere, and the reaction solution was conducted for 16 h at room temperature. After the reaction, the precipitate was removed by filtration, the filtrate was concentrated, and the resulting residue was purified by column chromatography to obtain cationic lipid E36-1 (1.14 g). ¹H NMR (400 MHz, CDCl₃) δ: 4.09 (t, 6H), 3.46 (t, 1H), 3.18-2.99 (m, 2H), 2.87-2.80 (t, 2H), 2.57 (q, 4H), 2.50 (t, 4H), 2.25-2.23 (m, 8H), 2.01-1.82 (m, 4H), 1.71-1.22 (m, 74H), 0.88 (t, 12H). MS (ESI): m/z=964.89 ([M+H] ⁺).

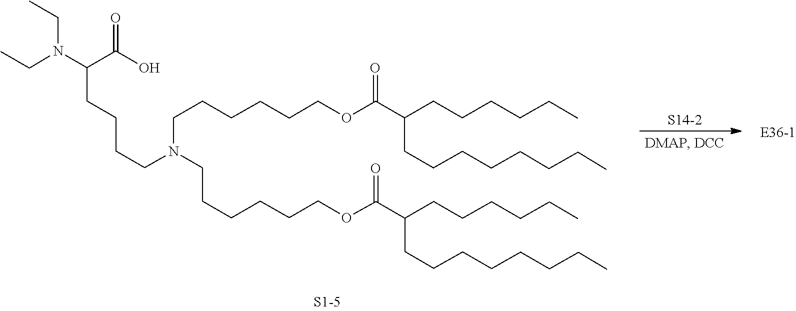

### Example 37.1 Preparation of PEGylated lipid E37-1

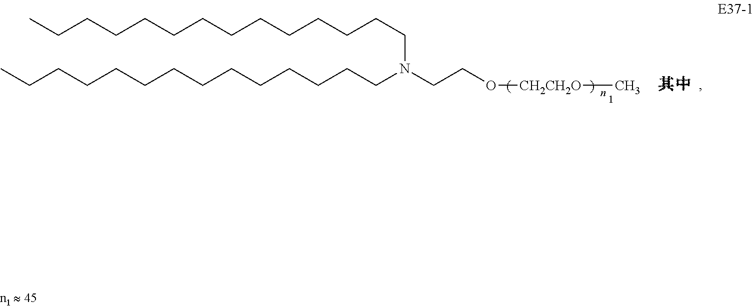

The preparation process is as follows:
Step a: compound S37-1 (20.00 g, 10.0 mmol, mPEG-OH, M_{W}≈2000, n₁≈45, PDI=1.03) and toluene (200 mL) underwent the azeotropic water removal at 140° C., after 60 mL of solvent was evaporated, the reaction was cooled to room temperature. TEA (2.02 g, 20.0 mmol) and MsCl (2.05 g, 18.0 mmol) were then added and stirred overnight at room temperature. After the reaction, the reaction solution was poured into water (200 mL), extracted twice with EtOAc (100 mL*2), the aqueous phase was retained, and the aqueous phase was extracted twice with dichloromethane (100 mL*2), the organic phases were combined, dried, filtered, concentrated, dissolved at 50° C. with isopropanol, recrystallized in an ice bath, and filtered to obtain compound S37-2 (18.00 g, 90%).
Step b: the above compound S37-2 (18.00 g, 9.0 mmol) was added to 80 mL of water and dissolved at room temperature with stirring. Potassium carbonate (12.42 g, 90.0 mmol), compound S37-3 (9.58 g, 45.0 mmol) and tetrabutylammonium bromide (0.29 g, 0.9 mmol) were added and the reaction solution was stirred at room temperature for 72 h. After the reaction, the reaction was extracted twice with dichloromethane (100 mL*2), the organic phases were combined, backwashed once with saturated sodium chloride aqueous solution (100 mL), the organic phase was retained, dried with anhydrous sodium sulfate, filtered, and the filtrate was concentrated to obtain the crude compound S37-4. The compound of interest S37-4 (12.00 g) was obtained by column chromatography purification, concentration, and oil pump drying.
Step c: the above compound S37-4 (12.00 g, 6.0 mmol) was dissolved in dried THF (120 mL). NaH (60%, 2.40 g, 60.0 mmol) was slowly added under an ice bath and reacted under an ice bath for 1 h. Compound S37-5 (8.28 g, 30.0 mmol) was added and the reaction was stirred under an ice bath for 1 h. After the reaction, the reaction was slowly cooled to room temperature and the reaction was continued overnight. At the end of the reaction, the reaction was quenched by slowly adding 2 mL of methanol under an ice bath. After stirring for 30 minutes, water (300 mL) was added and mixed. The reaction was extracted twice with EtOAc (150 mL*2), the aqueous phase was retained, and then extracted twice with dichloromethane (100 mL*2), the organic phases were collected and combined, and backwashed once with saturated sodium chloride (100 mL). The organic phase was dried with anhydrous sodium sulfate and filtered, and the filtrate was concentrated to obtain the crude product of PEGylated lipid E37-1. The crude product was purified by column chromatography, concentrated and dried by oil pump to obtain PEGylated lipid E37-1 (9.00 g). ¹H NMR (400 MHz, CDCl₃) δ: 3.85-3.45 (m, 182H), 3.37 (s, 3H), 3.15 (t, 2H), 2.94 (t, 2H), 2.62 (t, 2H), 1.58-1.48 (m, 4H), 1.36-1.19 (m, 44H), 0.86 (t, 6H). The molecular weight of E37-1 was determined to be 2447 Da by MALDI-TOF test, and PDI=1.03.

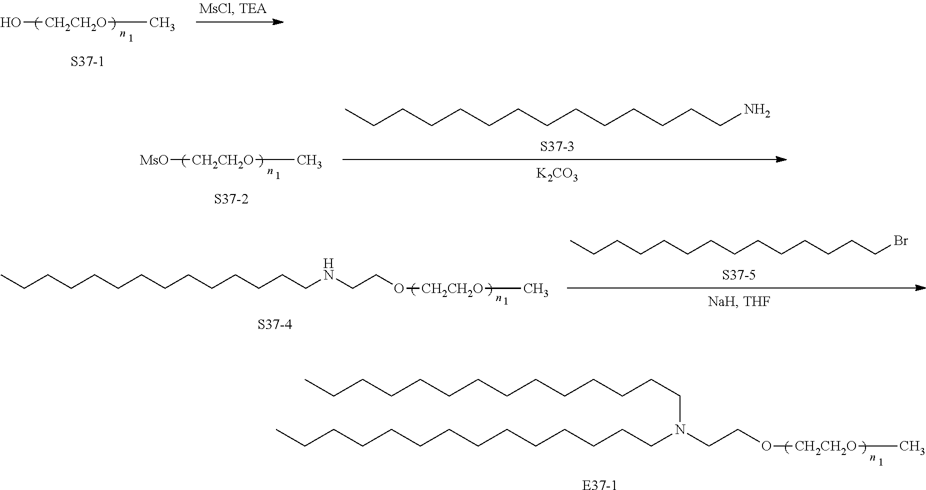

### Example 37.2: Preparation of PEG-Lipid E37-2

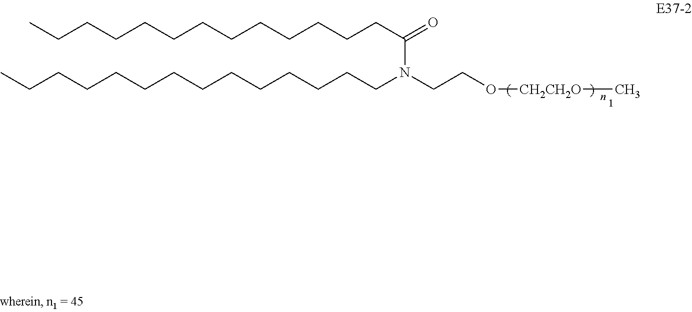

The preparation process is as follows:

The above compound S37-4 (11.26 g, 5.0 mmol), S37-6 (1.95 g, 6.0 mmol) and triethylamine (TEA, 0.76 g, 7.5 mmol) were dissolved in dichloromethane (100 mL) and the reaction was stirred at room temperature overnight. The reaction solution was concentrated and dissolved in 100 mL of water, extracted twice with EtOAc (100 mL*2) and the aqueous phase was retained. Sodium chloride was added and the reaction solution was extracted twice with dichloromethane (100 mL*2). The organic phases were combined and then backwashed once with saturated NaCl (100 mL), dried with anhydrous magnesium sulfate, filtered, and concentrated, and the crude product was purified by column chromatography, concentrated, and dried by oil pump to obtain PEGylated lipid E37-2 (10.1 g, 84.8%). ¹H NMR (400 MHz, CDCl₃) δ: 3.84-3.45 (m, 182H), 3.37 (s, 3H), 3.35 (t, 2H), 3.18 (t, 2H), 2.27 (t, 2H), 1.56-1.40 (m, 4H), 1.36-1.18 (m, 46H), 0.87 (t, 6H). The molecular weight of E37-2 was determined by MALDI-TOF test to be 2461 Da, PDI=1.03.

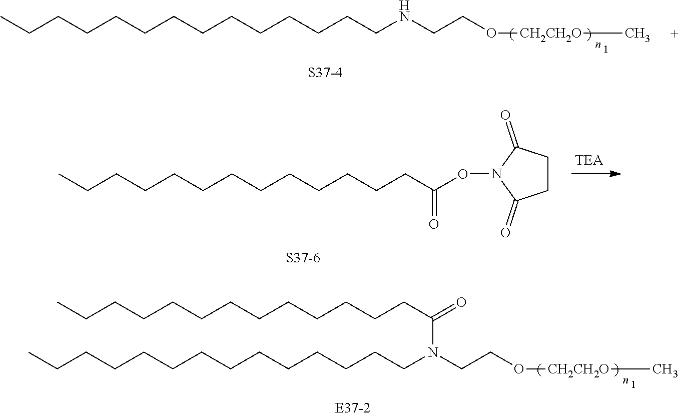

### Example 38: Preparation of LNP-mRNA Pharmaceutical Composition and Testing of its Physicochemical Properties

### Example 38.1: Preparation of LNP-mRNA Pharmaceutical Composition

In the present embodiment, multiple sets of LNP-mRNA pharmaceutical compositions containing Fluc-mRNA were prepared for comparison. The phospholipids contained in each group were DSPC, and the steroid lipids contained were cholesterol. The differences in each group were cationic lipids and PEGylated lipids. Wherein, the control group L-0: cationic lipids contained were amino acid-based cationic lipids in the prior art (referred to as CL-1, prepared with reference to the method disclosed in CN104168887A, with a structure of

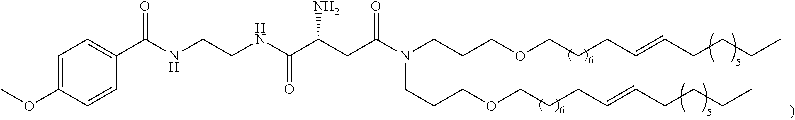

and containing the PEGylated lipid PEG2k-DMG (DMG). Experimental group series (L-1~L-38): cationic lipids were amino acid-based cationic lipids prepared in the embodiment of the present application, and the PEGylated lipids were PEG-DMG; and experimental group (L-37~L-38): cationic lipids were amino acid-based cationic lipids prepared in the embodiment of the present application, and PEGylated lipids were PEGylated lipid E37-1 or E37-2 prepared in the present application, specifically as shown in Table 1.

The preparation method of LNP-mRNA pharmaceutical composition is as follows:
Step a: cationic lipids, DSPC, cholesterol and PEGylated lipids were dissolved in ethanol at a molar ratio of 48:9:42:1.5 to obtain an ethanol phase solution;
Step b: Fluc-mRNA was added to 10-50 mM citrate buffer (pH=4) to obtain an aqueous solution;
Step c: ethanol phase solution and aqueous phase solution were mixed (1:3 v/v) to prepare LNP-mRNA and washed by multiple DPBS ultrafiltration to remove ethanol and free molecules, and finally, filtered through a sterile filter of 0.2 m to obtain the LNP-mRNA pharmaceutical compositions.

### Example 38.2: Testing of Physicochemical Properties of LNP-mRNA Pharmaceutical Compositions

Determination of encapsulation rate: encapsulation rate of LNP-mRNA compositions was determined using the Quant-it Ribogreen RNA Quantification Kit. The results showed that the lipid composition of the present application (L-1~L-38) had a high nucleic acid encapsulation rate for nucleic acid drugs, all in the range of 84%-97%, and most of the encapsulation rates were in the range of 900%-97%, the results were shown in Table 1 below. The results showed that the lipid compositions prepared from amino acid-based cationic lipids in each experimental group could encapsulate mRNA well, showing a better encapsulation rate than the existing amino acid-based cationic lipids, and there were also differences in the encapsulation rate among different amino acid-based cationic lipids.

Determination of particle size: in the present embodiment, the particle size of LNP-mRNA was determined by dynamic light scattering (DLS), and the results were shown in Table 1 below. The measured LNP-mRNA had high dimensional uniformity with PDJ values all less than 0.3. The particle size of LNP-mRNA prepared with the lipid composition of the present application were in the range of 90-110 nm.

TABLE 1
Summary of formulation of each lipid composition and the particle
size and encapsulation rate of the prepared LNP-mRNA

| LNP-mRNA | L-0 | L-1 | L-2 | L-3 | L-4 | L-5 |
|---|---|---|---|---|---|---|
| Cationic lipid CL | CL-1 | E1-2 | E2-2 | E3-1 | E4-2 | E5-2 |
| PEGylated lipid PL | DMG | | | | | |
| Encapsulation | 87.21 | 93.06 | 91.53 | 89.30 | 92.74 | 84.12 |
| efficiency/% | | | | | | |
| Particle size/nm | 102.91 | 96.04 | 92.18 | 93.67 | 91.56 | 107.11 |
| LNP-mRNA | L-6 | L-7 | L-8 | L-9 | L-10 | L-11 |
| Cationic lipid CL | E6-1 | E7-1 | E8-2 | E9-3 | E10-3 | E11-2 |
| PEGylated lipid PL | DMG | | | | | |
| Encapsulation | 97.23 | 95.86 | 89.43 | 90.27 | 89.81 | 88.23 |
| efficiency/% | | | | | | |
| Particle size/nm | 92.08 | 91.52 | 97.26 | 98.16 | 107.31 | 95.62 |
| LNP-mRNA | L-12 | L-13 | L-14 | L-15 | L-16 | L-17 |
| Cationic lipid CL | E12-2 | E13-1 | E14-1 | E15-1 | E16-1 | E16-2 |
| Encapsulation | 94.51 | 92.13 | 93.67 | 91.83 | 93.47 | 89.82 |
| efficiency/% | | | | | | |
| Particle size/nm | 91.33 | 95.57 | 96.85 | 94.10 | 93.23 | 102.10 |
| LNP-mRNA | L-18 | L-19 | L-20 | L-21 | L-22 | L-23 |
| Cationic lipid CL | E17-1 | E18-2 | E19-2 | E21-2 | E22-2 | E23-1 |
| PEGylated lipid PL | DMG | | | | | |
| Encapsulation | 92.06 | 92.30 | 95.62 | 90.01 | 89.76 | 92.37 |
| efficiency/% | | | | | | |
| Particle size/nm | 94.34 | 95.61 | 92.53 | 103.47 | 98.29 | 93.17 |
| LNP-mRNA | L-24 | L-25 | L-26 | L-27 | L-28 | L-29 |
| Cationic lipid CL | E24-1 | E25-1 | E26-1 | E27-2 | E28-2 | E29-1 |
| PEGylated lipid PL | DMG | | | | | |
| Encapsulation | 93.61 | 91.82 | 87.80 | 89.35 | 86.71 | 92.55 |
| efficiency/% | | | | | | |
| Particle size/nm | 97.33 | 93.26 | 95.88 | 97.40 | 93.10 | 108.20 |
| LNP-mRNA | L-30 | L-31 | L-32 | L-33 | L-34 | L-35 |
| Cationic lipid CL | E30-2 | E31-1 | E32-2 | E33-2 | E34-1 | E35-1 |
| PEGylated lipid PL | DMG | | | | | |
| Encapsulation | 91.10 | 88.31 | 85.58 | 92.86 | 90.01 | 87.20 |
| efficiency/% | | | | | | |
| Particle size/nm | 97.30 | 106.25 | 103.33 | 95.10 | 92.43 | 93.10 |

| LNP-mRNA | L-36 | L-37 | L-38 |
|---|---|---|---|
| Cationic lipid CL | E36-1 | E6-1 | E6-1 |
| PEGylated lipid PL | DMG | E37-1 | E37-2 |
| Encapsulation | 87.54 | 93.37 | 92.85 |
| efficiency/% | | | |
| Particle size/nm | 96.12 | 95.43 | 93.33 |

### Example 39: Biological Activity Test of Formulation of LNP-mRNA Pharmaceutical Composition

### (1) Assays for Cytotoxicity (Biocompatibility)

The cytotoxicity of the formulation of LNP-mRNA pharmaceutical composition of the present application was tested by MTT assay. The formulation of LNP-mRNA pharmaceutical composition was dissolved in the medium to prepare the required concentration, and if necessary, an appropriate amount of co-solvent could be added. Hela cells was used as a cell model, and 100 μL/well of cell suspension was seeded into 96-well plates at a density of 1×10⁴ cells/well. After inoculation, the cells were incubated in a cell culture incubator at 37° C., 4% CO₂ for 24 h and then the culture medium was discarded. Then, 100 L medium containing 0.1-0.3 ug/well mRNA (LNP-mRNA pharmaceutical composition prepared in Example 38) was added into each well of the experimental group; and 100 L of fresh medium was added to the blank control group. Each group had six replicate wells for each concentration (0.1 ug, 0.15 ug, 0.20 ug, 0.25 ug and 0.3 ug). After 24 h co-incubation of the formulation of LNP-mRNA pharmaceutical composition with Hela cells, 20 L of PBS buffer containing 5 mg/mL MTT was added to each well. After 4 h incubation of MTT with cancer cells, the mixture of medium and MTT buffer was discarded, followed by adding 150 L DMSO per well to dissolve the purple formazan crystals formed in living cells. After sufficient shaking, the absorbance was tested with a microplate reader and calculated from the measured absorbance value. The results showed that compared with the blank control group, the cell viability rate of the formulation of LNP-mRNA pharmaceutical composition prepared in the present application was greater than 95%, indicating that the formulations of LNP-mRNA pharmaceutical composition of the present application had good biocompatibility.

### (2) Serum Stability Evaluation

LNP-mRNA pharmaceutical compositions were added to the medium containing 10% fetal bovine serum (FBS) with agitation at 37° C. Samples was taken at regular intervals to determine the particle size change of LNP-mRNA, and the serum stability of the LNP-mRNA pharmaceutical composition formulation was analyzed by testing its particle size change. The experimental results showed that within 7 days, the particle size change of control groups and experimental groups were less than 10%; especially the particle size change of L-1, L-2, L-3, L-6, L-12, L-14, L-19 and L-23 in experimental groups was less than 5%, indicating that the formulation of LNP-mRNA pharmaceutical composition prepared by cationic lipids of the present application had good serum stability.

### (3) Study of Cell Transfection Activity

In order to investigate the mRNA transfection rate at the cellular level of each group of LNP-mRNA pharmaceutical compositions prepared in Example 38 of the present application, luciferase bioluminescence was used for testing. The LNP-mRNA pharmaceutical composition was dissolved in medium to prepare the required dose. Hela cells were used as the cell model, and cell suspensions of 100 μL/well were seeded in black-edged, clear-bottomed 96-well plates at an inoculum density of 6000 cells/well. After inoculation, the cells were cultured in a cell culture incubator for 24 h. The cells were then dosed with 0.2 ug mRNA per well, and the control group was added with the corresponding dose of free Fluc-mRNA. After 24 h of transfection, the old medium was replaced with fresh medium containing D-fluorescein sodium substrate (1.5 mg/mL). After 5 min of incubation, bioluminescence was detected using a microplate reader, the stronger the fluorescence indicates the more Fluc-mRNA transported into the cytoplasm and translated the corresponding fluorescent protein, as shown in Table 2. Among them, the relative value of fluorescence intensity was the ratio of the fluorescence intensity value of each group to the fluorescence intensity of the control group. The results showed that the LNP-mRNA pharmaceutical compositions prepared in the present application had excellent in vitro transfection effects, that was, the LNPs in the experimental group were effective nucleic acid delivery carriers, and they were basically superior to the L-0 group prepared by the prior art amino acid-based cationic lipids (except L-3, L-14, L-29 and L-36 groups). Among them, the relative fluorescence values of L-1, L-6, L-7, L-20 and L-23 in the experimental group were high, which might be because the compound contained two ionizable tertiary amine structures and multiple degradable ester bonds, the ionizable tertiary amine ionized part of the positive charge combined with negatively charged nucleic acids, the degradable ester bonds promoted the endosome escape of LNP-mRNA, and the mRNA was released into the cytoplasm to play a curative effect. Compared with L-2 and L-4, the relative fluorescence values of L-2 and L-4 were similar, but both were higher than L-0 in the control group with cationic lipids containing ether bonds. The relative fluorescence values of L-3 and L-29 in the experimental group were low, and the number of hydrophobic tails of the amino acid-based cationic lipids used was more, and the encapsulation rate would not be very low, which may be caused by the hindered endosomal escape of LNP-mRNA.

TABLE 2
Results of cell transfection test

| Number | Fluorescence relative value |
|---|---|
| Control | 1 |
| L-0 | 3.0 |
| L-1 | 10.5 |
| L-2 | 8.7 |
| L-3 | 3.6 |
| L-4 | 9.2 |
| L-5 | 4.3 |
| L-6 | 12.3 |
| L-7 | 11.6 |
| L-8 | 5.1 |
| L-9 | 6.7 |
| L-10 | 7.2 |
| L-11 | 8.5 |
| L-12 | 9.7 |
| L-13 | 8.1 |
| L-14 | 2.8 |
| L-15 | 8.3 |
| L-16 | 9.1 |
| L-17 | 7.4 |
| L-18 | 9.1 |
| L-19 | 8.9 |
| L-20 | 10.7 |
| L-21 | 6.2 |
| L-22 | 8.3 |
| L-23 | 10.2 |
| L-24 | 9.6 |
| L-25 | 9.3 |
| L-26 | 8.9 |
| L-27 | 6.7 |
| L-28 | 5.2 |
| L-29 | 3.2 |
| L-30 | 8.7 |
| L-31 | 9.1 |
| L-32 | 8.4 |
| L-33 | 9.6 |
| L-34 | 8.7 |
| L-35 | 8.0 |
| L-36 | 3.5 |
| L-37 | 11.8 |
| L-38 | 11.6 |

Those described above are only embodiments of the present application, and are not for the purpose of limitation. Any modification of equivalent structures or equivalent routes according to the present application, which may be applied in other related art in a direct or an indirect way, should be included in the scope of the present application.

For those skilled in the art, without departing from the spirit and scope of the present application, and without unnecessary experimentation, the present application can be implemented in a wider range under equivalent parameters, concentrations, and conditions. While the present application has given particular examples, it should be understood that the present application can be further modified. In conclusion, in accordance with the principles of the present application, the present application is intended to cover any alterations, uses, or improvements of the present application, including changes departing from the scope disclosed in this application but made using conventional techniques known in the art.

What is claimed is:

1. An amino acid-based cationic lipid, wherein the structure is represented by the general formula (1): 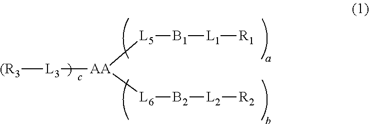
wherein, AA is a residue of an amino acid or amino acid derivative;
B₁ and B₂ are each independently a linking bond or a C₁₋₃₀ alkylene group at each occurrence;
L₁ and L₂ are each independently a linking bond or a divalent linking group at each occurrence;
L₅ and L₆ are each independently a linking bond or a divalent linking group at each occurrence;
L₃ is independently a linking bond or a divalent linking group at each occurrence;
R₁ and R₂ are each independently —(CH₂)ₜNRₑR_{f}, a linear C₁₋₃₀ alkylene group, a branched C₁₋₃₀ alkylene group or 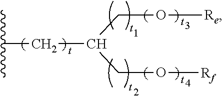
at each occurrence; wherein, t is an integer from 0 to 12; t₁ and t₂ are each independently an integer from 0 to 5; t₃ and t₄ are each independently 0 or 1; t₁, t₂, t₃, and t₄ are not 0 simultaneously; Rₑ and R_{f} are each independently a C₁₋₁₅ alkyl group;
R₃ is, at each occurrence, independently a hydrogen atom, -R_{d}, —OR_{d}, a C₃₋₆ carbocyclic group, a nitrogen-containing heterocyclic group, —NR_{d}R_{d}, —SR_{d}, —C(═O)R_{d}, —C(═O)OR_{d}, —OC(═O)R_{d}, —OC(═O)OR_{d} or a functional group R₀₁ that can interact with bio-related substances; wherein, R_{d} is independently a C₁₋₁₂ alkyl group at each occurrence;
a, b, and c are each independently 1 or 2; when fragments -L₅-B₁-L₁-R₁ and/or -L₆-B₂-L₂-R₂ and/or -L₃-R₃ are protruded from the amino terminus of the amino acid, a, b, and c are each independently 1 or 2; when fragments -L₅-B₁-L₁-R₁ and/or -L₆-B₂-L₂-R₂ and/or -L₃-R₃ are protruded from the carboxyl, hydroxyl, or thiol terminus of the amino acid, a, b, and c are each independently 1; when a is 2, two -L₅-B₁-L₁-R₁ fragments are the same or different; when b is 2, two -L₆-B₂-L₂-R₂ fragments are the same or different; when c is 2, two -L₃-R₃ fragments are the same or different;
or a salt, tautomer, stereoisomer, or solvate thereof.

2. The amino acid-based cationic lipid according to claim 1, wherein the amino acid and amino acid derivative is selected from the group consisting of arginine, aspartic acid, asparagine, cysteine, glutamic acid, glutamine, histidine, lysine, methionine, phenylalanine, serine, threonine, tryptophan, tyrosine and an amino acid derivative of any of the aforementioned amino acids.

3. The amino acid-based cationic lipid according to claim 1, wherein the AA is selected from the group consisting of the following structures: 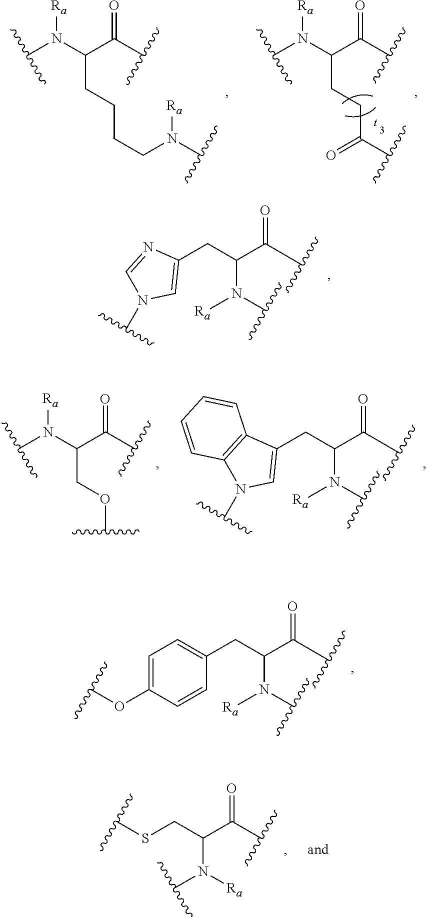 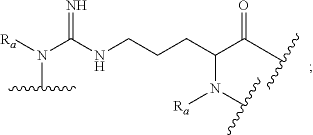
wherein, the t₃ of AA is 0 or 1; Rₐ is independently selected from the group consisting of a linking bond, H, a methyl group, an ethyl group, a propyl group, and an isopropyl group at each occurrence; or AA is the case where one or two carbonyl groups in any of the aforementioned structures are independently capped by an oxygen atom or a secondary amine group, including: 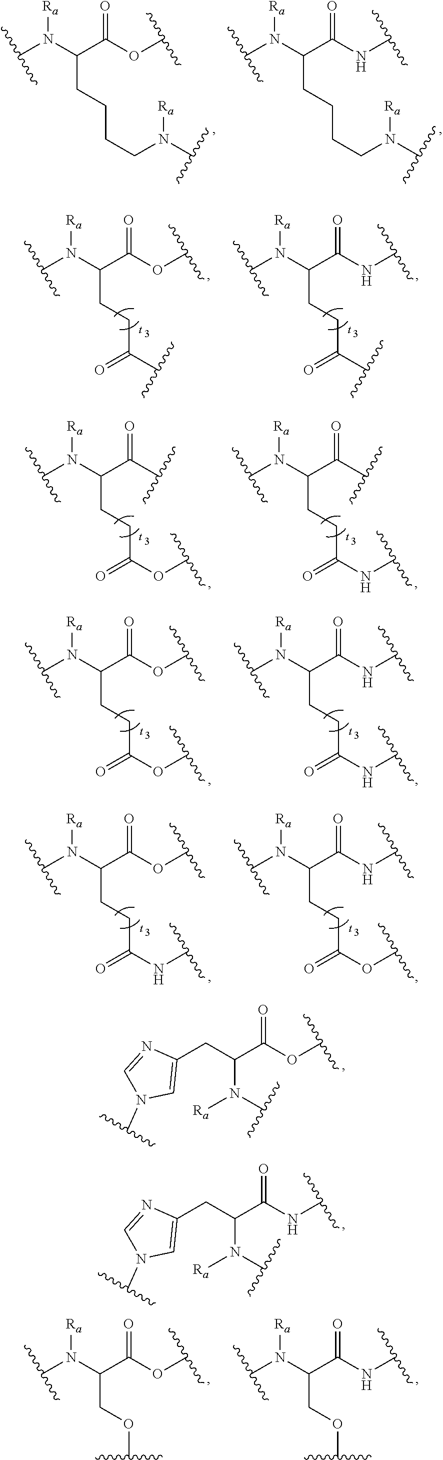 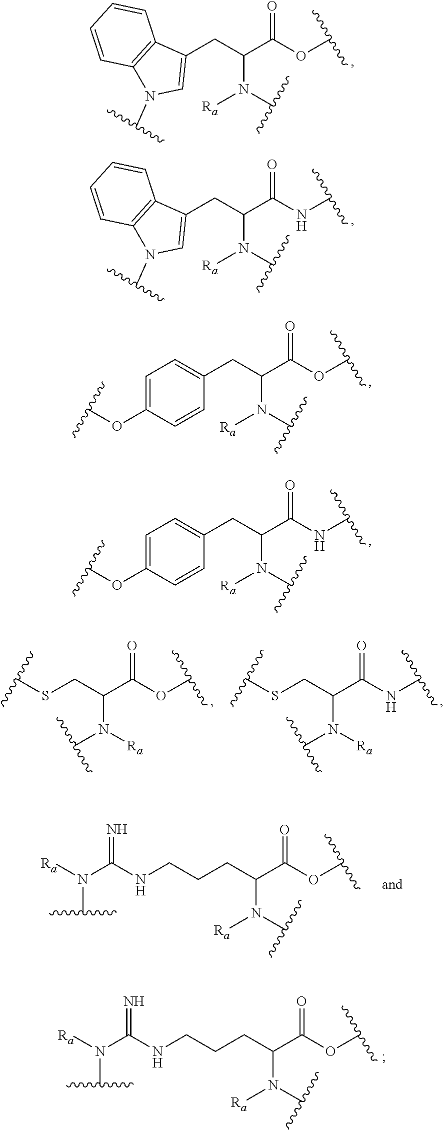
more specifically, AA is selected from the group consisting of the following structures:

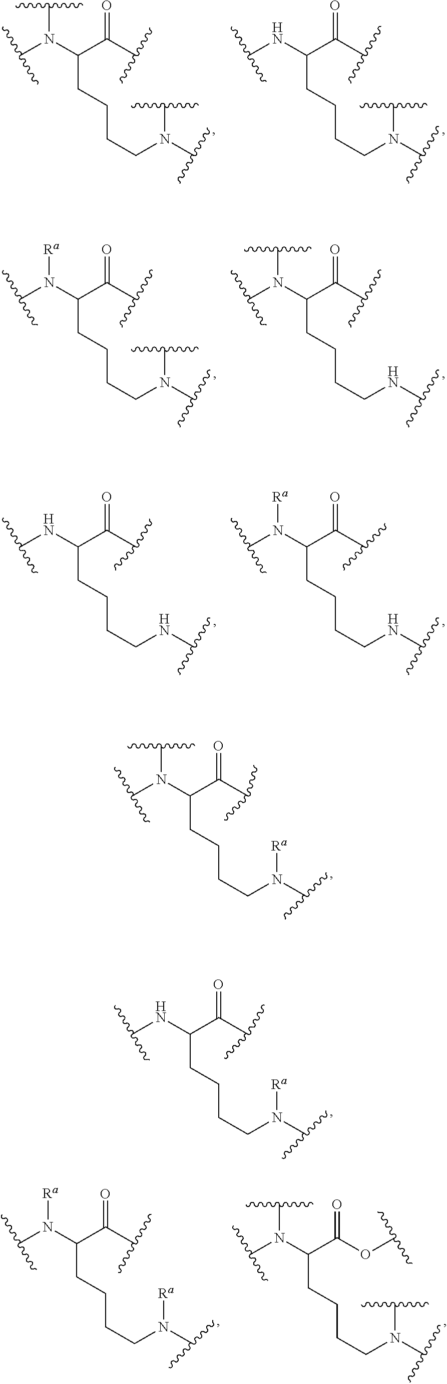

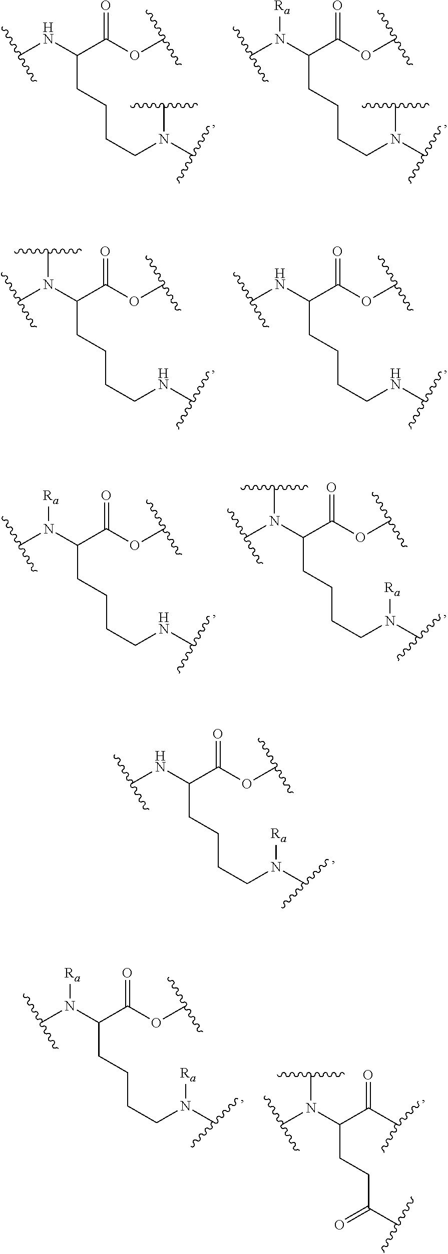

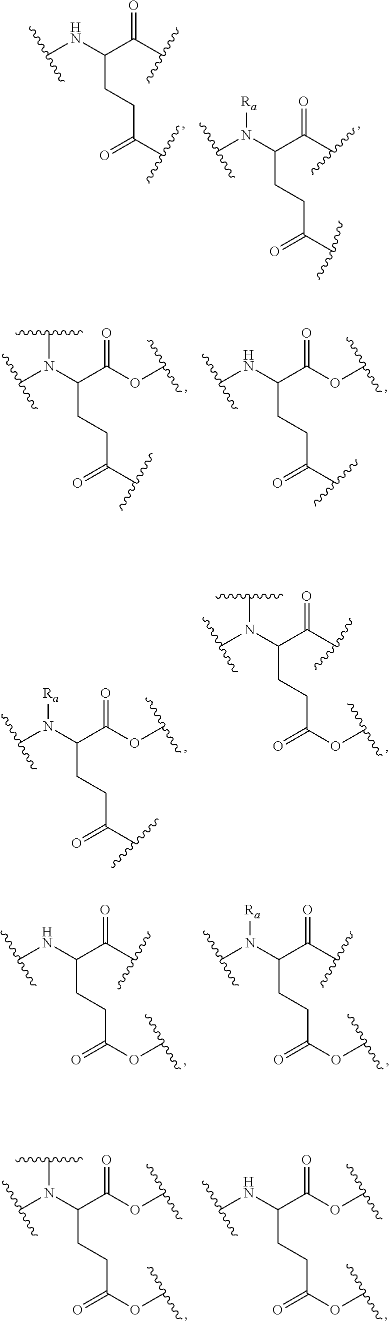

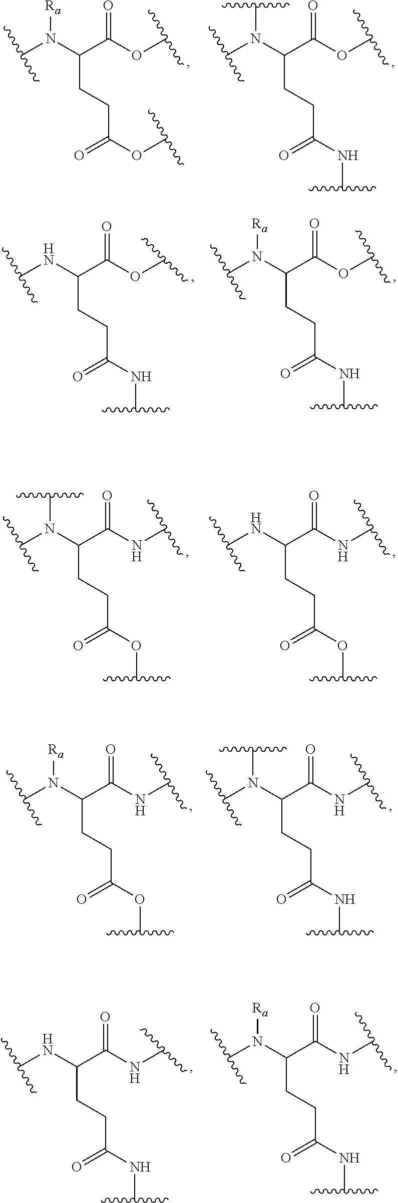

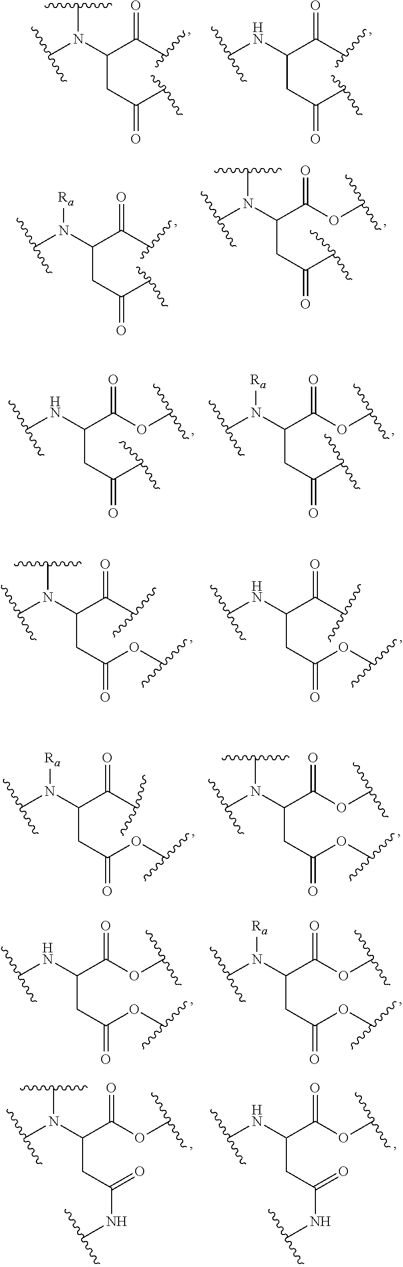

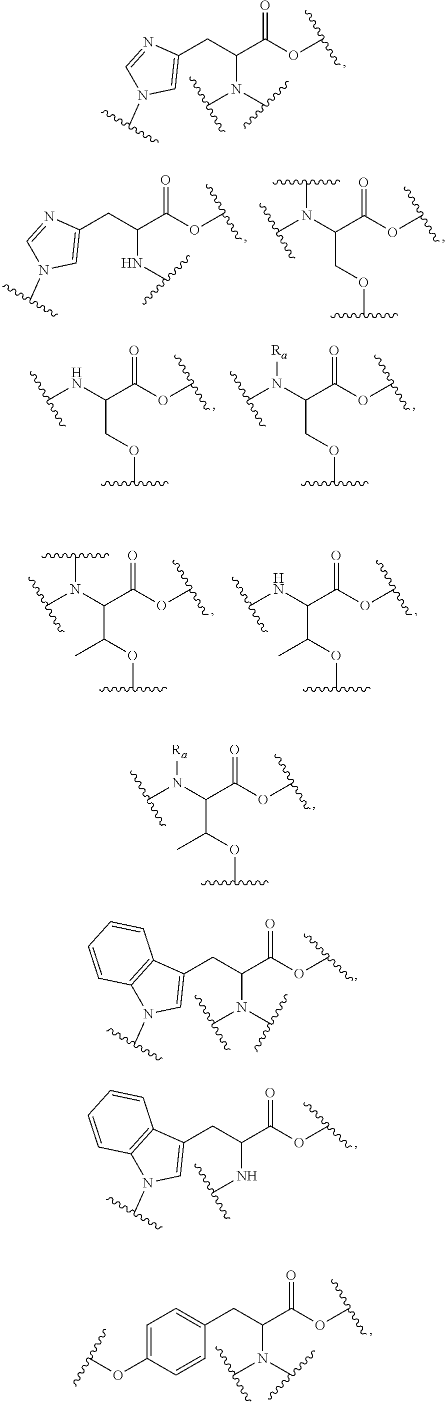

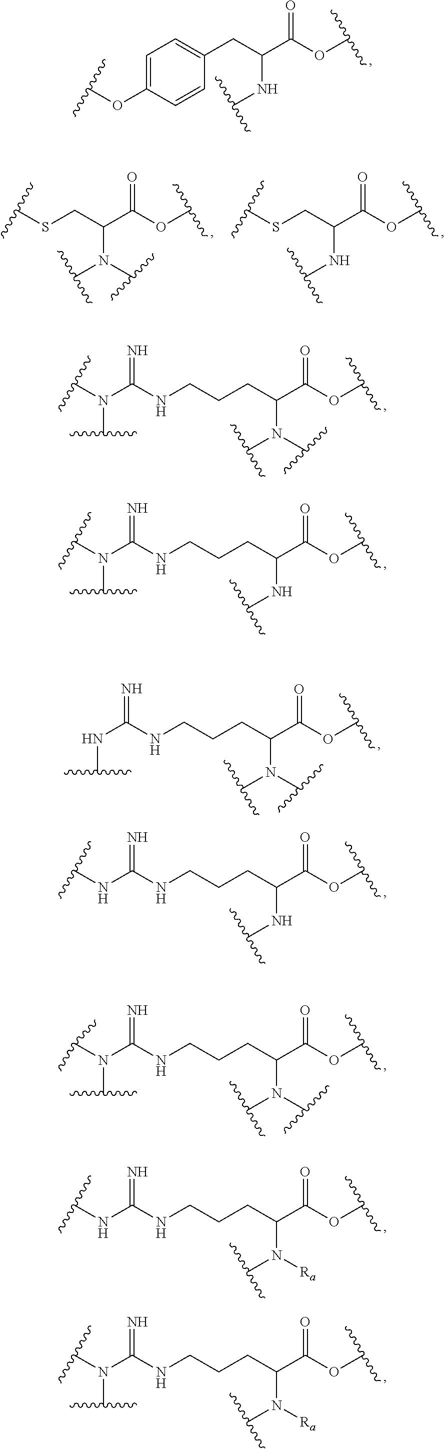

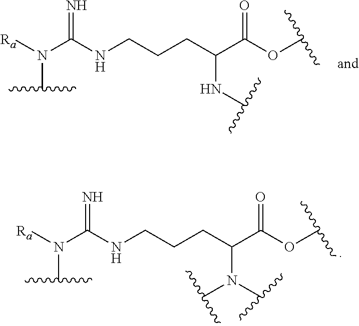

4. The amino acid-based cationic lipid according to claim 1, wherein the B₁ and B₂ are each independently a linking bond or a C₁₋₂₀ alkylene group, specifically selected from any one of the following cases:
Case (1): B₁ and B₂ are each independently a C₁₋₂₀ alkylene group, and specifically B₁ and B₂ are each independently a C₂₋₁₀ alkylene group;
Case (2): one of B₁ and B₂ is a linking bond, and the other is a C₁₋₂₀ alkylene group;
Case (3): both B₁ and B₂ are linking bonds.

5. The amino acid-based cationic lipid according to claim 1, wherein the L₁ and L₂ are each independently selected from the group consisting of a linking bond, —OC(═O)—, —C(═O)O—, —OC(═O)O—, —C(═O)—, —O—, —NH—, —O(CR_{c}R_{c})ₛO—, —S—, —C(═O)S—, —SC(═O)—, —NR_{c}C(═O)—, —C(═O)NR_{c}—, —NR_{c}C(═O)NRₑ—, —OC(═O)NRₑ—, —NR_{c}C(═O)O—, —SC(═O)NRₑ- and —NR_{c}C(═O)S—; wherein, Rₑ is a hydrogen atom or a C₁₋₂₀ alkylene group at each occurrence, and s is 1, 2, 3 or 4;
more specifically, L₁ and L₂ are selected from any one of the following cases:
Case (1): L₁ and L₂ are each independently selected from the group consisting of —OC(═O)—, —C(═O)O—, —OC(═O)O—, —C(═O)—, —O—, —O(CH₂)ₛO—, —S—, —C(═O)S—, —SC(═O)—, —NHC(═O)—, —C(═O)NH—, —NHC(═O)NH—, —OC(═O)NH—, —NHC(═O)O—, —SC(═O)NH- and —NHC(═O)S—;
Case (2): one of L₁ and L₂ is a linking bond, and the other is selected from the group consisting of —OC(═O)—, —C(═O)O—, —OC(═O)O—, —C(═O)—, —O—, —O(CH₂)ₛO—, -S-, —C(═O)S—, —SC(═O)—, —NHC(═O)—, —C(═O)NH—, —NHC(═O)NH—, —OC(═O)NH—, —NHC(═O)O—, —SC(═O)NH- and —NHC(═O)S—;
Case (3): both L₁ and L₂ are linking bonds.

6. The amino acid-based cationic lipid according to claim 1, wherein the R₁ and R₂ are each independently a linear alkyl group, a branched alkyl group or 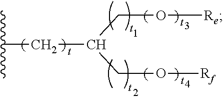
the linear alkyl group is a C₁₋₂₅ linear alkyl group, and specifically is selected from the group consisting of a pentyl group, a hexyl group, a heptyl group, an octyl group, a nonyl group, a decyl group, an undecyl group, a dodecyl group, a tridecyl group, a tetradecyl group, a pentadecyl group, a hexadecyl group, and a heptadecyl group;
the branched alkyl group is represented as 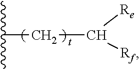
wherein Rₑ and R_{f} are each independently a C₁₋₁₅ alkyl group; wherein, t is an integer from 0 to 12; specifically, the branched alkyl group is selected from the group consisting of the following structures: 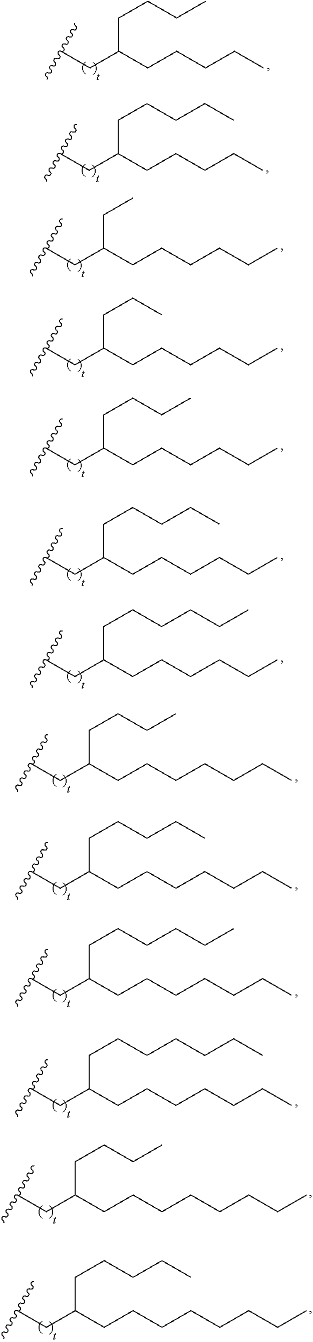 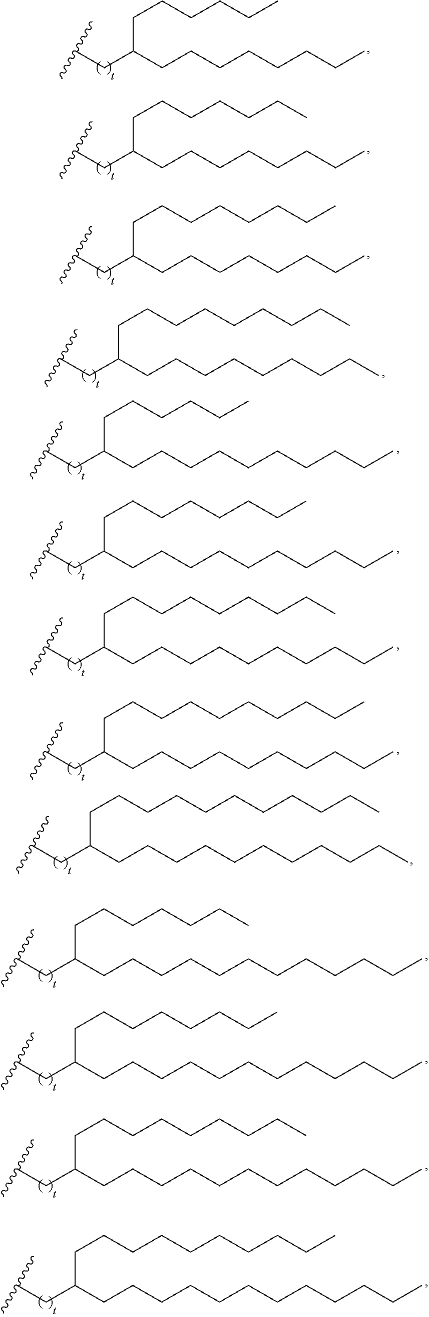 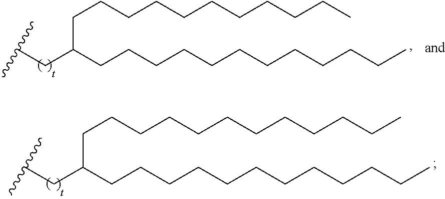 [SMILES: *CC(CCCCCCCCCCC)CCCCCCCCCCCC ; *CC(CCCCCCCCCCCC)CCCCCCCCCCCC]
the 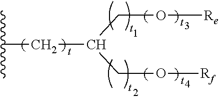
is selected from the group consisting of the following structures:

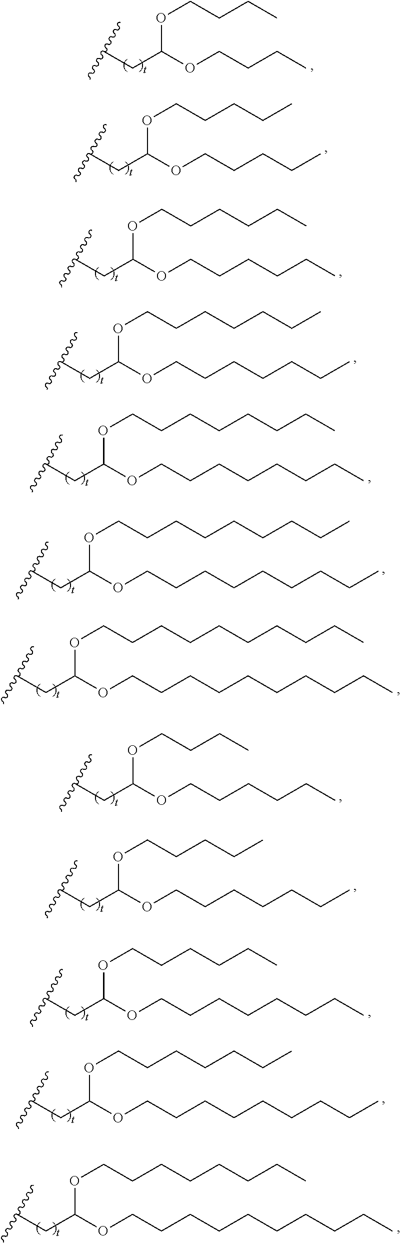

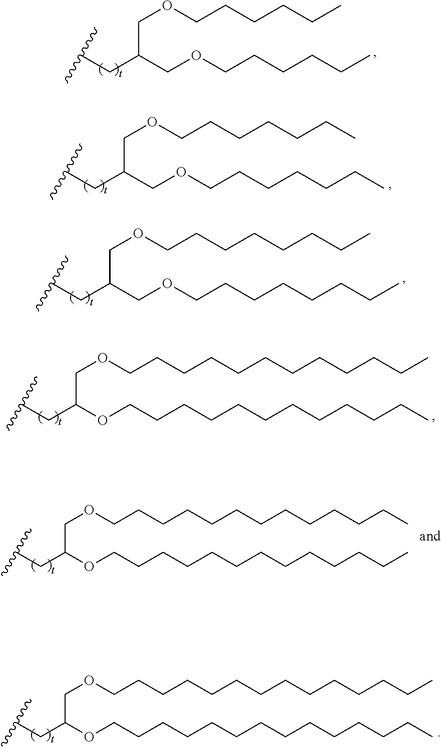

7. The amino acid-based cationic lipid according to claim 1, wherein the R₃ is selected from the group consisting of a hydrogen atom, an alkyl group, an alkoxy group, —C(═O)OR_{d}, —OC(═O)R_{d}, —OC(═O)OR_{d}, an epoxy group, a hydroxyl group, a protected hydroxyl group, a sulfhydryl group, a protected sulfhydryl group, a carboxyl group, a protected carboxyl group, an amino group, a protected amine group, an aldehyde group, a protected aldehyde group, an active ester group, a carbonate group, a carbamate group, an isocyanate group, a polysaccharide group,

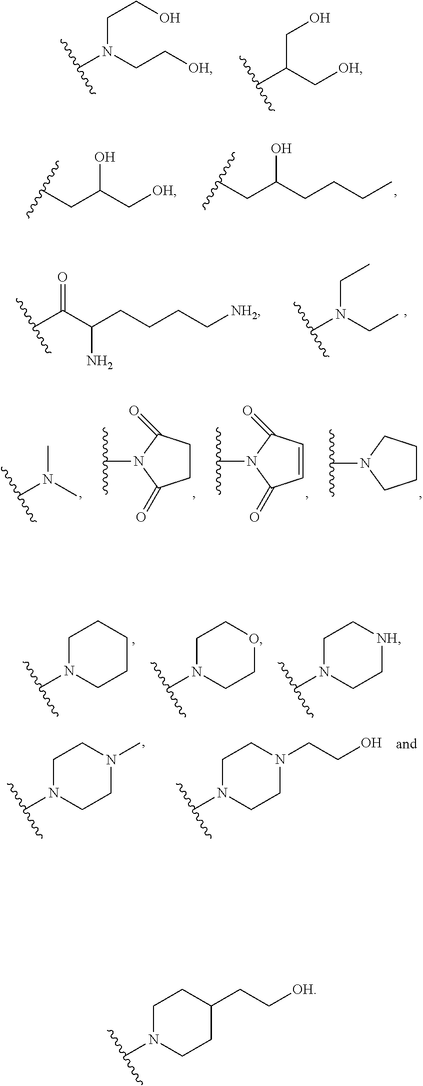

8. The amino acid-based cationic lipid according to claim 1, wherein the L₃ is a divalent linking group, selected from the group consisting of -L₇-, -L₇-Z—, -Z-L₇-, -Z-L₇-Z—, -L₇-Z-L₈-, -Z-L₇-Z-L₈-, -L₇-Z-L₈-Z—, -Z-L₇-Z-L₈-Z- and -L₇-Z-L₈-Z-L₇-Z-; wherein, the L₇ and L₈ are carbon chain linking groups, each independently represented by -(CRₐR_{b})ₜ-(CRₐR_{b})ₒ-(CRₐR_{b})ₚ-, wherein t, o, and p are each independently an integer of 0 to 12, not being 0 simultaneously; Rₐ and R_{b}are each independently a hydrogen atom or a C₁₋₁₂ alkyl group at each occurrence; the Z is independently selected from the group consisting of —C(═O)—, —NH—, —OC(═O)—, —C(═O)O—, —OC(═O)O—, —O—, -S-, —C(═O)S—, —SC(═O)—, —NR_{c}C(═O)—, —C(═O)NR_{c}—, —NR_{c}C(═O)NRₑ—, —OC(═O)NRₑ—, —NR_{c}C(═O)O—, —SC(═O)NRₑ- and —NR_{c}C(═O)S—; wherein, Rₑ is independently H or a C₁₋₁₂ alkyl group at each occurrence.

9. The amino acid-based cationic lipid according to claim 8, wherein the L₃ is selected from the group consisting of —(CH₂)ₜ—, —(CH₂)ₜZ—, —Z(CH₂)ₜ—, —(CH₂)ₜZ(CH₂)ₜ—, —Z(CH₂)ₜZ—, —(CH₂)ₜZ(CH₂)ₜZ—, —Z(CH₂)ₜZ(CH₂)ₜ- and —Z(CH₂)ₜZ(CH₂)ₜZ—; wherein, t is an integer of 1 to 12; specifically, L₃ is selected from the group consisting of —(CH₂)ₜ—, —(CH₂)ₜO—, —(CH₂)ₜC(═O)—, —(CH₂)ₜNH-, —(CH₂)ₜC(═O)O—, —(CH₂)ₜOC(═O)—, —(CH₂)ₜC(═O)NH—, —(CH₂)ₜNHC(═O)—, —(CH₂)ₜOC(═O)O—, —(CH₂)ₜNHC(═O)O—, —(CH₂)ₜOC(═O)NH—, —(CH₂)ₜNHC(═O)NH—, —O(CH₂)ₜ—, —C(═O)(CH₂)ₜ—, —C(═O)O(CH₂)ₜ—, —OC(═O)(CH₂)ₜ, —C(═O)NH(CH₂)ₜ—, —NHC(═O)(CH₂)ₜ—, —OC(═O)O(CH₂)ₜ, —NHC(═O)O(CH₂)ₜ—, —OC(═O)NH(CH₂)ₜ, —NHC(═O)NH(CH₂)ₜ—, —(CH₂)ₜO(CH₂)ₜ—, —(CH₂)ₜC(═O)(CH₂)ₜ, —(CH₂)ₜC(═O)O(CH₂)ₜ, —(CH₂)ₜOC(═O)(CH₂)ₜ, —(CH₂)ₜC(═O)NH(CH₂)ₜ, —O(CH₂)ₜO—, —(CH₂)ₜNHC(═O)(CH₂)ₜ, —(CH₂)ₜOC(═O)O(CH₂)ₜ, —(CH₂)ₜNHC(═O)O(CH₂)ₜ, —(CH₂)ₜOC(═O)NH(CH₂)ₜ, —(CH₂)ₜNHC(═O)NH(CH₂)ₜ—, —C(═O)(CH₂)ₜC(═O)—, —C(═O)O(CH₂)ₜC(═O)O—, —OC(═O)(CH₂)ₜOC(═O)—, —C(═O)O(CH₂)ₜOC(═O)—, —OC(═O)(CH₂)ₜC(═O)O—, —OC(═O)O(CH₂)ₜOC(═O)O—, —C(═O)NH(CH₂)ₜC(═O)NH—, —NHC(═O)(CH₂)ₜNHC(═O)—, —NHC(═O)(CH₂)ₜC(═O)NH—, —C(═O)NH(CH₂)ₜNHC(═O)—, —NHC(═O)O(CH₂)ₜNHC(═O)O—, —OC(═O)NH(CH₂)ₜOC(═O)NH—, —C(═O)(CH₂)ₜO—, —NHC(═O)O(CH₂)ₜOC(═O)NH—, —OC(═O)NH(CH₂)ₜNHC(═O)O—, —C(═O)(CH₂)ₜC(═O)O—, —NHC(═O)NH(CH₂)ₜNHC(═O)NH—, —C(═O)(CH₂)ₜOC(═O)—, —C(═O)(CH₂)ₜOC(═O)O—, —C(═O)(CH₂)ₜNHC(═O)O—, —C(═O)(CH₂)ₜOC(═O)NH—, —C(═O)(CH₂)ₜNHC(═O)NH—, —C(═O)(CH₂)ₜC(═O)O(CH₂)ₜ, —C(═O)(CH₂)ₜOC(═O)(CH₂), —C(═O)(CH₂)ₜOC(═O)O(CH₂)ₜ, —C(═O)(CH₂)ₜNHC(═O)O(CH₂), —C(═O)(CH₂)ₜOC(═O)NH(CH₂)ₜ, —C(═O)(CH₂)ₜNHC(═O)NH(CH₂)ₜ- and —C(═O)(CH₂)ₜC(═O)(CH₂)ₜNHC(═O)O—; more specifically, L₃ is selected from the group consisting of —(CH₂)ₜ—, —(CH₂)ₜNH-, —(CH₂)ₜO—, —(CH₂)ₜC(═O)O—, —(CH₂)ₜOC(═O)- and —O(CH₂)ₜ-.

10. The amino acid-based cationic lipid according to claim 9, wherein the -L₃-R₃ is selected from the group consisting of the following structures:

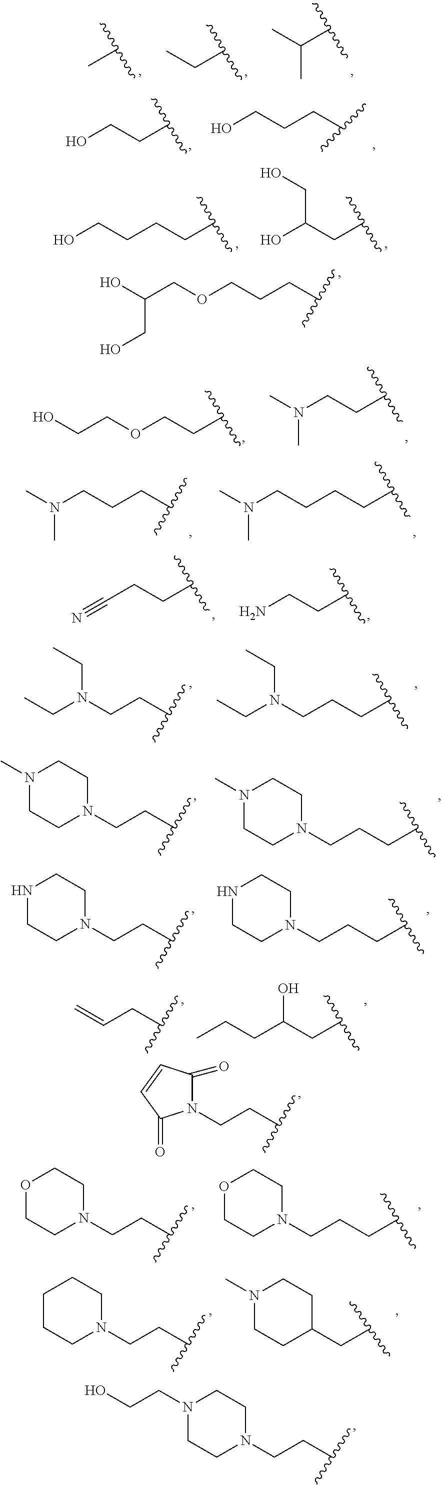

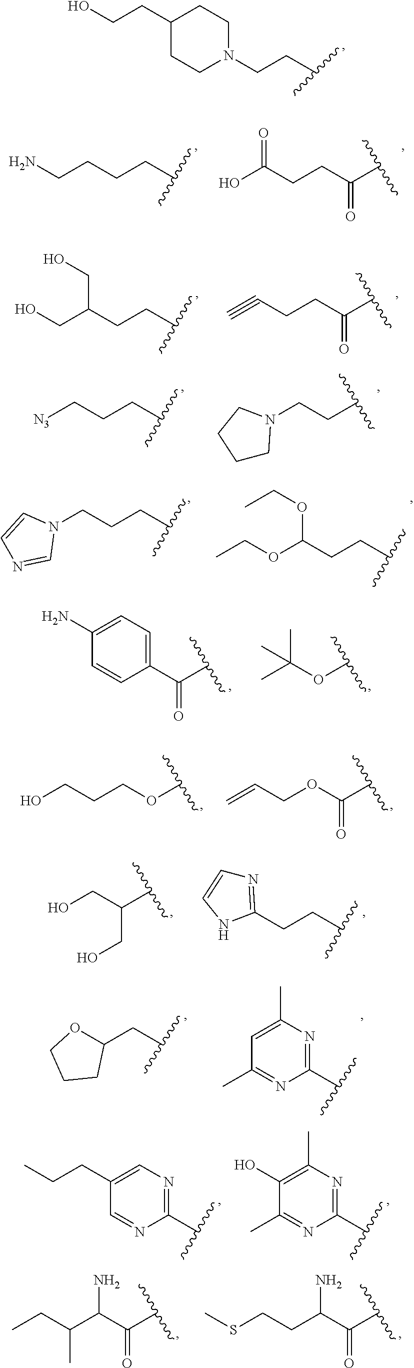

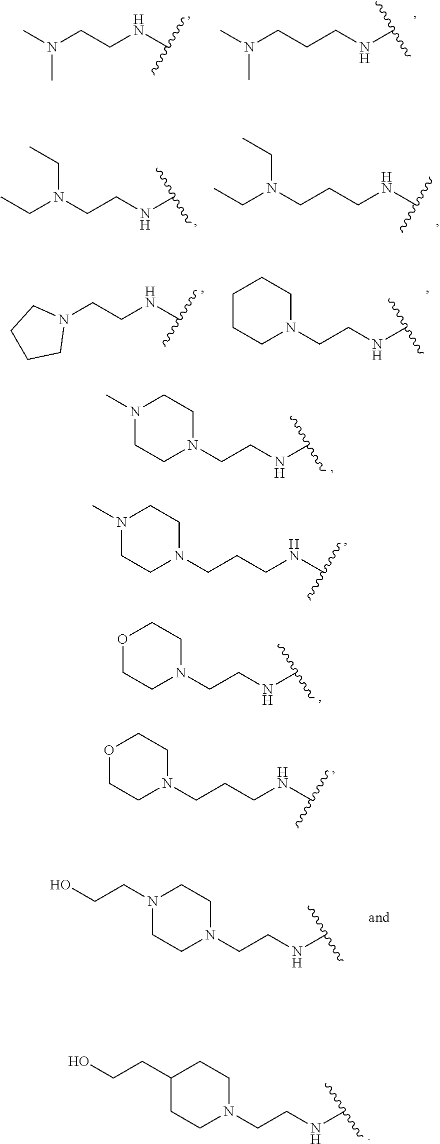

11. The amino acid-based cationic lipid according to claim 1, wherein the Lₛ and L₆ are each independently selected from the group consisting of a linking bond, —O—, —NH—, —C(═O)—, —OC(═O)— and —C(═O)O—; specifically, both L₅ and L₆ are simultaneously linking bonds, simultaneously -O—, simultaneously -NH-, or simultaneously -C(═O)O-.

12. The amino acid-based cationic lipid according to claim 11, wherein the -L₅-B₁-L₁-R₁ and -L₆-B₂-L₂-R₂ are each independently selected from the group consisting of the following structures at each occurrence:

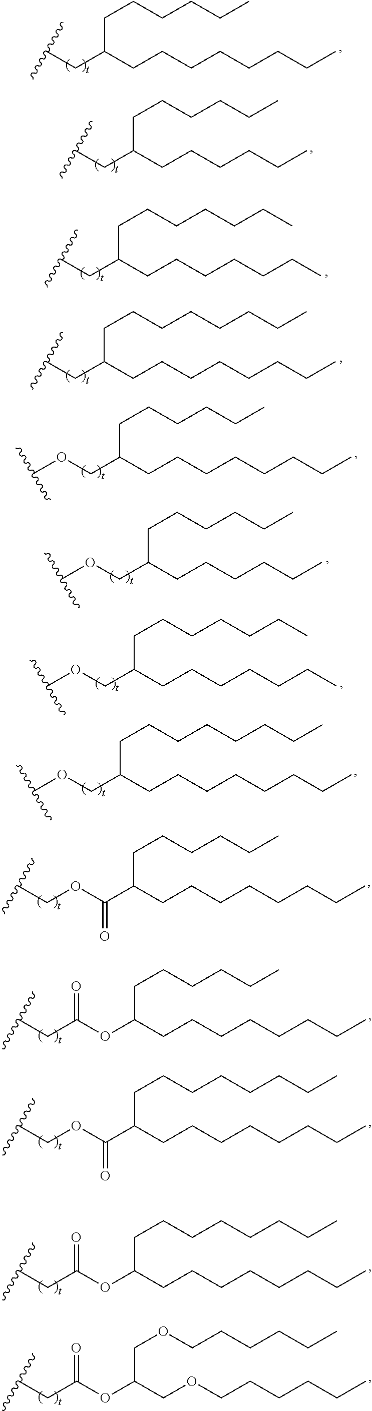

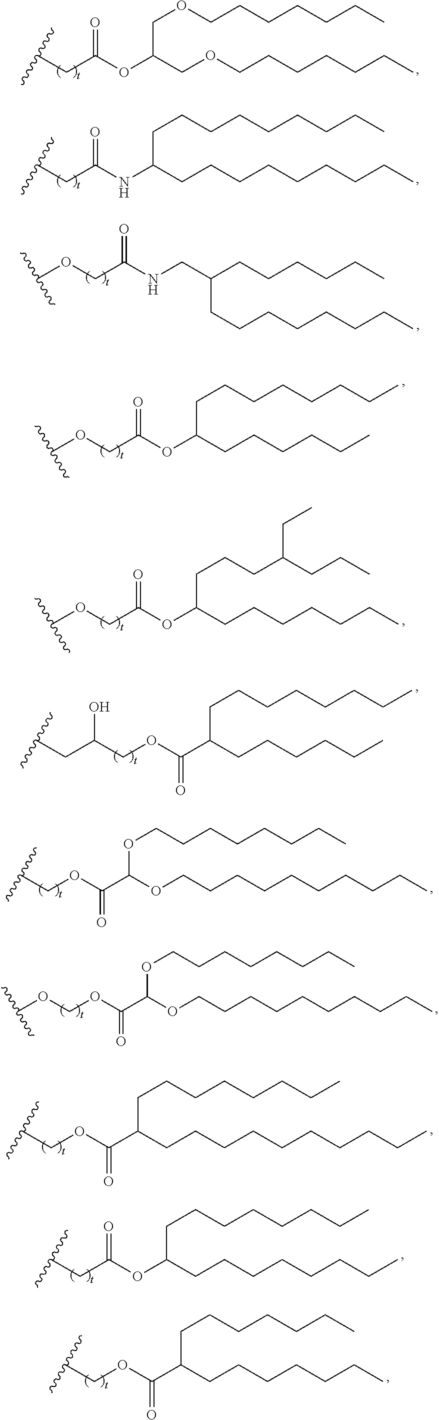

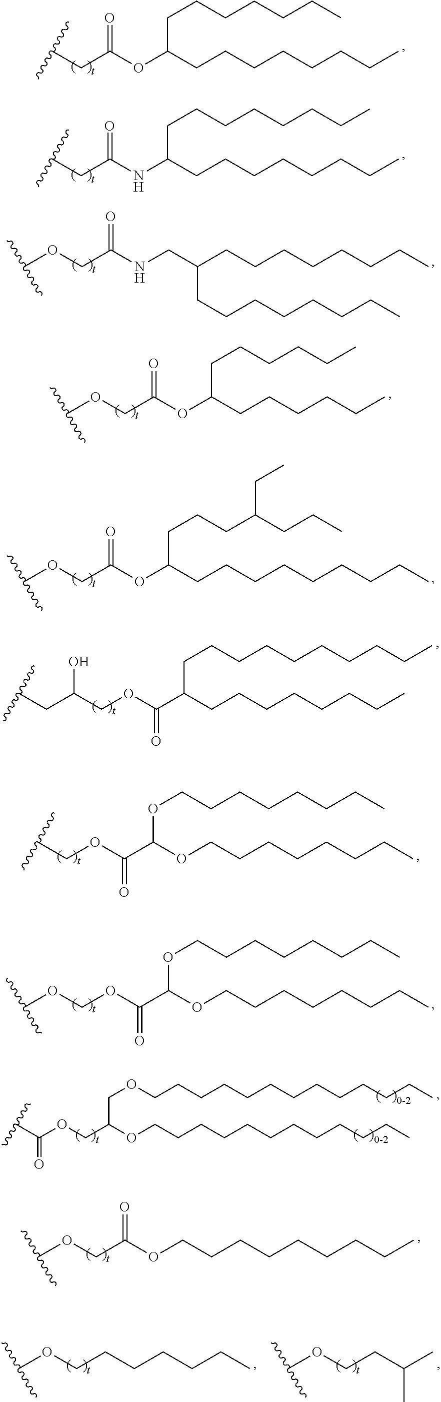

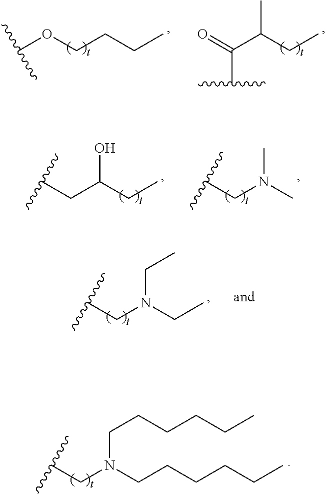

13. The amino acid-based cationic lipid according to claim 1, wherein the R₀₁ is a functional group with therapeutic targeting properties; specifically, R₀₁ is a residue of folic acid, N-acetylgalactosamine, or a functional derivative thereof;
more specifically, R₀₁ is selected from the group consisting of the following structures:

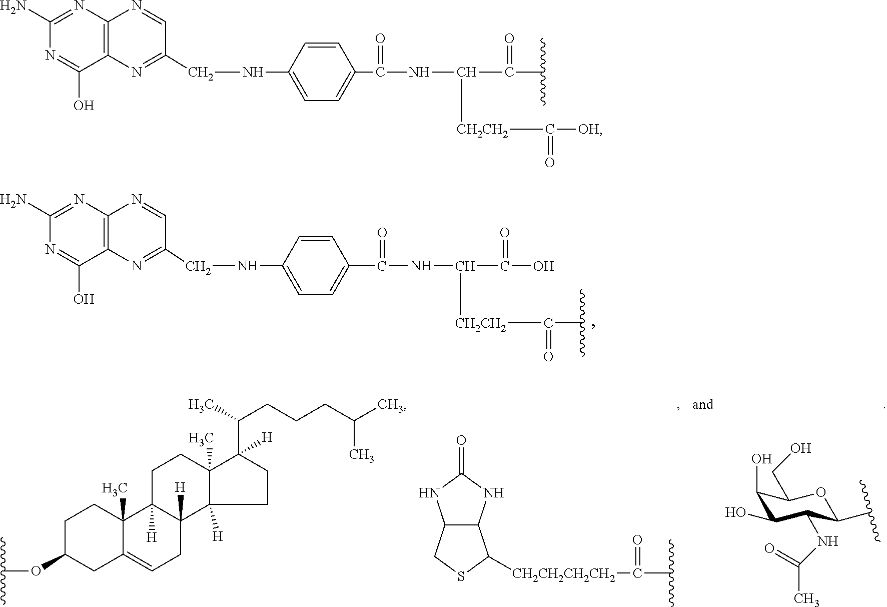

14. The amino acid-based cationic lipid according to claim 1, wherein the structure of the amino acid-based cationic lipid is selected from the group consisting of the following general formulas: 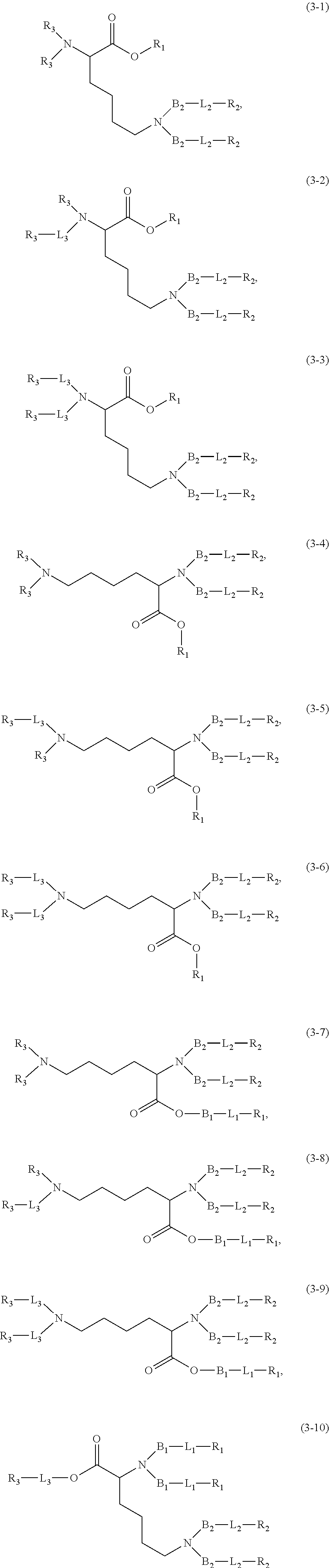 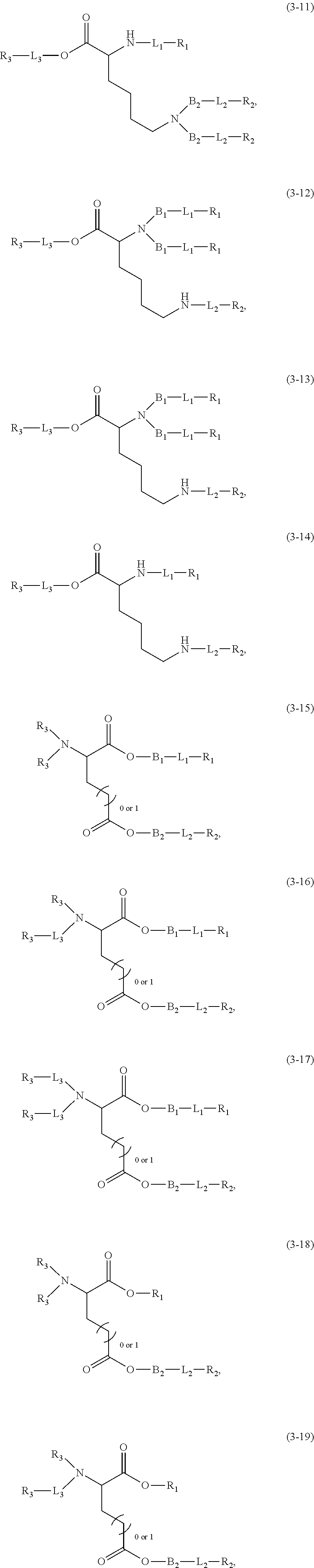 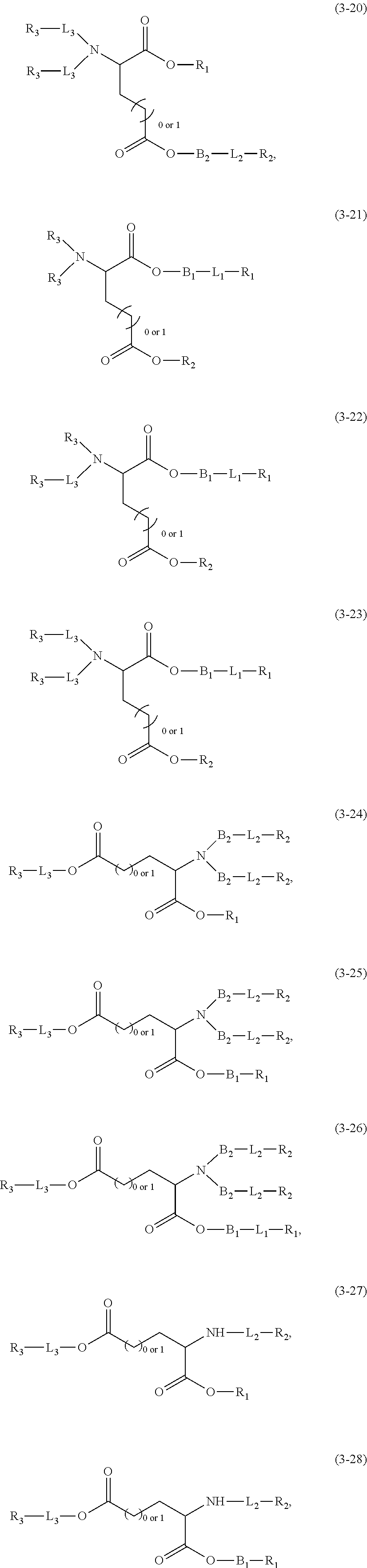 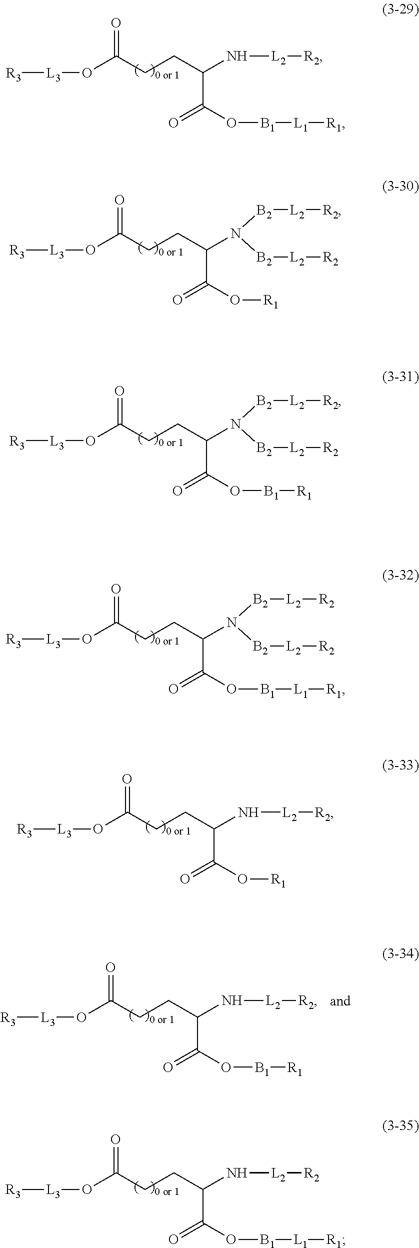
wherein, none of B₁, B₂, L₁, L₂, L₅, L₃ is a linking bond; specifically, L₁ and L₂ are each independently selected from the group consisting of —C(═O)—, —NH—, —C(═O)O—, —OC(═O)—, —OC(═O)O—, —C(═O)NH—, —NHC(═O)—, —OC(═O)NH—, and —NHC(═O)O— at each occurrence.

15. The amino acid-based cationic lipid according to claim 1, wherein the structure of the amino acid-based cationic lipid is any one of the following structures:

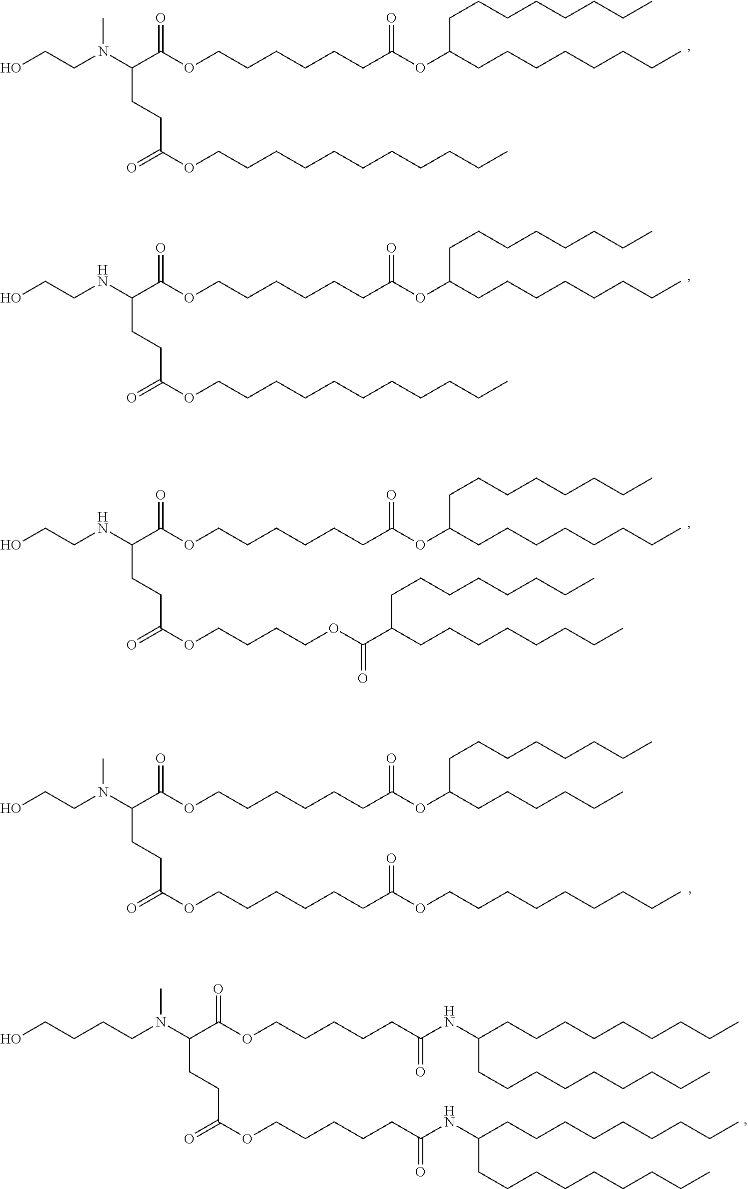 [SMILES: CCCCCCCCCCCOC(=O)CCC(C(=O)OCCCCCCC(=O)OC(CCCCCCCC)CCCCCCCC)N(C)CCO ; CCCCCCCCCCCOC(=O)CCC(NCCO)C(=O)OCCCCCCC(=O)OC(CCCCCCCC)CCCCCCCC ; CCCCCCCCC(CCCCCCCC)OC(=O)CCCCCCOC(=O)C(CCC(=O)OCCCCOC(=O)C(CCCCCCCC)CCCCCCCC)NCCO ; CCCCCCCCCOC(=O)CCCCCCOC(=O)CCC(C(=O)OCCCCCCC(=O)OC(CCCCCC)CCCCCCCC)N(C)CCO ; CCCCCCCCCC(CCCCCCCCC)NC(=O)CCCCCOC(=O)CCC(C(=O)OCCCCCC(=O)NC(CCCCCCCCC)CCCCCCCCC)N(C)CCCCO]

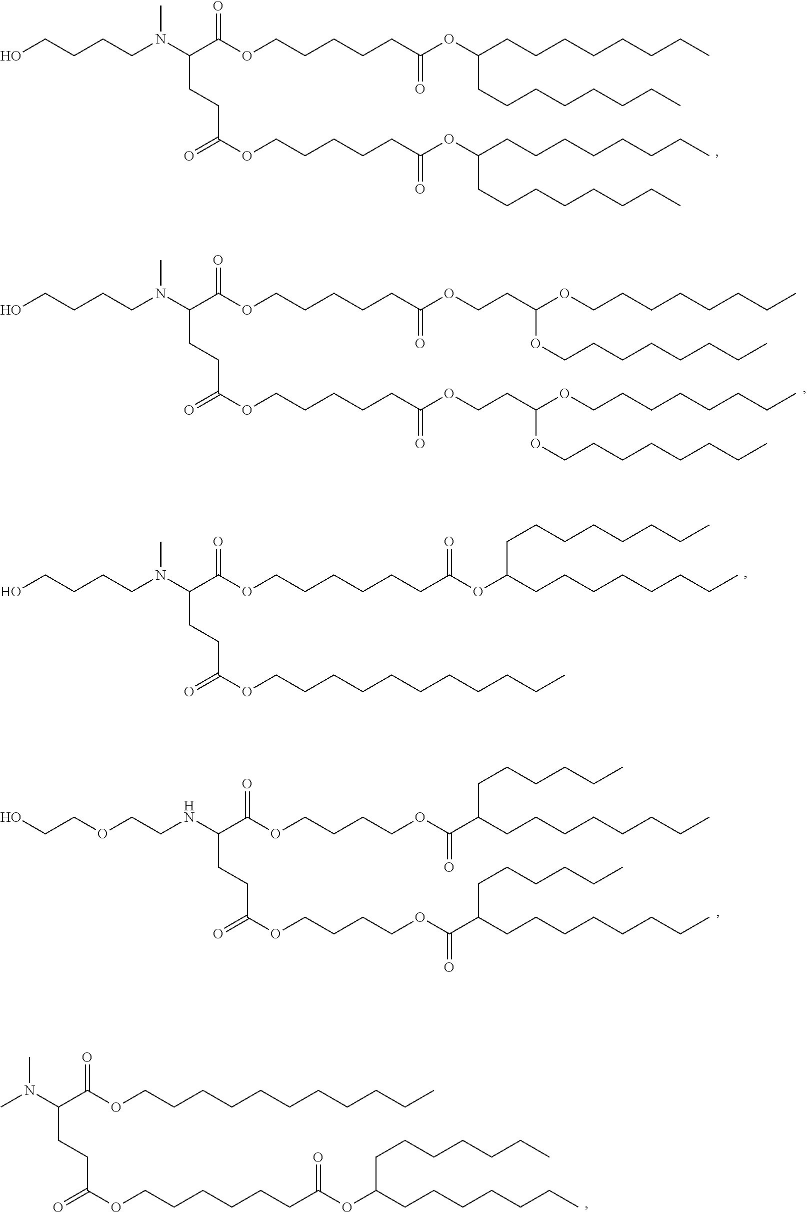

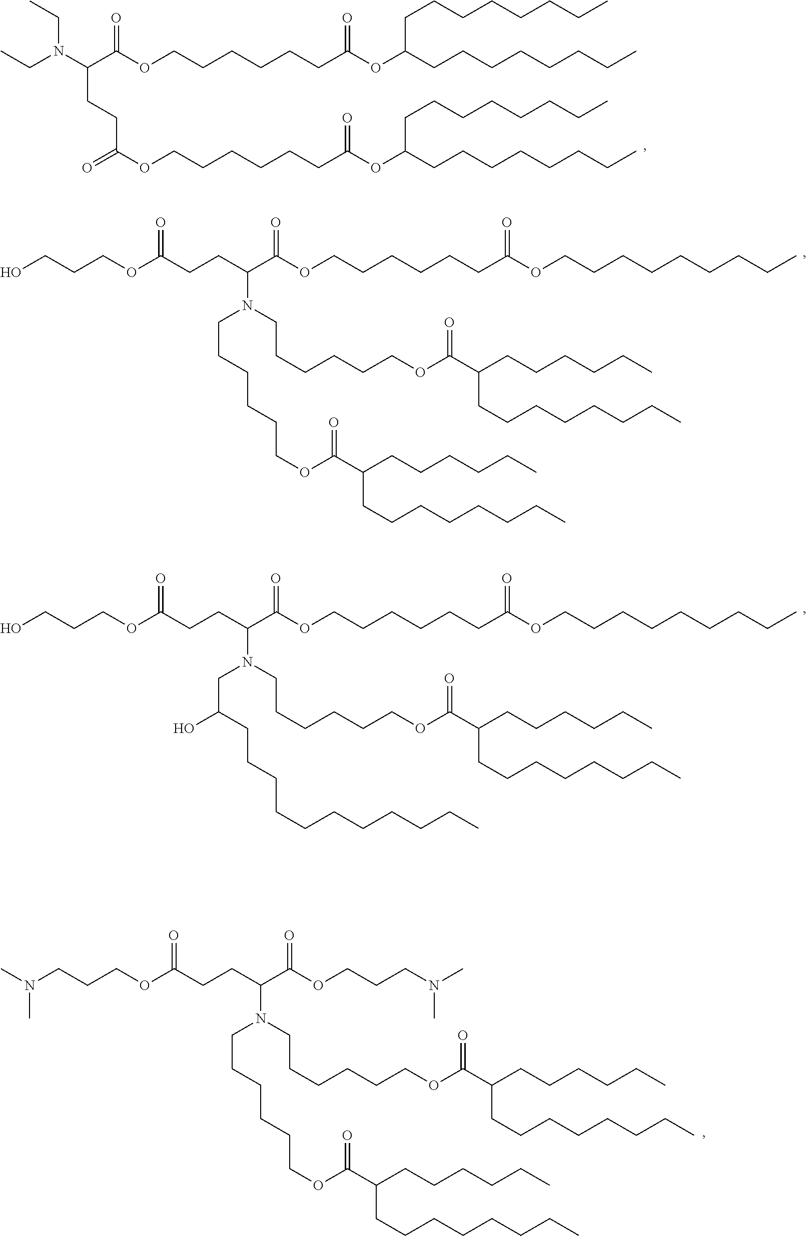

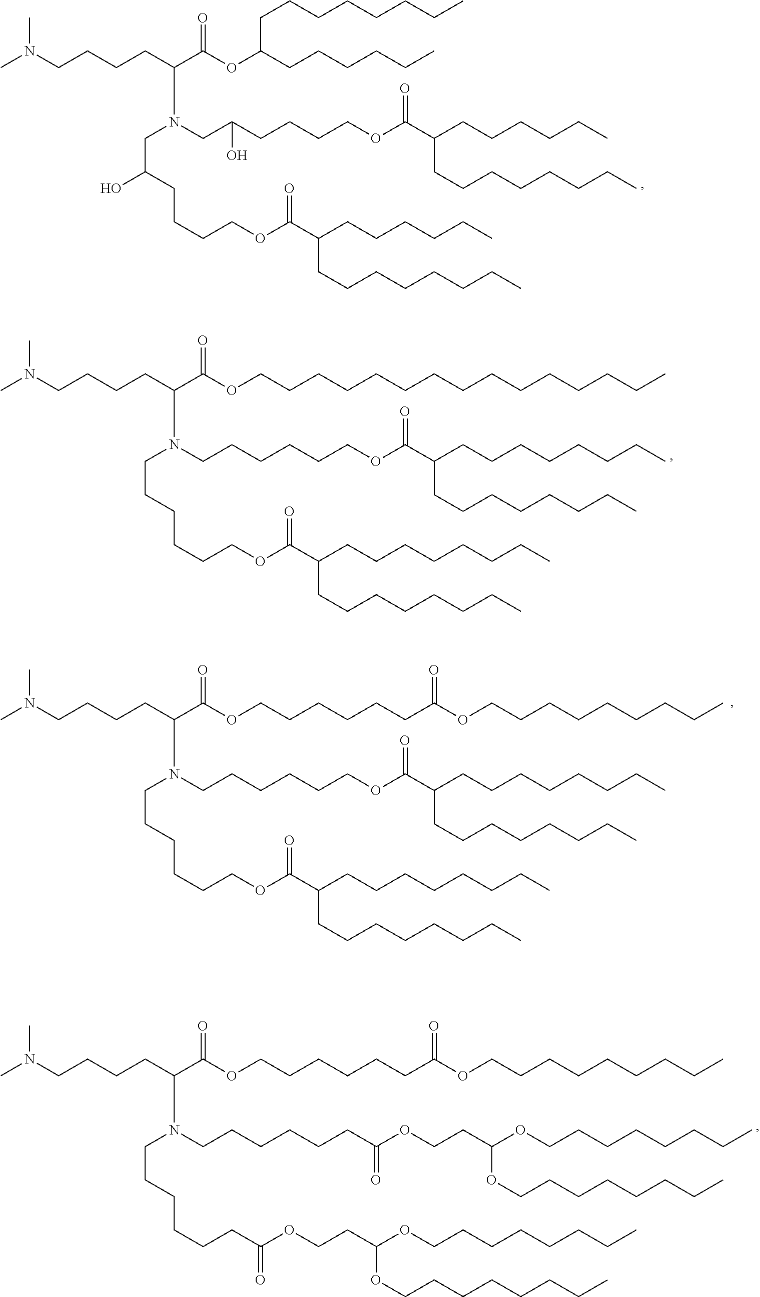

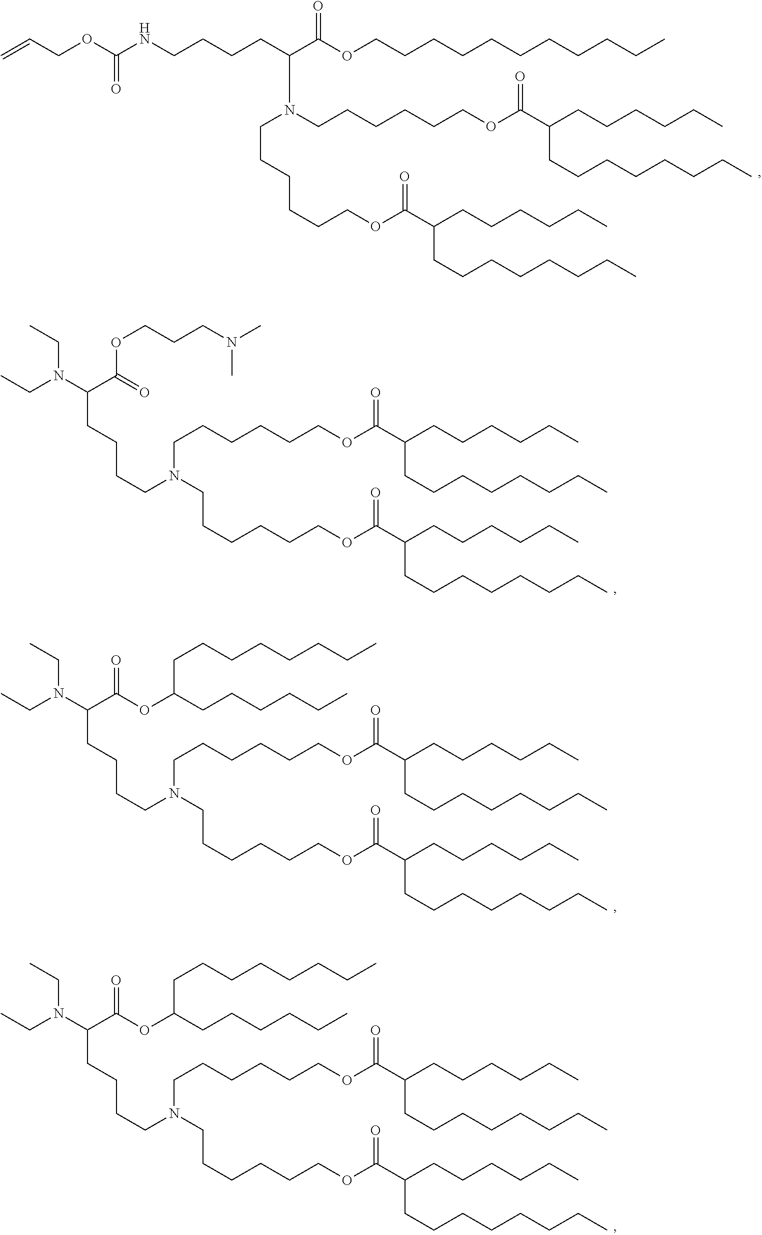

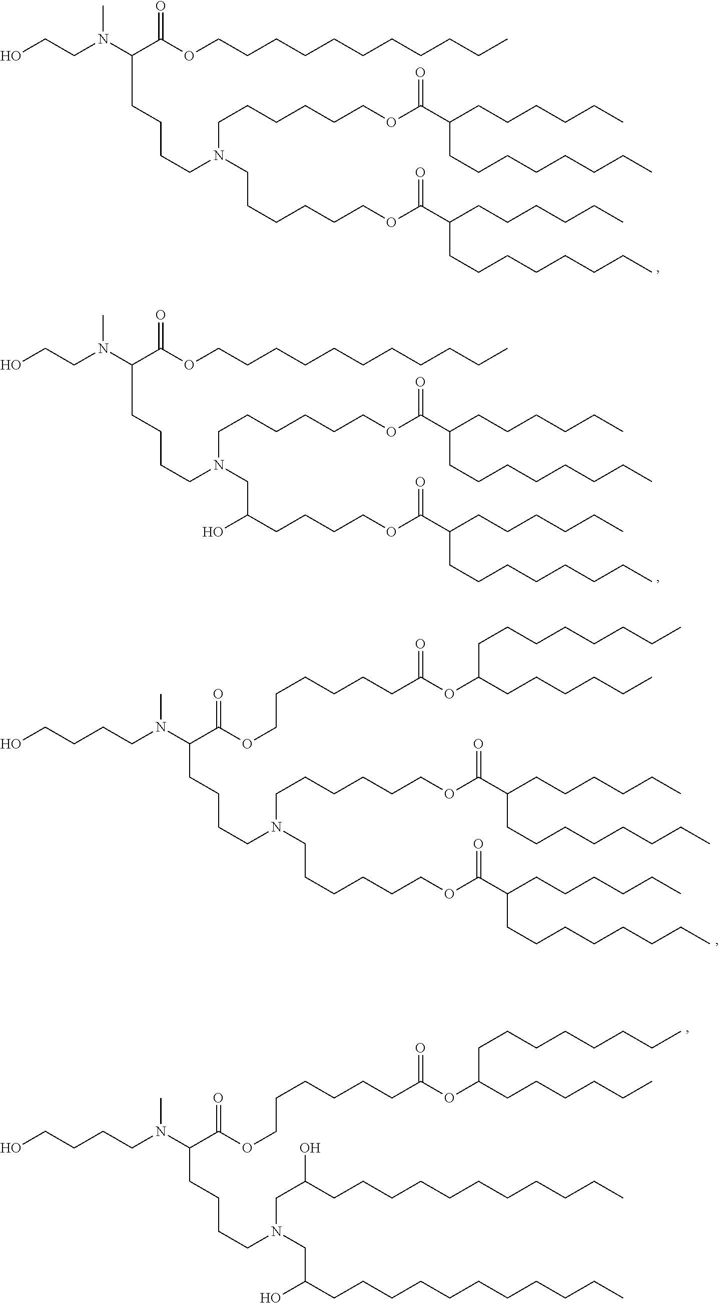 [SMILES: CCCCCCCCCCCOC(=O)C(CCCCN(CCCCCCOC(=O)C(CCCCCC)CCCCCCCC)CCCCCCOC(=O)C(CCCCCC)CCCCCCCC)N(C)CCO ; CCCCCCCCCCCOC(=O)C(CCCCN(CCCCCCOC(=O)C(CCCCCC)CCCCCCCC)CC(O)CCCCOC(=O)C(CCCCCC)CCCCCCCC)N(C)CCO ; CCCCCCCCC(CCCCCC)OC(=O)CCCCCCOC(=O)C(CCCCN(CCCCCCOC(=O)C(CCCCCC)CCCCCCCC)CCCCCCOC(=O)C(CCCCCC)CCCCCCCC)N(C)CCCCO ; CCCCCCCCCCCC(O)CN(CCCCC(C(=O)OCCCCCCC(=O)OC(CCCCCC)CCCCCCCC)N(C)CCCCO)CC(O)CCCCCCCCCCC]

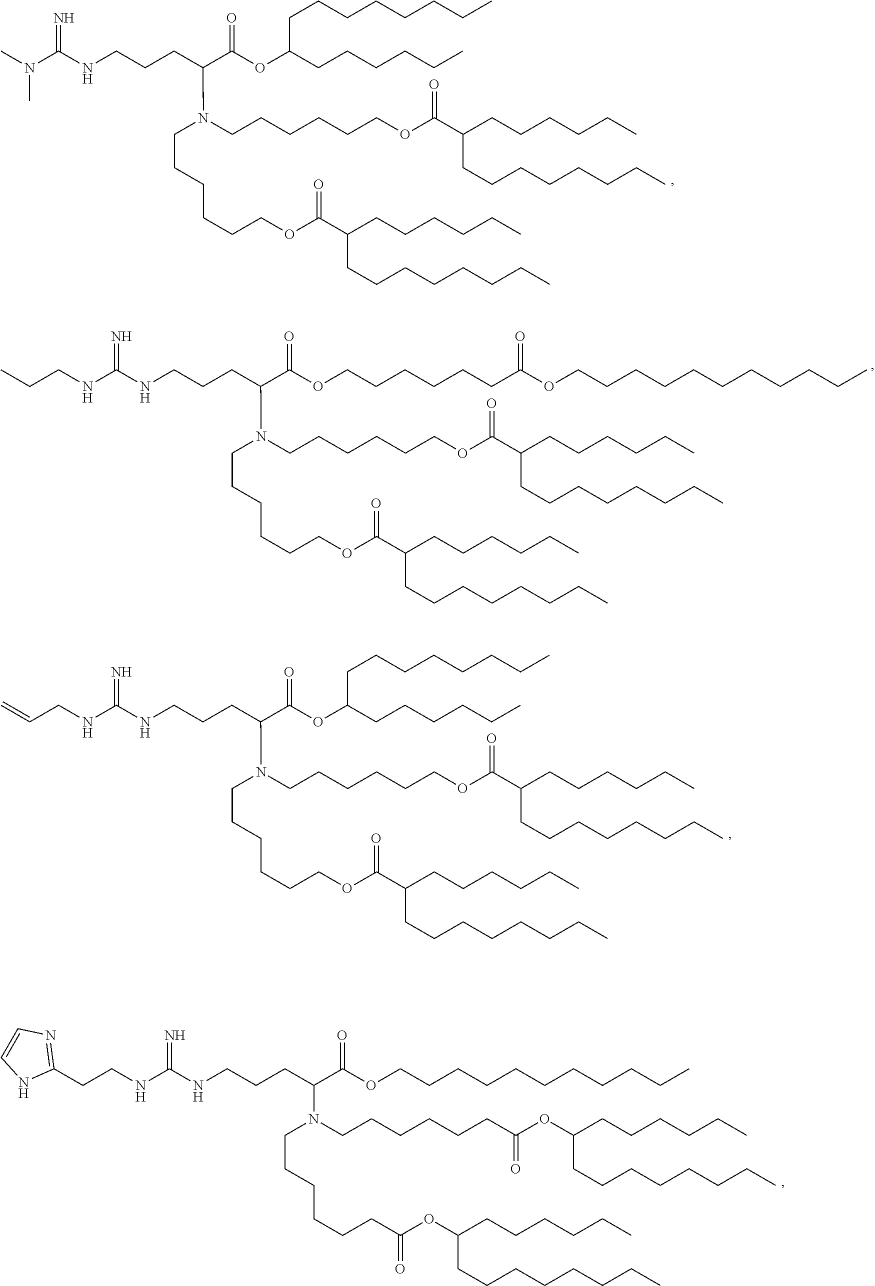

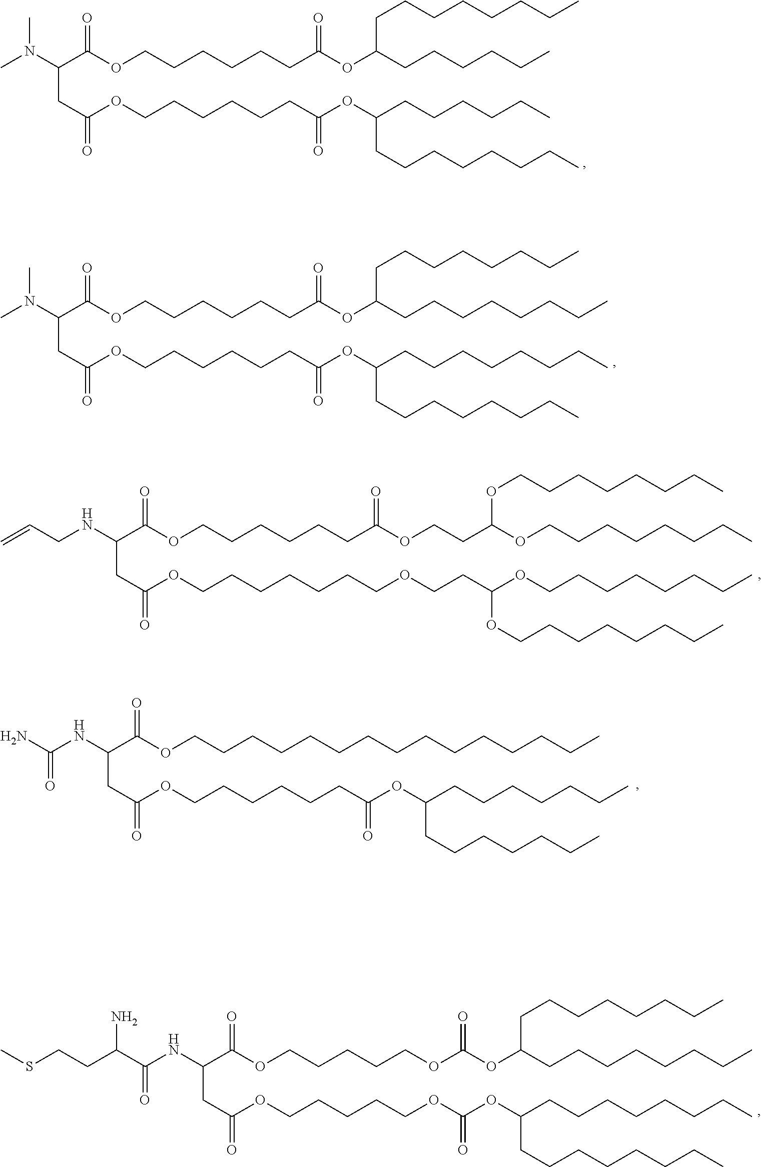

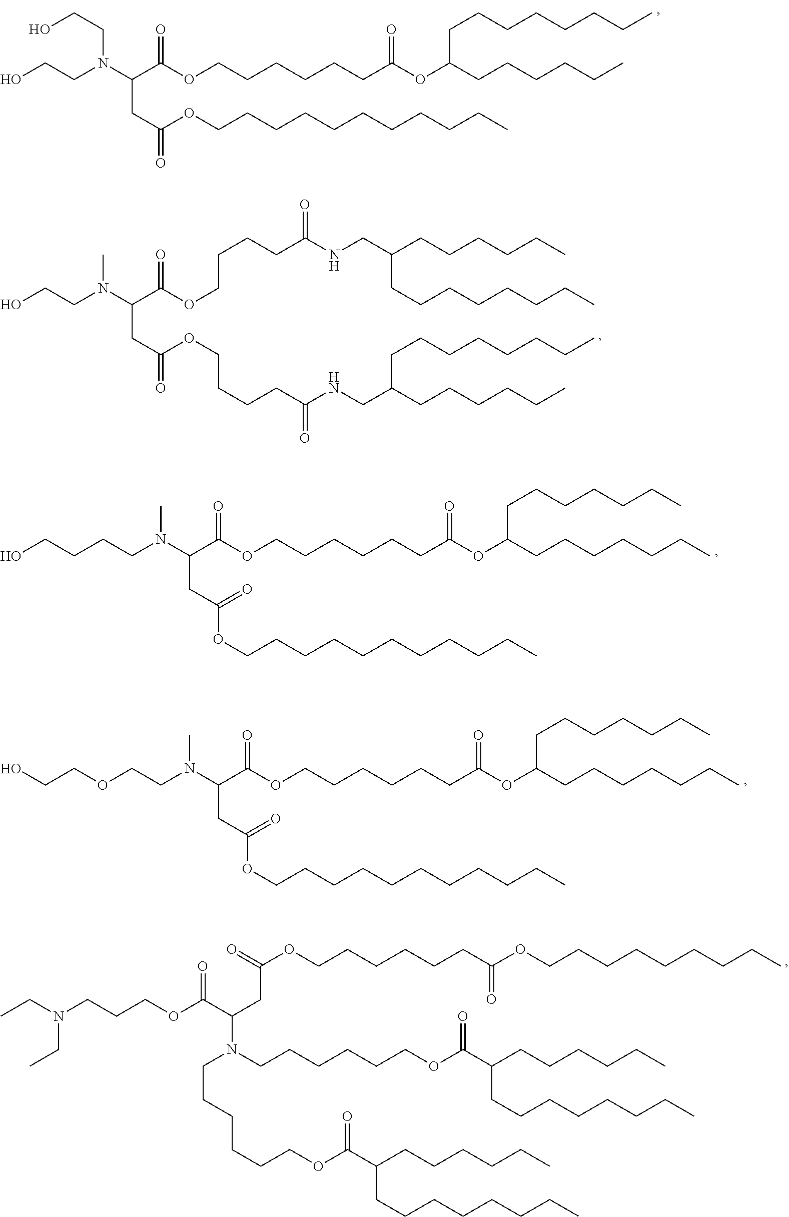

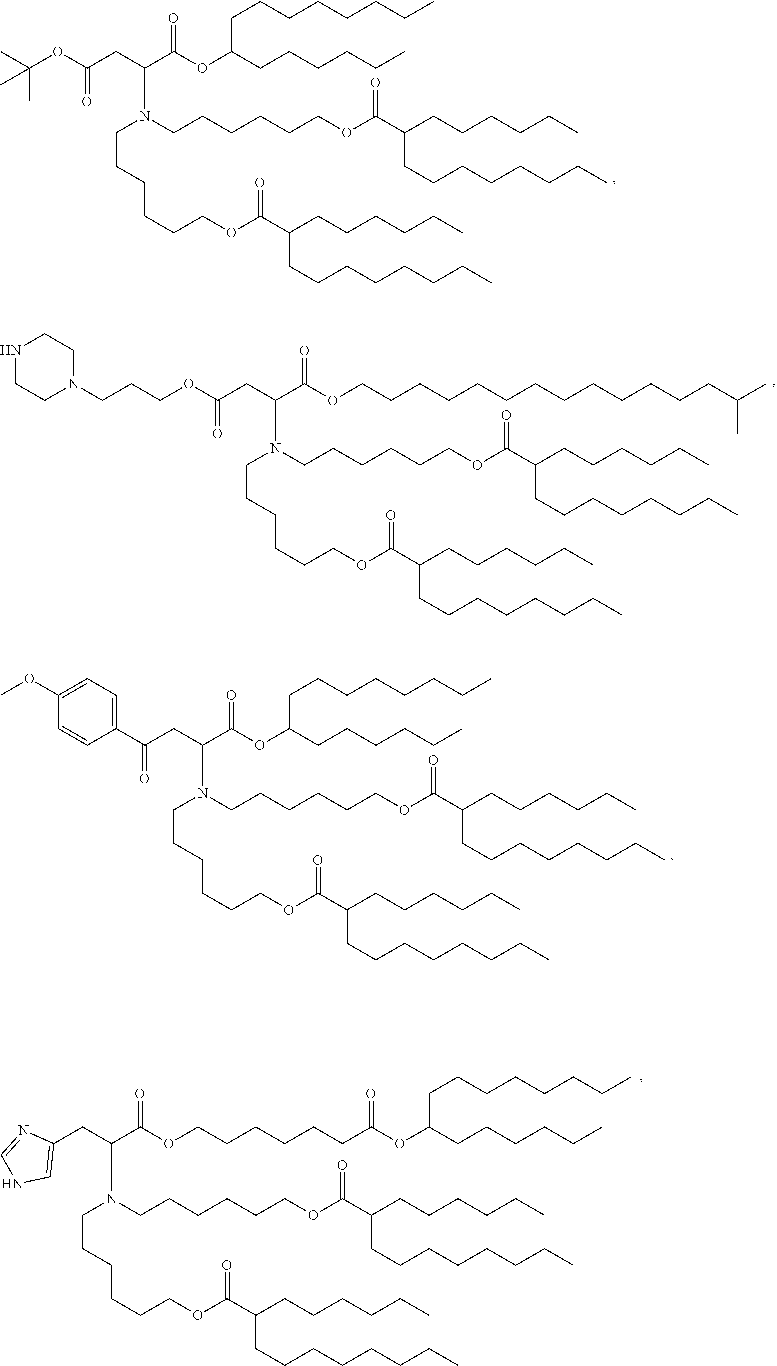

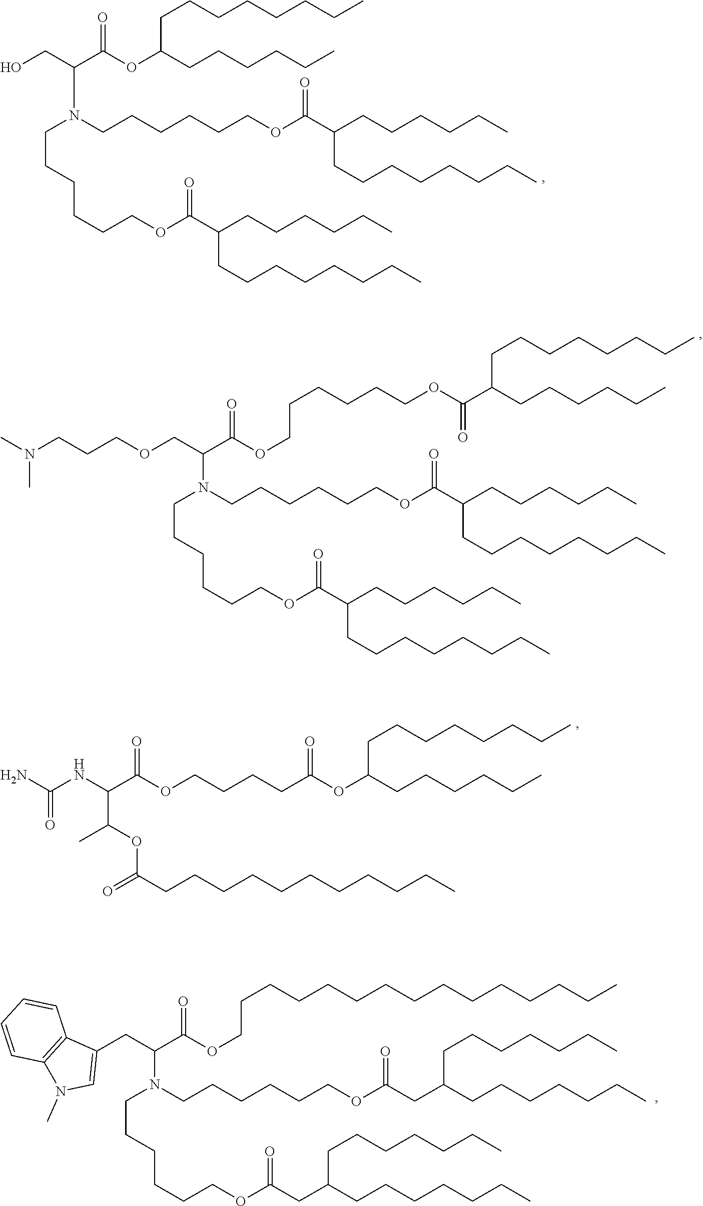

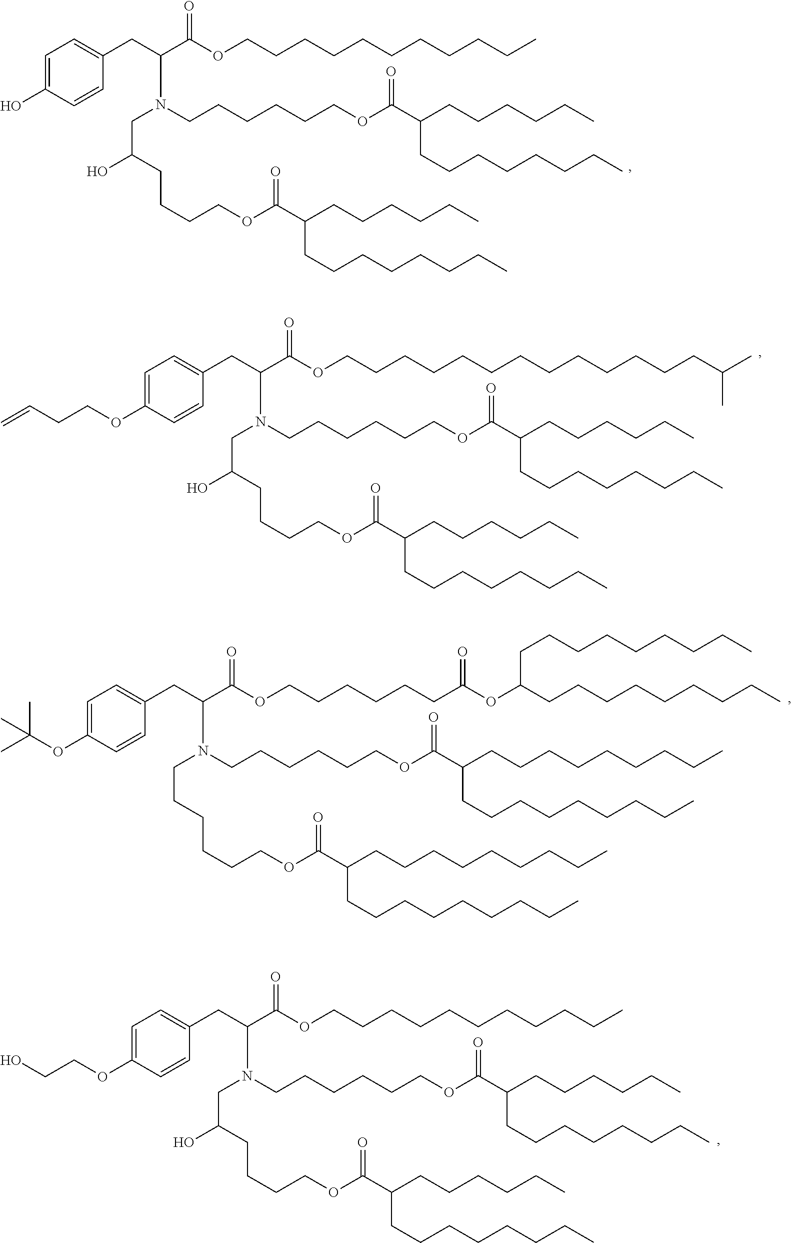

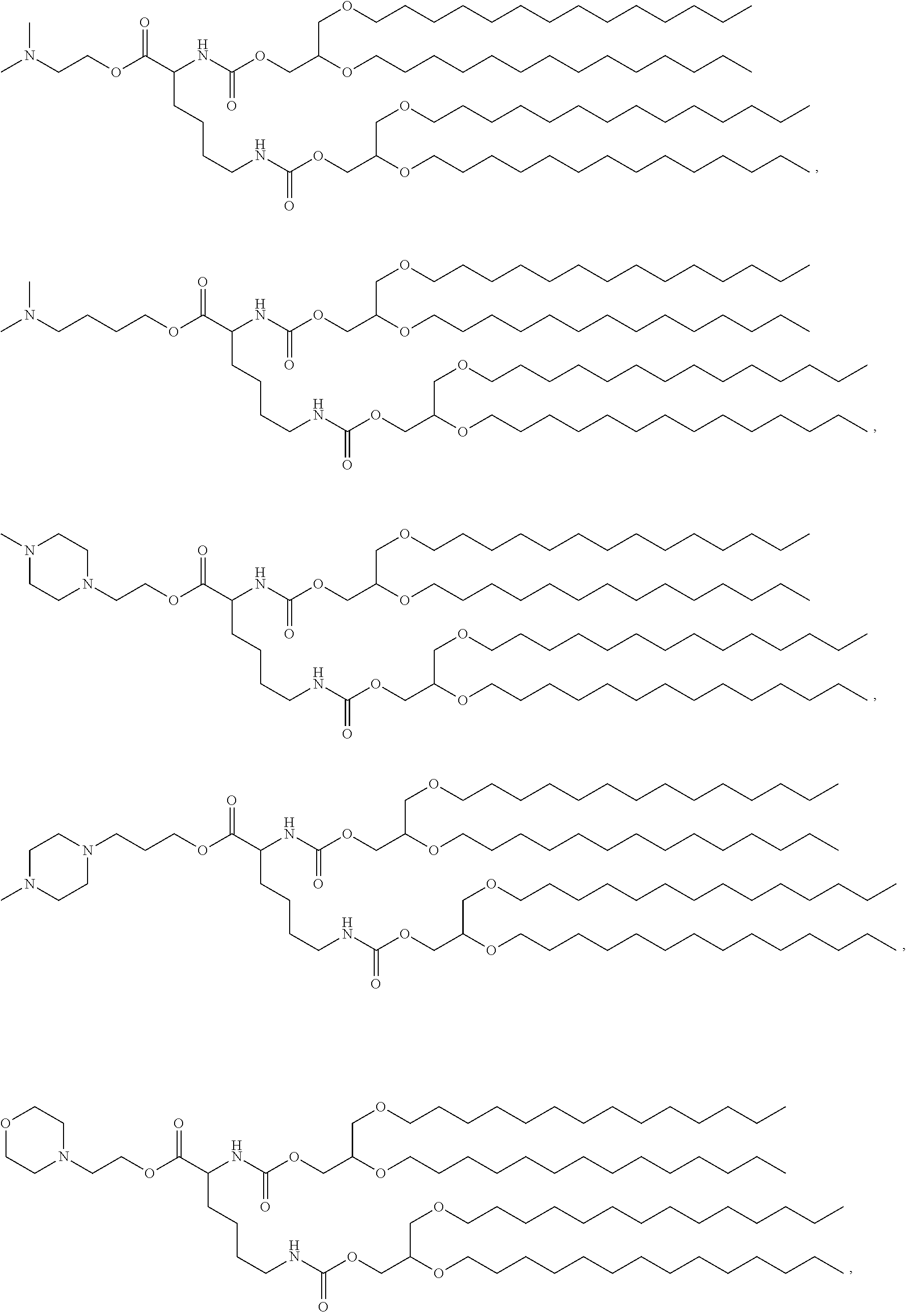

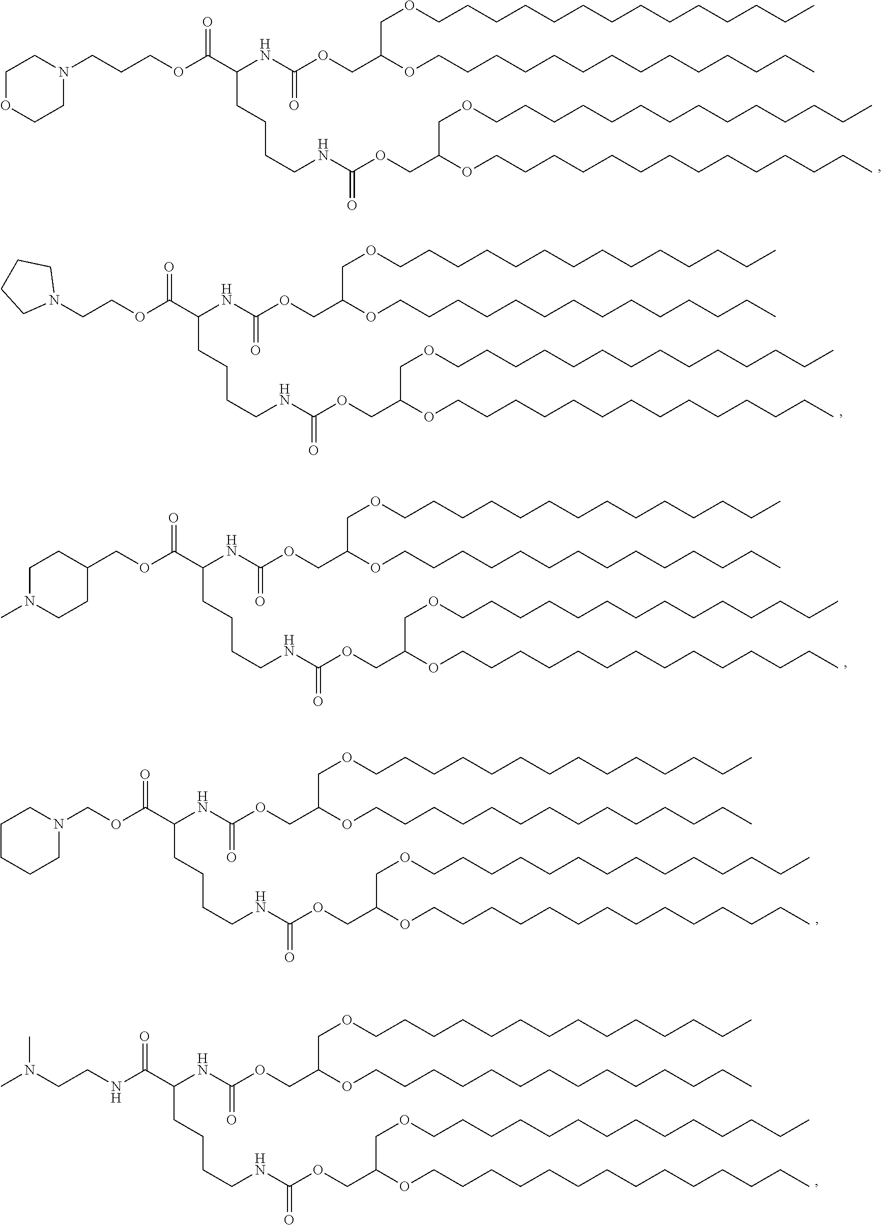

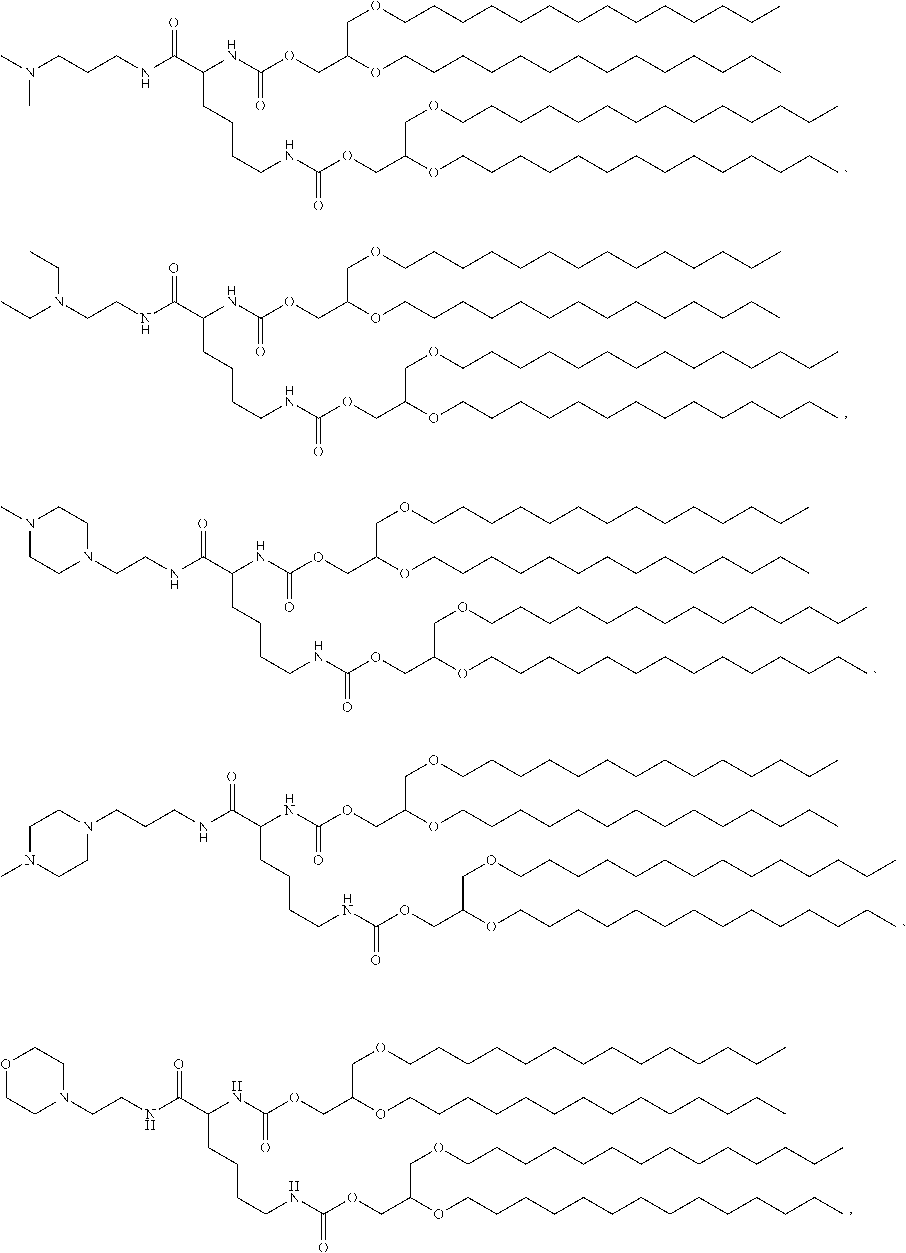

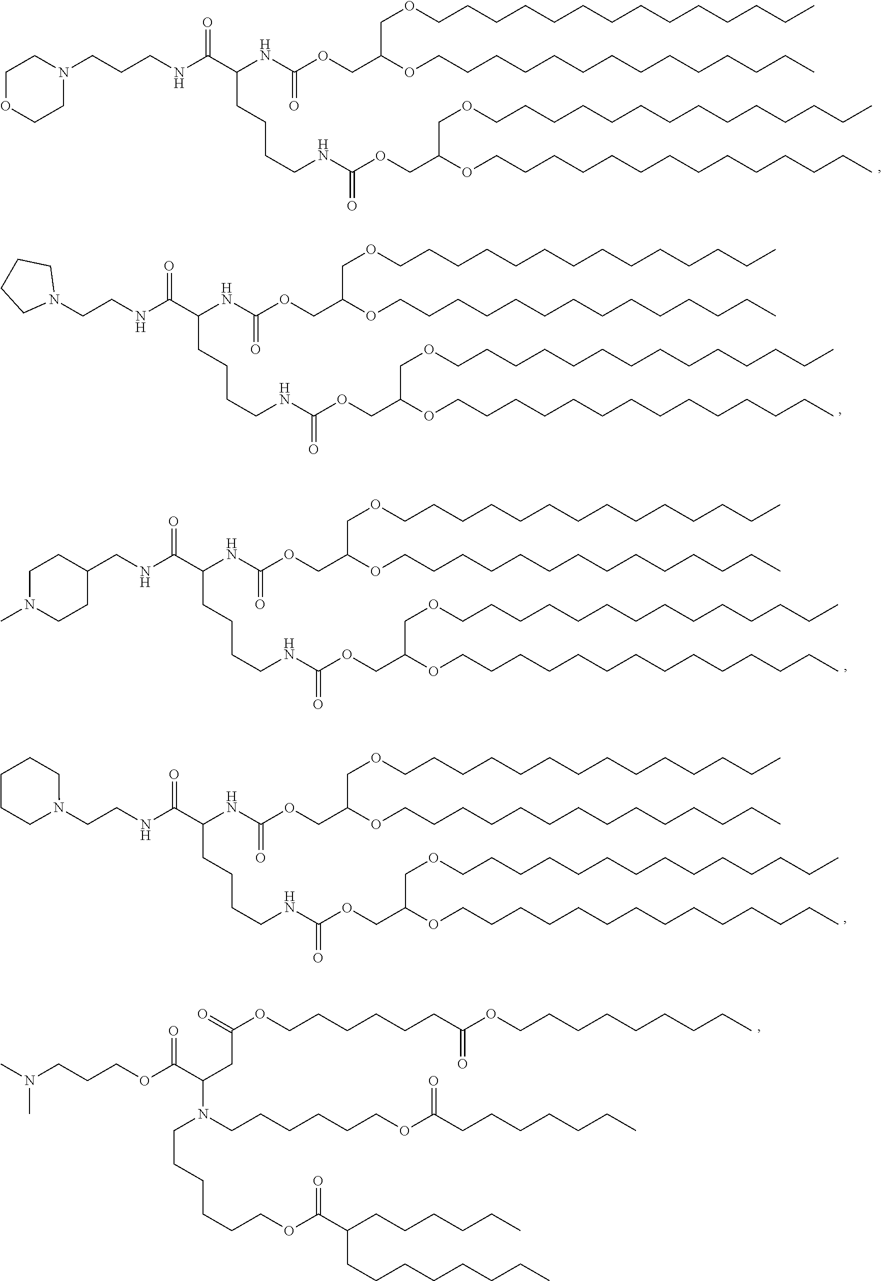

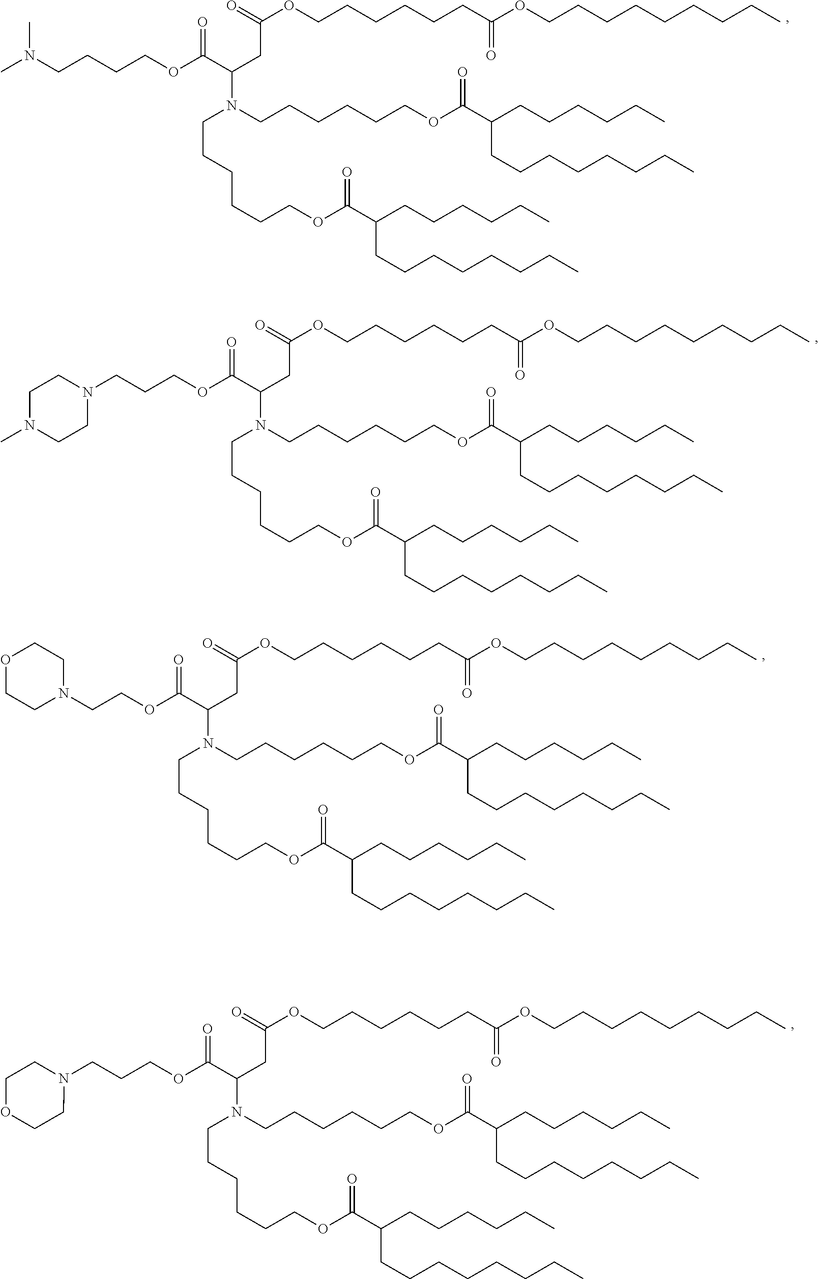

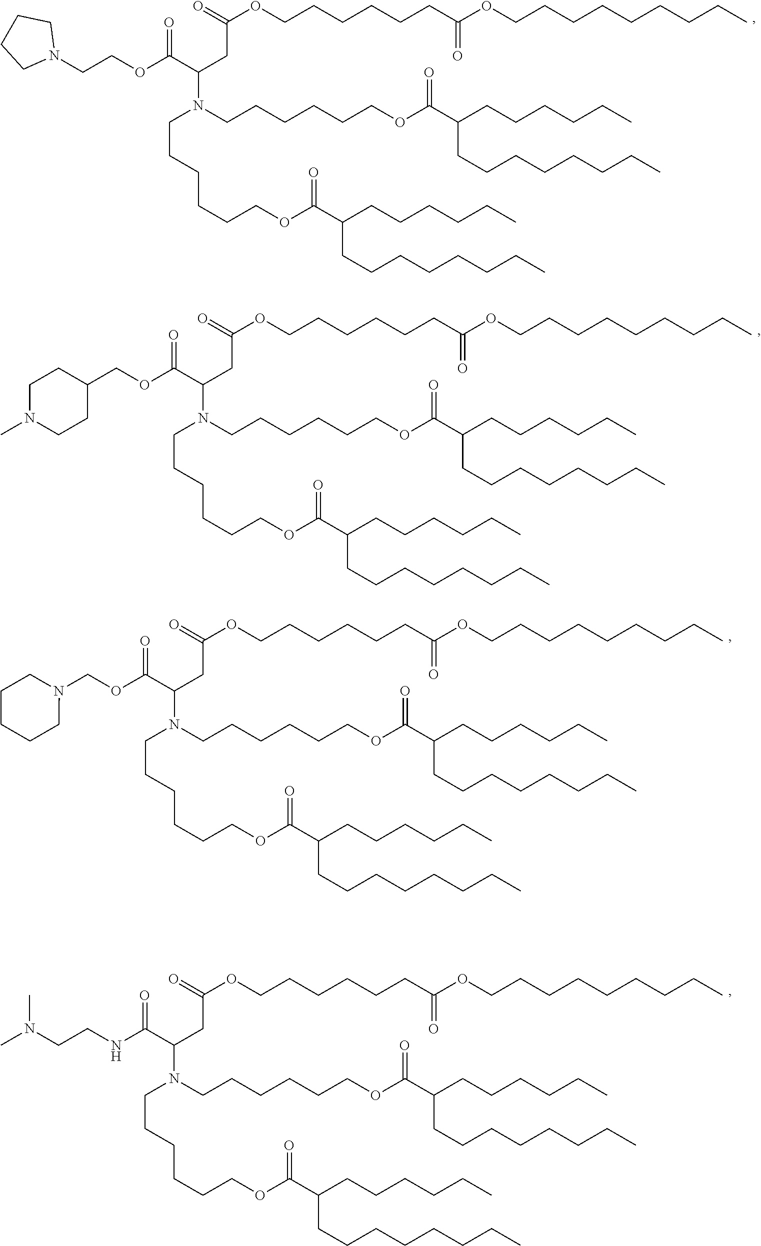

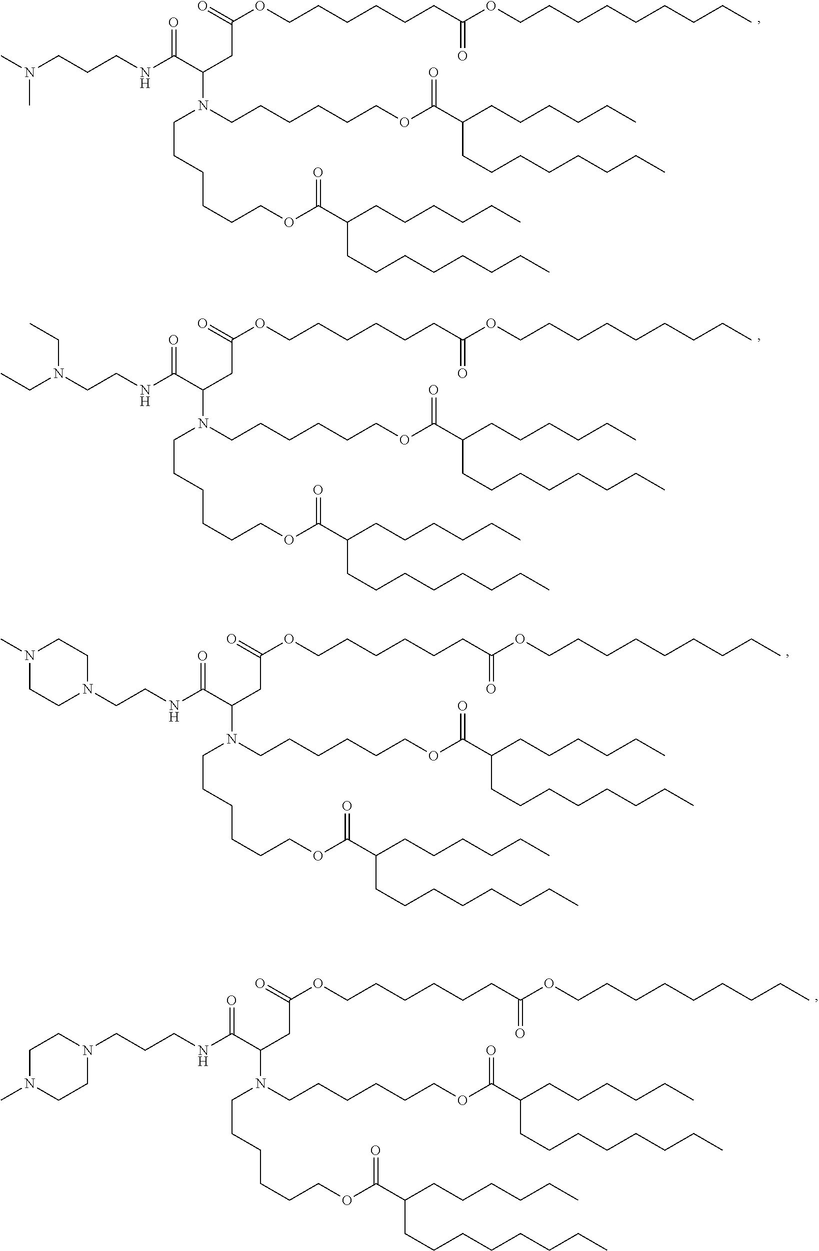

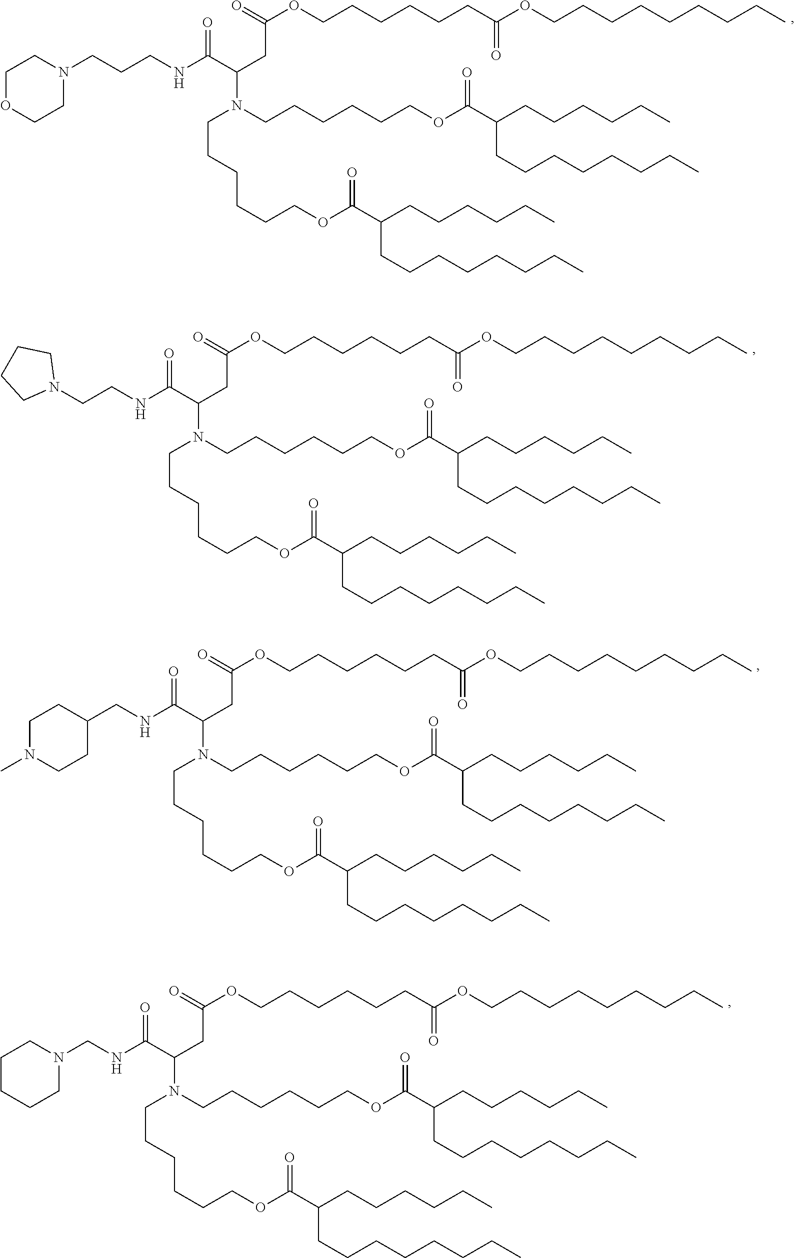

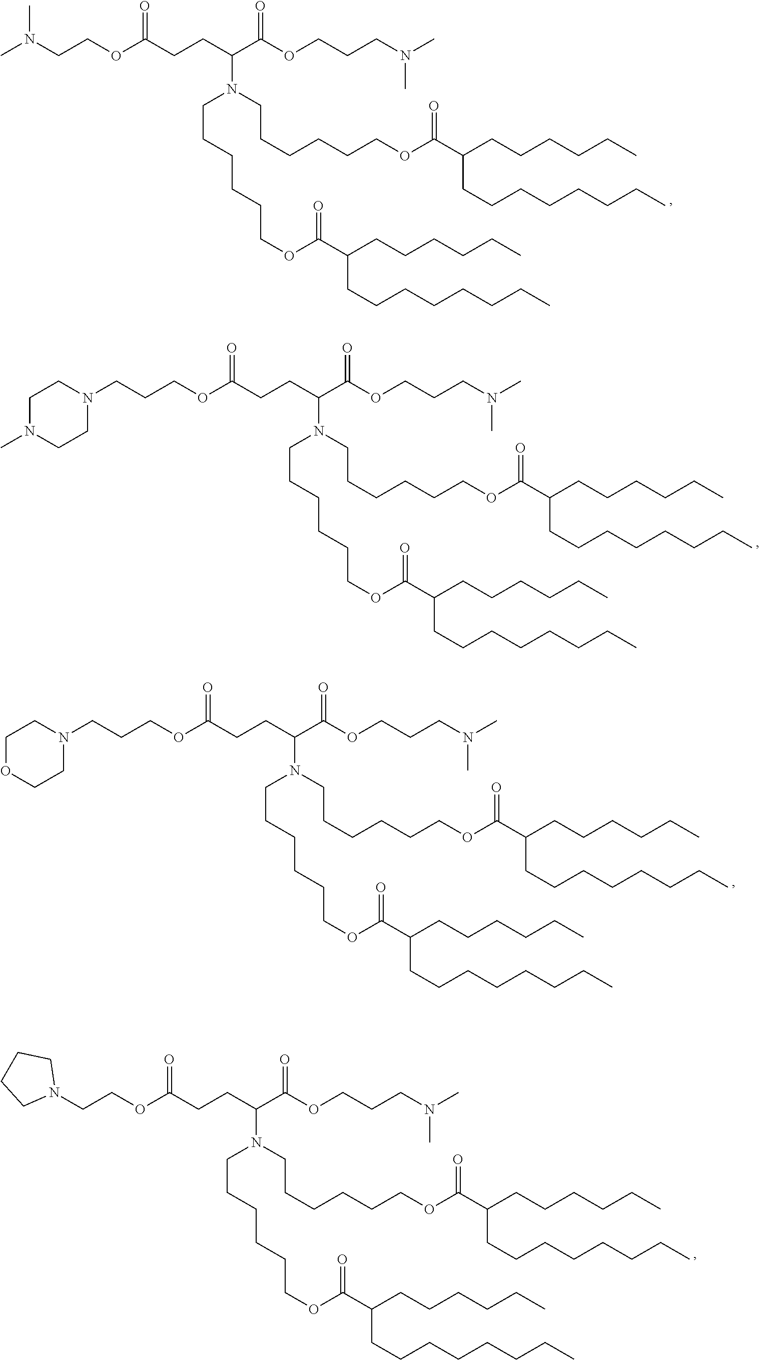

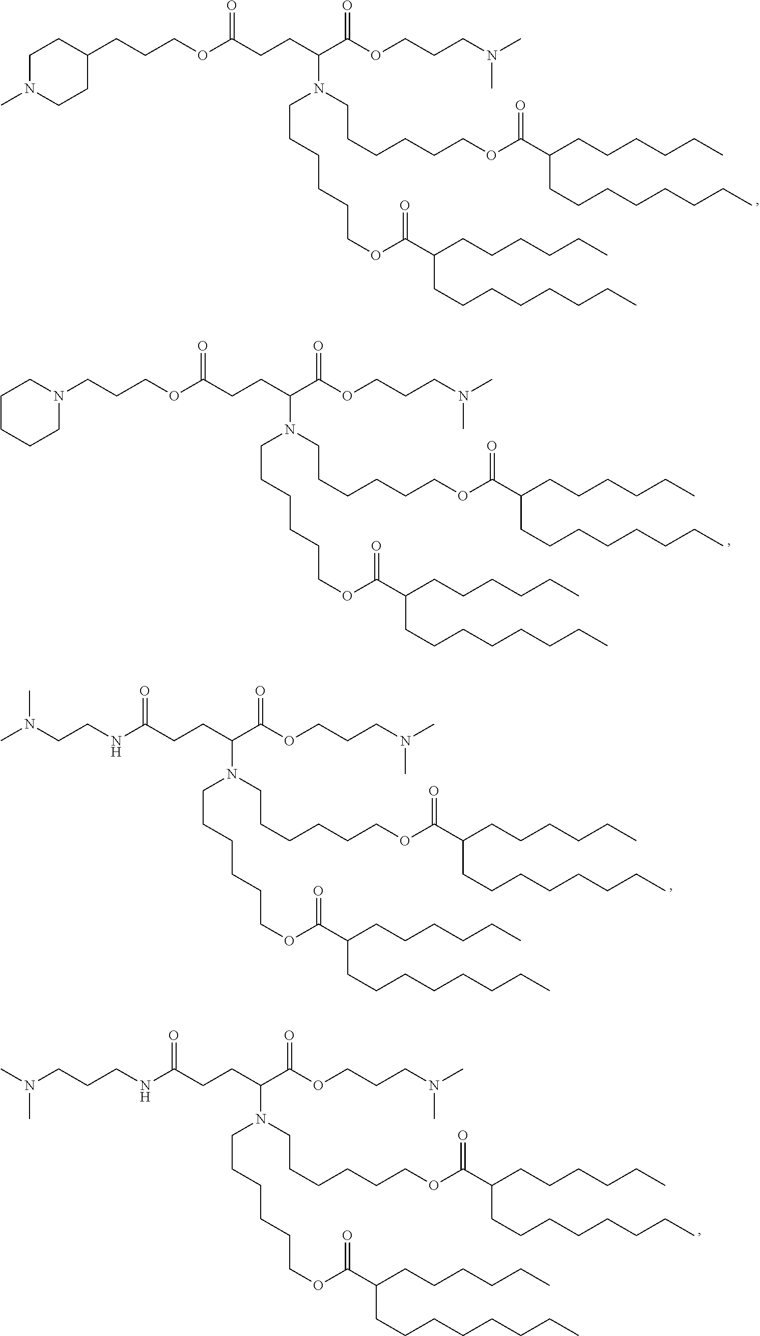

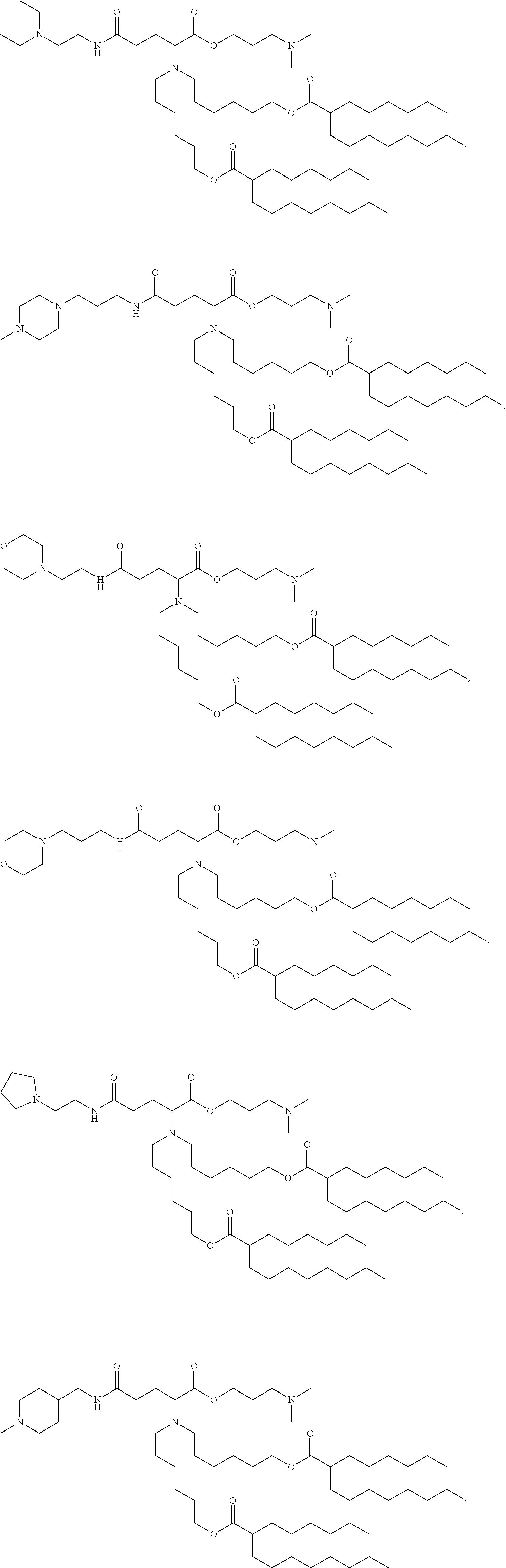

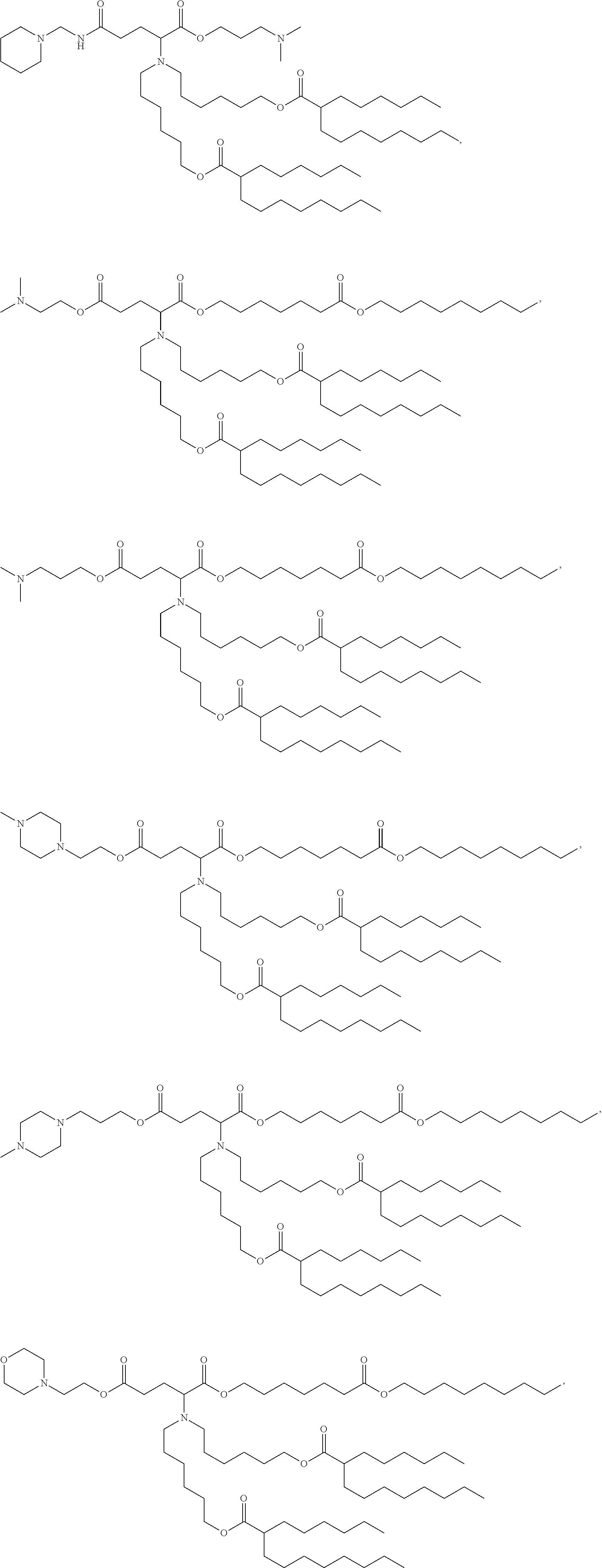

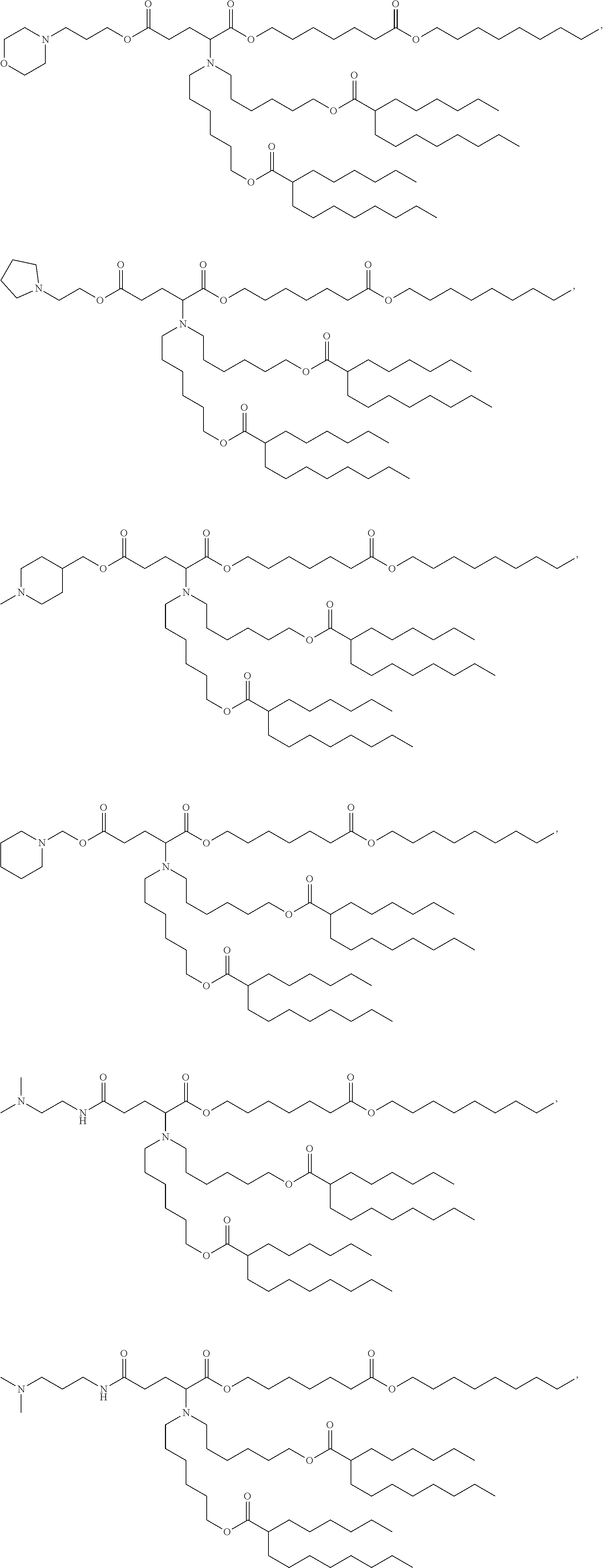

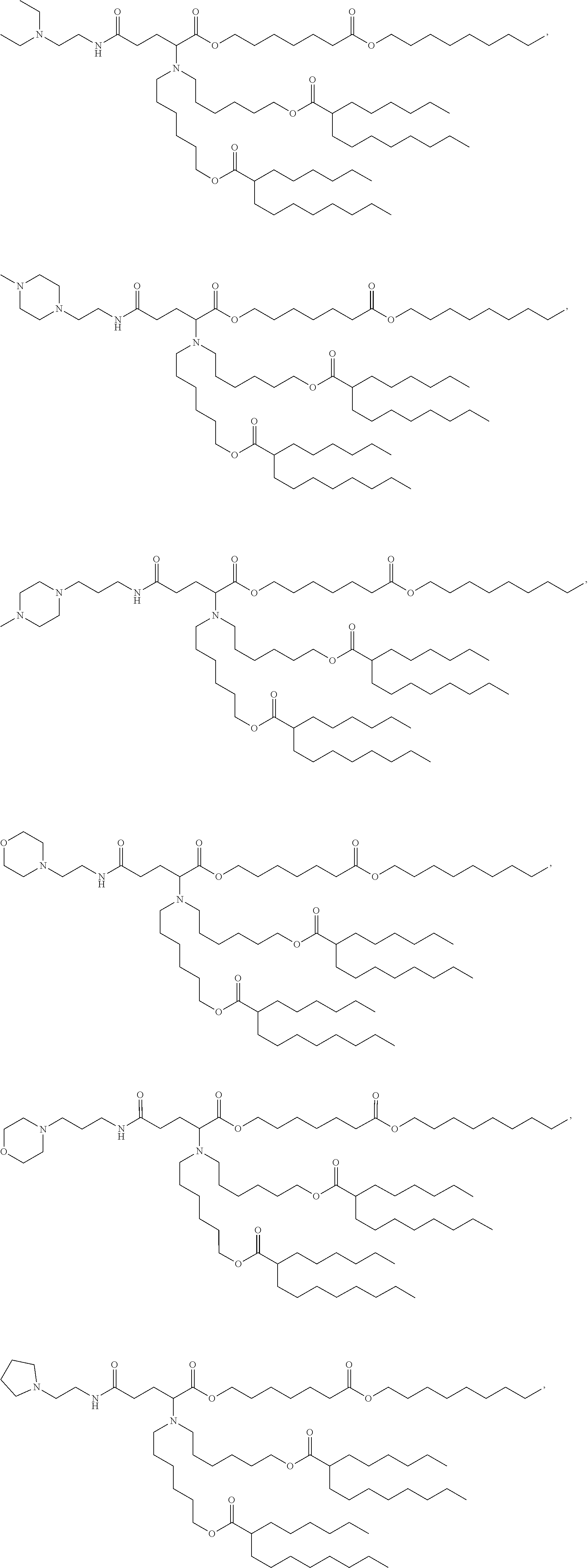

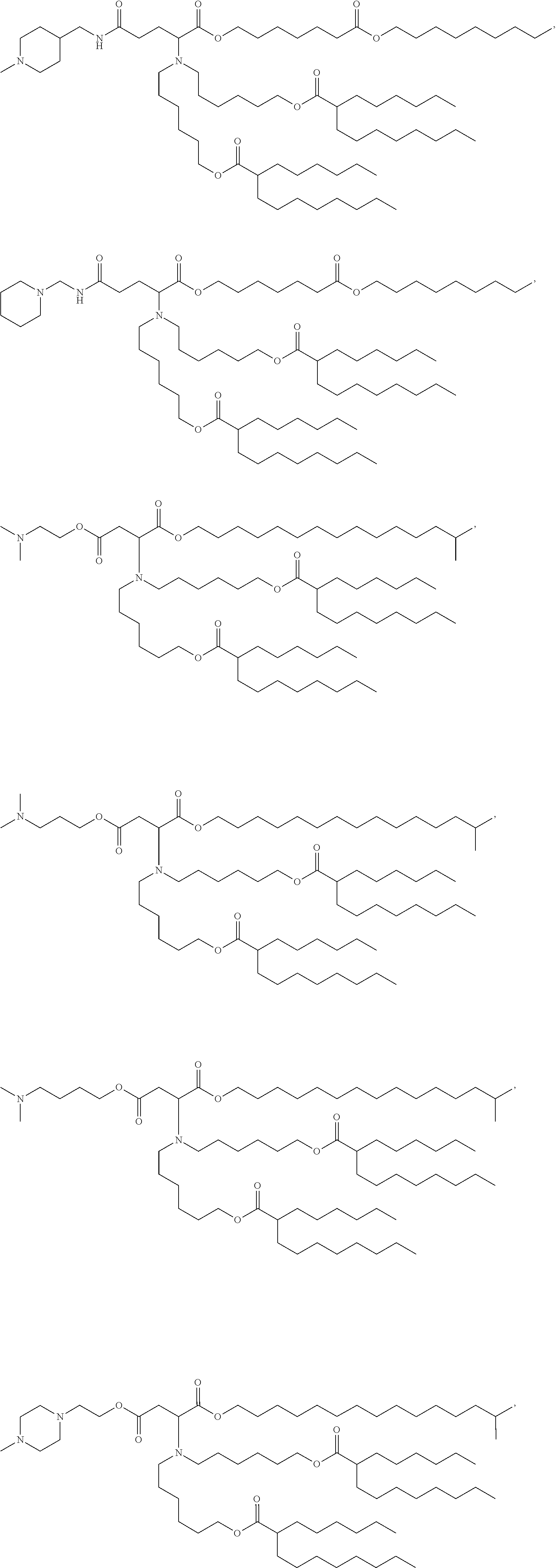

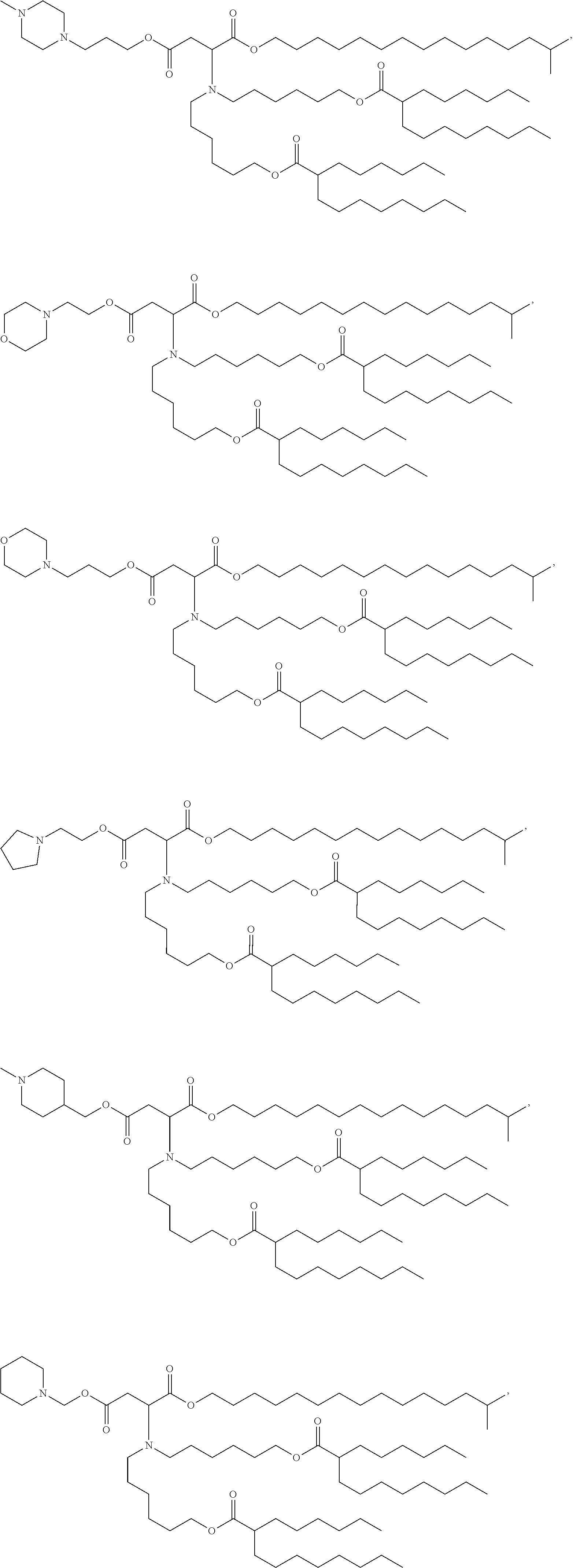

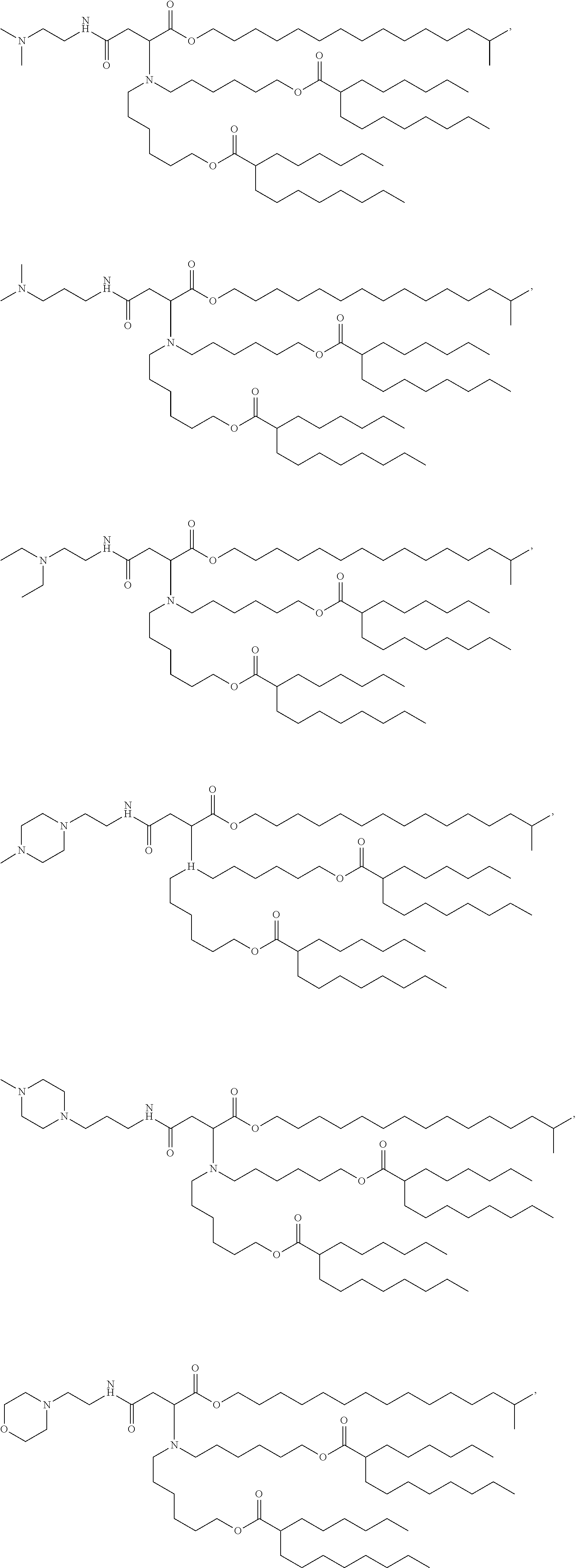

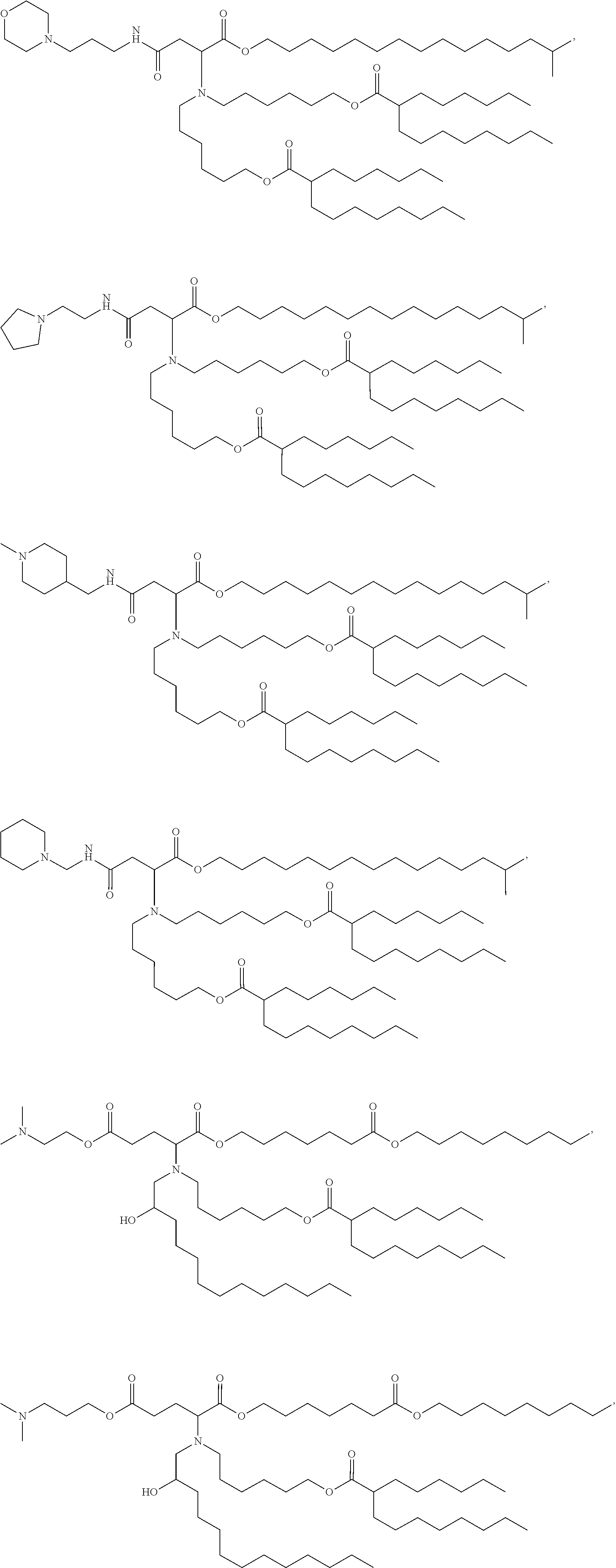

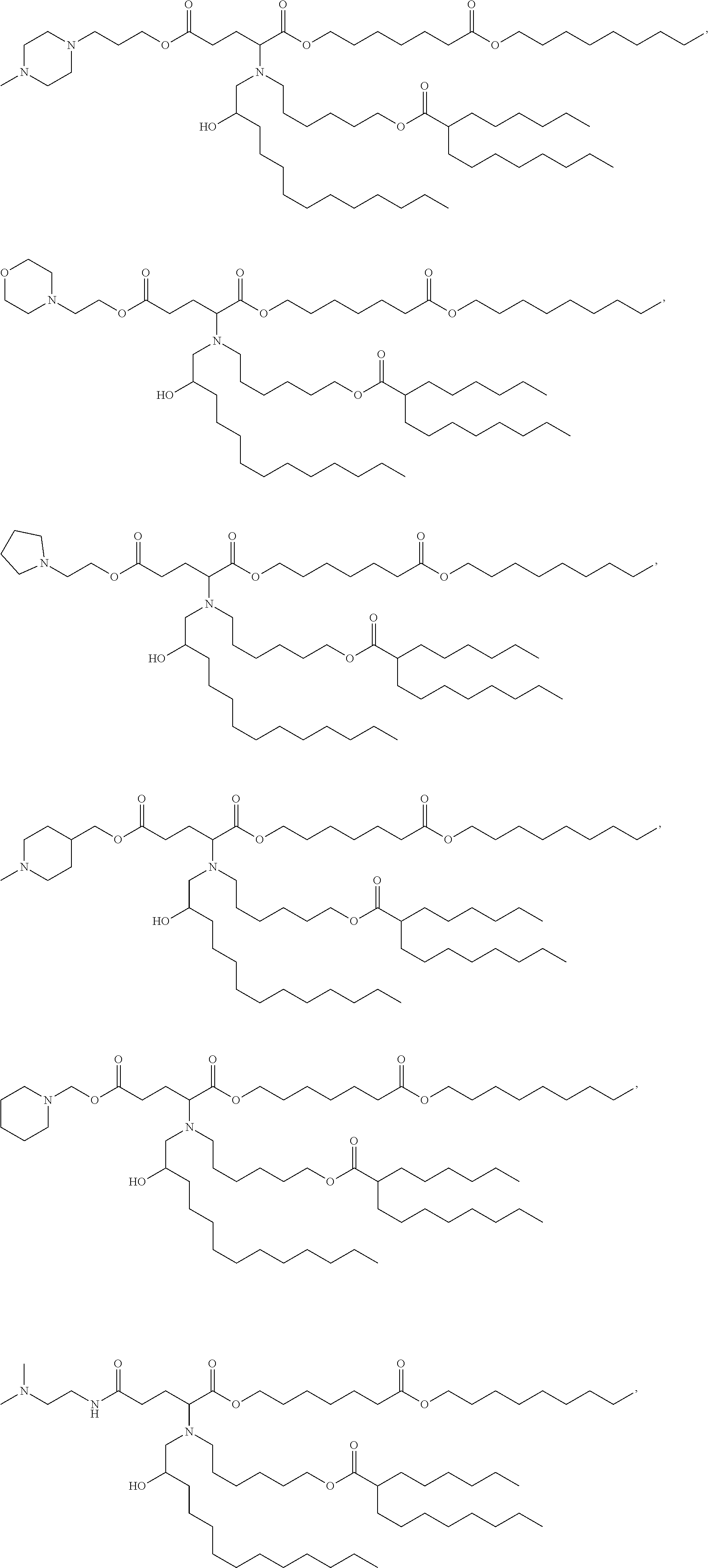

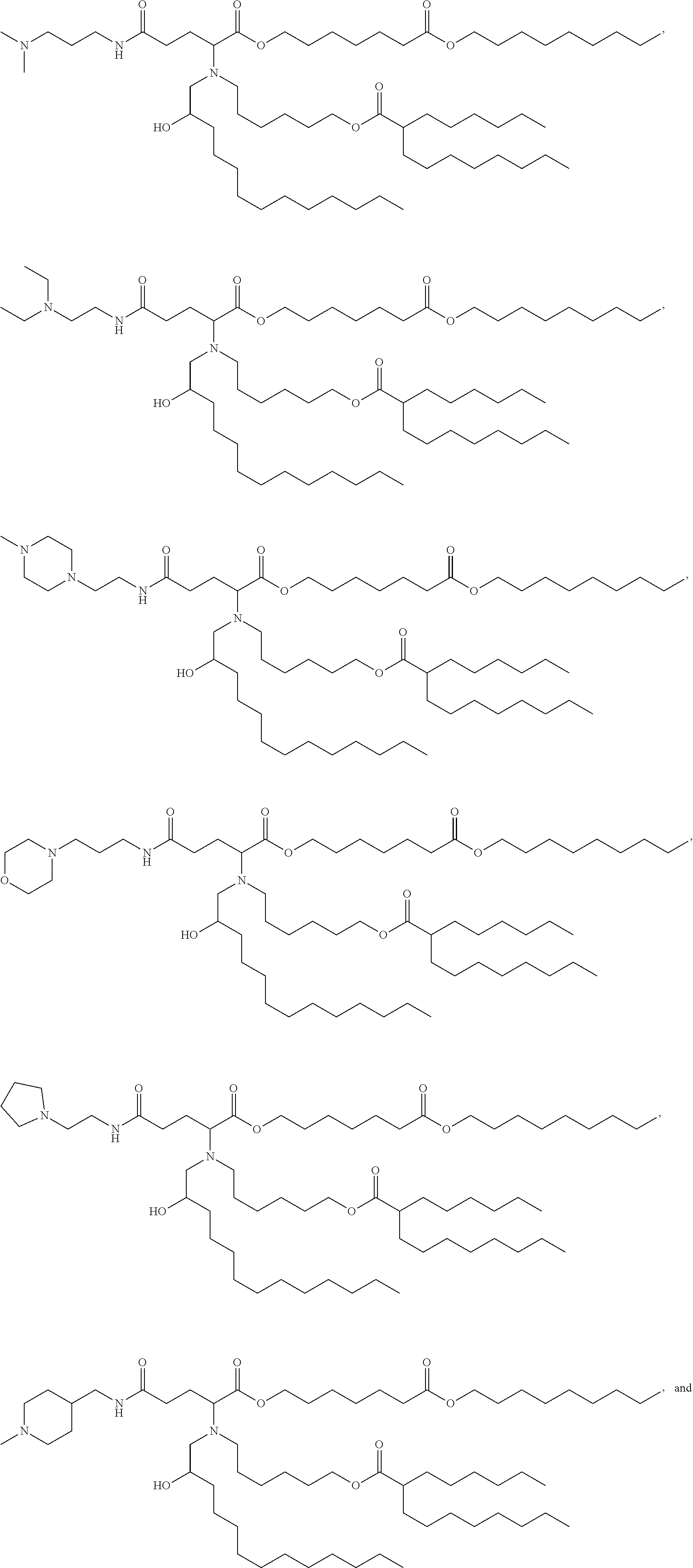

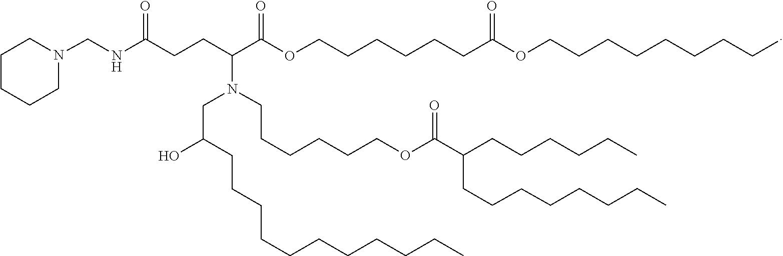

16. A lipid composition containing an amino acid-based cationic lipid of claim 1.

17. The lipid composition according to claim 16, wherein the lipid composition contains one or more types of lipids selected from the group consisting of phospholipid, steroid lipid, and PEGylated lipid; the lipid composition is selected from any of the following cases:
Case (1): also contains a phospholipid;
Case (2): also contains a steroid lipid;
Case (3): also contains a PEGylated lipid;
Case (4): also contains a phospholipid and a steroid lipid;
Case (5): also contains a phospholipid and a PEGylated lipid;
Case (6): also contains a steroid lipid and a PEGylated lipid;
Case (7): also contains a phospholipid, a steroid lipid, and a PEGylated lipid.

18. The lipid composition according to claim 17, wherein the phospholipid is selected from the group consisting of 1,2-dilinoleoyl-sn-glycero-3-phosphocholine, 1,2-dimyristoleoyl-sn-glycero-3-phosphocholine, 1,2-dioleoyl-sn-glycero-3-phosphocholine, 1,2-dipalmitoyl-sn-glycero-3-phosphocholine, 1,2-distearoyl-sn-glycero-3-phosphatidylcholine, 1,2-diundecanoyl-sn-glycero-3-phosphatidylcholine, 1-plamitoyl-2-oleoyl-sn-glycero-3-phosphocholine, 1,2-di-O-octadecenyl-sn-glycero-3-phosphatidylcholine, 1-oleoyl-2-cholesterylhemisuccinyl-sn-glycero-3-phosphocholine, 1-O-hexadecyl-sn-glycero-3-phosphatidylcholine, 1,2-dilinolenoyl-sn-glycero-3-phosphatidylcholine, 1,2-diarachidonoyl-sn-glycero-3-phosphatidylcholine, 1,2-didecosahexaenoyl-sn-glycero-3-phosphocholine, 1,2-dioleoyl-sn-glycero-3-phosphoethanolamine, 1,2-diphytanyl-sn-glycero-3-phosphoethanolamine, 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didecosahexaenoyl-sn-glycero-3-phosphoethanolamine, 1,2-dioleoyl-sn-glycero-3-phospho-rac-(1-glycerol) sodium salt, dioleoyl phosphatidylserine, dipalmitoyl phosphatidylglycerol, palmitoyloleoyl phosphatidylethanolamine, distearoyl phosphatidylethanolamine, dipalmitoyl phosphatidylethanolamine, dimyristoleoyl phosphoethanolamine, 1-stearoyl-2-oleoyl-stearoylethanolamine, 1-stearoyl-2-oleoyl-phosphatidylcholine, sphingomyelin, phosphatidylcholine, phosphatidylethnolamine, phosphatidylserine, phosphatidylinositol, phosphatidic acid, palmitoyloleoyl phosphatidylcholine, lysophosphatidylcholine, lysophosphatidylethanolamine, and combinations thereof;
or the steroid lipid is selected from the group consisting of cholesterol, fecal sterol, sitosterol, ergosterol, campesterol, stigmasterol, rapeseed sterol, lycopene, ursolic acid and α-tocopherol, and combinations thereof;
or the PEGylated lipid is selected from the group consisting of 1,2-dimyristoyl-sn-glycerol methoxypolyethylene glycol, 1,2-distearoyl-sn-glycero-3-phosphoethanolamine-N-[amino(polyethylene glycol)], PEG-cholesterol, PEG-diacylglycamide, PEG-dialkyloxypropyl, PEG500-dipalmitoylphosphatidylcholine, PEG2000-dipalmitoylphosphatidylcholine, PEG500-distearylphosphatidylethanolamine, PEG2000-distearylphosphatidylethanolamine, PEG500-1,2-dioleoylphosphatidylethanolamine, PEG2000-1,2-dioleoylphosphatidylethanolamine and PEG2000-2,3-distearoylglycerol, and combinations thereof;
or the structure of PEGylated lipid is selected from the group consisting of the following structures and combinations thereof: 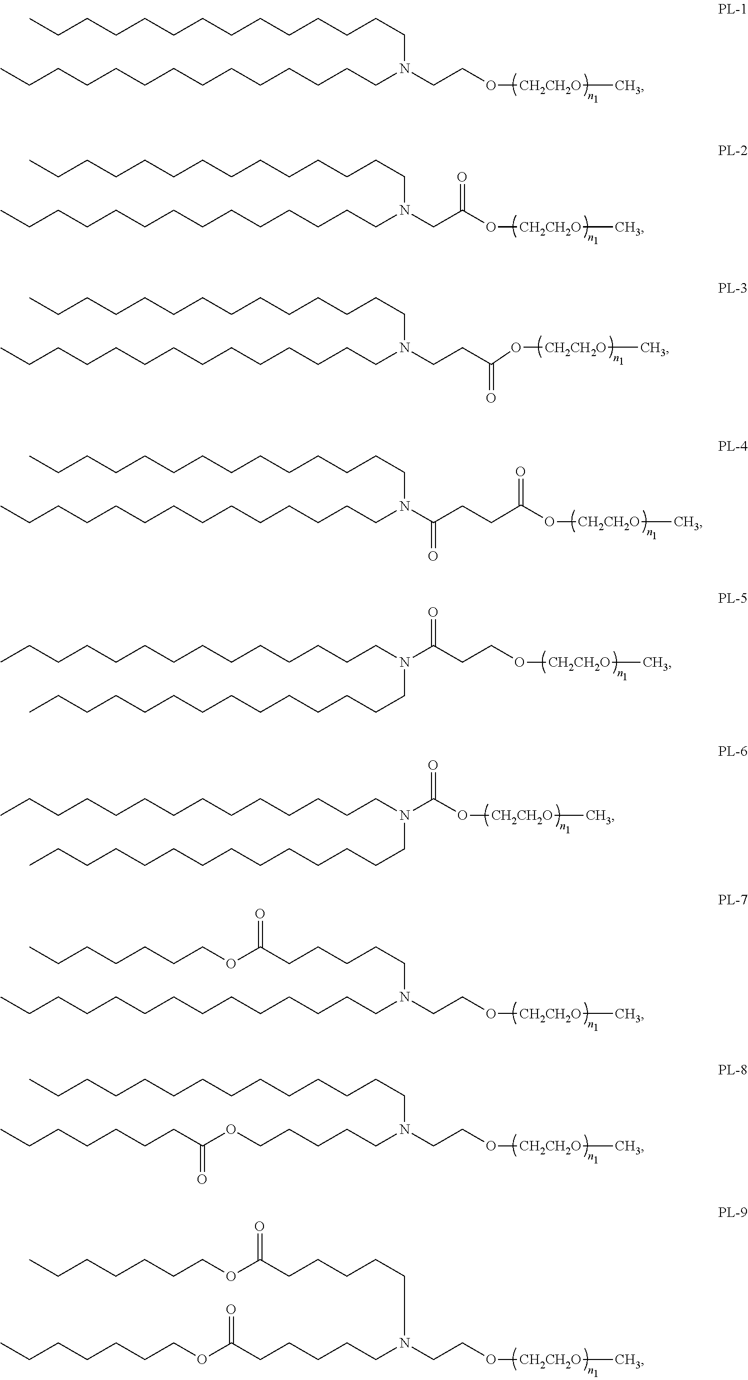 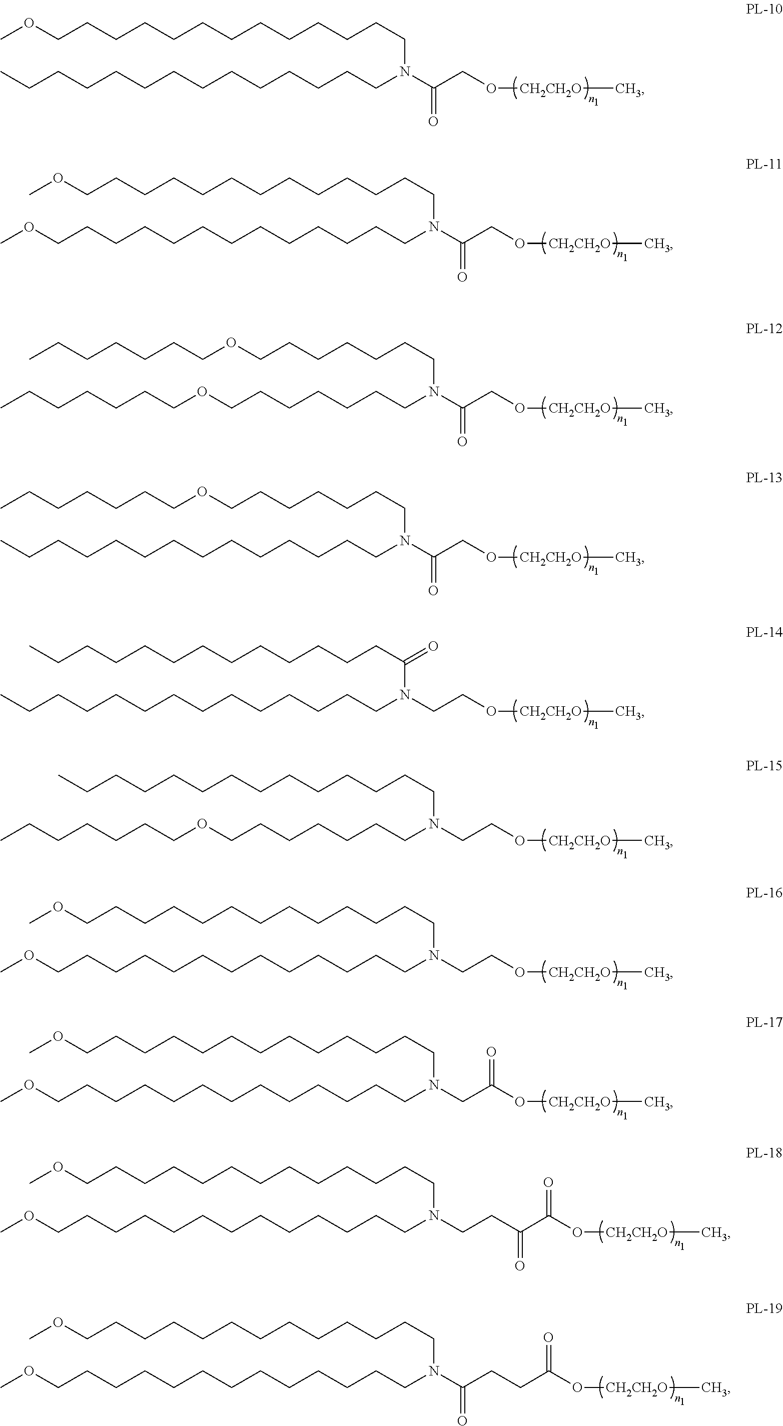 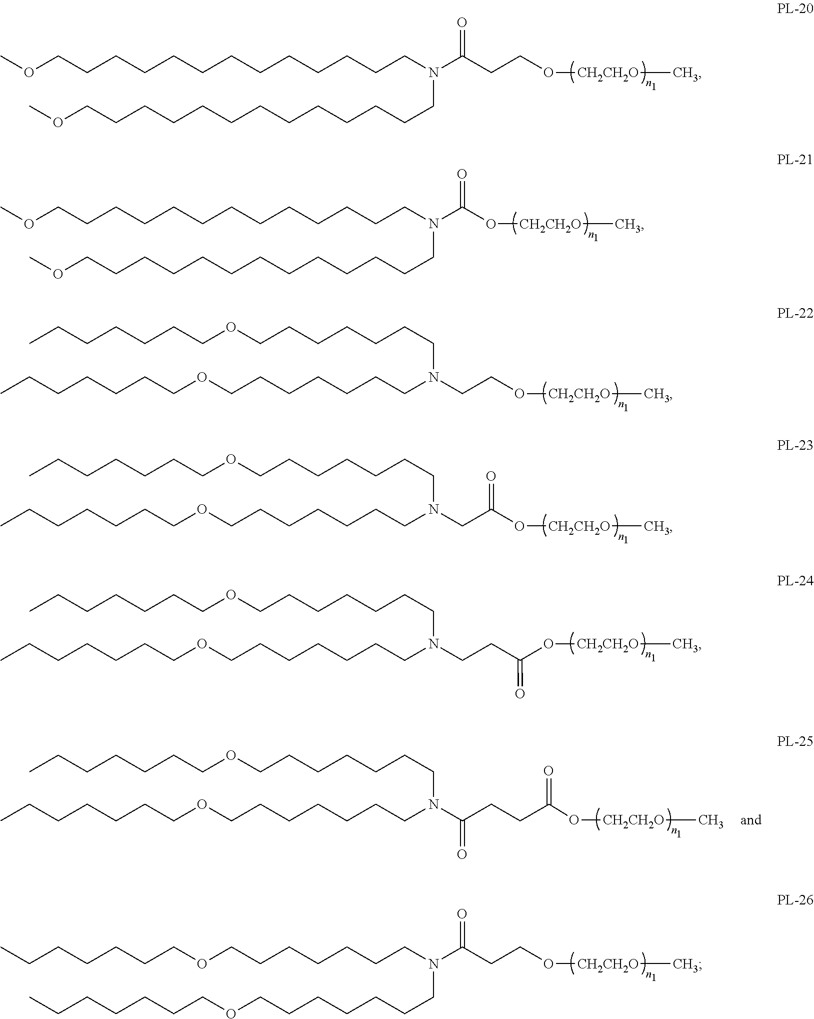
wherein, n₁ is an integer of 25 to 300.

19. The lipid composition according to claim 17, wherein the lipid composition comprises 20-80% amino acid-based cationic lipid, 5-15% phospholipid, 25-55% steroid lipid and 0.5-10% PEGylated lipid; the percentage is the molar percentage of each lipid in the total lipids in a solution containing solvent.

20. The lipid composition according to claim 19, wherein the molar percentage of cationic lipid in the total lipids in a solution containing solvent is 30-65%, and specifically is 35%, 40%, 45%, 46%, 47%, 48%, 49%, 50%, or 55%;
or the molar percentage of the phospholipid in the total lipids in a solution containing solvent is 7.5-13%, and specifically is 8%, 9%, 10%, 11%, or 12%;
or the molar percentage of the steroid lipid in the total lipids in a solution containing solvent is 35-50%, and specifically is 40%, 41%, 42%, 43%, 44%, 45%, 46%, 47%, 48%, 49%, or 50%;
or the molar percentage of PEGylated lipid in the total lipids in a solution containing solvent is 0.5-5%, and specifically is 1.5%, 1.6%, 1.7%, 1.8%, or 1.9%.

21. A lipid pharmaceutical composition containing a lipid composition of claim 16 and a drug, wherein the drug is selected from the group consisting of a nucleic acid drug, a gene vaccine, an antitumor drug, a small molecule drug, a peptide drug, and a protein drug.

22. The lipid pharmaceutical composition according to claim 21, wherein the nucleic acid drug is selected from the group consisting of RNA, DNA, antisense nucleic acid, plasmid, interfering nucleic acid, aptamer, antagomir and ribozyme, and the RNA is selected from the group consisting of mRNA, saRNA, circRNA, miRNA and siRNA.

23. The lipid pharmaceutical composition according to claim 21, for use in the treatment or prevention of a disease, wherein the use comprises using the lipid pharmaceutical composition as a drug selected from the group consisting of the following drugs: an antineoplastic agent, an antiviral agent, an antifungal agent and a vaccine.

24-26. (canceled)
